(19) United States
(12) Patent Application Publication
(10) Pub. No.: US 2022/0259145 A1
(43) Pub. Date: Aug. 18, 2022

(54) SARS-COV-2 MAIN PROTEASE INHIBITORS

(71) Applicant: TEXAS A&M UNIVERSITY SYSTEM, College Station, TX (US)

(72) Inventors: Wenshe LIU, College Station, TX (US); Carol A. FIERKE, College Station, TX (US); Shiqing XU, College Station, TX (US); Kai YANG, College Station, TX (US); Xinyu MA, College Station, TX (US); Yuying MA, College Station, TX (US); Yugendar Reddy ALUGUBELLI, College Station, TX (US); Erol Can VATANSEVER, College Station, TX (US); Chia-Chuan CHO, College Station, TX (US); Zhi GENG, College Station, TX (US); Kaustav KHATUA, College Station, TX (US)

(21) Appl. No.: 17/719,870

(22) Filed: Apr. 13, 2022

Related U.S. Application Data
(63) Continuation of application No. PCT/US2021/042956, filed on Jul. 23, 2021.
(60) Provisional application No. 63/056,210, filed on Jul. 24, 2020.

(51) Int. Cl. C07D 207/27; C07D 405/12; C07D 409/12; C07D 403/12; C07D 239/54; C07C 271/22

(52) U.S. Cl. CPC C07D 207/27; C07D 405/12; C07C 271/22; C07D 403/12; C07D 239/54; C07D 409/12

(57) ABSTRACT

The present disclosure relates to certain molecules, pharmaceutical compositions containing them, and methods of using them to treat viral infections.

### CROSS-REFERENCE TO RELATED APPLICATIONS

This application is a continuation of International Application no. PCT/US2021/042956, filed on Jul. 23, 2021, which claims priority to U.S. Provisional Application Ser. No. 63/056,210, filed on Jul. 24, 2020, the disclosures of which are expressly incorporated herein.

### REFERENCE TO SEQUENCE LISTING SUBMITTED ELECTRONICALLY

The official copy of the sequence listing is submitted electronically via EFS-Web as an ASCII formatted sequence listing with a file named 3353412_ST25Seq_List created on Apr. 13, 2022 and having a size of 4.096 kilobytes and is filed concurrently with the specification. The sequence listing comprised in this ASCII formatted document is part of the specification and is herein incorporated by reference in its entirety.

### TECHNICAL FIELD

The present disclosure relates to certain molecules, pharmaceutical compositions containing them, and methods of using them to treat viral infections.

### BACKGROUND

Coronaviruses (CoVs) are a group of related RNA viruses that cause diseases in a wide range of vertebrates including humans and domestic animals. Before 2003, there were only two CoVs, HCoV-229E and HCoV-OC43, known as human pathogens. The SARS pandemic in 2003 led to the revelation of SARS-CoV-1, a pathogen causing a severe respiratory infection. The subsequent surge in CoV research resulted in the discovery of two additional human CoVs, HCoV-NL63 and HCoV-HKU1, that are mildly pathogenic. One addition to this group was MERS-CoV that emerged in 2012 as a pathogen causing a severe respiratory infection. Although SARS-CoV-1 and MERS-CoV are highly lethal pathogens, the public health, social, and economic damages that they have caused are diminutive in comparison to that from SARS-CoV-2, a newly emerged human CoV pathogen that causes COVID-19. Rival only to the 1918 influenza pandemic, the COVID-19 pandemic has led to catastrophic impacts worldwide. As of Jul. 13, 2020, the total global COVID-19 cases have surpassed 12 million with more than 570,000 deaths. To alleviate catastrophic damages of COVID-19 on public health, society, and economy, finding timely treatment options is of paramount importance.

Similar to all other CoVs, SARS-CoV-2 is an enveloped, positive-sensed RNA virus with a genome of nearly 30 kb in size. Its genome encodes 10 open reading frames (ORFs). The largest ORF, ORF1ab encompasses more than two thirds of the whole genome. Its translated products, ORF1a (˜500 kDa) and ORF1ab (˜800 kDa), are very large polypeptides that undergo proteolytic cleavage to form 15 mature proteins. These are nonstructural proteins (Nsps) that are essential for the virus to modulate human cell hosts for efficient viral protein expression, viral genome replication, virion packaging, and viral genomic RNA processing. The proteolytic cleavage of ORF1a and ORF1ab is an autocatalytic process. Two internal polypeptide regions, Nsp3 and Nsp5, possess cysteine protease activity that cleaves themselves, and all other Nsps, from the two polypeptides. Nsp3 is commonly referred to as papain-like protease (PL^{Pro}), and Nsp5 as 3C-like protease (3CL^{Pro}) or, more recently, main protease (M^{Pro}). Previous studies of SARS-CoV-1 have established that activity of both PL^{Pro} and M^{Pro} is essential to viral replication and pathogenesis. Of the two proteases, M^{Pro} processes 12 out of the total 15 Nsps; inhibition of this enzyme is anticipated to have more significant impacts on the viral biology than that of PL^{Pro}. Therefore, small molecule medicines that potently inhibit SARS-CoV-2 M^{Pro} (SC2M^{Pro}) are potentially effective treatment options for COVID-19.

### SUMMARY

In one aspect, the disclosure relates to a compound of the formula I

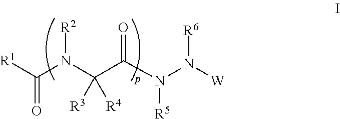

or a pharmaceutically acceptable salt thereof, wherein

R¹ is C₁-C₆ alkyl, C₂-C₆ alkenyl, C₁-C₆ alkoxy, 5-10 membered heteroaryl, —NH₂, —NHR^{a}, —NR^{a}R^{b}, or —C₁-C₆ alkyl-NR^{a}R^{b}, wherein each hydrogen atom in C₁-C₆ alkyl, C₂-C₆ alkenyl, C₁-C₆ alkoxy, and 5-10 membered heteroaryl is optionally substituted by an R^{a};

each of R² and R³ is independently H or C₁-C₆ alkyl; or R² and R³ together with the atoms to which they are attached combine to form;

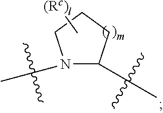

each R⁴ is H, C₁-C₆ alkyl, C₂-C₆ alkenyl, or C₃-C₈ cycloalkyl, wherein each hydrogen atom in C₁-C₆ alkyl, C₂-C₆ alkenyl, and C₃-C₈ cycloalkyl is optionally substituted by R^{b};

R⁵ is H;

R⁶ is C₁-C₆ alkyl, wherein each hydrogen atom in C₁-C₆ alkyl is optionally substituted by C(O)NR^{a}R^{b}, 3- to 8-membered heterocycloalkyl, or 5-10 membered heteroaryl;

W is C₁-C₆ alkyl-SO₄, C₁-C₆ alkyl-CN, —C(O)H, —C(O)OR^{d}, —C(O)C₁-C₆ alkyl, —C(O)C₂-C₆ alkenyl, —C(O)C₆-C₁₀ aryl, —C(O)C(O)NR^{a}R^{b}, C(O)COC(O)C₁-C₁₀ alkyl, —C(O)COC(O)C₆-C₁₀ aryl, —C(O)COC(O)-5-10 membered heteroaryl, or —CN, wherein each hydrogen atom in C₁-C₆ alkyl, C₂-C₆ alkenyl, and C₆-C₁₀ aryl is optionally substituted by halo, —OSO₄, —CN, —OR^{d}, —NO₂, C₆-C₁₀ aryl, or —NR^{a}R^{b};

each R^{a} and R^{b}, when present, is independently H, C₁-C₆ alkyl, C₃-C₈ cycloalkyl, C₁-C₆ alkoxy, or C₆-C₁₀ aryl, wherein each hydrogen atom in C₁-C₆ alkyl, C₃-C₈ cycloalkyl, C₁-C₆ alkoxy, and C₆-C₁₀ aryl is optionally substituted by halo, OR^{d}, 5-10 membered heteroaryl, C₂-C₆ alkenyl, C₆-C₁₀ aryl, or C₃-C₈ cycloalkyl;

each R^{c}, when present, is C₁-C₆ alkoxy or C₃-C₈ cycloalkyl, or two R^{c} combine together with the atom or atoms to which they are attached to form a C₃-C₈ cycloalkyl, wherein each hydrogen atom in C₃-C₈ cycloalkyl is optionally substituted by C₁-C₆ alkyl;

R^{d}, when present, is H, C₁-C₆ alkyl, —C(O)C₁-C₆ alkyl, or C₆-C₁₀ aryl, wherein each hydrogen atom in C₁-C₆ alkyl and C₆-C₁₀ aryl is optionally substituted by halo or —NO₂;

l is 0, 1, 2, or 3;

m is 1, 2, or 3; and

p is 1 or 2.

In another aspect, the disclosure provides compounds of the formula II

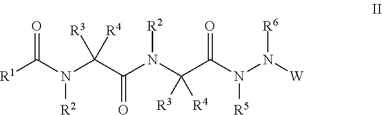

wherein R¹, R², each R³, each R⁴, R⁵, R⁶, and W are as defined herein.

In another aspect, the disclosure provides compounds of the formula III

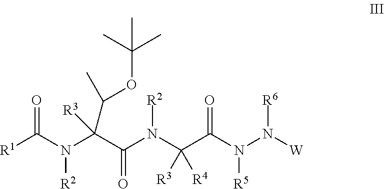

wherein R¹, R², each R³, R⁴, R⁵, R⁶, and W are as defined herein.

In another aspect, the disclosure provides compounds of the formula IV

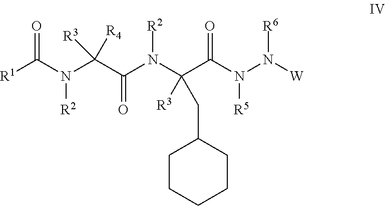

wherein R¹, R², each R³, R⁴, R⁵, R⁶, and W are as defined herein.

In another aspect, the disclosure provides compounds of the formula V

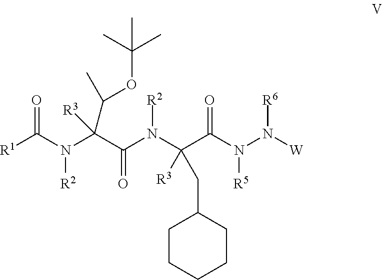

wherein R¹, R², each R³, R⁵, R⁶, and W are as defined herein.

In another aspect, the disclosure provides compounds of the formula VI

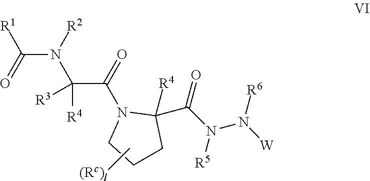

wherein R¹, R², R³, each R⁴, R⁵, R⁶, W, R^{c}, and 1 are as defined herein.

In another aspect, the disclosure provides compounds of the formula VII

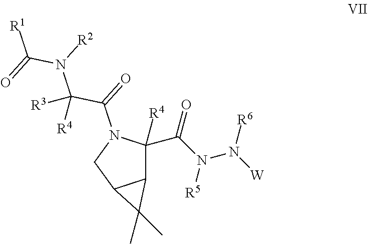

wherein R¹, R², R³, each R⁴, R⁵, R⁶, and W are as defined herein.

In another aspect, the disclosure provides compounds of the formula VIII

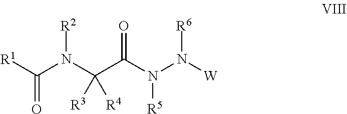

wherein R¹, R², R³, R⁴, R⁵, R⁶, and W are as defined herein.

In another aspect, the disclosure provides compounds of the formula IX

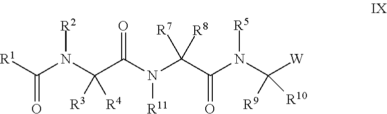

wherein R¹, R², R³, R⁴, R⁵, R⁷, R⁸, R⁹, R¹⁰, R¹¹, and W are as defined herein

In another aspect, the disclosure provides compounds of the formula X

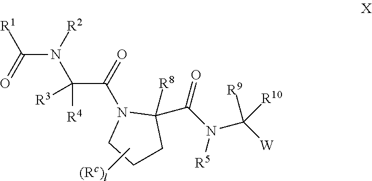

wherein R¹, R², R³, R⁴, R⁵, R⁸, R⁹, R¹⁰, W, R^{c}, and 1 are as defined herein.

In another aspect, the disclosure provides compounds of the formula XI

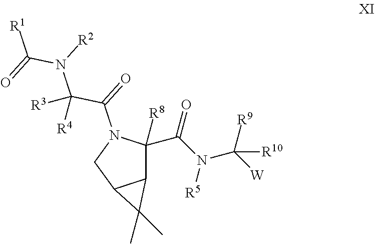

wherein R¹, R², R³, R⁴, R⁵, R⁸, R⁹, R¹⁰, and W are as defined herein.

In another aspect, the disclosure provides compounds of the formula XII

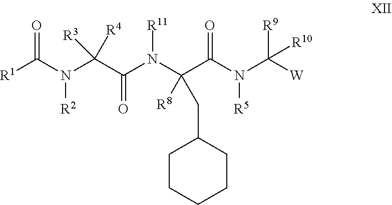

wherein R¹, R², R³, R⁴, R⁵, R⁸, R⁹, R¹⁰, R¹¹, and W are as defined herein.

In another aspect, the disclosure provides compounds of the formula XIII

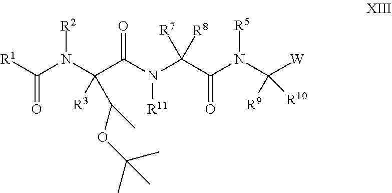

wherein R¹, R², R³, R⁵, R⁷, R⁸, R⁹, R¹⁰, R¹¹, and W are as defined herein.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of formula

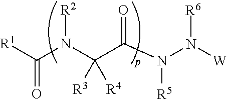

or a pharmaceutically acceptable salt thereof, wherein

R¹ is C₁-C₆ alkyl, C₂-C₆ alkenyl, C₁-C₆ alkoxy, 5-10 membered heteroaryl, —NH₂, —NHR^{a}, —NR^{a}R^{b}, or —C₁-C₆ alkyl-NR^{a}R^{b}, wherein each hydrogen atom in C₁-C₆ alkyl, C₂-C₆ alkenyl, C₁-C₆ alkoxy, and 5-10 membered heteroaryl is optionally substituted by an R^{a};

each of R² and R³ is independently H or C₁-C₆ alkyl; or R² and R³ together with the atoms to which they are attached combine to form

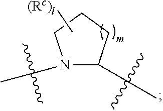

each R⁴ is H, C₁-C₆ alkyl, C₂-C₆ alkenyl, or C₃-C₈ cycloalkyl, wherein each hydrogen atom in C₁-C₆ alkyl, C₂-C₆ alkenyl, and C₃-C₈ cycloalkyl is optionally substituted by R^{b};

R⁵ is H;

R⁶ is C₁-C₆ alkyl, wherein each hydrogen atom in C₁-C₆ alkyl is optionally substituted by C(O)NR^{a}R^{b}, 3- to 8-membered heterocycloalkyl, or 5-10 membered heteroaryl;

W is C₁-C₆ alkyl-SO₄, C₁-C₆ alkyl-CN, —C(O)H, —C(O)OR^{d}, —C(O)C₁-C₆ alkyl, —C(O)C₂-C₆ alkenyl, —C(O)C₆-C₁₀ aryl, —C(O)C(O)NR^{a}R^{b}, C(O)COC(O)C₁-C₁₀ alkyl, —C(O)COC(O)C₆-C₁₀ aryl, —C(O)COC(O)-5-10 membered heteroaryl, or —CN, wherein each hydrogen atom in C₁-C₆ alkyl, C₂-C₆ alkenyl, and C₆-C₁₀ aryl is optionally substituted by halo, —OSO₄, —CN, —OR^{d}, —NO₂, C₆-C₁₀ aryl, or —NR^{a}R^{b};

each R^{a} and R^{b}, when present, is independently H, C₁-C₆ alkyl, C₃-C₈ cycloalkyl, C₁-C₆ alkoxy, or C₆-C₁₀ aryl, wherein each hydrogen atom in C₁-C₆ alkyl, C₃-C₈ cycloalkyl, C₁-C₆ alkoxy, and C₆-C₁₀ aryl is optionally substituted by halo, OR^{d}, 5-10 membered heteroaryl, C₂-C₆ alkenyl, C₆-C₁₀ aryl, or C₃-C₈ cycloalkyl;

each R^{c}, when present, is C₁-C₆ alkoxy or C₃-C₈ cycloalkyl, or two R^{c} combine together with the atom or atoms to which they are attached to form a C₃-C₈ cycloalkyl, wherein each hydrogen atom in C₃-C₈ cycloalkyl is optionally substituted by C₁-C₆ alkyl;

R^{d}, when present, is H, C₁-C₆ alkyl, —C(O)C₁-C₆ alkyl, or C₆-C₁₀ aryl, wherein each hydrogen atom in C₁-C₆ alkyl and C₆-C₁₀ aryl is optionally substituted by halo or —NO₂;

l is 0, 1, 2, or 3;

m is 1, 2, or 3; and

p is 1 or 2;

or a pharmaceutically acceptable salt thereof.

2. The compound or pharmaceutically acceptable salt of clause 1, wherein the compound is of formula

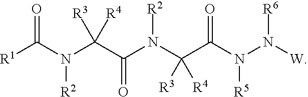

3. The compound or pharmaceutically acceptable salt of clause 1 or 2, wherein at least one R⁴ is C₁-C₆ alkyl, wherein each hydrogen atom in C₁-C₆ alkyl is optionally substituted by C₁-C₆ alkoxy or C₃-C₈ cycloalkyl.

4. The compound or pharmaceutically acceptable salt any one of clauses 1-3, wherein at least one R⁴ is C₁-C₆ alkyl, wherein at least one hydrogen atom in C₁-C₆ alkyl is substituted by C₁-C₆alkoxy.

5. The compound or pharmaceutically acceptable salt of any one of clauses 1-4, wherein the compound is of formula

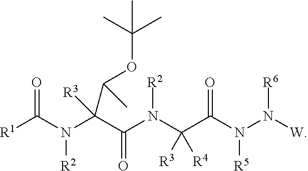

6. The compound or pharmaceutically acceptable salt any one of clauses 1-4, wherein at least one R⁴ is C₁-C₆ alkyl, wherein at least one hydrogen atom in C₁-C₆ alkyl is substituted by C₃-C₈ cycloalkyl.

7. The compound or pharmaceutically acceptable salt any one of clauses 1-4 or 5, wherein the compound is of formula

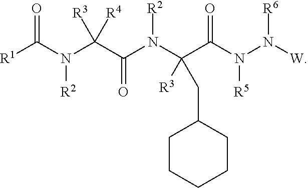

8. The compound or pharmaceutically acceptable of clause 1, wherein the compound is of formula

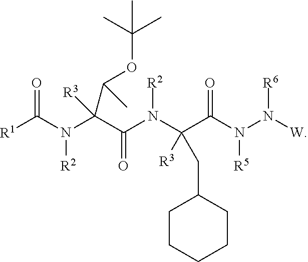

9. The compound or pharmaceutically acceptable salt of any one of clauses 1-7, wherein at least one R² and at least one R³ together with the atoms to which they are attached combine to form N

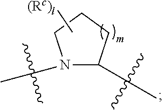

10. The compound or pharmaceutically acceptable salt of any one of clauses 1-6 or 9, wherein the compound is of formula

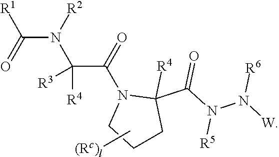

11. The compound or pharmaceutically acceptable salt of clause 10, wherein the compound is of formula

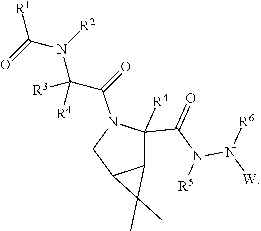

12. The compound or pharmaceutically acceptable of clause 1, wherein the compound is of formula

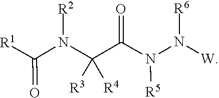

13. The compound or pharmaceutically acceptable salt of any one of clauses 1-7 or 9-12, wherein at least one R⁴ is C₁-C₆ alkyl.

14. The compound or pharmaceutically acceptable salt of clause 12, wherein at least one R⁴ is t-butyl or isopropyl.

15. The compound or pharmaceutically acceptable salt of clause 14, wherein at least one R⁴ is t-butyl.

16. The compound or pharmaceutically acceptable salt of clause 14, wherein at least one R⁴ is isopropyl.

17. The compound or pharmaceutically acceptable salt of any one of the preceding clauses, wherein R¹ is C₁-C₆ alkoxy, wherein each hydrogen atom in C₁-C₆ alkoxy is optionally substituted by an R^{a}.

18. The compound or pharmaceutically acceptable salt of any one of the preceding clauses, wherein R¹ is C₁-C₆ alkoxy, wherein at least one hydrogen atom in C₁-C₆ alkoxy is substituted by an R^{a}.

19. The compound or pharmaceutically acceptable salt of clause 18, wherein R^{a} is C₆-C₁₀ aryl, wherein each hydrogen atom in C₆-C₁₀ aryl is optionally substituted by halo.

20. The compound or pharmaceutically acceptable salt of any one of the preceding clauses, wherein R¹ is

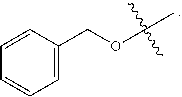 [SMILES: *OCc1ccccc1]

21. The compound or pharmaceutically acceptable salt of any one of the preceding clauses, wherein R¹ is NHR^{a}.

22. The compound or pharmaceutically acceptable salt of any one of the preceding clauses wherein R¹ is NHC(CH₃)₃.

23. The compound or pharmaceutically acceptable salt of any one of the preceding clauses, wherein R⁶ is C₁-C₆ alkyl, wherein at least one hydrogen atom in C₁-C₆ alkyl is substituted by —C(O)NR^{a}R^{b} or 3- to 8-membered heterocycloalkyl.

24. The compound or pharmaceutically acceptable salt of any one of the preceding clauses, wherein R⁶ is C₁-C₆ alkyl, wherein at least one hydrogen atom in C₁-C₆ alkyl is substituted by —C(O)NR^{a}R^{b}.

25. The compound or pharmaceutically acceptable salt of any one of the preceding clauses, wherein R⁶ is C₁-C₆ alkyl, wherein at least one hydrogen atom in C₁-C₆ alkyl is substituted by —C(O)NH₂.

26. The compound or pharmaceutically acceptable salt of any one of clauses 1-25, wherein R⁶ is C₁-C₆ alkyl, wherein at least one hydrogen atom in C₁-C₆ alkyl is substituted by 3- to 8-membered heterocycloalkyl.

27. The compound or pharmaceutically acceptable salt of any one of clauses 1-25, wherein R⁶ is C₁-C₆ alkyl, wherein at least one hydrogen atom in C₁-C₆ alkyl is substituted by

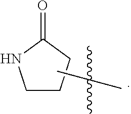

28. The compound or pharmaceutically acceptable salt of any one of the preceding clauses, wherein W is C(O)H.

29. The compound or pharmaceutically acceptable salt of any one of clauses 1-28, wherein W is —C(O)C₁-C₆ alkyl, wherein each hydrogen atom in C₁-C₆ alkyl is optionally substituted by halo or OR^{d}.

30. The compound or pharmaceutically acceptable salt of clause 29, wherein R^{d} is C₆-C₁₀ aryl, wherein each hydrogen atom in C₆-C₁₀ aryl is optionally substituted by halo or —NO₂.

31. The compound or pharmaceutically acceptable salt of any one of clauses 1-28, wherein W is C(O)C₂-C₆ alkenyl, wherein each hydrogen atom in C₂-C₆ alkenyl is optionally substituted by C₆-C₁₀ aryl or —NR^{a}R^{b}.

32. The compound or pharmaceutically acceptable salt of any one of clauses 1-28, wherein W is —C(O)C₆-C₁₀ aryl, wherein each hydrogen atom in C₆-C₁₀ aryl is optionally substituted by halo or —NO₂.

33. The compound or pharmaceutically acceptable salt of any one of clauses 1-28, wherein W is C(O)OR^{d}.

34 The compound or pharmaceutically acceptable salt of clause 33, wherein R^{d} is C₁-C₆ alkyl, wherein each hydrogen atom in C₁-C₆ alkyl is optionally substituted by halo or C₆-C₁₀ aryl optionally substituted by halo or —NO₂.

35. The compound or pharmaceutically acceptable salt of any one of the preceding clauses, wherein at least one R² is H.

36. The compound or pharmaceutically acceptable of any one of the preceding clauses, wherein R⁵ is H.

37. A compound of formula

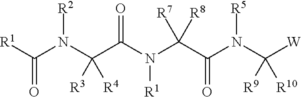

or a pharmaceutically acceptable salt thereof, wherein

R¹ is C₁-C₆ alkyl, C₂-C₆ alkenyl, C₁-C₆ alkoxy, 5-10 membered heteroaryl, —NH₂, —NHR^{a}, —NR^{a}R^{b}, or —C₁-C₆ alkyl-NR^{a}R^{b}, wherein each hydrogen atom in C₁-C₆ alkyl, C₂-C₆ alkenyl, C₁-C₆ alkoxy, and 5-10 membered heteroaryl is optionally substituted by an R^{a};

each of R² and R³ is independently H or C₁-C₆ alkyl; or R² and R³ together with the atoms to which they are attached combine to form N;

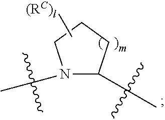

R⁴ is C₁-C₆ alkyl, C₆-C₁₀ aryl, or C₃-C₈ cycloalkyl, wherein each hydrogen atom in C₁-C₆ alkyl, C₆-C₁₀ aryl, and C₃-C₈ cycloalkyl is optionally substituted by R^{b} or C₁-C₆ alkoxy; or R³ and R⁴ together with the carbon atom to which they are attached form C₃-C₈ cycloalkyl, wherein each hydrogen atom in C₃-C₈ cycloalkyl is optionally substituted by R^{b};

R⁵ is H or C₁-C₆ alkyl, wherein each hydrogen atom in C₁-C₆ alkyl is optionally substituted by —C(O)NR^{a}R^{b};

each of R⁷ and R⁸ is independently H, C₁-C₆ alkyl, C₂-C₆ alkenyl, or C₃-C₈ cycloalkyl, wherein each hydrogen atom in C₁-C₆ alkyl, C₂-C₆ alkenyl, and C₃-C₈ cycloalkyl is optionally substituted by R^{b};

each of R⁹ and R¹⁰ is independently H or C₁-C₆ alkyl, wherein each hydrogen atom in C₁-C₆ alkyl is optionally substituted by C(O)NR^{a}R^{b}, 3- to 8-membered heterocycloalkyl, or 5-10 membered heteroaryl;

R¹¹ is H or R¹¹ and one of R⁷ or R⁸ together with the atoms to which they are attached combine to form

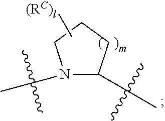

W is C₁-C₆ alkyl-SO₄, C₁-C₆ alkyl-CN, —C(O)H, —C(O)C₁-C₆ alkyl, —C(O)C(O)NR^{a}R^{b}, C(O)COC(O)C₁-C₁₀ alkyl, —C(O)COC(O)C₆-C₁₀ aryl, —C(O)COC(O)-5-10 membered heteroaryl, or —CN, wherein each hydrogen atom in C₁-C₆ alkyl, and C₆-C₁₀ aryl is optionally substituted by halo, —OSO₄, —OR^{d}, —CN, —NO₂, C₆-C₁₀ aryl, or —NR^{a}R^{b};

each R^{a} and R^{b}, when present, is independently H, C₁-C₆ alkyl, C₃-C₈ cycloalkyl, C₁-C₆ alkoxy, C₆-C₁₀ aryl, or 5-10 membered heteroaryl, wherein each hydrogen atom in C₁-C₆ alkyl, C₃-C₈ cycloalkyl, C₁-C₆ alkoxy, C₆-C₁₀ aryl, and 5-10 membered heteroaryl is optionally substituted by halo, —OR^{d}, 5-10 membered heteroaryl, C₆-C₁₀ aryl, C₂-C₆ alkenyl, or C₃-C₈ cycloalkyl;

each R^{c}, when present, is C₁-C₆ alkoxy or C₃-C₈ cycloalkyl, or two R^{c} combine together with the atom or atoms to which they are attached to form a C₃-C₈ cycloalkyl, wherein each hydrogen atom in C₃-C₈ cycloalkyl is optionally substituted by C₁-C₆ alkyl;

R^{d}, when present, is H, C₁-C₆ alkyl, —C(O)C₁-C₆ alkyl, or C₆-C₁₀ aryl, wherein each hydrogen atom in C₁-C₆ alkyl and C₆-C₁₀ aryl is optionally substituted by halo or —NO₂;

l is 0, 1, 2, or 3; and

m is 1, 2, or 3;

provided that if R⁴ is

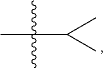 [SMILES: *C(C)C]

R¹ is —OCH₂-phenyl, one of R⁹ and R¹⁰ is

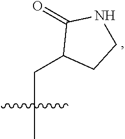 [SMILES: *CC1CCNC1=O]

and one of R⁷ and R⁸ is

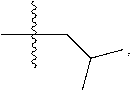 [SMILES: *CC(C)C]

then W is not —C(O)H,

provided that if R⁴ is

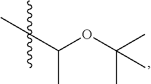 [SMILES: *C(C)OC(C)(C)C]

R¹ is —OCH₂-phenyl, one of R⁹ and R¹⁰ is

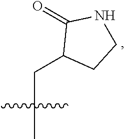 [SMILES: *CC1CCNC1=O]

and one of R⁷ and R⁸ is

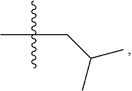 [SMILES: *CC(C)C]

then W is not —C(O)H, and

provided that if R⁴ is

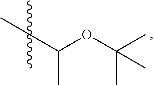 [SMILES: *C(C)OC(C)(C)C]

R¹ is —OCH₂-phenyl, one of R⁹ and R¹⁰ is

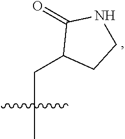 [SMILES: *CC1CCNC1=O]

and one of R⁷ and R⁸ is —CH₂-cyclohexyl, then W is not —C(O)H.

38. The compound or pharmaceutically acceptable salt of clause 37, wherein R¹¹ and one of R⁷ together with the atoms to which they are attached combine to form

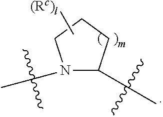

39. The compound or pharmaceutically acceptable salt of clause 37 or 38, wherein the compound is of formula

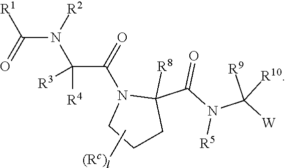

40. The compound or pharmaceutically acceptable salt of any one of clauses 37-39, wherein the compound is of formula

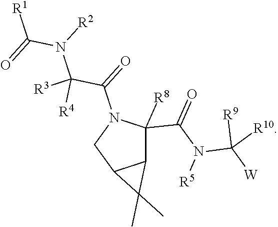

41. The compound or pharmaceutically acceptable salt of clause 37, wherein R⁷ is C₁-C₆ alkyl, wherein at least one hydrogen atom in C₁-C₆ alkyl is substituted by C₃-C₈ cycloalkyl, C₆-C₁₀ aryl, or 5-10 membered heteroaryl.

42. The compound or pharmaceutically acceptable salt of clause 37 or 40, wherein the compound is of formula

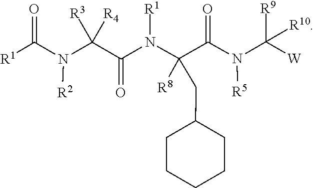

43. The compound or pharmaceutically acceptable salt of clause 37, wherein R⁷ is

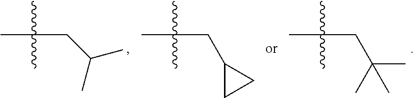

44. The compound or pharmaceutically acceptable salt of any one of clauses 37-43, wherein R⁴ is C₁-C₆ alkyl, wherein at least one hydrogen atom in C₁-C₆ alkyl is substituted by C₁-C₆ alkoxy.

45. The compound or pharmaceutically acceptable salt of clause 37, wherein the compound is of formula

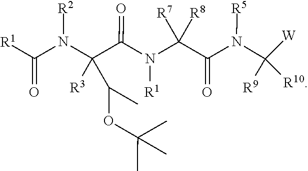

46. The compound or pharmaceutically acceptable salt of any one of clauses 37-43, wherein R⁴ is C₁-C₆ alkyl or C₃-C₈ cycloalkyl.

47. The compound or pharmaceutically acceptable salt of any one of clauses 37-43 or 46, wherein R⁴ is t-butyl.

48. The compound or pharmaceutically acceptable salt of any one of clauses 37-43 or 46, wherein R⁴ is isopropyl.

49. The compound or pharmaceutically acceptable salt of any one of clauses 37-43 or 46, wherein R⁴ is cyclopropyl.

50. The compound or pharmaceutically acceptable salt of any one of clauses 37-49, wherein R³ is H or methyl.

51. The compound or pharmaceutically acceptable salt of any one of clauses 37-50, wherein R¹ is C₁-C₆ alkoxy or C₂-C₆ alkenyl, wherein each hydrogen atom in C₁-C₆ alkoxy or C₂-C₆ alkenyl is optionally substituted by an R^{a}.

52. The compound or pharmaceutically acceptable salt of clause 51, wherein R^{a} is C₆-C₁₀ aryl, wherein each hydrogen atom in C₆-C₁₀ aryl is optionally substituted by halo.

53. The compound or pharmaceutically acceptable salt of any one of clauses 37-52, wherein R¹ is

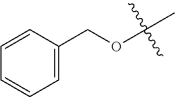 [SMILES: *OCc1ccccc1]

wherein each hydrogen atom in

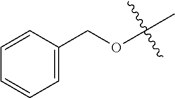 [SMILES: *OCc1ccccc1]

is optionally substituted by halo.

54. The compound or pharmaceutically acceptable salt of any one of clauses 37-50, wherein R¹ is NHR^{a}.

55. The compound or pharmaceutically acceptable salt of any one of clauses 37-50 or 54, wherein R¹ is NHC(CH₃)₃.

56. The compound or pharmaceutically acceptable salt of any one of clauses 37-56, wherein at least one of R⁹ and R¹⁰ is C₁-C₆ alkyl, wherein at least one hydrogen atom in C₁-C₆ alkyl is substituted by C(O)NR^{a}R^{b} or 3- to 8-membered heterocycloalkyl.

57. The compound or pharmaceutically acceptable salt of any one of clauses 37-57, wherein at least one of R⁹ and R¹⁰ is C₁-C₆ alkyl, wherein at least one hydrogen atom in C₁-C₆ alkyl is substituted by C(O)NR^{a}R^{b}.

58. The compound or pharmaceutically acceptable salt of any one of clauses 37-57, wherein at least one of R⁹ and R¹⁰ is C₁-C₆ alkyl, wherein at least one hydrogen atom in C₁-C₆ alkyl is substituted by C(O)NH₂.

59. The compound or pharmaceutically acceptable salt of of any one of clauses 37-56, wherein at least one of R⁹ and R¹⁰ is C₁-C₆ alkyl, wherein at least one hydrogen atom in C₁-C₆ alkyl is substituted by 3- to 8-membered heterocycloalkyl.

60. The compound or pharmaceutically acceptable salt of any one of clauses 37-56 or 59, wherein at least one of R⁹ and R¹⁰ is C₁-C₆ alkyl, wherein at least one hydrogen atom in C₁-C₆ alkyl is substituted by

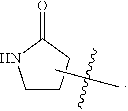

61. The compound or pharmaceutically acceptable salt of any one of clauses 37-60, wherein W is C(O)H.

62. The compound or pharmaceutically acceptable salt of any one of clauses 37-60, wherein W is —C(O)C₁-C₆ alkyl, wherein each hydrogen atom in C₁-C₆ alkyl is optionally substituted by halo or OR^{d}.

63. The compound or pharmaceutically acceptable salt of any one of clauses 37-60 or 62, wherein W is —C(O)C₁-C₆ alkyl, wherein at least one hydrogen atom in C₁-C₆ alkyl is substituted by halo.

64. The compound or pharmaceutically acceptable salt of clause 62, wherein R^{d} is C₆-C₁₀ aryl, wherein at least one hydrogen atom in hydrogen atom in C₆-C₁₀ aryl is optionally substituted by halo or —NO₂.

65. The compound or pharmaceutically acceptable salt of any one of clauses 37-60, wherein W is —C(O)C₂-C₆ alkenyl, wherein each hydrogen atom in C₂-C₆ alkenyl is optionally substituted by C₆-C₁₀ aryl or —NR^{a}R^{b}.

66. The compound or pharmaceutically acceptable salt of any one of clauses 37-60, wherein W is —C(O)C₆-C₁₀ aryl, wherein each hydrogen atom in C₆-C₁₀ aryl is optionally substituted by halo or —NO₂.

67. The compound or pharmaceutically acceptable salt of any one of clauses 37-60, wherein W is C(O)OR^{d}.

68. The compound or pharmaceutically acceptable salt of clause 67, wherein R^{d} is C₁-C₆ alkyl, wherein each hydrogen atom in C₁-C₆ alkyl is optionally substituted by halo or C₆-C₁₀ aryl, wherein each hydrogen atom in C₆-C₁₀ aryl is optionally substituted by halo or —NO₂.

69. The compound or pharmaceutically acceptable salt of any one of clauses 37-60, wherein W is C₁-C₆ alkyl-SO₄, wherein each hydrogen atom in C₁-C₆ alkyl is optionally substituted by hydroxy.

70. The compound or pharmaceutically acceptable salt of any one of clauses 37-60, wherein W is —C(O)C(O)NR^{a}R^{b}.

71. The compound or pharmaceutically acceptable salt of clause 70, wherein each of R^{a} and R^{b} is independently H or C₁-C₆ alkyl, wherein each hydrogen atom in C₁-C₆ alkyl is optionally substituted by C₆-C₁₀ aryl.

72. The compound or pharmaceutically acceptable salt of any one of clauses 37-60, wherein W is C₁-C₆ alkyl-CN, wherein each hydrogen atom in C₁-C₆ alkyl is optionally substituted by hydroxy.

73. The compound or pharmaceutically acceptable salt of any one of clauses 37-72, wherein R² is H.

74. The compound or pharmaceutically acceptable salt of any one of clauses 37-73, wherein R³ is H.

75. The compound or pharmaceutically acceptable salt of any one of clauses 37-74, wherein R⁵ is H.

76. The compound or pharmaceutically acceptable salt of any one of clauses 37-75, wherein R⁸ is H.

77. The compound or pharmaceutically acceptable salt of any one of clauses 37-76, wherein R⁹ is H.

78. The compound or pharmaceutically acceptable salt of any one of clauses 37-77, wherein R¹⁰ is H.

79. A compound selected from the group consisting of:
benzyl ((S)-3-methyl-1-oxo-1-(((S)-1-oxo-1-(((S)-1-oxo-3-((S)-2-oxopyrrolidin-3-yl)propan-2-yl)amino)-3-phenylpropan-2-yl)amino)butan-2-yl)carbamate,
benzyl ((S)-1-(((S)-3-cyclohexyl-1-oxo-1-(((S)-1-oxo-3-((S)-2-oxopyrrolidin-3-yl)propan-2-yl)amino)propan-2-yl)amino)-3-methyl-1-oxobutan-2-yl)carbamate,
benzyl ((2S,3R)-3-(tert-butoxy)-1-oxo-1-(((S)-1-oxo-1-(((S)-1-oxo-3-((S)-2-oxopyrrolidin-3-yl)propan-2-yl)amino)-3-phenylpropan-2-yl)amino)butan-2-yl)carbamate,
(S)-2-((S)-2-((E)-3-(4-chloro-2-fluorophenyl)acrylamido)-3-methylbutanamido)-4-methyl-N—((S)-1-oxo-3-((S)-2-oxopyrrolidin-3-yl)propan-2-yl)pentanamide,
benzyl ((S)-1-(((S)-3-(furan-2-yl)-1-oxo-1-(((S)-1-oxo-3-((S)-2-oxopyrrolidin-3-yl)propan-2-yl)amino)propan-2-yl)amino)-3-methyl-1-oxobutan-2-yl)carbamate,
benzyl ((S)-1-(((S)-3-(furan-2-yl)-1-oxo-1-(((S)-1-oxo-3-((S)-2-oxopyrrolidin-3-yl)propan-2-yl)amino)propan-2-yl)amino)-3-methyl-1-oxobutan-2-yl)carbamate,
N—((S)-3-methyl-1-(((S)-4-methyl-1-oxo-1-(((S)-1-oxo-3-((S)-2-oxopyrrolidin-3-yl)propan-2-yl)amino)pentan-2-yl)amino)-1-oxobutan-2-yl)-1H-indole-2-carboxamide,
benzyl ((S)-1-(((S)-4,4-dimethyl-1-oxo-1-(((S)-1-oxo-3-((S)-2-oxopyrrolidin-3-yl)propan-2-yl)amino)pentan-2-yl)amino)-3-methyl-1-oxobutan-2-yl)carbamate,
benzyl ((S)-3-methyl-1-(((S)-4-methyl-1-oxo-1-(((S)-1-oxo-3-((S)-2-oxopyrrolidin-3-yl)propan-2-yl)amino)pent-4-en-2-yl)amino)-1-oxobutan-2-yl)carbamate,
benzyl ((S)-1-(((S)-4,4-dimethyl-1-oxo-1-(((S)-1-oxo-3-((S)-2-oxopyrrolidin-3-yl)propan-2-yl)amino)pentan-2-yl)amino)-3,3-dimethyl-1-oxobutan-2-yl)carbamate,
benzyl ((S)-1-cyclopropyl-2-(((S)-4,4-dimethyl-1-oxo-1-(((S)-1-oxo-3-((S)-2-oxopyrrolidin-3-yl)propan-2-yl)amino)pentan-2-yl)amino)-2-oxoethyl)carbamate,
benzyl ((2S,3R)-3-(tert-butoxy)-1-(((S)-3-cyclopropyl-1-oxo-1-(((S)-1-oxo-3-((S)-2-oxopyrrolidin-3-yl)propan-2-yl)amino)propan-2-yl)amino)-1-oxobutan-2-yl)carbamate,
benzyl ((2S,3R)-3-(tert-butoxy)-1-(((S)-4,4-dimethyl-1-oxo-1-(((S)-1-oxo-3-((S)-2-oxopyrrolidin-3-yl)propan-2-yl)amino)pentan-2-yl)amino)-1-oxobutan-2-yl)carbamate,
benzyl ((S)-1-(((S)-3-cyclopropyl-1-oxo-1-(((S)-1-oxo-3-((S)-2-oxopyrrolidin-3-yl)propan-2-yl)amino)propan-2-yl)amino)-3-methyl-1-oxobutan-2-yl)carbamate,
benzyl ((S)-1-(((S)-4,4-dimethyl-1-oxo-1-(((S)-1-oxo-3-((S)-2-oxopyrrolidin-3-yl)propan-2-yl)amino)pentan-2-yl)amino)-2,3-dimethyl-1-oxobutan-2-yl)carbamate,
benzyl (1-(((S)-4,4-dimethyl-1-oxo-1-(((S)-1-oxo-3-((S)-2-oxopyrrolidin-3-yl)propan-2-yl)amino)pentan-2-yl)amino)-2-methyl-1-oxopropan-2-yl)carbamate,
benzyl (1-(((S)-3-cyclohexyl-1-oxo-1-(((S)-1-oxo-3-((S)-2-oxopyrrolidin-3-yl)propan-2-yl)amino)propan-2-yl)carbamoyl)cyclopropyl)carbamate,
3-chlorobenzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate,
3-chlorobenzyl ((S)-1-(((S)-3-cyclohexyl-1-oxo-1-(((S)-1-oxo-3-((S)-2-oxopyrrolidin-3-yl)propan-2-yl)amino)propan-2-yl)amino)-3-methyl-1-oxobutan-2-yl)carbamatebenzyl ((S)-3-cyclohexyl-1-oxo-1-(((S)-1-oxo-3-((S)-2-oxopyrrolidin-3-yl)propan-2-yl)amino)propan-2-yl)carbamate,
benzyl ((S)-1-(((S)-3-cyclopropyl-1-oxo-1-(((S)-1-oxo-3-((S)-2-oxopyrrolidin-3-yl)propan-2-yl)amino)propan-2-yl)amino)-3,3-dimethyl-1-oxobutan-2-yl)carbamate,
benzyl ((S)-1-cyclopropyl-2-(((S)-3-cyclopropyl-1-oxo-1-(((S)-1-oxo-3-((S)-2-oxopyrrolidin-3-yl)propan-2-yl)amino)propan-2-yl)amino)-2-oxoethyl)carbamate,
benzyl ((S)-1-(((S)-1-cyclopropyl-3-(((S)-1-oxo-3-((S)-2-oxopyrrolidin-3-yl)propan-2-yl)amino)propan-2-yl)amino)-3,3-dimethyl-1-oxobutan-2-yl)carbamate,
3-chlorobenzyl ((2S,3R)-3-(tert-butoxy)-1-(((S)-4-methyl-1-oxo-1-(((S)-1-oxo-3-((S)-2-oxopyrrolidin-3-yl)propan-2-yl)amino)pentan-2-yl)amino)-1-oxobutan-2-yl)carbamate,
benzyl ((S)-1-(((S)-1-(2-acryloyl-2-((2,6-dioxo-1,2,3,6-tetrahydropyrimidin-4-yl)methyl)hydrazineyl)-4-methyl-1-oxopentan-2-yl)amino)-3-methyl-1-oxobutan-2-yl)carbamate,
benzyl (S)-(1-(2-(3-amino-3-oxopropyl)-2-propionylhydrazineyl)-4-methyl-1-oxopentan-2-yl)carbamate,
benzyl (S)-(1-(2-(3-amino-3-oxopropyl)-2-(2-chloroacetyl)hydrazineyl)-4-methyl-1-oxopentan-2-yl)carbamate,
benzyl (S)-(1-(2-acryloyl-2-(3-amino-3-oxopropyl)hydrazineyl)-4-methyl-1-oxopentan-2-yl)carbamate,
benzyl ((S)-1-(2-(3-amino-3-oxopropyl)-2-((R)-2-chloropropanoyl)hydrazineyl)-4-methyl-1-oxopentan-2-yl)carbamate,
benzyl ((S)-1-(2-(3-amino-3-oxopropyl)-2-((S)-2-chloropropanoyl)hydrazineyl)-4-methyl-1-oxopentan-2-yl)carbamate,
benzyl (S)-(1-(2-(3-amino-3-oxopropyl)-2-(2-fluoro-5-nitrobenzoyl)hydrazineyl)-4-methyl-1-oxopentan-2-yl)carbamate,
benzyl (S)-(1-(2-(3-amino-3-oxopropyl)-2-(2-benzylacryloyl)hydrazineyl)-4-methyl-1-oxopentan-2-yl)carbamate,
benzyl (S,E)-(1-(2-(3-amino-3-oxopropyl)-2-(4-(dimethylamino)but-2-enoyl)hydrazineyl)-4-methyl-1-oxopentan-2-yl)carbamate,
benzyl (S)-(1-(2-(3-amino-3-oxopropyl)-2-(2-(4-nitrophenoxy)acetyl)hydrazineyl)-4-methyl-1-oxopentan-2-yl)carbamate,
benzyl (S)-(1-(2-(3-amino-3-oxopropyl)-2-(2-(4-fluorophenoxy)acetyl)hydrazineyl)-4-methyl-1-oxopentan-2-yl)carbamate,
2,2,2-trifluoroethyl 1-(3-amino-3-oxopropyl)-2-(((benzyloxy)carbonyl)-L-leucyl)hydrazine-1-carboxylate,
4-fluorophenyl 1-(3-amino-3-oxopropyl)-2-(((benzyloxy)carbonyl)-L-leucyl)hydrazine-1-carboxylate,
benzyl ((2S,3R)-1-(((S)-1-(2-acryloyl-2-(3-amino-3-oxopropyl)hydrazineyl)-4-methyl-1-oxopentan-2-yl)amino)-3-(tert-butoxy)-1-oxobutan-2-yl)carbamate,
benzyl ((S)-1-(((S)-1-(2-acryloyl-2-(3-amino-3-oxopropyl)hydrazineyl)-4-methyl-1-oxopentan-2-yl)amino)-3-methyl-1-oxobutan-2-yl)carbamate,
benzyl ((2S,3R)-1-(((S)-1-(2-acryloyl-2-(3-amino-3-oxopropyl)hydrazineyl)-3-cyclohexyl-1-oxopropan-2-yl)amino)-3-(tert-butoxy)-1-oxobutan-2-yl)carbamate,
benzyl ((2S,3R)-1-(((S)-1-(2-(3-amino-3-oxopropyl)-2-(2-chloroacetyl)hydrazineyl)-3-cyclohexyl-1-oxopropan-2-yl)amino)-3-(tert-butoxy)-1-oxobutan-2-yl)carbamate,
(1R,2S,5S)-3-((S)-2-(3-(tert-butyl)ureido)-3,3-dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide
(S)-2-((S)-2-(3-(tert-butyl)ureido)-3,3-dimethylbutanamido)-4,4-dimethyl-N—((S)-1-,oxo-3-((S)-2-oxopyrrolidin-3-yl)propan-2-yl)pentanamide,
(S)-5-((S)-2-(3-(tert-butyl)ureido)-3,3-dimethylbutanoyl)-N—((S)-1-oxo-3-((S)-2-oxopyrrolidin-3-yl)propan-2-yl)-5-azaspiro[2.4]heptane-6-carboxamide,
(1R,2S,5S)—N—((S)-4-amino-3,4-dioxo-1-((S)-2-oxopyrrolidin-3-yl)butan-2-yl)-3-((S)-2-(3-(tert-butyl)ureido)-3,3-dimethylbutanoyl)-6,6-dimethyl-3-azabicyclo[3.1.0]hexane-2-carboxamide,
(1R,2S,5S)-3-((S)-2-(3-(tert-butyl)ureido)-3,3-dimethylbutanoyl)-N—((S)-4-(ethylamino)-3,4-dioxo-1-((S)-2-oxopyrrolidin-3-yl)butan-2-yl)-6,6-dimethyl-3-azabicyclo[3.1.0]hexane-2-carboxamide,
(1R,2S,5S)-3-((S)-2-(3-(tert-butyl)ureido)-3,3-dimethylbutanoyl)-N—((S)-4-(cyclopropylamino)-3,4-dioxo-1-((S)-2-oxopyrrolidin-3-yl)butan-2-yl)-6,6-dimethyl-3-azabicyclo[3.1.0]hexane-2-carboxamide,
(1R,2S,5S)-3-((S)-2-(3-(tert-butyl)ureido)-3,3-dimethylbutanoyl)-N—((S)-4-((cyclopropylmethyl)amino)-3,4-dioxo-1-((S)-2-oxopyrrolidin-3-yl)butan-2-yl)-6,6-dimethyl-3-azabicyclo[3.1.0]hexane-2-carboxamide,
(1R,2S,5S)-3-((S)-2-(3-(tert-butyl)ureido)-3,3-dimethylbutanoyl)-N—((S)-4-(butylamino)-3,4-dioxo-1-((S)-2-oxopyrrolidin-3-yl)butan-2-yl)-6,6-dimethyl-3-azabicyclo[3.1.0]hexane-2-carboxamide,
(1R,2S,5S)-3-((S)-2-(3-(tert-butyl)ureido)-3,3-dimethylbutanoyl)-N—((S)-4-(hexylamino)-3,4-dioxo-1-((S)-2-oxopyrrolidin-3-yl)butan-2-yl)-6,6-dimethyl-3-azabicyclo[3.1.0]hexane-2-carboxamide,
(1R,2S,5S)—N—((S)-4-(benzylamino)-3,4-dioxo-1-((S)-2-oxopyrrolidin-3-yl)butan-2-yl)-3-((S)-2-(3-(tert-butyl)ureido)-3,3-dimethylbutanoyl)-6,6-dimethyl-3-azabicyclo[3.1.0]hexane-2-carboxamide,
(S)-2-(3-(tert-butyl)ureido)-N—((S)-3-cyclopropyl-1-oxo-1-(((S)-1-oxo-3-((S)-2-oxopyrrolidin-3-yl)propan-2-yl)amino)propan-2-yl)-3-methylbutanamide,
sodium (5S,8S,11S)-5-((R)-1-(tert-butoxy)ethyl)-8-(cyclohexylmethyl)-12-hydroxy-3,6,9-trioxo-11-(((S)-2-oxopyrrolidin-3-yl)methyl)-1-phenyl-2-oxa-4,7,10-triazadodecane-12-sulfonate,
benzyl ((2S)-1-(((2S)-1-(((2S)-1-cyano-1-hydroxy-3-((S)-2-oxopyrrolidin-3-yl)propan-2-yl)amino)-4-methyl-1-oxopentan-2-yl)amino)-3-methyl-1-oxobutan-2-yl)carbamate,
benzyl ((2S,3R)-3-(tert-butoxy)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-1-oxobutan-2-yl)carbamate,
(1R,2S,5S)-3-(O-(tert-butyl)-N-(tert-butylcarbamoyl)-L-threonyl)-6,6-dimethyl-N—((S)-1-oxo-3-((S)-2-oxopyrrolidin-3-yl)propan-2-yl)-3-azabicyclo[3.1.0]hexane-2-carboxamide,
1-((2S,3R)-1-((1R,2S,5S)-2-(2-acryloyl-2-(((S)-5-oxopyrrolidin-2-yl)methyl)hydrazine-1-carbonyl)-6,6-dimethyl-3-azabicyclo[3.1.0]hexan-3-yl)-3-(tert-butoxy)-1-oxobutan-2-yl)-3-(tert-butyl)urea,
3-(1-acryloyl-2-((1R,2S,5S)-3-(O-(tert-butyl)-N-(tert-butylcarbamoyl)-L-threonyl)-6,6-dimethyl-3-azabicyclo[3.1.0]hexane-2-carbonyl)hydrazineyl)propanamide,
tert-butyl ((S)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-3,3-dimethyl-1-oxobutan-2-yl)carbamate,
benzyl ((S)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-3,3-dimethyl-1-oxobutan-2-yl)carbamate,
(2S,3R)-3-(tert-butoxy)-2-(3-(tert-butyl)ureido)-N—((S)-3-cyclohexyl-1-oxo-1-(((S)-1-oxo-3-((S)-2-oxopyrrolidin-3-yl)propan-2-yl)amino)propan-2-yl)butanamide,
(S)-2-(3-(tert-butyl)ureido)-N—((S)-3-cyclopropyl-1-oxo-1-(((S)-1-oxo-3-((S)-2-oxopyrrolidin-3-yl)propan-2-yl)amino)propan-2-yl)-3-methylbutanamide,
(1R,2S,5S)-3-((S)-3,3-dimethyl-2-(3-methylbutanamido)butanoyl)-6,6-dimethyl-N—((S)-1-oxo-3-((S)-2-oxopyrrolidin-3-yl)propan-2-yl)-3-azabicyclo[3.1.0]hexane-2-carboxamide,
(1R,2S,5S)—N—((S)-1-cyano-2-((S)-2-oxopyrrolidin-3-yl)ethyl)-3-((S)-2-(3,3-dimethylbutanamido)-3,3-dimethylbutanoyl)-6,6-dimethyl-3-azabicyclo[3.1.0]hexane-2-carboxamide,
(1R,2S,5S)—N—((S)-4-amino-3,4-dioxo-1-((S)-2-oxopyrrolidin-3-yl)butan-2-yl)-3-((S)-2-(3,3-dimethylbutanamido)-3,3-dimethylbutanoyl)-6,6-dimethyl-3-azabicyclo[3.1.0]hexane-2-carboxamide,
benzyl ((2S,3R)-1-(((S)-1-(((S)-4-amino-3,4-dioxo-1-((S)-2-oxopyrrolidin-3-yl)butan-2-yl)amino)-3-cyclohexyl-1-oxopropan-2-yl)amino)-3-(tert-butoxy)-1-oxobutan-2-yl)carbamate,
(1R,2S,5S)-3-(2,2-bis(4-chlorophenoxy)acetyl)-6,6-dimethyl-N—((S)-1-oxo-3-((S)-2-oxopyrrolidin-3-yl)propan-2-yl)-3-azabicyclo[3.1.0]hexane-2-carboxamide,
benzyl ((S)-2-(((S)-3-cyclohexyl-1-oxo-1-(((S)-1-oxo-3-((S)-2-oxopyrrolidin-3-yl)propan-2-yl)amino)propan-2-yl)amino)-2-oxo-1-phenylethyl)carbamate,
benzyl ((S)-1-cyclohexyl-2-(((S)-3-cyclohexyl-1-oxo-1-(((S)-1-oxo-3-((S)-2-oxopyrrolidin-3-yl)propan-2-yl)amino)propan-2-yl)amino)-2-oxoethyl)carbamate,
3-hydroxybenzyl ((S)-3-cyclohexyl-1-oxo-1-(((S)-1-oxo-3-((S)-2-oxopyrrolidin-3-yl)propan-2-yl)amino)propan-2-yl)carbamate,
3-(((((S)-3-cyclohexyl-1-oxo-1-(((S)-1-oxo-3-((S)-2-oxopyrrolidin-3-yl)propan-2-yl)amino)propan-2-yl)carbamoyl)oxy)methyl)phenyl acetate,
(1R,2S,5S)-3-((S)-2-(3,3-dimethylbutanamido)-3,3-dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide,
benzyl ((S)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-1-oxo-3-phenylpropan-2-yl)carbamate,
benzyl ((S)-3-(4-chlorophenyl)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-1-oxopropan-2-yl)carbamate,
benzyl ((2S,3R)-1-(((2S)-1-(2-acryloyl-2-((2-oxopyrrolidin-3-yl)methyl)hydrazineyl)-3-cyclohexyl-1-oxopropan-2-yl)amino)-3-(tert-butoxy)-1-oxobutan-2-yl)carbamate,
benzyl ((2S,3R)-3-(tert-butoxy)-1-(((2S)-1-(2-(2-chloroacetyl)-2-((2-oxopyrrolidin-3-yl)methyl)hydrazineyl)-3-cyclohexyl-1-oxopropan-2-yl)amino)-1-oxobutan-2-yl)carbamate,
3-fluorobenzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate, and
4-fluorobenzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate.

80. A compound, or pharmaceutical acceptable salt thereof, selected from Table A.

81. A pharmaceutical composition comprising at least one compound of any one of clauses 1 to 80, or a pharmaceutically acceptable salt thereof, and optionally one or more pharmaceutically acceptable excipients.

82. A method of treating disease, such as a viral infection, comprising administering to a subject in need of such treatment an effective amount of a compound of any one of clauses 1 to 80, or a pharmaceutically acceptable salt thereof.

83. A compound of any one of clauses 1 to 80, or a pharmaceutically acceptable salt thereof, for use in a method of treating a viral infection in a subject.

84. A compound of any one of clauses 1 to 80, or a pharmaceutically acceptable salt thereof, for treating a viral infection in a subject.

85. Use of a compound of any one of clauses 1 to 80, or a pharmaceutically acceptable salt thereof, in the manufacture of a medicament for treating a viral infection in a subject.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Definitions

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo, (═O), thiocarbonyl, O-carbamyl, N-carbamyl, O-thiocarbamyl, N-thiocarbamyl, C-amido, N-amido, C-carboxy, O-carboxy, —NO₂, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “alkenyl” includes a chain of carbon atoms, which is optionally branched, and contains from 2 to 20 carbon atoms, and also includes at least one carbon-carbon double bond (i.e., C═C). It will be understood that in certain embodiments, alkenyl may be advantageously of limited length, including C₂-C₁₂, C₂-C₉, C₂-C₈, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “alkynyl” includes a chain of carbon atoms, which is optionally branched, and contains from 2 to 20 carbon atoms, and also includes at least one carbon-carbon triple bond (i.e., C≡C). It will be understood that in certain embodiments, alkynyl may each be advantageously of limited length, including C₂-C₁₂, C₂-C₉, C₂-C₈, C₂-C₇, C₂-C₆, and C₂-C₄. Illustratively, such particularly limited length alkynyl groups, including C₂-C₈, C₂-C₇, C₂-C₆, and C₂-C₄ may be referred to as lower alkynyl. Alkynyl may be unsubstituted, or substituted as described for alkyl or as described in the various embodiments provided herein. Illustrative alkynyl groups include, but are not limited to, ethynyl, 1-propynyl, 2-propynyl, 1-, 2-, or 3-butynyl, and the like.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthylenyl and anthracenyl. The aryl group may be unsubstituted, or substituted as described for alkyl or as described in the various embodiments provided herein.

As used herein, the term “cycloalkyl” refers to a 3 to 15 member all-carbon monocyclic ring, including an all-carbon 5-member/6-member or 6-member/6-member fused bicyclic ring, or a multicyclic fused ring (a “fused” ring system means that each ring in the system shares an adjacent pair of carbon atoms with each other ring in the system) group, or a carbocyclic ring that is fused to another group such as a heterocyclic, such as ring 5- or 6-membered cycloalkyl fused to a 5- to 7-membered heterocyclic ring, where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₉, C₃-C₆ and C₄-C₆. Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like. Illustrative examples of cycloalkyl groups shown in graphical representations include the following entities, in the form of properly bonded moieties:

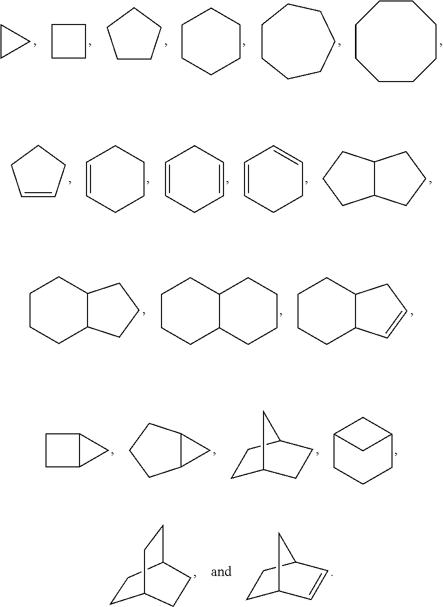

As used herein, the term “heterocycloalkyl” refers to a monocyclic or fused ring group having in the ring(s) from 3 to 12 ring atoms, in which at least one ring atom is a heteroatom, such as nitrogen, oxygen or sulfur, the remaining ring atoms being carbon atoms. Heterocycloalkyl may optionally contain 1, 2, 3 or 4 heteroatoms. A heterocycloalkyl group may be fused to another group such as another heterocycloalkyl, or a heteroaryl group. Heterocycloalkyl may also have one of more double bonds, including double bonds to nitrogen (e.g., C═N or N═N) but does not contain a completely conjugated pi-electron system. It will be understood that in certain embodiments, heterocycloalkyl may be advantageously of limited size such as 3- to 7-membered heterocycloalkyl, 5- to 7-membered heterocycloalkyl, 3-, 4-, 5- or 6-membered heterocycloalkyl, and the like. Heterocycloalkyl may be unsubstituted, or substituted as described for alkyl or as described in the various embodiments provided herein. Illustrative heterocycloalkyl groups include, but are not limited to, oxiranyl, thianaryl, azetidinyl, oxetanyl, tetrahydrofuranyl, pyrrolidinyl, tetrahydropyranyl, piperidinyl, 1,4-dioxanyl, morpholinyl, 1,4-dithianyl, piperazinyl, oxepanyl, 3,4-dihydro-2H-pyranyl, 5,6-dihydro-2H-pyranyl, 2H-pyranyl, 1, 2, 3, 4-tetrahydropyridinyl, and the like. Illustrative examples of heterocycloalkyl groups shown in graphical representations include the following entities, in the form of properly bonded moieties:

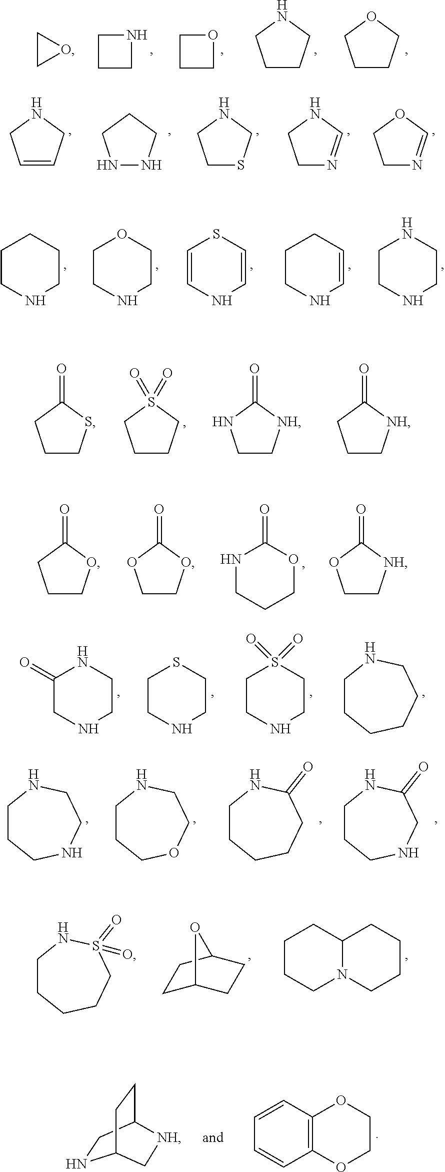

As used herein, the term “heteroaryl” refers to a monocyclic or fused ring group of 5 to 12 ring atoms containing one, two, three or four ring heteroatoms selected from nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, 5- to 10-membered heteroaryl and the like. Heteroaryl may be unsubstituted, or substituted as described for alkyl or as described in the various embodiments provided herein. Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like. Illustrative examples of heteroaryl groups shown in graphical representations, include the following entities, in the form of properly bonded moieties:

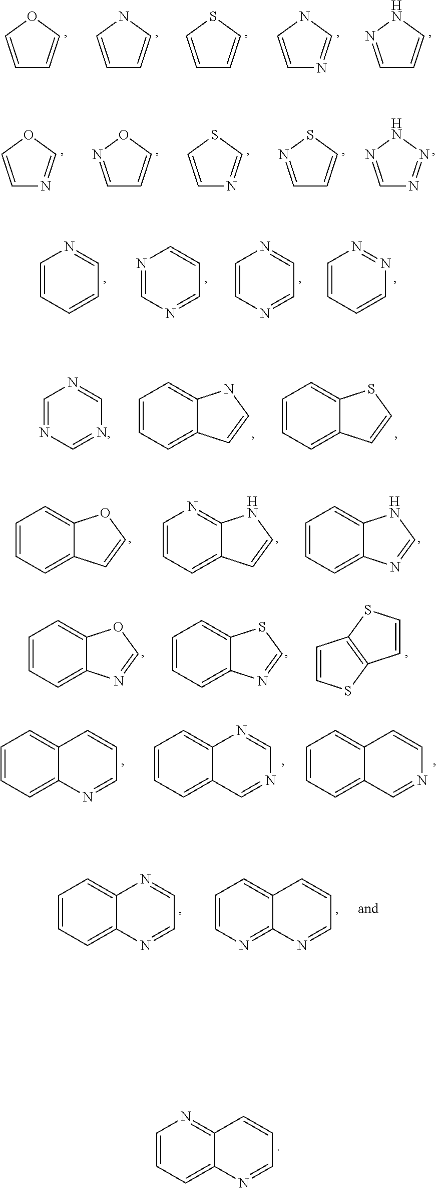

In a particular embodiment, the heteroaryl group is

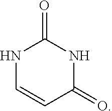

As used herein, “hydroxy” or “hydroxyl” refers to an —OH group.

As used herein, “alkoxy” refers to both an —O-(alkyl) or an —O-(unsubstituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like.

As used herein, “halo” or “halogen” refers to fluorine, chlorine, bromine or iodine.

As used herein, “cyano” refers to a —CN group.

The term “oxo” represents a carbonyl oxygen. For example, a cyclopentyl substituted with oxo is cyclopentanone.

As used herein, “bond” refers to a covalent bond.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

As used herein, “optional” or “optionally” means that the subsequently described event or circumstance may but need not occur, and that the description includes instances where the event or circumstance occurs and instances in which it does not. For example, “wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is independently optionally substituted by C₁-C₆ alkyl” means that an alkyl may be but need not be present on any of the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl by replacement of a hydrogen atom for each alkyl group, and the description includes situations where the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is substituted with an alkyl group and situations where the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is not substituted with the alkyl group.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the phrase “taken together with the atoms to which they are attached” or “taken together with the atom to which they are attached” means that two substituents (e.g., R² and R³) attached to two separate atoms or attached to the same atom form the groups that are defined by the claim, such as

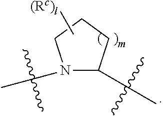

In particular, the phrase “taken together with the atoms to which they are attached” means that when, for example, R² and R³, and the nitrogen and carbon atom respectively to which each are attached form a

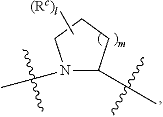

then the formed ring will be attached at the nitrogen and carbon atoms. For example, the phrase “R² and R³ together with the atoms to which they are attached combine to form” used in connection with the embodiments described herein includes the compounds represented as follows:

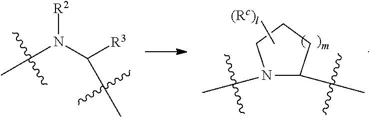

Alternatively, the phrase can be directed to two substituents on the same atom. For example, the phrase “R² and R³ together with the atom to which they are attached combine to form” used in connection with the embodiments described herein includes the compounds represented as follows

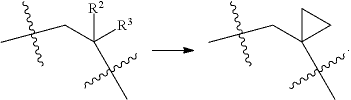

As used herein, the term “pharmaceutically acceptable salt” refers to those salts which counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:

(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Pharmaceutically acceptable salts are well known to those skilled in the art, and any such pharmaceutically acceptable salt may be contemplated in connection with the embodiments described herein. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, 7-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula I-XIII that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula I-XIII, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula I-XIII). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of compounds of Formula I-XIII, and uses of such metabolites in the methods of the disclosure. A “pharmaceutically active metabolite” means a pharmacologically active product of metabolism in the body of a compound of Formula I-XIII, or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof. For example, it will be appreciated that compounds depicted by a structural formula containing the symbol “ [symbol] ” include both stereoisomers for the carbon atom to which the symbol “ [symbol] ” is attached, specifically both the bonds “ [symbol] ” and “ [symbol] ” are encompassed by the meaning of “ [symbol] ”. For example, in some [symbol] exemplary embodiments, certain compounds provided herein can be described by the formula

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O. ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. For example, isotope-labeled compounds and salts can be used as medicaments. Further, substitution with heavier isotopes such as deuterium (i.e., ²H) may afford certain therapeutic advantages resulting from greater metabolic stability, for example increased in vivo half-life or reduced dosage requirements. For example, deuterium (²H)-labeled compounds and salts may be therapeutically useful with potential therapeutic advantages over the non-²H-labeled compounds. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Any disubstituent referred to herein is meant to encompass the various attachment possibilities when more than one of such possibilities are allowed. For example, reference to disubstituent -A-B—, where A≠B, refers herein to such disubstituent with A attached to a first substituted member and B attached to a second substituted member, and it also refers to such disubstituent with A attached to the second substituted member and B attached to the first substituted member.

### REPRESENTATIVE EMBODIMENTS

In some embodiments, compounds described herein comprise a moiety of the formula I

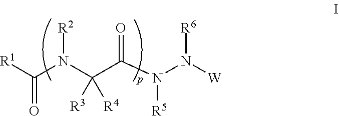

wherein R¹, R², each R³, each R⁴, R⁵, R⁶, p, and W are as defined herein.

In some embodiments, compounds described herein comprise a moiety of the formula II

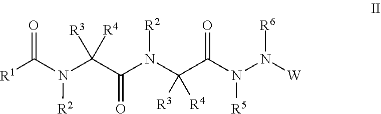

wherein R¹, R², each R³, each R⁴, R⁵, R⁶, and W are as defined herein.

In some embodiments, compounds described herein comprise a moiety of the formula III

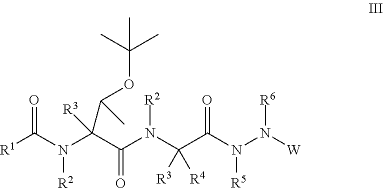

wherein R¹, R², each R³, R⁴, R⁵, R⁶, and W are as defined herein.

In some embodiments, compounds described herein comprise a moiety of the formula IV

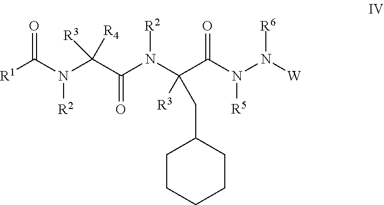

wherein R¹, R², each R³, R⁴, R⁵, R⁶, and W are as defined herein.

In some embodiments, compounds described herein comprise a moiety of the formula V

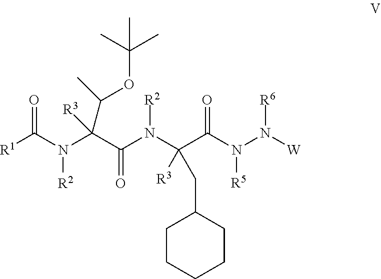

wherein R¹, R², each R³, R⁵, R⁶, and W are as defined herein.

In some embodiments, compounds described herein comprise a moiety of the formula VI

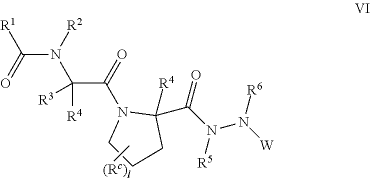

wherein R¹, R², R³, each R⁴, R⁵, R⁶, W, R^{c}, and 1 are as defined herein.

In some embodiments, compounds described herein comprise a moiety of the formula VII

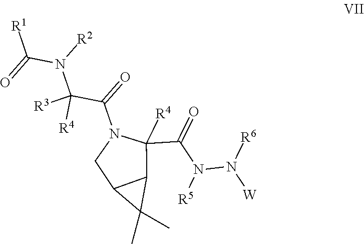

wherein R¹, R², R³, each R⁴, R⁵, R⁶, and W are as defined herein.

In some embodiments, compounds described herein comprise a moiety of the formula VIII

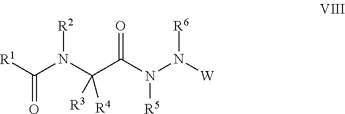

wherein R¹, R², R³, R⁴, R⁵, R⁶, and W are as defined herein.

In some embodiments, compounds described herein comprise a moiety of the formula IX

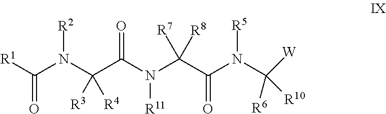

wherein R¹, R², R³, R⁴, R⁵, R⁷, R⁸, R⁹, R¹⁰, R¹¹, and W are as defined herein.

In some embodiments, compounds described herein comprise a moiety of the formula X

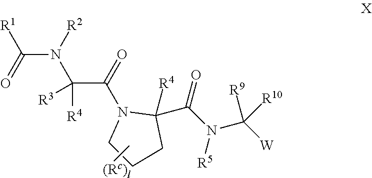

wherein R¹, R², R³, R⁴, R⁵, R⁸, R⁹, R¹⁰, W, R^{c}, and 1 are as defined herein.

In some embodiments, compounds described herein comprise a moiety of the formula XI

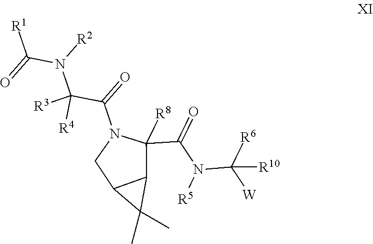

wherein R¹, R², R³, R⁴, R⁵, R⁸, R⁹, R¹⁰, and W are as defined herein.

In some embodiments, compounds described herein comprise a moiety of the formula XII

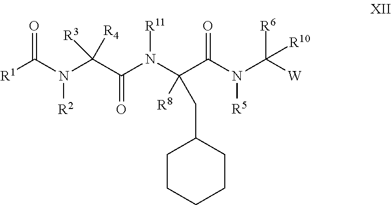

wherein R¹, R², R³, R⁴, R⁵, R⁸, R⁹, R¹⁰, R¹¹, and W are as defined herein.

In some embodiments, compounds described herein comprise a moiety of the formula XIII

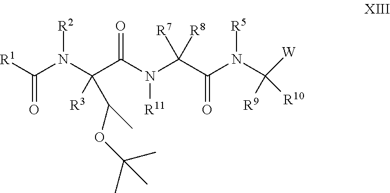

wherein R¹, R², R³, R⁵, R⁷, R⁸, R⁹, R¹⁰, R¹¹, and W are as defined herein.

The compounds described and exemplified herein by comprise one or two amino acids. As used herein, the term “amino acid” may refer generally to beta, gamma, and longer amino acids, such as amino acids of the formula: —N(R)—(CR′R″)_{q}—C(O)— where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. In some embodiments, the stereoisomeric configuration is the L-configuration. For example, the amino acid may be selected from asparagine, aspartic acid, cysteine, glutamic acid, lysine, glutamine, arginine, serine, ornitine, threonine, and the like. In some embodiments, the amino acid may be threonine or a hydroxy-protected threonine.

As used herein, the term “amino acid derivative” generally refers to an amino acid as defined herein where either, or both, the amino group and/or the side chain is substituted or a non-naturally occurring amino acid. Illustrative amino acid derivatives include prodrugs and protecting groups of the amino group and/or the side chain, such as amine, amide, hydroxy, carboxylic acid, and thio prodrugs and protecting groups. Additional illustrative amino acid derivatives include substituted variations of the amino acid as described herein, such as, but not limited to, ethers and esters of hydroxy groups, amides, carbamates, and ureas of amino groups, esters, amides, and cyano derivatives of carboxylic acid groups, and the like. In some embodiments, the amino acid derivative comprises a a sidechain that is not naturally occurring. In some embodiments, the side chain is t-butyl, methyl-cyclohexyl, hydroxy-protected threonines, methyl-pyrrolidone, etc. for example.

In some embodiments, R¹ is C₁-C₆ alkyl, C₂-C₆ alkenyl, C₁-C₆ alkoxy, 5-10 membered heteroaryl, —NH₂, —NHR^{a}, —NR^{a}R^{b}, or —C₁-C₆ alkyl-NR^{a}R^{b}, wherein each hydrogen atom in C₁-C₆ alkyl, C₂-C₆ alkenyl, and 5-10 membered heteroaryl is optionally substituted by an R^{a}. In some embodiments, R¹ is C₁-C₆ alkoxy or C₂-C₆ alkenyl, wherein each hydrogen atom in C₁-C₆ alkoxy or C₂-C₆ alkenyl is optionally substituted by an R^{a}. In some embodiments, R¹ is C₁-C₆ alkoxy, wherein each hydrogen atom in C₁-C₆ alkoxy is optionally substituted by an R^{a}. In some embodiments, C₁-C₆ alkoxy is substituted with an R^{a}. In some embodiments, the R^{a} is C₆-C₁₀ aryl, wherein each hydrogen atom in C₆-C₁₀ aryl is optionally substituted by halo. In some embodiments, the R^{a} is C₆-C₁₀ aryl substituted with at least one halo. In some embodiments, the R^{a} is C₆-C₁₀ aryl substituted with one halo. In some embodiments, R¹ is

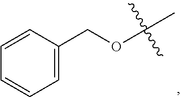 [SMILES: *OCc1ccccc1]

wherein each hydrogen atom in

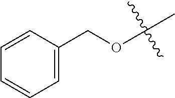 [SMILES: *OCc1ccccc1]

is optionally substituted. In some embodiments, R¹ is

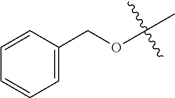 [SMILES: *OCc1ccccc1]

wherein at least one hydrogen atom in

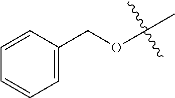 [SMILES: *OCc1ccccc1]

is substituted by halo. The halo may be fluoro, chloro, or bromo. In some embodiments, R¹ is NHR^{a}. In some embodiments, R¹ is NHC(CH₃)₃.

In some embodiments, R² is H or C₁-C₆ alkyl. In some embodiments, R³ is H or C₁-C₆ alkyl. In some embodiments, R² and R³ together with the atoms to which they are attached combine to form

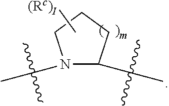

In some embodiments, R² and R³ together with the atoms to which they are attached combine to form

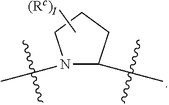

In some embodiments, R² and R³ together with the atoms to which they are attached combine to form

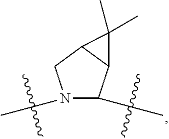 [SMILES: *C1C2C(CN1*)C2(C)C]

wherein each hydrogen atom in

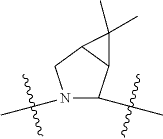 [SMILES: *C1C2C(CN1*)C2(C)C]

is optionally substituted. In some embodiments, R² and R³ together with the atoms to which they are attached combine to form

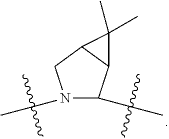 [SMILES: *C1C2C(CN1*)C2(C)C]

In some embodiments, R³ is methyl.

In some embodiments, the compounds described herein may have one or at least one R⁴. The following description applies to each individual R⁴, if present. In some embodiments, R⁴ is H, C₁-C₆ alkyl, C₂-C₆ alkenyl, or C₃-C₈ cycloalkyl, wherein each hydrogen atom in C₁-C₆ alkyl, C₂-C₆ alkenyl, and C₃-C₈ cycloalkyl is optionally substituted by R^{b}. In some embodiments, R⁴ is C₁-C₆ alkyl. In some embodiments, R⁴ is C₁-C₆ alkyl, wherein each hydrogen atom in C₁-C₆ alkyl is optionally substituted by C₁-C₆ alkoxy or C₃-C₈ cycloalkyl. In some embodiments, R⁴ is C₁-C₆ alkyl, wherein at least one hydrogen atom in C₁-C₆ alkyl is substituted by C₁-C₆ alkoxy. In some embodiments, R⁴ is C₁-C₆ alkyl, wherein at least on hydrogen atom in C₁-C₆ alkyl is substituted by C₃-C₈ cycloalkyl. In some embodiments, R⁴ is t-butyl or isopropyl. In some embodiments, R⁴ is

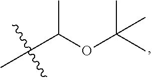 [SMILES: *C(C)OC(C)(C)C]

which may be optionally substituted. In some embodiments, R⁴ is

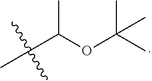 [SMILES: *C(C)OC(C)(C)C]

In some embodiments, R⁴ is C₁-C₆ alkyl, C₆-C₁₀ aryl, or C₃-C₈ cycloalkyl, wherein each hydrogen atom in C₁-C₆ alkyl, C₆-C₁₀ aryl, and C₃-C₈ cycloalkyl is optionally substituted by R^{b} or C₁-C₆ alkoxy. In some embodiments, R⁴ is C₁-C₆ alkyl or C₃-C₈ cycloalkyl. In some embodiments, R⁴ is phenyl.

In some embodiments, R³ and R⁴ together with the carbon atom to which they are attached form C₃-C₈ cycloalkyl, wherein each hydrogen atom in C₃-C₈ cycloalkyl is optionally substituted by R^{b}.

In some embodiments, R⁵ is H or C₁-C₆ alkyl, wherein each hydrogen atom in C₁-C₆ alkyl is optionally substituted by —C(O)NR^{a}R^{b}. In some embodiments, R⁵ is H.

In some embodiments, R⁶ is C₁-C₆ alkyl, wherein each hydrogen atom in C₁-C₆ alkyl is optionally substituted by C(O)NR^{a}R^{b}, 3- to 8-membered heterocycloalkyl, or 5-10 membered heteroaryl. In some embodiments, R⁶ is C₁-C₆ alkyl, wherein C₁-C₆ alkyl is substituted with an C(O)NR^{a}R^{b}, a 3- to 8-membered heterocycloalkyl, or a 5-10 membered heterocycle. In some embodiments, R⁶ is C₁-C₆ alkyl, wherein at least one hydrogen atom in C₁-C₆ alkyl is substituted by C(O)NR^{a}R^{b} or 3- to 8-membered heterocycloalkyl. In some embodiments, R⁶ is C₁-C₆ alkyl, wherein at least one hydrogen atom in C₁-C₆ alkyl is substituted by C(O)NR^{a}R^{b}. In some embodiments, R⁶ is C₁-C₆ alkyl substituted with a C(O)NR^{a}R^{b}. In some embodiments, R⁶ is C₁-C₆ alkyl substituted with an, wherein at least one hydrogen atom in C₁-C₆ alkyl is substituted by C(O)NH₂. In some embodiments, R⁶ is C₁-C₆ alkyl substituted with an —C(O)NH₂. In some embodiments, R⁶ is C₁-C₆ alkyl, wherein at least one hydrogen atom in C₁-C₆ alkyl is substituted by 3- to 8-membered heterocycloalkyl. In some embodiments, R⁶ is C₁-C₆ alkyl substituted with a 3- to 8-membered heterocycloalkyl. In some embodiments, R⁶ is C₁-C₆ alkyl, wherein at least one hydrogen atom is substituted by

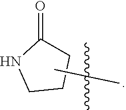

In some embodiments, R⁶ is C₁-C₆ alkyl substituted with

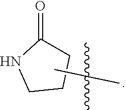

In some embodiments, R⁶ is

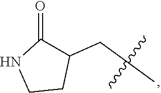 [SMILES: *CC1CCNC1=O]

wherein each hydrogen atom in

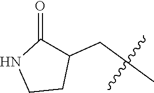 [SMILES: *CC1CCNC1=O]

may be optionally substituted. In some embodiments, R⁶ is

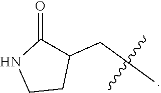 [SMILES: *CC1CCNC1=O]

In some embodiments, each of R⁷ and R⁸ is independently H, C₁-C₆ alkyl, C₂-C₆ alkenyl, or C₃-C₈ cycloalkyl, wherein each hydrogen atom in C₁-C₆ alkyl, C₂-C₆ alkenyl, and C₃-C₈ cycloalkyl is optionally substituted by R^{b}. In some embodiments, R⁷ or R⁸ is C₁-C₆ alkyl, wherein each hydrogen atom in C₁-C₆ alkyl is optionally substituted by C₃-C₈ cycloalkyl, C₆-C₁₀ aryl, or 5-10 membered heteroaryl. In some embodiments, R⁷ or R⁸ is C₁-C₆ alkyl substituted with a C₃-C₈ cycloalkyl, a C₆-C₁₀ aryl, or a 5-10 membered heteroaryl. In some embodiments, R⁷ or R⁸ is

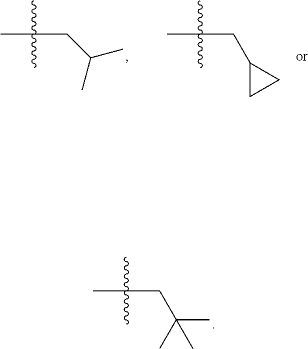

In some embodiments, at least one of R⁷ and R⁸ is not

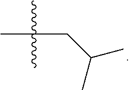 [SMILES: *CC(C)C]

In some embodiments, each of R⁹ and R¹⁰ is independently H or C₁-C₆ alkyl, wherein each hydrogen atom in C₁-C₆ alkyl is optionally substituted by C(O)NR^{a}R^{b}, 3- to 8-membered heterocycloalkyl, or 5-10 membered heteroaryl. In some embodiments, at least one of R⁹ and R¹⁰ is C₁-C₆ alkyl, wherein at least one hydrogen atom in C₁-C₆ alkyl is substituted by C(O)NR^{a}R^{b} or 3- to 8-membered heterocycloalkyl. In some embodiments, at least one of R⁹ and R¹⁰ is C₁-C₆ alkyl substituted with a C(O)NR^{a}R^{b} or a 3- to 8-membered heterocycloalkyl. In some embodiments, at least one of R⁹ and R¹⁰ is C₁-C₆ alkyl, wherein each hydrogen atom in C₁-C₆ alkyl is optionally substituted by C(O)NR^{a}R^{b}. In some embodiments, at least one of R⁹ and R¹⁰ is C₁-C₆ alkyl, wherein at least one hydrogen atom in C₁-C₆ alkyl is substituted by C(O)NR^{a}R^{b}. In some embodiments, at least one of R⁹ and R¹⁰ is C₁-C₆ alkyl substituted with a C(O)NR^{a}R^{b}. In some embodiments, at least one of R⁹ and R¹⁰ is C₁-C₆ alkyl, wherein each hydrogen atom in is C₁-C₆ alkyl is optionally substituted by a C(O)NH₂. In some embodiments, at least one of R⁹ and R¹⁰ is C₁-C₆ alkyl, wherein at least one hydrogen atom in is C₁-C₆ alkyl is optionally substituted by a C(O)NH₂. In some embodiments, at least one of R⁹ and R¹⁰ is C₁-C₆ alkyl substituted with a C(O)NH₂. In some embodiments, at least one of R⁹ and R¹⁰ is C₁-C₆ alkyl, wherein at least one hydrogen atom in C₁-C₆ alkyl is substituted by 3- to 8-membered heterocycloalkyl. In some embodiments, at least one of R⁹ and R¹⁰ is C₁-C₆ alkyl substituted with a 3- to 8-membered heterocycloalkyl. In some embodiments, at least one of R⁸ and R⁹ is C₁-C₆ alkyl substituted with a

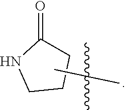

In some embodiments, at least one of R⁸ and R⁹ is

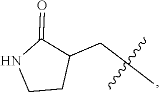 [SMILES: *CC1CCNC1=O]

which may be optionally substituted. In some embodiments, at least one of R⁸ and R⁹ is

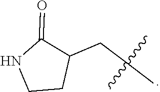 [SMILES: *CC1CCNC1=O]

In some embodiments, at least one of R⁹ and R¹⁰ is not

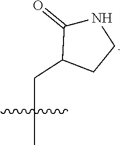 [SMILES: *CC1CCNC1=O]

In some embodiments, R⁹ is H.

In some embodiments, R¹¹ is H. In some embodiments, R¹¹ and one of R⁷ or R⁸ together with the atoms to which they are attached combine to form

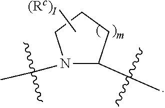

In some embodiments, R¹¹ and one of R⁷ or R⁸ together with the atoms to which they are attached combine to form

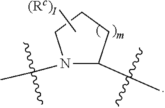

In some embodiments, R¹¹ and one of R⁷ or R⁸ together with the atoms to which they are attached combine to form

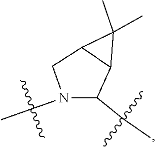 [SMILES: *C1C2C(CN1*)C2(C)C]

which may be optionally substituted. In some embodiments, R¹¹ and one of R⁷ or R⁸ together with the atoms to which they are attached combine to form

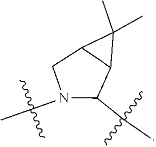 [SMILES: *C1C2C(CN1*)C2(C)C]

In some embodiments, W is C₁-C₆ alkyl-SO₄, C₁-C₆ alkyl-CN, —C(O)H, —C(O)OR^{d}, —C(O)C₁-C₆alkyl, —C(O)C₂-C₆ alkenyl, —C(O)C₆-C₁₀ aryl, —C(O)C(O)NR^{a}R^{b}, C(O)COC(O)C₁-C₁₀ alkyl, —C(O)COC(O)C₆-C₁₀ aryl, —C(O)COC(O)-5-10 membered heteroaryl, or —CN, wherein each hydrogen atom in C₁-C₆ alkyl, C₂-C₆ alkenyl, and C₆-C₁₀ aryl is optionally substituted by halo, —OSO₄, —CN, —OR^{d}, —NO₂, C₆-C₁₀ aryl, or —NR^{a}R^{b}. In some embodiments, W is C(O)H. In some embodiments, W is not C(O)H. In some embodiments, W is —C(O)C₁-C₆ alkyl, wherein each hydrogen atom in C₁-C₆ alkyl is optionally substituted by halo or OR^{d}. In some embodiments, R^{d} is C₆-C₁₀ aryl, wherein each hydrogen atom in C₆-C₁₀ aryl is optionally substituted by halo or —NO₂. In some embodiments, W is —C(O)C₁-C₆ alkyl, wherein each hydrogen atom in C₁-C₆ alkyl is optionally substituted by halo. In some embodiments, W is —C(O)C₁-C₆ alkyl, wherein at least one hydrogen atom in C₁-C₆ alkyl is substituted by halo. In some embodiments, W is —C(O)C₁-C₆ alkyl wherein the C₁-C₆ alkyl is substituted with a halo. In some embodiments, W is C(O)C₂-C₆ alkenyl, wherein each hydrogen atom in C₂-C₆ alkenyl is optionally substituted by C₆-C₁₀ aryl or —NR^{a}R^{b}. In some embodiments, W is C(O)C₂-C₆ alkenyl, wherein at least one hydrogen atom in C₂-C₆ alkenyl is substituted by C₆-C₁₀ aryl or —NR^{a}R^{b}. In some embodiments, W is C(O)C₂-C₆ alkenyl, wherein C₂-C₆ alkenyl is substituted with a C₆-C₁₀ aryl or a —NR^{a}R^{b}. In some embodiments, W is —C(O)C₆-C₁₀ aryl, wherein each hydrogen atom in C₆-C₁₀ aryl is optionally substituted by halo or —NO₂. In some embodiments, W is —C(O)C₆-C₁₀ aryl, wherein at least one hydrogen atom in C₆-C₁₀ aryl is substituted by halo or —NO₂. In some embodiments, W is —C(O)C₆-C₁₀ aryl, wherein at C₆-C₁₀ aryl is substituted with a halo or a —NO₂. In some embodiments, W is C(O)OR^{d}. In some embodiments, R^{d} is C₁-C₆ alkyl, wherein each hydrogen atom in C₁-C₆ alkyl is optionally substituted by halo or C₆-C₁₀ aryl, wherein each hydrogen atom in C₆-C₁₀ aryl is optionally substituted by halo or —NO₂. In some embodiments, W is —C(O)C₂-C₆ alkenyl optionally substituted by C₆-C₁₀ aryl or —NR^{a}R^{b}. In some embodiments, W is C₁-C₆ alkyl-SO₄, wherein each hydrogen atom in C₁-C₆ alkyl is optionally substituted by hydroxy. In some embodiments, W is C₁-C₆ alkyl-SO₄, wherein C₁-C₆ alkyl is substituted with a hydroxy. In some embodiments, W is —C(O)C(O)NR^{a}R^{b}, where each of R^{a} and R^{b} is independently H or C₁-C₆ alkyl, wherein each hydrogen atom in C₁-C₆ alkyl is optionally substituted by C₆-C₁₀ aryl. In some embodiments, W is C₁-C₆ alkyl-CN, wherein each hydrogen atom in C₁-C₆ alkyl is optionally substituted by hydroxy. In some embodiments, W is C₁-C₆ alkyl-CN, wherein C₁-C₆ alkyl is substituted with a hydroxy.

In some embodiments, each R^{a} and R^{b} is independently H, C₁-C₆ alkyl, C₃-C₈ cycloalkyl, C₁-C₆alkoxy, or C₆-C₁₀ aryl, wherein each hydrogen atom in C₁-C₆ alkyl, C₃-C₈ cycloalkyl, C₁-C₆ alkoxy, and C₆-C₁₀ aryl is optionally substituted by halo, OR^{d}, 5-10 membered heteroaryl, C₂-C₆ alkenyl, C₆-C₁₀ aryl, or C₃-C₈ cycloalkyl. In some embodiments, R^{a} is C₆-C₁₀ aryl, wherein each hydrogen atom in C₆-C₁₀ aryl is optionally substituted by halo. In some embodiments, each of R^{a} and R^{b} is H. In some embodiments, each of R^{a} and R^{b} is C₁-C₆ alkyl. In some embodiments, the C₁-C₆ alkyl is substituted with an optionally substituted phenyl. In some embodiments, each of R^{a} and R^{b} is independently H or C₁-C₆ alkyl, wherein each hydrogen atom in C₁-C₆ alkyl is optionally substituted by C₆-C₁₀ aryl. In some embodiments, each of R^{a} and R^{b} is independently H or C₁-C₆ alkyl, wherein at least one hydrogen atom in C₁-C₆ alkyl is optionally substituted by C₆-C₁₀ aryl. In some embodiments, each of R^{a} and R^{b} is independently H or C₁-C₆ alkyl, wherein the C₁-C₆ alkyl is substituted with a C₆-C₁₀ aryl.

In some embodiments, R^{c} is C₁-C₆ alkoxy or C₃-C₈ cycloalkyl. In some embodiments two R^{c} combine together with the atom or atoms to which they are attached to form a C₃-C₈ cycloalkyl, wherein each hydrogen atom in C₃-C₈ cycloalkyl is optionally substituted by C₁-C₆ alkyl. In some embodiments two R^{c} combine together with the atom or atoms to which they are attached to form a C₃-C₈ cycloalkyl, wherein at least one hydrogen atom in C₃-C₈ cycloalkyl is optionally substituted by C₁-C₆ alkyl. In some embodiments two R^{c} combine together with the atom or atoms to which they are attached to form a C₃-C₈ cycloalkyl, wherein the C₃-C₈ cycloalkyl is substituted with a C₁-C₆alkyl.

In some embodiments, R^{d} is H, C₁-C₆ alkyl, —C(O)C₁-C₆ alkyl, or C₆-C₁₀ aryl, wherein each hydrogen atom in C₁-C₆ alkyl and C₆-C₁₀ aryl is optionally substituted by halo or —NO₂. In some embodiments, R^{d} is H, C₁-C₆ alkyl, —C(O)C₁-C₆ alkyl, or C₆-C₁₀ aryl, wherein at least one hydrogen atom in C₁-C₆ alkyl and C₆-C₁₀ aryl is optionally substituted by halo or —NO₂

In some embodiments, 1 is 0, 1, 2, or 3. In some embodiments, 1 is 1. In some embodiments, 1 is 2. In some embodiments, 1 is 3.

In some embodiments, p is 1 or 2. In some embodiments, p is 1. In some embodiments, p is 2.

In some embodiments, m is 1, 2, or 3. In some embodiments, m is 1. In some embodiments, m is 2. In some embodiments, m is 3.

In some embodiments, if R⁴ is

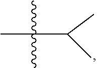 [SMILES: *C(C)C]

R¹ is —OCH₂-phenyl, one of R⁹ and R¹⁰ is

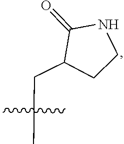 [SMILES: *CC1CCNC1=O]

and one of R⁷ and R⁸ is

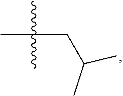 [SMILES: *CC(C)C]

then W is not —C(O)H.

In some embodiments, if R⁴ is

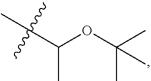 [SMILES: *C(C)OC(C)(C)C]

R¹ is —OCH₂-phenyl, one of R⁹ and R¹⁰ is

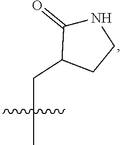 [SMILES: *CC1CCNC1=O]

and one of R⁷ and R⁸ is

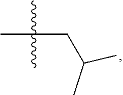 [SMILES: *CC(C)C]

then W is not —C(O)H.

In some embodiments, if R⁴ is

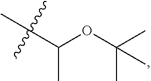 [SMILES: *C(C)OC(C)(C)C]

R¹ is —OCH₂-phenyl, one of R⁹ and R¹⁰ is

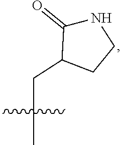 [SMILES: *CC1CCNC1=O]

and one of R⁷ and R⁸ is —CH₂-cyclohexyl, then W is not —C(O)H

The following represent illustrative embodiments of compounds of the Formula I-XIII:

TABLE A

| Compound | Structure | Name |
|---|---|---|
| MPI4 | 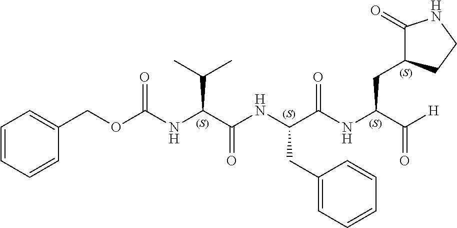 | benzyl ((S)-3-methyl-1-oxo-1-(((S)-1- oxo-1-(((S)-1-oxo-3-((S)-2- oxopyrrolidin-3-yl)propan-2-yl)amino)- 3-phenylpropan-2-yl)amino)butan-2- yl)carbamate |
| MPI5 | 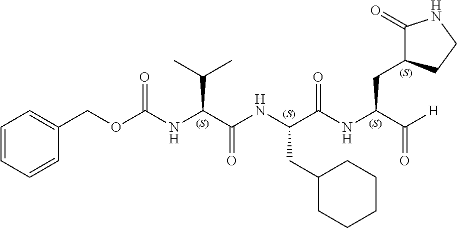 | benzyl ((S)-1-(((S)-3-cyclohexyl-1-oxo- 1-(((S)-1-oxo-3-((S)-2-oxopyrrolidin-3- yl)propan-2-yl)amino)propan-2- yl)amino)-3-methyl-1-oxobutan-2- yl)carbamate |
| MPI7 | 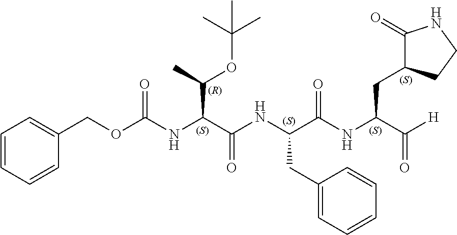 | benzyl ((2S,3R)-3-(tert-butoxy)-1-oxo- 1-(((S)-1-oxo-1-(((S)-1-oxo-3-((S)-2- oxopyrrolidin-3-yl)propan-2-yl)amino)- 3-phenylpropan-2-yl)amino)butan-2- yl)carbamate |
| MPI9 | 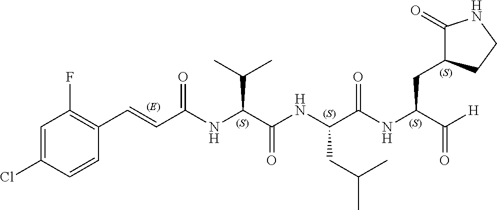 | (S)-2-((S)-2-((E)-3-(4-chloro-2- fluorophenyl)acrylamido)-3- methylbutanamido)-4-methyl-N-((S)-1- oxo-3-((S)-2-oxopyrrolidin-3- yl)propan-2-yl)pentanamide |
| MPI12 | 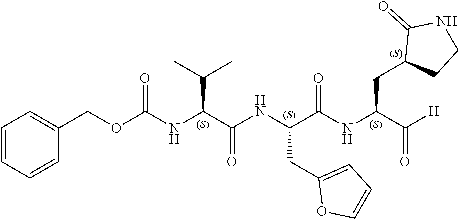 | benzyl ((S)-1-(((S)-3-(furan-2-yl)-1- oxo-1-(((S)-1-oxo-3-((S)-2- oxopyrrolidin-3-yl)propan-2- yl)amino)propan-2-yl)amino)-3-methyl- 1-oxobutan-2-yl)carbamate |
| MPI13 | 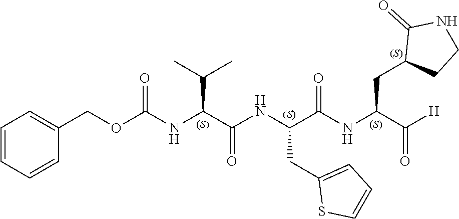 | benzyl ((S)-1-(((S)-3-(furan-2-yl)-1- oxo-1-(((S)-1-oxo-3-((S)-2- oxopyrrolidin-3-yl)propan-2- yl)amino)propan-2-yl)amino)-3-methyl- 1-oxobutan-2-yl)carbamate |
| MPI14 | 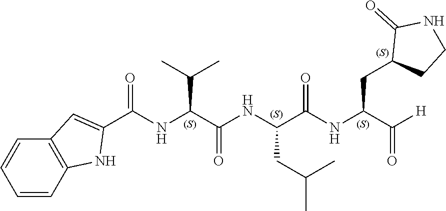 | N-((S)-3-methyl-1-(((S)-4-methyl-1- oxo-1-(((S)-1-oxo-3-((S)-2- oxopyrrolidin-3-yl)propan-2- yl)amino)pentan-2-yl)amino)-1- oxobutan-2-yl)-1H-indole-2- carboxamide |
| MPI15 | 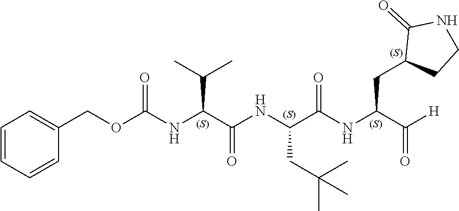 | benzyl ((S)-1-(((S)-4,4-dimethyl-1-oxo- 1-(((S)-1-oxo-3-((S)-2-oxopyrrolidin-3- yl)propan-2-yl)amino)pentan-2- yl)amino)-3-methyl-1-oxobutan-2- yl)carbamate |
| MPI16 | 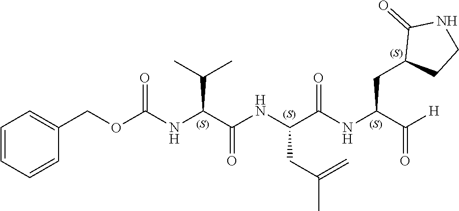 | benzyl ((S)-3-methyl-1-(((S)-4-methyl- 1-oxo-1-(((S)-1-oxo-3-((S)-2- oxopyrrolidin-3-yl)propan-2- yl)amino)pent-4-en-2-yl)amino)-1- oxobutan-2-yl)carbamate |
| MPI17 | 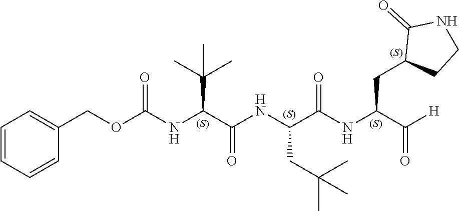 | benzyl ((S)-1-(((S)-4,4-dimethyl-1-oxo- 1-(((S)-1-oxo-3-((S)-2-oxopyrrolidin-3- yl)propan-2-yl)amino)pentan-2- yl)amino)-3,3-dimethyl-1-oxobutan-2- yl)carbamate |
| MPI18 | 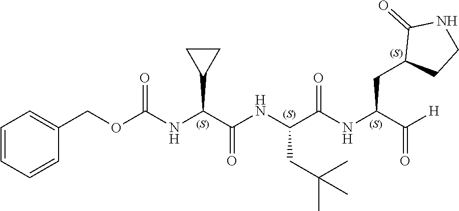 | benzyl ((S)-1-cyclopropyl-2-(((S)-4,4- dimethyl-1-oxo-1-(((S)-1-oxo-3-((S)-2- oxopyrrolidin-3-yl)propan-2- yl)amino)pentan-2-yl)amino)-2- oxoethyl)carbamate |
| MPI19 | 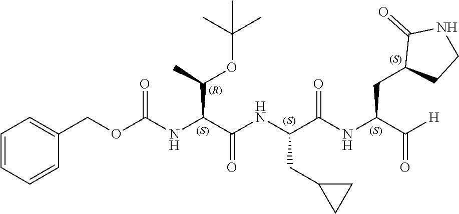 | benzyl ((2S,3R)-3-(tert-butoxy)-1-(((S)- 3-cyclopropyl-1-oxo-1-(((S)-1-oxo-3- ((S)-2-oxopyrrolidin-3-yl)propan-2- yl)amino)propan-2-yl)amino)-1- oxobutan-2-yl)carbamate |
| MPI20 | 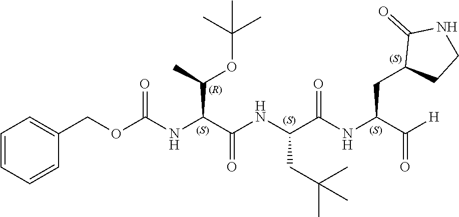 | benzyl ((2S,3R)-3-(tert-butoxy)-1-(((S)- 4,4-dimethyl-1-oxo-1-(((S)-1-oxo-3- ((S)-2-oxopyrrolidin-3-yl)propan-2- yl)amino)pentan-2-yl)amino)-1- oxobutan-2-yl)carbamate |
| MPI21 | 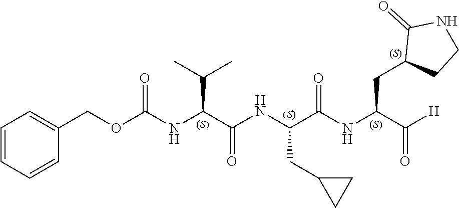 | benzyl ((S)-1-(((S)-3-cyclopropyl-1- oxo-1-(((S)-1-oxo-3-((S)-2- oxopyrrolidin-3-yl)propan-2- yl)amino)propan-2-yl)amino)-3-methyl- 1-oxobutan-2-yl)carbamate |
| MPI22 | 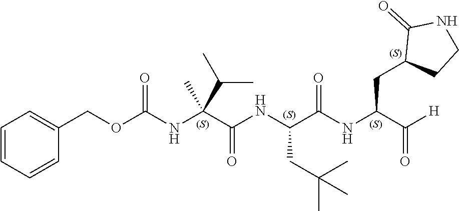 | benzyl ((S)-1-(((S)-4,4-dimethyl-1-oxo- 1-(((S)-1-oxo-3-((S)-2-oxopyrrolidin-3- yl)propan-2-yl)amino)pentan-2- yl)amino)-2,3-dimethyl-1-oxobutan-2- yl)carbamate |
| MPI23 | 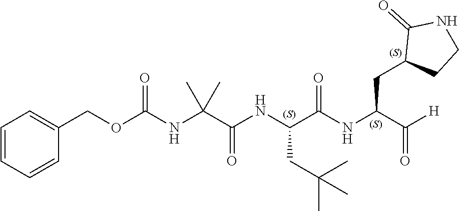 | benzyl (1-(((S)-4,4-dimethyl-1-oxo-1- (((S)-1-oxo-3-((S)-2-oxopyrrolidin-3- yl)propan-2-yl)amino)pentan-2- yl)amino)-2-methyl-1-oxopropan-2- yl)carbamate |
| MPI26 | 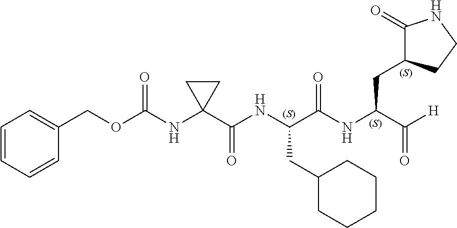 | benzyl (1-(((S)-3-cyclohexyl-1-oxo-1- (((S)-1-oxo-3-((S)-2-oxopyrrolidin-3- yl)propan-2-yl)amino)propan-2- yl)carbamoyl)cyclopropyl)carbamate |
| MPI27 | 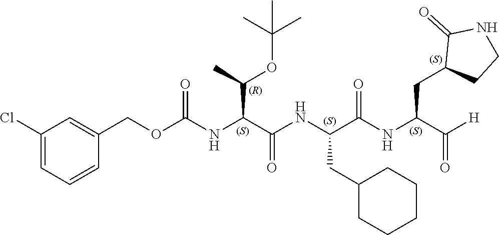 | 3-chlorobenzyl ((2S,3R)-3-(tert- butoxy)-1-(((S)-3-cyclohexyl-1-oxo-1- (((S)-1-oxo-3-((S)-2-oxopyrrolidin-3- yl)propan-2-yl)amino)propan-2- yl)amino)-1-oxobutan-2-yl)carbamate |
| MPI28 | 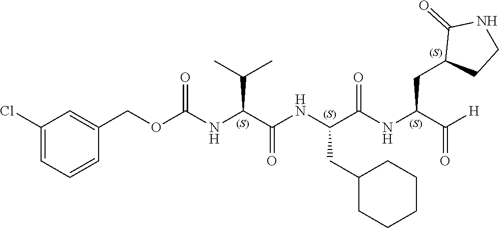 | 3-chlorobenzyl ((S)-1-(((S)-3- cyclohexyl-1-oxo-1-(((S)-1-oxo-3-((S)- 2-oxopyrrolidin-3-yl)propan-2- yl)amino)propan-2-yl)amino)-3-methyl- 1-oxobutan-2-yl)carbamatebenzyl ((S)- 3-cyclohexyl-1-oxo-1-(((S)-1-oxo-3- ((S)-2-oxopyrrolidin-3-yl)propan-2- yl)amino)propan-2-yl)carbamate |
| MPI31 | 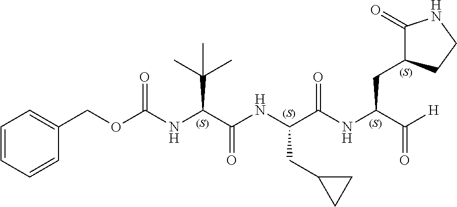 | benzyl ((S)-1-(((S)-3-cyclopropyl-1- oxo-1-(((S)-1-oxo-3-((S)-2- oxopyrrolidin-3-yl)propan-2- yl)amino)propan-2-yl)amino)-3,3- dimethyl-1-oxobutan-2-yl)carbamate |
| MPI32 | 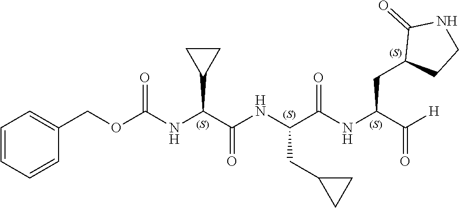 | benzyl ((S)-1-cyclopropyl-2-(((S)-3- cyclopropyl-1-oxo-1-(((S)-1-oxo-3- ((S)-2-oxopyrrolidin-3-yl)propan-2- yl)amino)propan-2-yl)amino)-2- oxoethyl)carbamate |
| MPI33 | 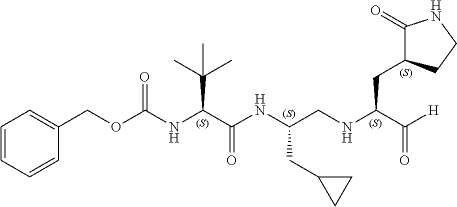 | benzyl ((S)-1-(((S)-1-cyclopropyl-3- (((S)-1-oxo-3-((S)-2-oxopyrrolidin-3- yl)propan-2-yl)amino)propan-2- yl)amino)-3,3-dimethyl-1-oxobutan-2- yl)carbamate |
| MPI34 | 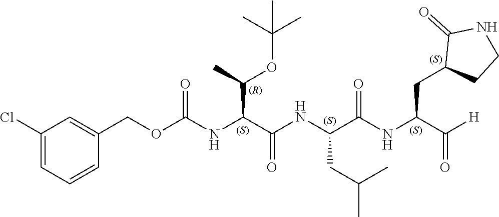 | 3-chlorobenzyl ((2S,3R)-3-(tert- butoxy)-1-(((S)-4-methyl-1-oxo-1- (((S)-1-oxo-3-((S)-2-oxopyrrolidin-3- yl)propan-2-yl)amino)pentan-2- yl)amino)-1-oxobutan-2-yl)carbamate |
| MPI35 | 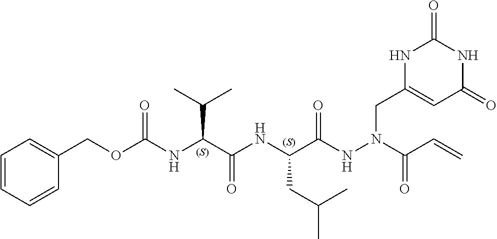 | benzyl ((S)-1-(((S)-1-(2-acryloyl-2- ((2,6-dioxo-1,2,3,6- tetrahydropyrimidin-4- yl)methyl)hydrazineyl)-4-methyl-1- oxopentan-2-yl)amino)-3-methyl-1- oxobutan-2-yl)carbamate |
| MPI36 | 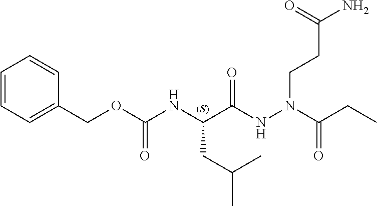 | benzyl (S)-(1-(2-(3-amino-3- oxopropyl)-2-propionylhydrazineyl)-4- methyl-1-oxopentan-2-yl)carbamate |
| MPI37 | 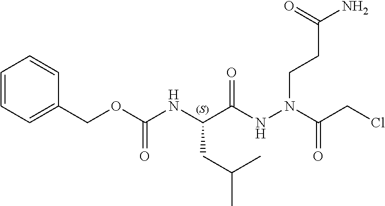 | benzyl (S)-(1-(2-(3-amino-3- oxopropyl)-2-(2- chloroacetyl)hydrazineyl)-4-methyl-1- oxopentan-2-yl)carbamate |
| MPI38 | 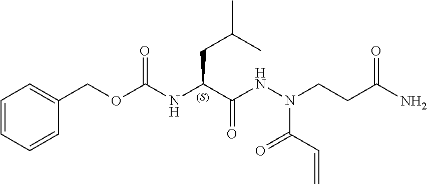 | benzyl (S)-(1-(2-acryloyl-2-(3-amino-3- oxopropyl)hydrazineyl)-4-methyl-1- oxopentan-2-yl)carbamate |
| MPI39 | 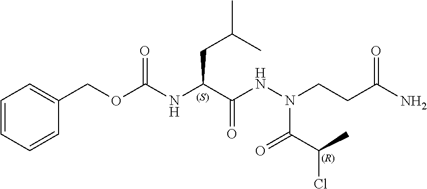 | benzyl ((S)-1-(2-(3-amino-3- oxopropyl)-2-((R)-2- chloropropanoyl)hydrazineyl)-4- methyl-1-oxopentan-2-yl)carbamate |
| MPI40 | 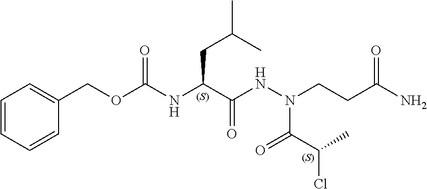 | benzyl ((S)-1-(2-(3-amino-3- oxopropyl)-2-((S)-2- chloropropanoyl)hydrazineyl)-4- methyl-1-oxopentan-2-yl)carbamate |
| MPI41 | 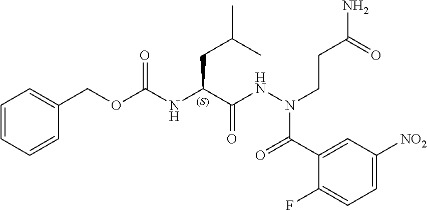 | benzyl (S)-(1-(2-(3-amino-3- oxopropyl)-2-(2-fluoro-5- nitrobenzoyl)hydrazineyl)-4-methyl-1- oxopentan-2-yl)carbamate |
| MPI42 | 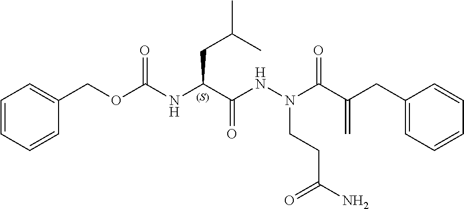 | benzyl (S)-(1-(2-(3-amino-3- oxopropyl)-2-(2- benzylacryloyl)hydrazineyl)-4-methyl- 1-oxopentan-2-yl)carbamate |
| MPI43 | 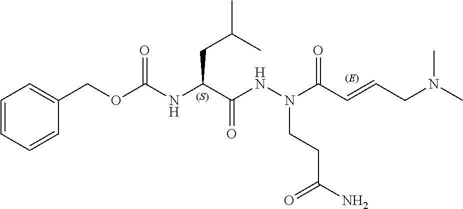 | benzyl (S,E)-(1-(2-(3-amino-3- oxopropyl)-2-(4-(dimethylamino)but-2- enoyl)hydrazineyl)-4-methyl-1- oxopentan-2-yl)carbamate |
| MPI44 | 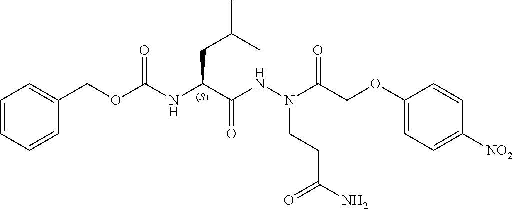 | benzyl (S)-(1-(2-(3-amino-3- oxopropyl)-2-(2-(4- nitrophenoxy)acetyl)hydrazineyl)-4- methyl-1-oxopentan-2-yl)carbamate |
| MPI45 | 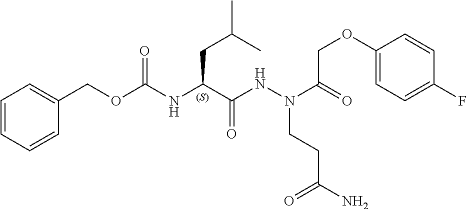 | benzyl (S)-(1-(2-(3-amino-3- oxopropyl)-2-(2-(4- fluorophenoxy)acetyl)hydrazineyl)-4- methyl-1-oxopentan-2-yl)carbamate |
| MPI46 | 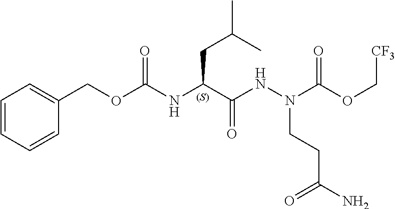 | 2,2,2-trifluoroethyl 1-(3-amino-3- oxopropyl)-2-(((benzyloxy)carbonyl)- L-leucyl)hydrazine-1-carboxylate |
| MPI47 | 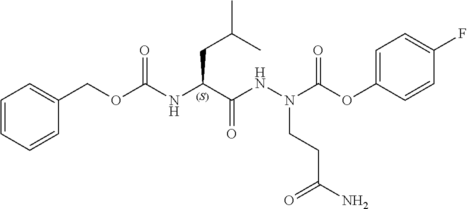 | 4-fluorophenyl 1-(3-amino-3- oxopropyl)-2-(((benzyloxy)carbonyl)- L-leucyl)hydrazine-1-carboxylate |
| MPI50 | 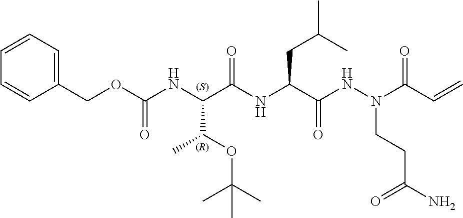 | benzyl ((2S,3R)-1-(((S)-1-(2-acryloyl- 2-(3-amino-3-oxopropyl)hydrazineyl)- 4-methyl-1-oxopentan-2-yl)amino)-3- (tert-butoxy)-1-oxobutan-2- yl)carbamate |
| MPI51 | 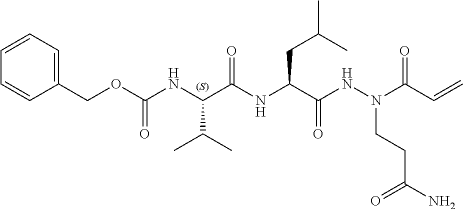 | benzyl ((S)-1-(((S)-1-(2-acryloyl-2-(3- amino-3-oxopropyl)hydrazineyl)-4- methyl-1-oxopentan-2-yl)amino)-3- methyl-1-oxobutan-2-yl)carbamate |
| MPI52 | 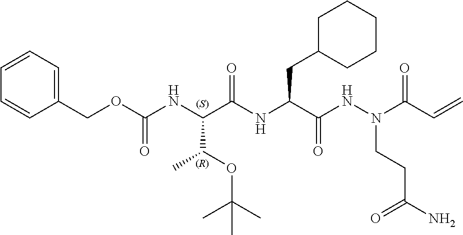 | benzyl ((2S,3R)-1-(((S)-1-(2-acryloyl- 2-(3-amino-3-oxopropyl)hydrazineyl)- 3-cyclohexyl-1-oxopropan-2-yl)amino)- 3-(tert-butoxy)-1-oxobutan-2- yl)carbamate |
| MPI53 | 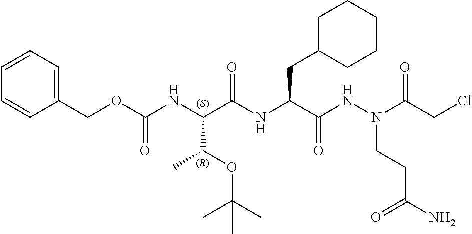 | benzyl ((2S,3R)-1-(((S)-1-(2-(3-amino- 3-oxopropyl)-2-(2- chloroacetyl)hydrazineyl)-3- cyclohexyl-1-oxopropan-2-yl)amino)-3- (tert-butoxy)-1-oxobutan-2- yl)carbamate |
| MPI54 | 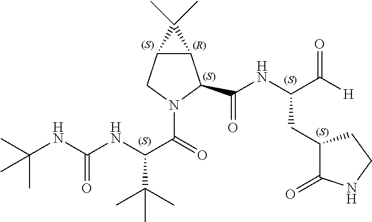 | (1R,2S,5S)-3-((S)-2-(3-(tert- butyl)ureido)-3,3-dimethylbutanoyl)- 6,6-dimethyl-N-((S)-1-oxo-3-((S)-2- oxopyrrolidin-3-yl)propan-2-yl)-3- azabicyclo[3.1.0]hexane-2-carboxamide |
| MPI55 | 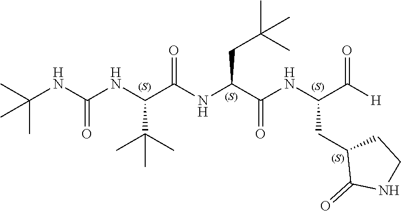 | (S)-2-((S)-2-(3-(tert-butyl)ureido)-3,3- dimethylbutanamido)-4,4-dimethyl-N- ((S)-1-oxo-3-((S)-2-oxopyrrolidin-3- yl)propan-2-yl)pentanamide |
| MPI56 | 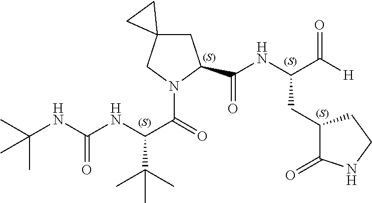 | (S)-5-((S)-2-(3-(tert-butyl)ureido)-3,3- dimethylbutanoyl)-N-((S)-1-oxo-3-((S)- 2-oxopyrrolidin-3-yl)propan-2-yl)-5- azaspiro[2.4]heptane-6-carboxamide |
| MPI57 | 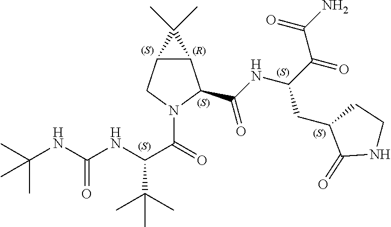 | (1R,2S,5S)-N-((S)-4-amino-3,4-dioxo- 1-((S)-2-oxopyrrolidin-3-yl)butan-2- yl)-3-((S)-2-(3-(tert-butyl)ureido)-3,3- dimethylbutanoyl)-6,6-dimethyl-3- azabicyclo[3.1.0]hexane-2-carboxamide |
| MPI58 | 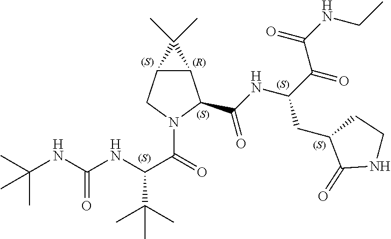 | (1R,2S,5S)-3-((S)-2-(3-(tert- butyl)ureido)-3,3-dimethylbutanoyl)-N- ((S)-4-(ethylamino)-3,4-dioxo-1-((S)-2- oxopyrrolidin-3-yl)butan-2-yl)-6,6- dimethyl-3-azabicyclo[3.1.0]hexane-2- carboxamide |
| MPI59 | 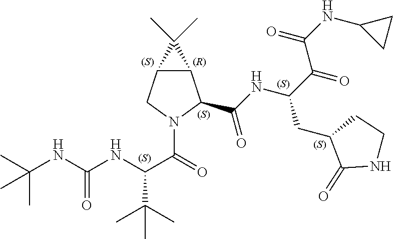 | (1R,2S,5S)-3-((S)-2-(3-(tert- butyl)ureido)-3,3-dimethylbutanoyl)-N- ((S)-4-(cyclopropylamino)-3,4-dioxo-1- ((S)-2-oxopyrrolidin-3-yl)butan-2-yl)- 6,6-dimethyl-3- azabicyclo[3.1.0]hexane-2-carboxamide |
| MPI60 | 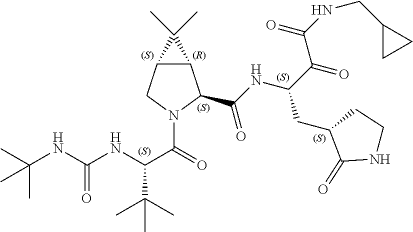 | (1R,2S,5S)-3-((S)-2-(3-(tert- butyl)ureido)-3,3-dimethylbutanoyl)-N- ((S)-4-((cyclopropylmethyl)amino)-3,4- dioxo-1-((S)-2-oxopyrrolidin-3- yl)butan-2-yl)-6,6-dimethyl-3- azabicyclo[3.1.0]hexane-2- carboxamide |
| MPI61 | 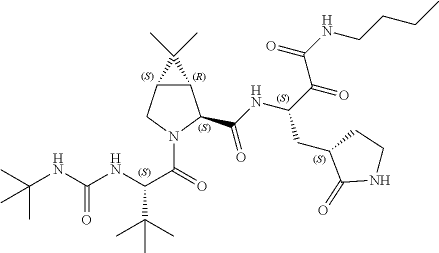 | (1R,2S,5S)-3-((S)-2-(3-(tert- butyl)ureido)-3,3-dimethylbutanoyl)-N- ((S)-4-(butylamino)-3,4-dioxo-1-((S)-2- oxopyrrolidin-3-yl)butan-2-yl)-6,6- dimethyl-3-azabicyclo[3.1.0]hexane-2- carboxamide |
| MPI62 | 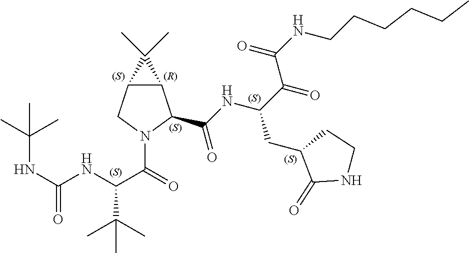 | (1R,2S,5S)-3-((S)-2-(3-(tert- butyl)ureido)-3,3-dimethylbutanoyl)-N- ((S)-4-(hexylamino)-3,4-dioxo-1-((S)- 2-oxopyrrolidin-3-yl)butan-2-yl)-6,6- dimethyl-3-azabicyclo[3.1.0]hexane-2- carboxamide |
| MPI63 | 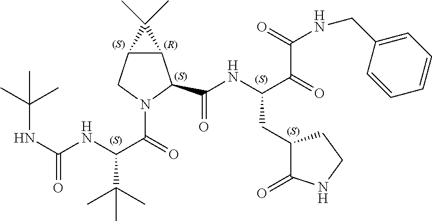 | (1R,2S,5S)-N-((S)-4-(benzylamino)- 3,4-dioxo-1-((S)-2-oxopyrrolidin-3- yl)butan-2-yl)-3-((S)-2-(3-(tert- butyl)ureido)-3,3-dimethylbutanoyl)- 6,6-dimethyl-3- azabicyclo[3.1.0]hexane-2-carboxamide |
| MPI64 | 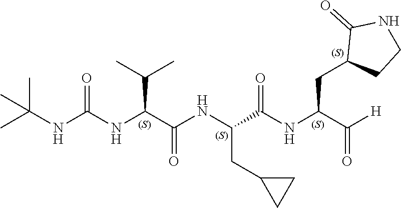 | (S)-2-(3-(tert-butyl)ureido)-N-((S)-3- cyclopropyl-1-oxo-1-(((S)-1-oxo-3- ((S)-2-oxopyrrolidin-3-yl)propan-2- yl)amino)propan-2-yl)-3- methylbutanamide |
| MPI65 | 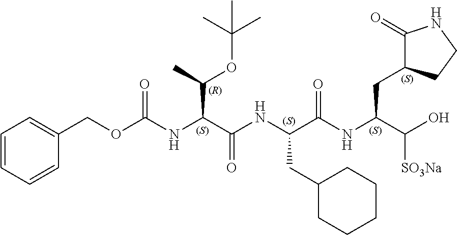 | sodium (5S,8S,11S)-5-((R)-1-(tert- butoxy)ethyl)-8-(cyclohexylmethyl)-12- hydroxy-3,6,9-trioxo-11-(((S)-2- oxopyrrolidin-3-yl)methyl)-1-phenyl-2- oxa-4,7,10-triazadodecane-12-sulfonate |
| MPI66 | 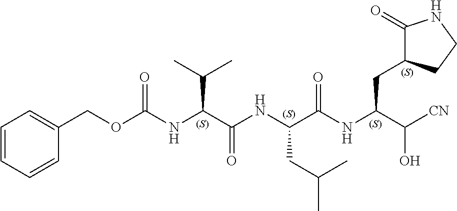 | benzyl ((2S)-1-(((2S)-1-(((2S)-1-cyano- 1-hydroxy-3-((S)-2-oxopyrrolidin-3- yl)propan-2-yl)amino)-4-methyl-1- oxopentan-2-yl)amino)-3-methyl-1- oxobutan-2-yl)carbamate |
| MPI67 | 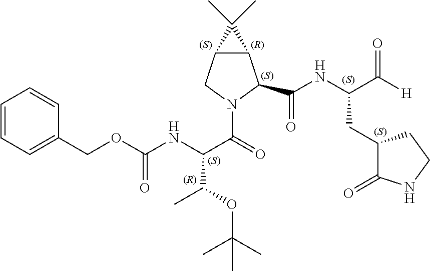 | benzyl ((2S,3R)-3-(tert-butoxy)-1- ((1R,2S,5S)-6,6-dimethyl-2-(((S)-1- oxo-3-((S)-2-oxopyrrolidin-3- yl)propan-2-yl)carbamoyl)-3- azabicyclo[3.1.0]hexan-3-yl)-1- oxobutan-2-yl)carbamate |
| MPI68 | 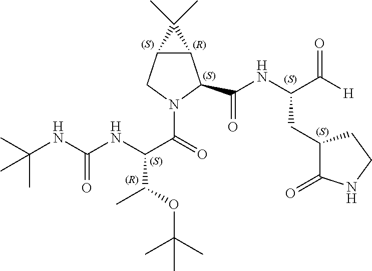 | (1R,2S,5S)-3-(O-(tert-butyl)-N-(tert- butylcarbamoyl)-L-threonyl)-6,6- dimethyl-N-((S)-1-oxo-3-((S)-2- oxopyrrolidin-3-yl)propan-2-yl)-3- azabicyclo[3.1.0]hexane-2-carboxamide |
| MPI69 | 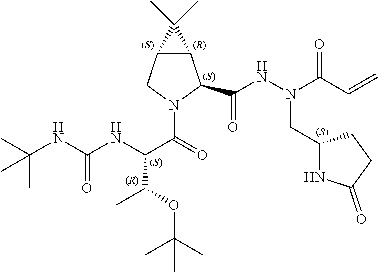 | 1-((2S,3R)-1-((lR,2S,5S)-2-(2-acryloyl- 2-(((S)-5-oxopyrrolidin-2- yl)methyl)hydrazine-1-carbonyl)-6,6- dimethyl-3-azabicyclo[3.1.0]hexan-3- yl)-3-(tert-butoxy)-1-oxobutan-2-yl)-3- (tert-butyl)urea |
| MPI70 | 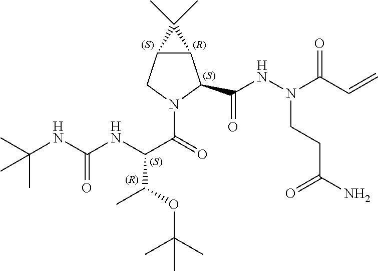 | 3-(1-acryloyl-2-((1R,2S,5S)-3-(O-(tert- butyl)-N-(tert-butylcarbamoyl)-L- threonyl)-6,6-dimethyl-3- azabicyclo[3.1.0]hexane-2- carbonyl)hydrazineyl)propanamide |
| MPI72 | 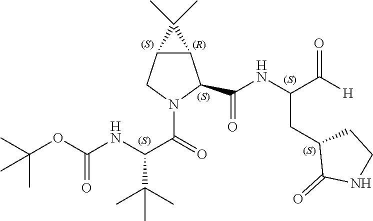 | tert-butyl ((S)-1-((1R,2S,5S)-6,6- dimethyl-2-(((S)-1-oxo-3-((S)-2- oxopyrrolidin-3-yl)propan-2- yl)carbamoyl)-3- azabicyclo[3.1.0]hexan-3-yl)-3,3- dimethyl-1-oxobutan-2-yl)carbamate |
| MPI73 | 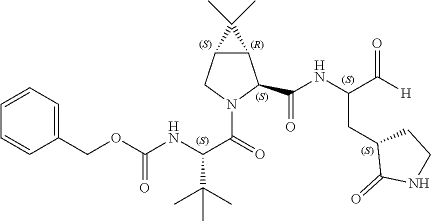 | benzyl ((S)-1-((1R,2S,5S)-6,6- dimethyl-2-(((S)-1-oxo-3-((S)-2- oxopyrrolidin-3-yl)propan-2- yl)carbamoyl)-3- azabicyclo[3.1.0]hexan-3-yl)-3,3- dimethyl-1-oxobutan-2-yl)carbamate |
| MPI74 | 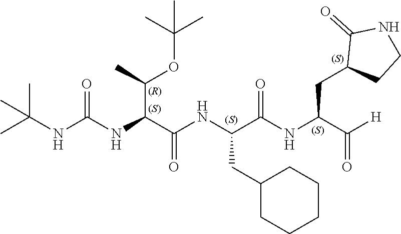 | (2S,3R)-3-(tert-butoxy)-2-(3-(tert- butyl)ureido)-N-((S)-3-cyclohexyl-1- oxo-1-(((S)-1-oxo-3-((S)-2- oxopyrrolidin-3-yl)propan-2- yl)amino)propan-2-yl)butanamide |
| MPI75 | 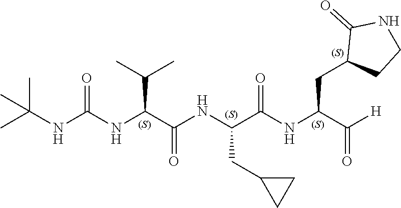 | (S)-2-(3-(tert-butyl)ureido)-N-((S)-3- cyclopropyl-1-oxo-1-(((S)-1-oxo-3- ((S)-2-oxopyrrolidin-3-yl)propan-2- yl)amino)propan-2-yl)-3- methylbutanamide |
| MPI76 | 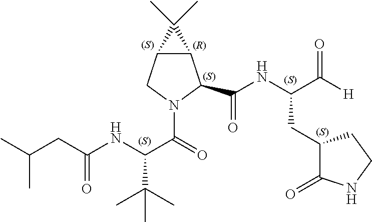 | (1R,2S,5S)-3-((S)-3,3-dimethyl-2-(3- methylbutanamido)butanoyl)-6,6- dimethyl-N-((S)-1-oxo-3-((S)-2- oxopyrrolidin-3-yl)propan-2-yl)-3- azabicyclo[3.1.0]hexane-2-carboxamide |
| MPI84 | 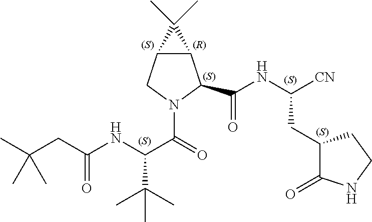 | (1R,2S,5S)-N-((S)-1-cyano-2-((S)-2- oxopyrrolidin-3-yl)ethyl)-3-((S)-2-(3,3- dimethylbutanamido)-3,3- dimethylbutanoyl)-6,6-dimethyl-3- azabicyclo[3.1.0]hexane-2-carboxamide |
| MPI85 | 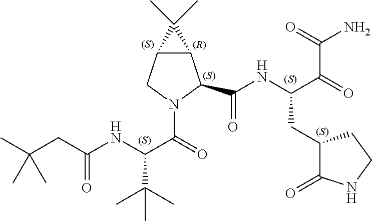 | (1R,2S,5S)-N-((S)-4-amino-3,4-dioxo- 1-((S)-2-oxopyrrolidin-3-yl)butan-2- yl)-3-((S)-2-(3,3-dimethylbutanamido)- 3,3-dimethylbutanoyl)-6,6-dimethyl-3- azabicyclo[3.1.0]hexane-2-carboxamide |
| MPI86 | 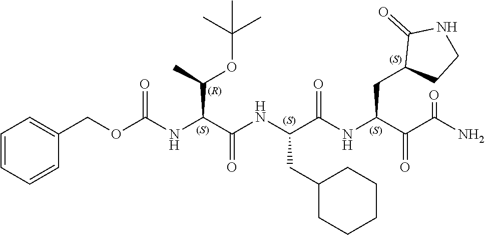 | benzyl ((2S,3R)-1-(((S)-1-(((S)-4- amino-3,4-dioxo-1-((S)-2- oxopyrrolidin-3-yl)butan-2-yl)amino)- 3-cyclohexyl-1-oxopropan-2-yl)amino)- 3-(tert-butoxy)-1-oxobutan-2- yl)carbamate |
| MPI87 | 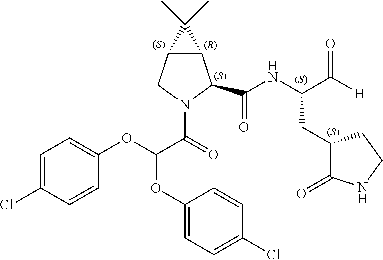 | (1R,2S,5S)-3-(2,2-bis(4- chlorophenoxy)acetyl)-6,6-dimethyl-N- ((S)-1-oxo-3-((S)-2-oxopyrrolidin-3- yl)propan-2-yl)-3- azabicyclo[3.1.0]hexane-2-carboxamide |
| MPI88 | 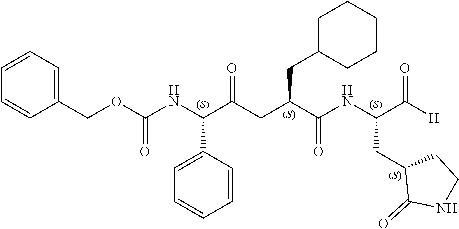 | benzyl ((S)-2-(((S)-3-cyclohexyl-1-oxo- 1-(((S)-1-oxo-3-((S)-2-oxopyrrolidin-3- yl)propan-2-yl)amino)propan-2- yl)amino)-2-oxo-1- phenylethyl)carbamate |
| MPI89 | 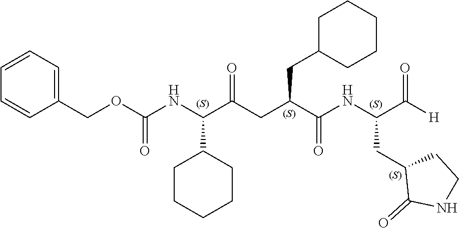 | benzyl ((S)-1-cyclohexyl-2-(((S)-3- cyclohexyl-1-oxo-1-(((S)-1-oxo-3-((S)- 2-oxopyrrolidin-3-yl)propan-2- yl)amino)propan-2-yl)amino)-2- oxoethyl)carbamate |
| MPI90 | 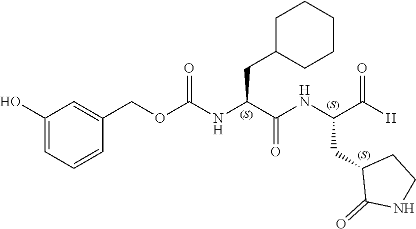 | 3-hydroxybenzyl ((S)-3-cyclohexyl-1- oxo-1-(((S)-1-oxo-3-((S)-2- oxopyrrolidin-3-yl)propan-2- yl)amino)propan-2-yl)carbamate |
| MPI91 | 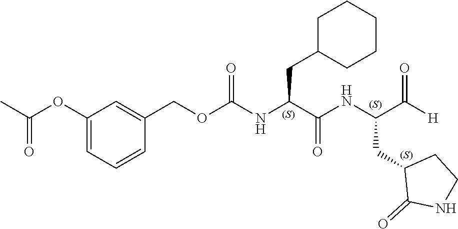 | 3-(((((S)-3-cyclohexyl-1-oxo-1-(((S)-1- oxo-3-((S)-2-oxopyrrolidin-3- yl)propan-2-yl)amino)propan-2- yl)carbamoyl)oxy)methyl)phenyl acetate |
| MPI92 | 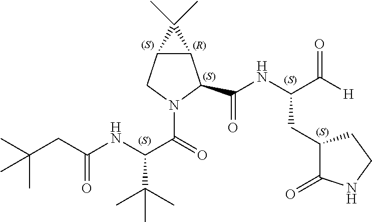 | (1R,2S,5S)-3-((S)-2-(3,3- dimethylbutanamido)-3,3- dimethylbutanoyl)-6,6-dimethyl-N- ((S)-1-oxo-3-((S)-2-oxopyrrolidin-3- yl)propan-2-yl)-3- azabicyclo[3.1.0]hexane-2-carboxamide |
| MPI94 | 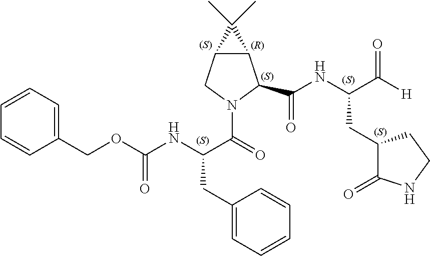 | benzyl ((S)-1-((1R,2S,5S)-6,6- dimethyl-2-(((S)-1-oxo-3-((S)-2- oxopyrrolidin-3-yl)propan-2- yl)carbamoyl)-3- azabicyclo[3.1.0]hexan-3-yl)-1-oxo-3- phenylpropan-2-yl)carbamate |
| MPI95 | 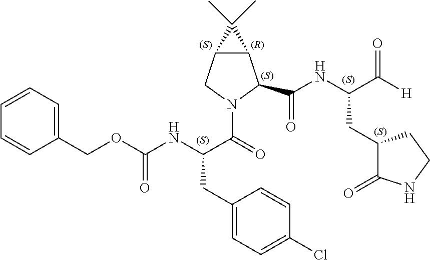 | benzyl ((S)-3-(4-chlorophenyl)-1- ((1R,2S,5S)-6,6-dimethyl-2-(((S)-1- oxo-3-((S)-2-oxopyrrolidin-3- yl)propan-2-yl)carbamoyl)-3- azabicyclo[3.1.0]hexan-3-yl)-1- oxopropan-2-yl)carbamate |
| MPI100 | 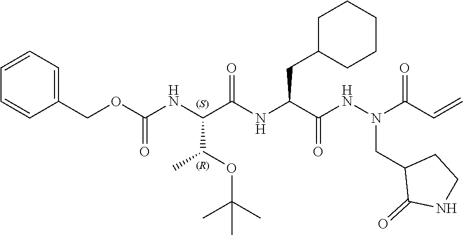 | benzyl ((2S,3R)-1-(((2S)-1-(2-acryloyl- 2-((2-oxopyrrolidin-3- yl)methyl)hydrazineyl)-3-cyclohexyl-1- oxopropan-2-yl)amino)-3-(tert-butoxy)- 1-oxobutan-2-yl)carbamate |
| MPI101 | 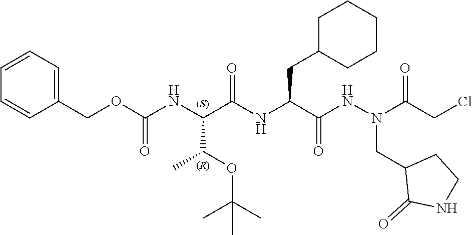 | benzyl ((2S,3R)-3-(tert-butoxy)-1- (((2S)-1-(2-(2-chloroacetyl)-2-((2- oxopyrrolidin-3- yl)methyl)hydrazineyl)-3-cyclohexyl-1- oxopropan-2-yl)amino)-1-oxobutan-2- yl)carbamate |
| MPI102 | 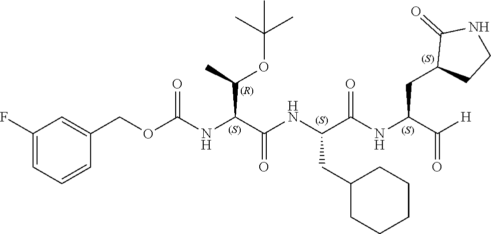 | 3-fluorobenzyl ((2S,3R)-3-(tert- butoxy)-1-(((S)-3-cyclohexyl-1-oxo-1- (((S)-1-oxo-3-((S)-2-oxopyrrolidin-3- yl)propan-2-yl)amino)propan-2- yl)amino)-1-oxobutan-2-yl)carbamate |
| MPI103 | 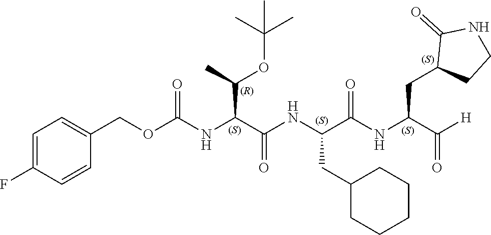 | 4-fluorobenzyl ((2S,3R)-3-(tert- butoxy)-1-(((S)-3-cyclohexyl-1-oxo-1- (((S)-1-oxo-3-((S)-2-oxopyrrolidin-3- yl)propan-2-yl)amino)propan-2- yl)amino)-1-oxobutan-2-yl)carbamate |

Additional compounds discussed herein can be found in the following Table B.

TABLE B

| Compound | Structure | Name |
|---|---|---|
| MPI1 | 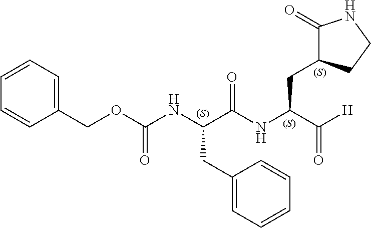 | benzyl ((S)-1-oxo-1-(((S)-1- oxo-3-((S)-2-oxopyrrolidin-3- yl)propan-2-yl)amino)-3- phenylpropan-2-yl)carbamate |
| MPI2 | 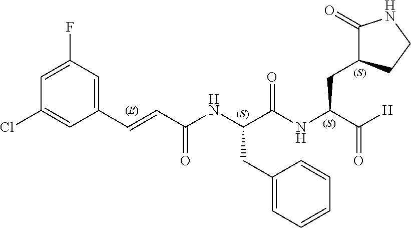 | (E)-3-(3-chloro-5- fluorophenyl)-N-((S)-1-oxo-1- (((S)-1-oxo-3-((S)-2- oxopyrrolidin-3-yl)propan-2- yl)amino)-3-phenylpropan-2- yl)acrylamide |
| MPI3 | 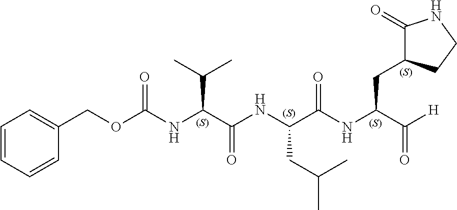 | benzyl ((S)-3-methyl-1-(((S)- 4-methyl-1-oxo-1-(((S)-1-oxo- 3-((S)-2-oxopyrrolidin-3- yl)propan-2-yl)amino)pentan- 2-yl)amino)-1-oxobutan-2- yl)carbamate |
| MPI6 | 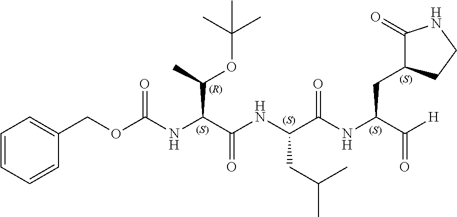 | benzyl ((2S,3R)-3-(tert- butoxy)-1-(((S)-4-methyl-1- oxo-1-(((S)-1-oxo-3-((S)-2- oxopyrrolidin-3-yl)propan-2- yl)amino)pentan-2-yl)amino)- 1-oxobutan-2-yl)carbamate |
| MPI8 | 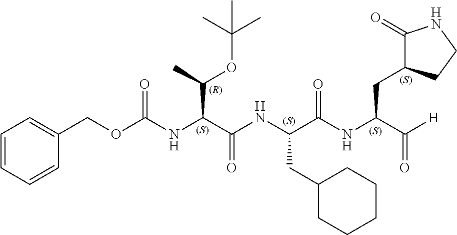 | benzyl ((2S,3R)-3-(tert- butoxy)-1-(((S)-3-cyclohexyl- 1-oxo-1-(((S)-1-oxo-3-((S)-2- oxopyrrolidin-3-yl)propan-2- yl)amino)propan-2-yl)amino)- 1-oxobutan-2-yl)carbamate |
| MPI10 | 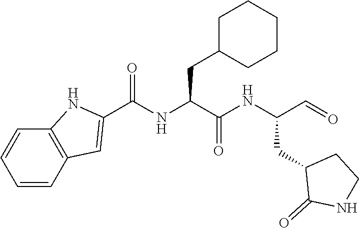 [SMILES: O=C[C@H](C[C@@H]1CCNC1=O)NC(=O)[C@H](CC1CCCCC1)NC(=O)c1cc2ccccc2[nH]1] | N-((S)-3-cyclohexyl-1-oxo-1- (((S)-1-oxo-3-((S)-2- oxopyrrolidin-3-yl)propan-2- yl)amino)propan-2-yl)-1H- indole-2-carboxamide |
| GC376 | 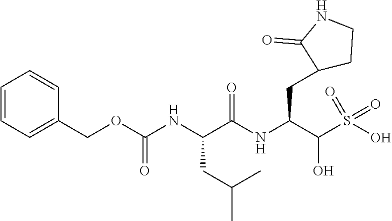 [SMILES: CC(C)C[C@H](NC(=O)OCc1ccccc1)C(=O)N[C@@H](CC1CCNC1=O)C(O)S(=O)(=O)O] | (2S)-2-((S)-2- (((benzyloxy)carbonyl)amino)- 4-methylpentanamido)-1- hydroxy-3-(2-oxopyrrolidin-3- yl)propane-1-sulfonic acid |

Those skilled in the art will recognize that the species listed or illustrated herein are not exhaustive, and that additional species within the scope of these defined terms may also be

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the invention are sterile compositions. Pharmaceutical compositions may be prepared using compounding techniques known or that become available to those skilled in the art.

Sterile compositions are also contemplated by the invention, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the invention may be administered by a suitable route of delivery, such as oral, parenteral, rectal, nasal, topical, or ocular routes, or by inhalation. Preferably, the compositions are formulated for intravenous or oral administration.

For oral administration, the compounds the invention may be provided in a solid form, such as a tablet or capsule, or as a solution, emulsion, or suspension. To prepare the oral compositions, the compounds of the invention may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present invention are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the invention may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include those caused by SARS-CoV-2, SARS-CoV, MERS-CoV, Ebola virus, Paramyxoviruses, Bunyaviruses (Bunyavirales), Togaviruses, Filoviruses, Picornaviruses, Flaviviruses. In some example, the disease is caused by SARS-CoV-2.

In one aspect, the compounds and pharmaceutical compositions of the invention specifically target SC2M^{Pro}. Thus, these compounds and pharmaceutical compositions can be used to prevent, reverse, slow, or inhibit the activity of this protease. In preferred embodiments, methods of treatment include treating viral infections. In other embodiments, methods are for treating viral infections caused by COVID-19.

In the inhibitory methods of the invention, an “effective amount” means an amount sufficient to inhibit the target protein. Measuring such target modulation may be performed by routine analytical methods such as those described below. Such modulation is useful in a variety of settings, including in vitro assays.

In treatment methods according to the invention, an “effective amount” means an amount or dose sufficient to generally bring about the desired therapeutic benefit in subjects needing such treatment. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present invention or may be included with a compound of the present invention in a single pharmaceutical composition. The additional active ingredients may be administered simultaneously with, prior to, or after administration of a compound of the present invention.

Combination agents include additional active ingredients are those that are known or discovered to be effective in treating the diseases and disorders described herein, including those active against another target associated with the disease. For example, compositions and formulations of the invention, as well as methods of treatment, can further comprise other drugs or pharmaceuticals, e.g., other active agents useful for treating or palliative for the target diseases or related symptoms or conditions. For viral infections, additional such agents include, but are not limited to, remdesivir, favipiravir, ribavirin, monoclonal antibodies, dexamethasone, interferon, umifenovir, oseltamivir, lopinavir, and ritonavir. The pharmaceutical compositions of the invention may additionally comprise one or more of such active agents, and methods of treatment may additionally comprise administering an effective amount of one or more of such active agents.

The COVID-19 pathogen, SARS-CoV-2, requires its main protease (SC2M^{Pro}) to digest two of its translated long polypeptides to form a number of mature proteins that are essential for viral replication and pathogenesis. Inhibition of this vital proteolytic process is effective in preventing the virus from replication in infected cells and therefore provides a potential COVID-19 treatment option. A series of SC2M^{Pro} inhibitors were synthesized that contain β-(S-2-oxopyrrolidin-3-yl)-alaninal (Opal) for the formation of a reversible covalent bond with the SC2M^{Pro} active site cysteine C145. Most inhibitors display high potency with Ki values at or below 100 nM. One of the most potent compound MPI3 has as a Ki value as 8.3 nM. Crystallographic analyses of SC2M^{Pro} bound to many inhibitors indicated both formation of a covalent bond with C145 and structural rearrangement from the apoenzyme to accommodate the inhibitors. Virus inhibition assays revealed that several inhibitors have high potency in inhibiting the SARS-CoV-2-induced cytopathogenic effect in both Vero E6 and A549/ACE2 cells. Two inhibitors MPI5 and MPI8 completely prevented the SARS-CoV-2-induced cytopathogenic effect in Vero E6 cells at 2.5-5 μM and A549/ACE2 cells at 0.16-0.31 μM. These results also revealed that MPI5, 6, 7, and 8 have high cellular and antiviral potency with both IC₅₀ and EC₅₀ values respectively below 1 μM. As the one with the highest cellular and antiviral potency among all tested compounds, MPI8 has a remarkable cellular M^{Pro} inhibition IC₅₀ value of 31 nM that matches closely to its strong antiviral effect with an EC₅₀ value of 30 nM.

Chemical Synthesis

Exemplary chemical entities useful in methods of the description will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

### Abbreviations

The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc or EA | ethyl acetate |
| MHz | megahertz |
| Ppm | parts per million |
| Δ | chemical shift |
| S | singlet |
| D | doublet |
| T | triplet |
| Q | quartet |
| Quin | quintet |
| Br | broad |
| M | multiplet |
| Hz | hertz |
| THF | tetrahydrofuran |
| ° C. | degrees Celsius |
| R_{f} | retardation factor |
| N | normal |
| J | coupling constant |
| DMSO-d₆ | deuterated dimethyl sulfoxide |
| EtOH | ethanol |
| DIPEA | N,N-diisopropylethylamine |
| min | minutes |
| hr | hours |
| TLC | thin layer chromatography |
| M | molar |
| MS | mass spectrum |
| m/z | mass-to-charge ratio |
| DMAP | 4-(dimethylamino)pyridine |
| μM | micromolar |
| IC₅₀ | half maximal inhibitory concentration |
| U/mL | units of activity per milliliter |
| MOM-Cl | methoxymethyl chloride |
| DCM | dichloromethane |
| DMF | N,N-dimethylformamide |
| Cbz | Benzyl formate |
| PEG | polyethylene glycol |
| HMDS | hexamethyldisilazide |
| THF | tetrahydrofuran |
| HATU | Hexafluorophosphate Azabenzotriazole |
| | Tetramethyl Uronium |
| DMP or Dess-Martin reagent | Dess-Martin periodinane |
| RT | room temperature |

### Example 1 The Design of β-(S-2-oxopyrrolidin-3-yl)-alaninal (Opal)-Based, Reversible Covalent Inhibitors for SC2M^{Pro}

SC1M^{Pro} has a large active site that consists of several smaller pockets for the recognition of residues at P1, P2, P4, and P3′ positions in a protein substrate. P4 is typically a small hydrophobic residue while P2 and P3′ are large. For all Nsps that are processed by SC1M^{Pro} and SC2M^{Pro}, Gln is the P1 residue at their cleavage sites. In order to bind the P1 Gln, SC1M^{Pro} forms strong van der Waals interactions with the Gln side chain, and also utilizes two hydrogen bonds with the Gln side chain amide oxygen and α-carbonyl oxygen atoms. The enhanced potency from the use of the β-S-2-oxopyrrolidine-containing Gln analog is most probably due to the reduction of entropy loss during the binding of SC1M^{pro} to the more rigid lactam compared to the flexible Gln. Although converting the scissile backbone amide to a Michael acceptor in a SC1M^{Pro} ligand turns it into a covalent inhibitor, it eliminates the critical hydrogen bond between the P1 α-carbonyl oxygen and SC1M^{Pro}. Therefore, most Michael acceptor inhibitors developed for SC1M^{Pro} and recently for SC2M^{Pro} tend to have efficacy with low micromolar or submicromolar IC₅₀ values rather than low nanomolar levels.

Synthesis and IC₅₀ Characterization of SC2M^{Pro} Inhibitors (MPIs).

GC376 (available from Cayman Chemical) has confirmed potency against SC1M^{Pro}. Two similar dipeptidyl compounds were designed (MPI1-2) and synthesized. Both MPI1 and MPI2 have Phe at the P2 site, which was previously shown to contribute to strong bonding to SC1M^{Pro}. MPI2 has also an o-fluoro-p-chlorocinnamyl group as an N-terminal cap. This group is more rigid than the CBZ group and therefore possibly introduces a strong interaction with the P4-binding pocket in SC2M^{Pro}. To characterize IC₅₀ values of all three molecules for inhibition of SC2M^{Pro}, a 6×His-SUMO-SC2M^{Pro} fusion protein was expressed in E. coli and purified and digested this protein with SUMO protease to obtain intact SC2M^{Pro} with more than 95% purity. A fluorescent peptide assay was used to measure the IC₅₀ values for GC376, MPI1, and MPI2 as 31±4, 100±23, and 103±14 nM, respectively. The IC₅₀ value for GC376 agrees well with that from Ma et al. By adding one more residue to the design of inhibitors, additional interactions with SC2M^{Pro} might be achieved to improve potency. In the design of SC1M^{Pro} inhibitors, Leu, Phe, and Cha (cyclohexylalanine) are three residues used at the P2 site and Val and Thr(tBu) (O-tert-butyl-threonine) are two residues used at the P3 site. Installation of these residues at two sites and including CBZ as a N-terminal cap led to the design of six compounds MPI3-8. MPI9 was added that has an o-fluoro-p-chlorocinnamyl cap to this series to compare the effect of the two N-terminal caps on the inhibitor potency for SC2M^{Pro}. Seven compounds were synthesized as described herein and their IC₅₀ values were characterized using the fluorescent peptide assay. All inhibitors have IC₅₀ values below 100 nM, except for MPI8 that has an IC₅₀ value as 105±22 nM. MPI3 had an IC₅₀ value as 8.5±1.5 nM, followed by MPI4 and MPI5 with IC₅₀ values as 15±5 and 33±2 nM, respectively. MPI10 (available from Cayman Chemical) was prepared according to the procedure in Dai et al. and used it as a positive control in our enzyme and viral inhibition analyses. Using the fluorescent peptide assay, the IC₅₀ value of MPI10 was 31±3 nM. From the perspective of enzyme inhibition, Leu and Val residues at P2 and P3 sites in an inhibitor showed improved affinity for SC2M^{Pro} and CBZ also enhances affinity compared to the o-fluoro-p-chlorocinnamyl as a N-terminal capping group.

Structural Characterization of SC2M^{Pro} Interactions with Opal-Based Inhibitors.

In order to understand how our designed inhibitors interact with SC2M^{Pro} at its active site, crystallization conditions were screened for apo-SC2M^{Pro}, soaked apo-SC2M^{Pro} crystals with different inhibitors, and determined the crystal structures of these inhibitors in complex with SC2M^{Pro}. A Hampton Research Crystal Screen and Index kits was used to perform initial screening and identified several conditions that yielded single crystals of apo-SC2M^{Pro}. For all conditions, crystals were in a thin plate shape. The best crystallization condition contained 0.2 M dibasic ammonium phosphate and 17% PEG 3,350. The structure of apo-SC2M^{Pro} against diffraction data was refined to 1.6 Å resolution (PDB: 7JPY). In the apoenzyme crystals, SC2M^{Pro} existed as a monomer in the crystallographic asymmetric unit and packed relatively densely. The active site of each monomer stacked upon another monomer. This close contact and dense protein packing made the diffusion of inhibitors to the active site quite slow. Apo SC2M^{Pro} crystals were soaked with 9 synthesized inhibitors and their X-ray diffraction data was collected and processed for structural determination. For crystals soaked with the inhibitors for just 2 h, no observable ligand electron density was found at the enzyme active site. For seven inhibitors including MPI1 and MPI3-8, a two-day soaking was performed and clear electron density was observed in the difference maps in the active site of the enzyme. For MPI2 and MPI9, it was not able to determine structures of their complexes with SC2M^{Pro} due to cracking of the crystals upon soaking with the inhibitors. For MPI3, the electron density around the P1, P2, and P3 residues were well defined, and the covalent interaction between the C₁₄₅ side chain thiolate and the Opal aldehyde to form a hemiacetal was clearly observable (PDB: 7JQ0). The electron density around CBZ was very weak indicating flexible CBZ binding around the enzyme P4-binding pocket. A superposition of apo SC2M^{Pro} and the SC2M^{Pro}-MPI3 complex structures display very little overall variation with RMSD as 0.2 Å. Around the active site in the two structures, large structural rearrangements exist for residues M49 and N142 and the loop region that contains P168. In apoenzyme, the side chain of M49 folds into the P2-binding pocket. It flips toward the solvent to make space available for the binding of the P2 Leu in MPI3. The side chain of N142 rotates by almost 1800 between the two structures and adopts a conformation in the SC2M^{Pro}-MPI3 complex that closely caps the P1-binding site for strong van der Waals interactions with the Opal residue in MPI3. In the SC2M^{Pro}-MPI3 complex, the P168-containing loop is pushed away from its original position in the apoenzyme, probably by interaction with the CBZ group, which triggers a position shift for the whole loop. Except for M49, N142, and the P168-containing loop, structural orientations of all other residues at the active site closely resemble each other in the two structures. In the active site, MPI3 occupies the P1, P2, and P4-binding pockets and leaves the large P3′-binding pocket empty. Extensive hydrogen bonding and van der Waals interactions in addition to the covalent interaction with C145 contribute to the strong binding of MPI3 to SC2M^{Pro}. Residues F140, N142, H163, E166, and H172 form a small cage to accommodate the Opal side chain. Three hydrogen bonds form between the Opal lactam amide and the E166 side chain carboxylate, H163 imidazole, and F140 backbone carbonyl oxygen. The precise fitting of Opal into the P1-binding pocket and the formation of three hydrogen bonds explain the preferential binding of the Opal side chain to this pocket. In the SC2M^{Pro}-MPI3 complex, M49 flips from the P2-binding pocket to leave space for the binding of the P2 Leu in MPI3. Residues H41, M49, M165 and D187, backbones of the M165-containing strand, and the D-187-containing loop form a hydrophobic pocket that is in a close range of van der Waals interactions with the P2 Leu in MPI3. Leu showed strong results in this position probably due to this close van der Waals interaction range for the recognition of the P2 Leu side chain. The enzyme has no P3-binding pocket. However, the P3 Val in MPI3 positions its side chain in van der Waals interaction distance to E166 and P168. In the structure, CBZ narrowly fits into the P4-binding pocket and the channel formed between the P168- and Q192-containing loops. The P168 loop rearranges its position from that in apoenzyme to accommodate the CBZ group. The CBZ group also has weak electron density. These observations indicate that CBZ is not an optimal structural moiety for interaction at these sites. Besides interactions involving side chains and the CBZ group in MPI3, its two backbone amides and carbamate form 6 hydrogen bonds with the enzyme. Two of them are formed between the P3 Val in MPI3 and the backbone amino and carbonyl groups of E166 in SC2M^{Pro}. One water molecule mediates a hydrogen bond bridge between the P2 Leu amino group in MPI3 and the Q189 side chain amide in SC2M^{Pro}. For the P1 Opal residue in MPI3, its a-amino group forms a hydrogen bond with the H164 α-carbonyl oxygen in the enzyme. The original aldehyde oxygen in MPI3 forms two hydrogen bonding interactions, one with the α-amino group of G143 and the other the C145 α-amine in SC2M^{Pro}. The two hydrogen bonds may be a reason that Opal-based reversible covalent inhibitors are typically stronger than Michael acceptor inhibitors, in which the original scissile amide is replaced with an alkene, for inhibition of M^{Pro} enzymes. In the structures of SC2M^{Pro} complexes with the other 6 inhibitors, we observed similar structure rearrangements at M49, N142, and the P168-containing loop to accommodate inhibitors and a covalent interaction (PDB: 7JPZ, 7JQ1, 7JQ2, 7JQ3, 7JQ4, 7JQ5).

Representative Synthetic Procedure I

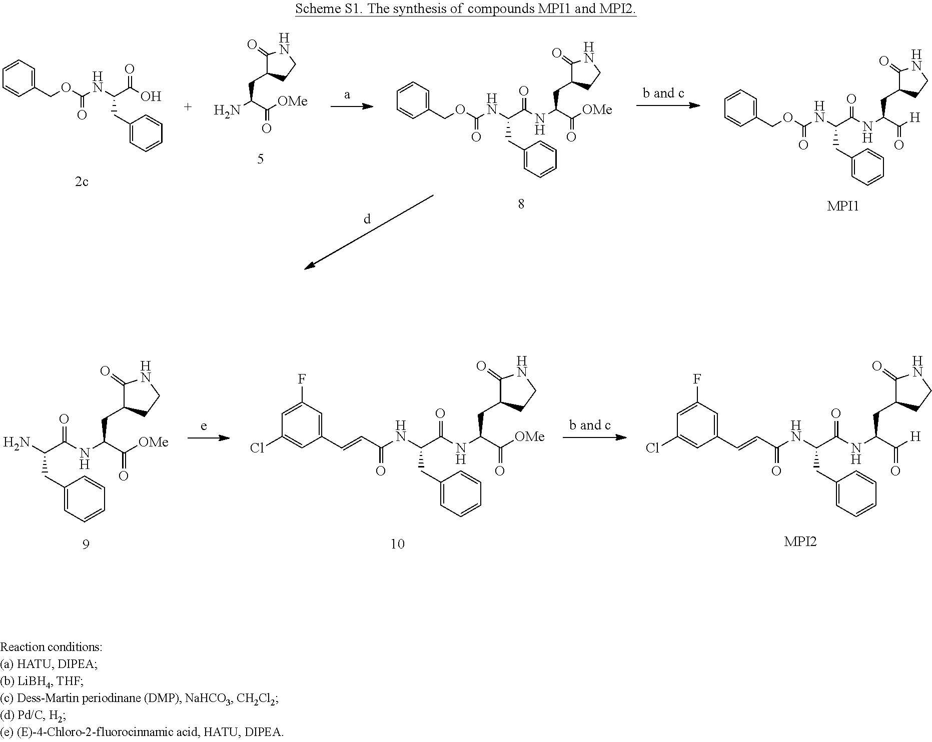

Representative Synthetic Procedure II

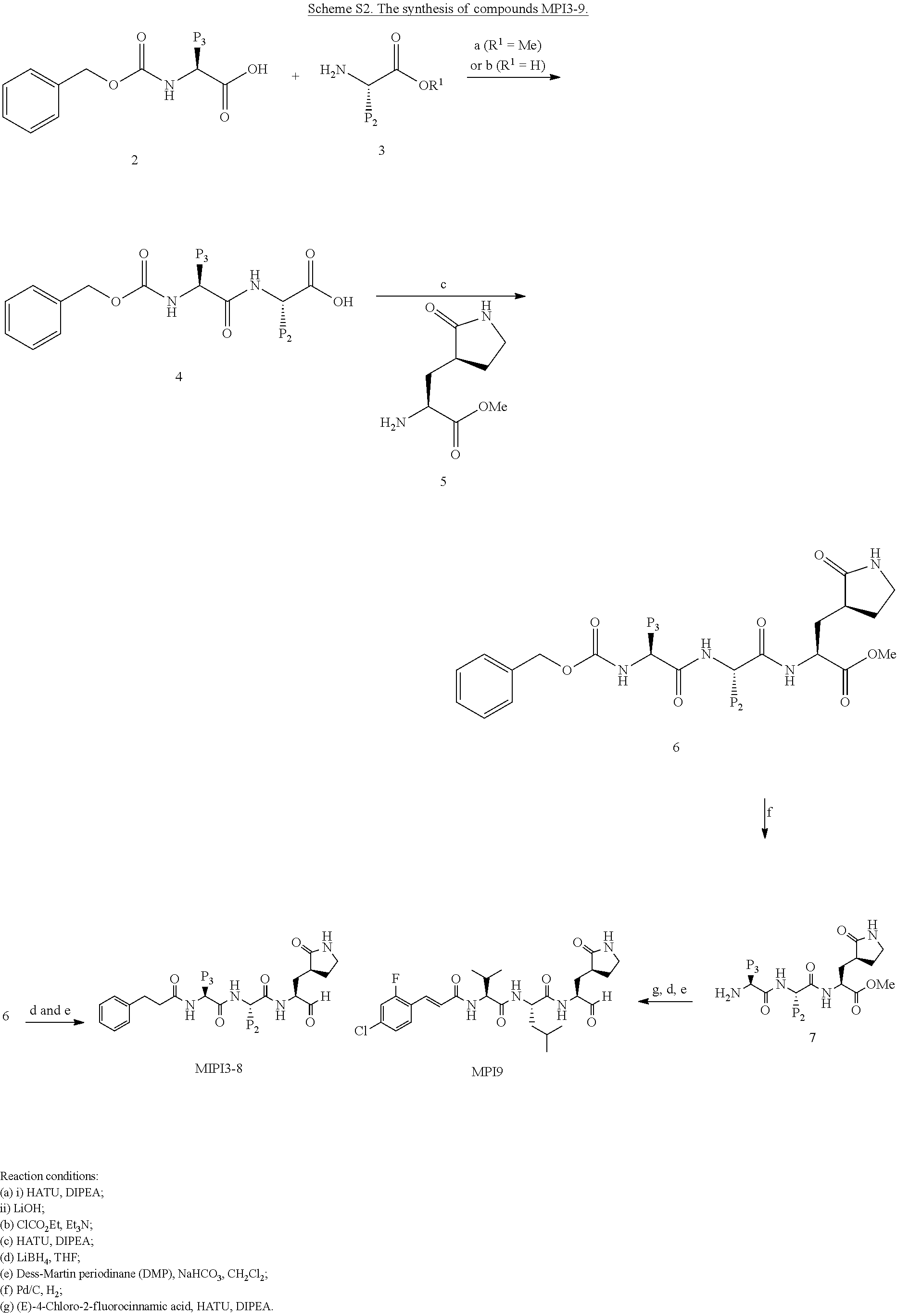

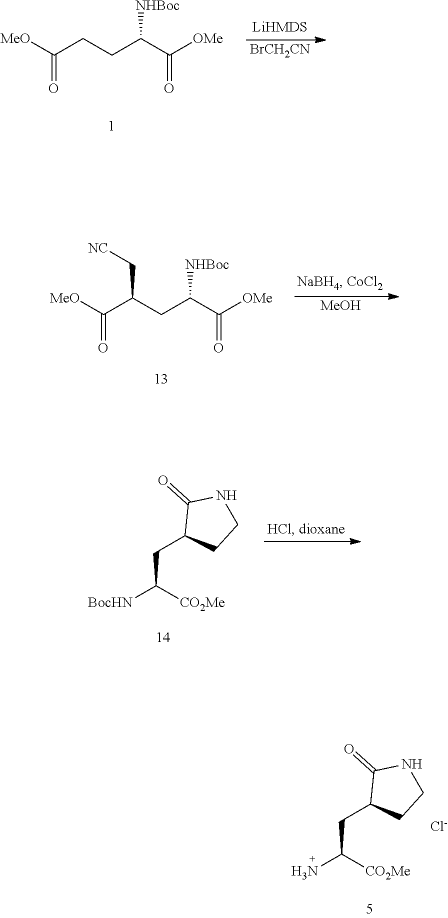

### Dimethyl (2S,4R)-2-((tert-butoxycarbonyl)amino)-4-(cyanomethyl)pentanedioate (13)

A solution of N-Boc-glutamic acid dimethyl ester (3 g, 11 mmol, 1 equiv.) in anhydrous THF (20 mL) was cooled under −78° C. Then, 24 mL of 1 M LiHMDS solution in THF (24 mmol, 2.18 equiv.) was added to the solution dropwise. After addition, the solution was stirred under −78° C. for 1 h. Meanwhile, boromoacetonitrile was stirred with activated basic alumina for 2 h and then filtered. Freshly dried and filtered bromoacetonitrle (1.4 g, 11.8 mmol, 1.06 equiv.) was then added dropwise to the dianion solution. The solution was then stirred under −78° C. for 3-5 h, until TLC confirms complete consumption of the starting material. Then the reaction was quenched with pre-cooled methanol (1 mL) in one portion and stirred under the same temperature for 30 min. The methoxide solution was then quenched with pre-cooled AcOH/THF (1 mL in 6 mL THF) in one portion and stirred for another 30 min under the same temperature. Then the cooling bath was removed. The reaction mixture was allowed to warm up to room temperature and poured into 50 mL of saturated brine solution. The layers were separated, and the organic layer was then concentrated to give dark oil. Then to the residue was added 4 g of silica gel, 1 g of activated charcoal and 50 mL of dichloromethane. The slurry was stirred for 1 h, and then filtered and washed with another 50 mL of dichloromethane. The filtrate was then concentrated to give brown oil, which was used without further purification.

### Methyl (S)-2-((tert-butoxycarbonyl)amino)-3-((S)-2-oxopyrrolidin-3-yl)propanoate (14)

To a pre-cooled solution of CoCl₂.6H₂O (1.54 g, 6.5 mmol) and 13 (11 mmol, crude) in methanol under 0° C. was added NaBH₄ (44 mmol, 1.67 g) in portions over 30 min. The reaction was exothermic and produces copious amount of hydrogen and black precipitate. The reaction mixture was stirred under room temperature for 24 h, and then concentrated on vacuo. The residue oil was then poured into 10% citric acid and filtered. The filtrate was then extracted with ethyl acetate twice. The organic layer was then dried over anhydrous Na₂SO₄ and concentrated. The residue was purified by flash chromatography to afford 14 as light-yellow oil (2.1 g, 66%). ¹H NMR (400 MHz, CDCl₃): δ 6.58 (s, 1H), 5.58 (d, J=8.5 Hz, 1H), 4.19-4.36 (m, 1H), 3.71 (s, 3H), 3.23-3.39 (m, 2H), 2.36-2.54 (m, 2H), 2.04-2.19 (m, 1H), 1.73-1.90 (m, 1H), 1.41 (s, 9H).

### (S)-1-Methoxy-1-oxo-3-((S)-2-oxopyrrolidin-3-yl)propan-2-amine hydrochloride (5)

To a solution of 14 (2.1 g, 7.3 mmol) in 1,4-dioxane (10 mL) was added dropwise a HCl solution in 1,4-dioxane (4 M, 10 mL). The resulting solution was stirred at room temperature for 1 h. Then residue was then concentrated on vacuo to afford 5 as light-yellow hydroscopic crystal (1.5 g, 92%). ¹H NMR (400 MHz, d₆-DMSO): δ 8.72 (s, 3H), 7.97 (s, 1H), 4.13-4.24 (m, 1H), 3.76 (s, 3H), 3.12-3.24 (m, 2H), 2.54-2.65 (m, 1H), 2.23-2.34 (m, 1H), 2.01-2.10 (m, 1H), 1.83-1.92 (m, 1H), 1.62-1.73 (m, 1H).

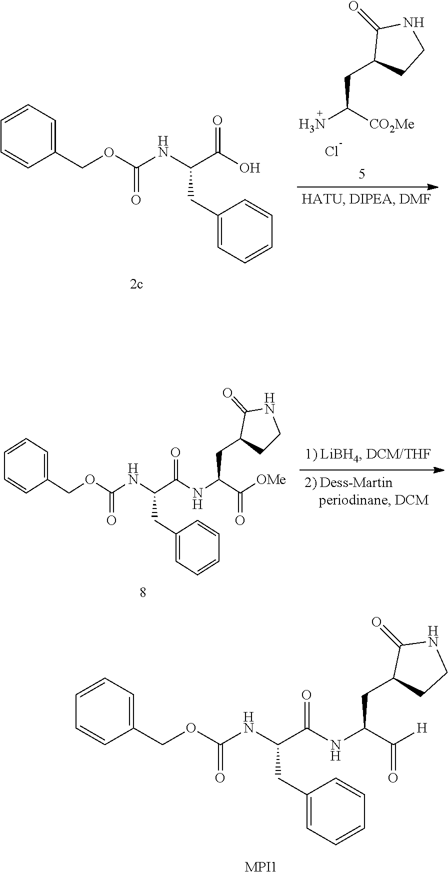 [SMILES: O=C(N[C@@H](Cc1ccccc1)C(=O)O)OCc1ccccc1 ; COC(=O)[C@@H](N)C[C@@H]1CCNC1=O ; Cl ; COC(=O)[C@H](C[C@@H]1CCNC1=O)NC(=O)[C@H](Cc1ccccc1)NC(=O)OCc1ccccc1 ; O=C[C@H](C[C@@H]1CCNC1=O)NC(=O)[C@H](Cc1ccccc1)NC(=O)OCc1ccccc1]

### Methyl (S)-2-((S)-2-(((benzyloxy)carbonyl)amino)-3-phenylpropanamido)-3-((S)-2-oxopyrrolidin-3-yl)propanoate (8)

To a solution of 2c (2 mmol, 0.44 g) and 5 (2 mmol, 0.44 g) in anhydrous DMF (10 mL) was added DIPEA (4 mmol, 0.52 g) and was cooled to 0° C. HATU (2.2 mmol, 0.84 g) was added to the solution under 0° C. and then stirred at room temperature overnight. The reaction mixture was then diluted with ethyl acetate (50 mL) and washed with saturated NaHCO₃ solution (2×20 mL), 1 M HCl solution (2×20 mL), and saturated brine solution (2×20 mL) sequentially. The organic layer was dried over anhydrous Na₂SO₄ and then concentrated on vacuo. The residue was then purified with flash chromatography (50-100% EtOAc in hexanes as the eluent) to afford 8 as white solid (520 mg, 56%). ¹H NMR (400 MHz, d₆-DMSO): δ 8.59 (d, J=7.9 Hz, 1H), 7.66 (s, 1H), 7.53 (d, J=8.5 Hz, 1H), 7.03-7.43 (m, 10H), 4.93 (q, J=12.3, 11.8 Hz, 2H), 4.31-4.42 (m, 1H), 4.22-4.31 (m, 1H), 3.63 (s, 3H), 2.91-3.18 (m, 3H), 2.68-2.79 (m, 1H), 2.23-2.36 (m, 1H), 2.01-2.17 (m, 2H), 1.53-1.67 (m, 2H).

### Benzyl ((S)-1-oxo-1-(((S)-1-oxo-3-((S)-2-oxopyrrolidin-3-yl)propan-2-yl)amino)-3-phenylpropan-2-yl)carbamate (MPI1)

To a solution of 8 (0.1 mmol, 47 mg) in anhydrous dichloromethane (5 mL) was added a solution of LiBH₄ in anhydrous THF (2 M, 0.1 mL, 0.2 mmol) at 0° C. The resulting solution was stirred at the same temperature for 3 h. Then a saturated solution of NH₄Cl (5 mL) was added dropwise to quench the reaction. The layers were separated, and the organic layer was washed with saturated brine solution (2×10 mL), dried over anhydrous Na₂SO₄ and evaporated to dryness. The residue was then dissolved in anhydrous dichloromethane (5 mL) and cooled to 0° C. Dess-Martin periodinane (0.2 mmol, 85 mg) was added to the solution. The reaction mixture was then stirred at room temperature overnight. Then the reaction was quenched with a saturated NaHCO₃ solution containing 10% Na₂S₂O₃. The layers were separated. The organic layer was then washed with saturated brine solution (2×10 mL), dried over anhydrous Na₂SO₄ and evaporated on vacuo. The residue was then purified with flash chromatography (1-10% methanol in dichloromethane as the eluent) to afford 1i as white solid (30 mg, 65%). ¹H-NMR (400 MHz, CDCl₃): δ 9.26 (s, 1H), 8.18 (s, 1H), 7.46-7.08 (m, 10H), 5.54 (s, 1H), 5.44 (d, J=9.2 Hz, 1H), 5.11 (s, 2H), 4.65-4.53 (m, 1H), 4.30-4.16 (m, 1H), 3.37-3.24 (m, 2H), 3.23-3.14 (m, 1H), 3.07 (dd, J=13.6, 6.5 Hz, 1H), 2.40-2.28 (m, 1H), 2.27-2.19 (m, 1H), 1.92-1.73 (m, 3H); ¹³C NMR (100 MHz, CDCl₃) δ 199.98, 180.16, 172.13, 155.96, 136.39, 129.62, 128.73, 128.65, 128.29, 128.15, 127.14, 67.11, 58.05, 56.14, 40.72, 38.96, 38.17, 31.08, 29.53, 28.88; ESI-MS calcd for C₂₄H₂₈N₃O₅ (M+H⁺): 438.2; found 438.3.

### Representative Synthetic Procedure I

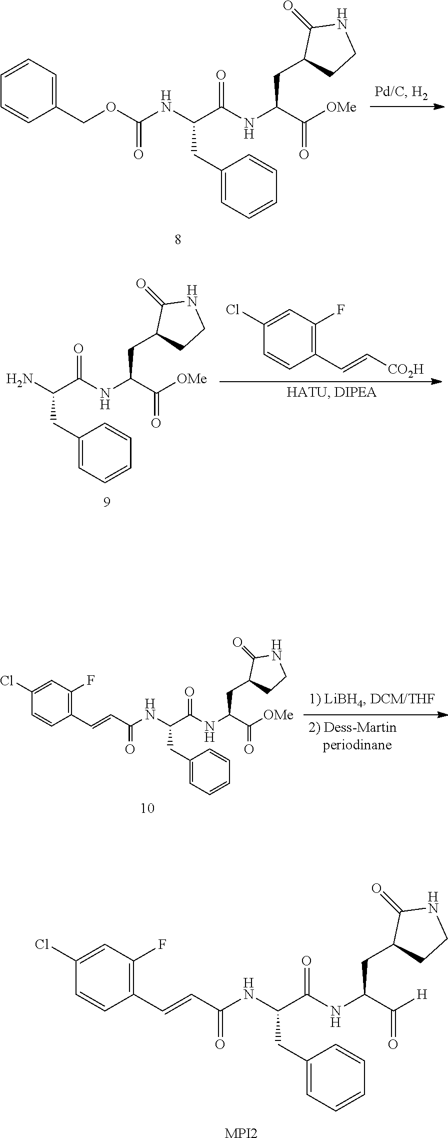

### Methyl (S)-2-((S)-2-((E)-3-(3-chloro-5-fluorophenyl)acrylamido)-3-phenylpropanamido)-3-((S)-2-oxopyrrolidin-3-yl)propanoate (10)

To a solution of 8 (0.25 mmol, 116 mg) in methanol was added 10% Pd/C (26 mg). The mixture was then stirred with hydrogen balloon at room temperature for 3 h. The catalyst was then filtered off and the solution was evaporated on vacuo to afford 9 as white solid, which was used without purification. To a solution of 9 in 2 mL dry DMF was added DIPEA (0.5 mmol, 65 mg) and cooled to 0° C. Then HATU (0.3 mmol, 114 mg) was added to the solution at the same temperature. The solution was stirred at room temperature overnight. The reaction mixture was then diluted with ethyl acetate (20 mL) and washed with saturated NaHCO₃ solution (2×10 mL), 1 M HCl solution (2×10 mL), and saturated brine solution (2×10 mL) sequentially. The organic layer was dried over anhydrous Na₂SO₄ and then concentrated on vacuo. The residue was then purified with flash chromatography (1-10% methanol in dichloromethane as the eluent) to afford 10 as white solid (80 mg, 62%). ¹H-NMR (400 MHz, CDCl₃): δ 7.92 (d, J=6.7 Hz, 1H), 7.62 (d, J=15.8 Hz, 1H), 7.40 (t, J=8.3 Hz, 1H), 7.32-7.17 (m, 5H), 7.11 (td, J=8.7, 7.4, 3.0 Hz, 2H), 6.61-6.43 (m, 2H), 5.89 (s, 1H), 5.02 (dd, J=8.2, 5.9 Hz, 1H), 4.51-4.33 (m, 1H), 3.72 (s, 3H), 3.38-3.25 (m, 2H), 3.25-3.11 (m, 2H), 2.44-2.31 (m, 1H), 2.27-2.19 (m, 1H), 2.17-2.05 (m, 1H), 1.95-1.74 (m, 2H).

### (E)-3-(3-chloro-5-fluorophenyl)-N—((S)-1-oxo-1-(((S)-1-oxo-3-((S)-2-oxopyrrolidin-3-yl)propan-2-yl)amino)-3-phenylpropan-2-yl)acrylamide (MPI2)

To a solution of 10 (0.1 mmol, 52 mg) in anhydrous dichloromethane (5 mL) was added a solution of LiBH₄ in anhydrous THF (2 M, 0.1 mL, 0.2 mmol) at 0° C. The resulting solution was stirred at the same temperature for 3 h. Then a saturated solution of NH₄Cl (5 mL) was added dropwise to quench the reaction. The layers were separated, and the organic layer was washed with saturated brine solution (2×10 mL), dried over anhydrous Na₂SO₄, and evaporated to dryness. The residue was then dissolved in anhydrous dichloromethane (5 mL) and cooled to 0° C. Dess-Martin periodinane (0.2 mmol, 85 mg) was added to the solution. The reaction mixture was then stirred at room temperature overnight. Then the reaction was quenched with a saturated NaHCO₃ solution containing 10% Na₂S₂O₃. The layers were separated. The organic layer was then washed with saturated brine solution (2×10 mL), dried over anhydrous Na₂SO₄ and evaporated on vacuo. The residue was then purified with flash chromatography (1-10% methanol in dichloromethane as the eluent) to afford MPI2 as white solid (27 mg, 55%). ¹H-NMR (400 MHz, DMSO-d₆): δ 9.30 (s, 1H), 8.67 (d, J=7.6 Hz, 1H), 8.58 (d, J=8.2 Hz, 1H), 7.68-7.61 (m, 2H), 7.52 (dd, J=10.8, 2.1 Hz, 1H), 7.41-7.32 (m, 2H), 7.30-7.15 (m, 5H), 6.81 (d, J=16.0 Hz, 1H), 4.75-4.67 (m, 1H), 4.21-4.12 (m, 1H), 3.18-3.04 (m, 3H), 2.89 (dd, J=13.8, 9.3 Hz, 1H), 2.25-2.05 (m, 2H), 1.88 (ddt, J=13.9, 11.3, 5.6 Hz, 1H), 1.67-1.55 (m, 2H); ¹³C NMR (101 MHz, CDCl3) δ 199.87, 179.97, 171.97, 165.18, 162.26, 159.71, 136.30, 133.45, 130.13, 129.55, 128.59, 127.05, 124.96, 123.37, 121.40, 116.82, 57.99, 54.27, 40.62, 38.83, 38.10, 29.42, 28.75; ESI-MS: calcd for C₂₅H₂₆ClFN₃O₄ (M+H⁺): 486.1; found 486.1.

### Representative Synthetic Procedure II

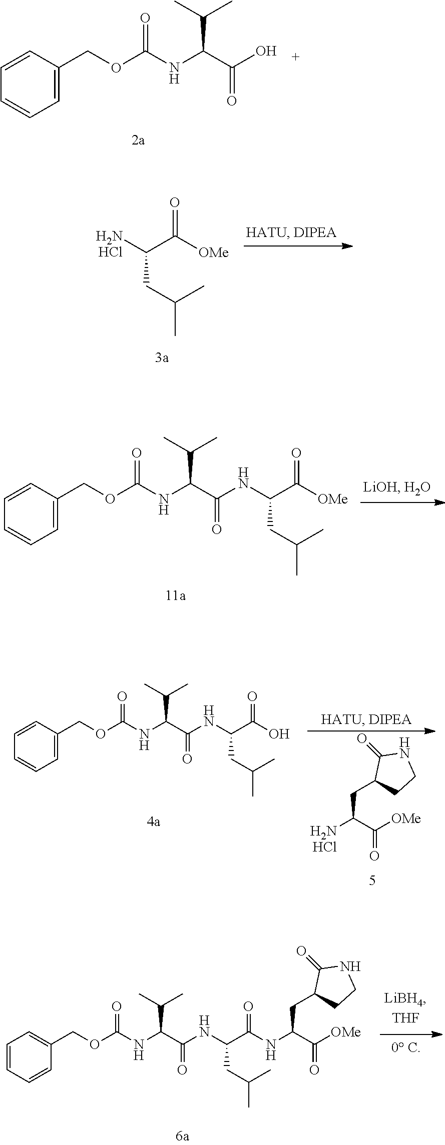

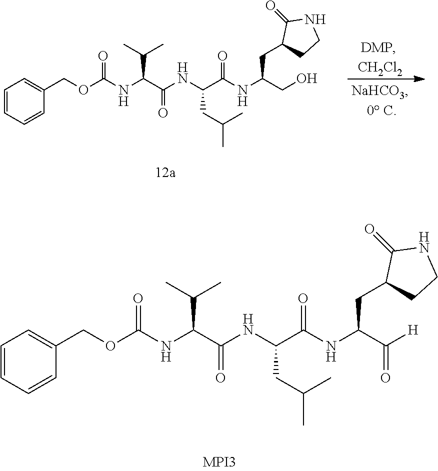 [SMILES: CC(C)C[C@H](NC(=O)[C@@H](NC(=O)OCc1ccccc1)C(C)C)C(=O)N[C@H](CO)C[C@@H]1CCNC1=O ; CC(C)C[C@H](NC(=O)[C@@H](NC(=O)OCc1ccccc1)C(C)C)C(=O)N[C@H](C=O)C[C@@H]1CCNC1=O]

### (S)-Methyl 2-((S)-2-(((benzyloxy)carbonyl)amino)-3-methylbutanamido)-4-methylpentanoate (11a)

The amino acid methyl ester hydrochloride 3a (1.0 g, 5.52 mmol) and the Cbz-protected amino acid 2a (1.88 g, 6.08 mmol) were dissolved in dry DMF (20 mL) and the reaction was cooled to 0° C. HATU (2.52 g, 6.62 mmol) and DIPEA (3.92 mL, 22.08 mmol) were added, and the reaction mixture was allowed warm up to room temperature and stirred for 12 h. The mixture was then poured into water (50 mL) and extracted with ethyl acetate (4×20 mL). The organic layer was washed with aqueous hydrochloric acid 10% v/v (2×20 mL), saturated aqueous NaHCO₃ (2×20 mL), brine (2×20 mL) and dried over Na₂SO₄. The organic phase was evaporated to dryness and the crude material purified by silica gel column chromatography (15-50% EtOAc in n-hexane as the eluent) to afford 11a white solid (1.82, 69%). ¹H NMR (CDCl₃, 400 MHz) δ 7.28-7.22 (m, 5H), 6.28 (d, J=7.72 Hz, 1H), 5.35 (d, =864 Hz, 1H), 5.03 (s, 2H), 4.56-451 (m, 1H), 3.97 (t, J=8.12 Hz, 1H), 3.65 (s, 3H), 2.19-1.98 (m, 1H), 1.62-1.43 (m, 3H), 0.92-0.83 (m, 12H); ¹³C NMR (CDCl₃, 100 MHz) δ 173.2, 171.1, 156, 136.2, 128.5 (2C), 128.2, 128 (2C), 67.0, 60.2, 52.3, 50.7, 1, 31.3, 2.8, 22.7, 21.9, 19.1, 17.8.

### (S)-2-((S)-2-(((Benzyloxy)carbonyl)amino)-3-methylbutanamido)-4-methylpentanoic acid (4a)

The peptide 11a (500 mg, 1.14 mmol) was dissolved in THF/H₂O (1:1, 10 mL).LiOH (114 mg, 2.86 mmol) was added at 0° C. The mixture was stirred at room temperature overnight. Then THF was removed on vacuum and the aqueous layer was acidified with 1 M HCl and extracted with dichloromethane (3×10 mL). The organic layer was dried over anhydrous Na₂SO₄ and concentrated to yield 4a as white solid (315 mg, 65%). ¹H NMR (CDCl₃, 400 MHz): δ 7.26-7.23 (m, 5H), 6.65 (d, J=7.88, 1H), 5.68 (d, J=4.64 Hz, 1H), 5.03 (s, 2H), 4.56-4.45 (m, 1H), 3.97 (t, J=7.92 Hz, 1H), 2.02-1.96 (m, 1H), 1.66-1.46 (m, 3H), 0.85 (dd, J=7.36, 13.3 Hz, 12H); ¹³C NMR (CDCl₃, 100 MHz): δ 176.0, 171.8, 156.7, 136.1, 128.5 (2C), 128.2, 128.0 (2C), 67.2, 60, 50.8, 41.1, 31.1, 24.8, 22.8, 21.8, 19.1, 18.0.

### (5S,8S,11S)-Methyl 8-isobutyl-5-isopropyl-3,6,9-trioxo-11-(((S)-2-oxopyrrolidin-3-yl)methyl)-1-phenyl-2-oxa-4,7,10-triazadodecan-12-oate (6a)

The methyl (S)-2-amino-3-((S)-2-oxopyrrolidin-3-yl)propanoate hydrochloride 5 (150 mg, 0.657 mmol) and the peptide 11a (270 mg, 0.743 mmol) were dissolved in dry DMF (10 mL) and the reaction was cooled to 0° C. HATU (308 mg, 0.788 mmol) and DIPEA (0.48 mL, 2.63 mmol) were added, and the reaction mixture was allowed warm up to room temperature and stirred for 12 h. The mixture was then poured into water (20 mL) and extracted with ethyl acetate (4×20 mL). The organic layer was washed with aqueous hydrochloric acid 10% v/v (2×20 mL), saturated aqueous NaHCO₃ (2×20 mL), brine (2×20 mL) and dried over Na₂SO₄. The organic phase was evaporated to dryness and the crude material purified by silica gel column chromatography afford 6a as white solid (250 mg, 70%). ¹H NMR (CDCl₃, 400 MHz) δ 7.89 (d, J=7.16 Hz, 1H), 7.28-7.20 (m, 5H), 7.00 (d, J=8.24 Hz, 1H), 6.65 (Brs, 1H), 5.47 (d, J=8.8 Hz, 1H), 5.01 (s, 2H), 4.59-4.51 (m, 1H), 4.46-4.38 (m, 1H), 3.94 (t, J=7.84 Hz, 1H), 3.63 (s, 3H), 3.28-3.18 (m, 2H), 2.38-2.22 (m, 2H), 2.29-1.98 (m, 2H), 1.79-1.70 (m, 1H), 1.68-1.51 (m, 2H), 1.50-1.25 (m, 2H), 0.86-0.81 (m, 12H); ¹³C NMR (CDCl₃, 100 MHz) δ 179.8, 172.5, 172.1, 171.2, 156.5, 136.2, 128.5 (2C), 128.2, 128.0 (2C), 67.1, 60.5, 52.4, 51.7, 51.1, 42.0, 40.5, 38.3, 33.1, 31.1, 28.1, 24.6, 22.8, 22.0, 19.7, 19.1.

### Benzyl ((S)-1-(((S)-1-(((S)-1-hydroxy-3-((S)-2-oxopyrrolidin-3-yl)propan-2-yl)amino)-4-methyl-1-oxopentan-2-yl)amino)-3-methyl-1-oxobutan-2-yl)carbamate (12a)

To a stirred solution of compound 6a (120 mg, 0.254 mmol) in THF (8 mL) was added LiBH₄ (2.0 M in THF, 0.636 mL, 1.27 mmol) in several portions at 0° C. under a nitrogen atmosphere. The reaction mixture was stirred at 0° C. for 1 h, then allowed to warm up to room temperature, and stirred for an additional 2 h. The reaction was quenched by the drop wise addition of 1.0 M HCl (aq) (1.2 mL) with cooling in an ice bath. The solution was diluted with ethyl acetate and H₂O. The phases were separated, and the aqueous layer was extracted with ethyl acetate (3×15 mL). The organic phases were combined together, dried over MgSO₄, filtered, and concentrated on a rotorvap to give a yellow oily residue. Column chromatographic purification of the residue (6% MeOH in CH₂Cl₂ as the eluent) afforded a white solid (80 mg, 70%). ¹H NMR (CDCl₃) δ 7.62 (d, J=6.48 Hz, 1H), 7.30-7.27 (m, 5H), 6.57 (d, J=7.6 Hz, 1H), 5.76 (Brs, 1H), 5.30 (d, J=7.84 Hz, 1H), 5.05 (s, 2H), 4.4-4.38 (m, 1H), 3.93 (t, J=7.0 Hz, 2H), 3.60-3.53 (m, 2H), 3.43 (s, 3H), 3.27-3.25 (m, 2H), 2.39-2.32 (m, 2H), 2.15-2.01 (m, 1H), 1.95-1.75 (m, 3H), 1.68-1.49 (m, 6H), 0.92-0.84 (m, 12H).

### Benzyl ((S)-3-methyl-1-(((S)-4-methyl-1-oxo-1-(((S)-1-oxo-3-((S)-2-oxopyrrolidin-3-yl)propan-2-yl)amino)pentan-2-yl)amino)-1-oxobutan-2-yl)carbamate (MPI3)

To a solution of 12a (70 mg, 0.142 mmol) in CH₂Cl₂ (6 mL) was added NaHCO₃ (48 mg, 4 equiv) and the Dess-Martin reagent (180 mg, 0.427 mmol, 3 equiv). The resulting mixture was stirred at rt for 12 h. Then the reaction was quenched with a saturated NaHCO₃ solution containing 10% Na₂S₂O₃. The layers were separated. The organic layer was then washed with saturated brine solution, dried over anhydrous Na₂SO₄ and concentrated on vacuum. The residue was then purified with flash chromatography afford MPI3 as white solid (45 mg, 64%). ¹H NMR (CDCl₃, 400 MHz): δ 9.41 (s, 1H), 8.18 (d, J=5.12 Hz, 1H), 7.28-7.26 (m, 5H), 6.63 (d, J=7.0 Hz, 1H), 6.15 (Brs, 1H), 5.40 (d, J=6.88 Hz, 1H), 5.02 (s, 2H), 4.52-4.50 (m, 1H), 4.27-4.25 (m, 1H), 3.94-3.92 (m, 2H), 3.27-3.23 (m, 2H), 2.35-2.27 (m, 2H), 2.07-2.05 (m, 1H), 1.90-1.81 (m, 2H), 1.76-1.71 (m, 1H), 1.57-1.43 (m, 3H), 0.85 (dd, J=6.76, 14.72 Hz, 12H); ¹³C NMR (CDCl₃, 100 MHz): δ 199.6, 180.0, 173.2, 171.4, 156.6, 136.2, 128.6 (2C), 128.2, 128.1 (2C), 67.1, 60.6, 57.5, 51.2, 41.7, 40.6, 38.0, 31.0, 29.8, 28.4, 24.8, 22.9, 21.9, 19.2, 17.8.

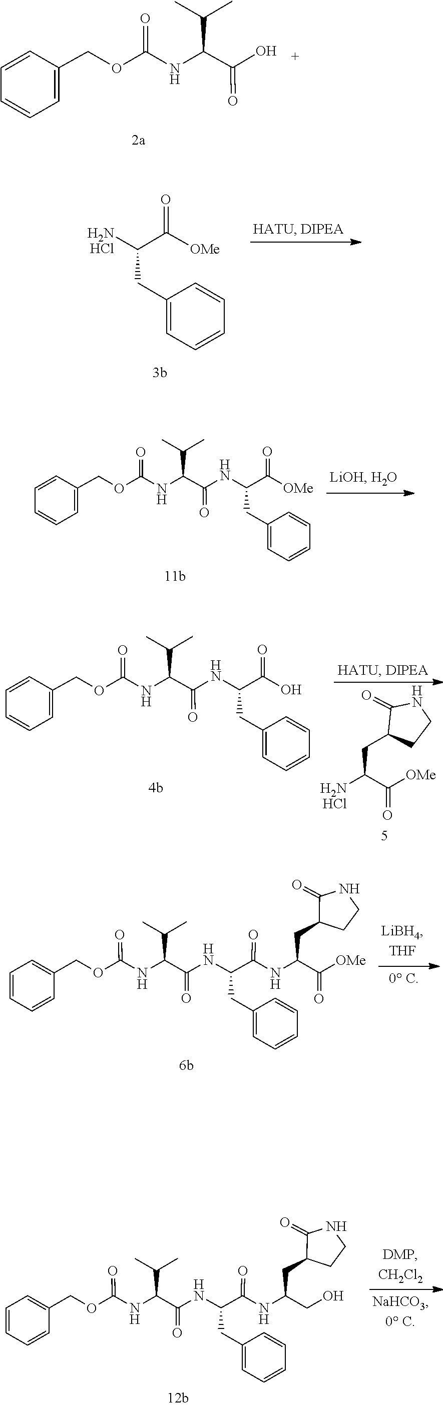

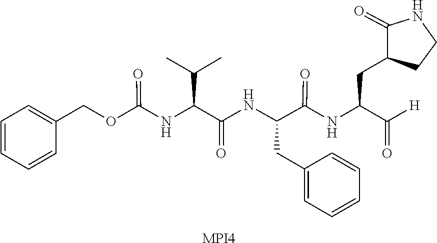

### Methyl ((benzyloxy)carbonyl)-L-valyl-L-phenylalaninate (11b)

To a solution of 2a (5 mmol, 1.25 g) and 3b (5 mmol, 1.07 g) in anhydrous DMF (20 mL) was added DIPEA (10 mmol, 1.29 g) and was cooled to 0° C. HATU (5.5 mmol, 2.09 g) was added to the solution under 0° C. and then stirred at room temperature overnight. The reaction mixture was then diluted with ethyl acetate (100 mL) and washed with saturated NaHCO₃ solution (2×50 mL), 1 M HCl solution (2×50 mL), and saturated brine solution (2×50 mL) sequentially. The organic layer was dried over anhydrous Na₂SO₄ and then concentrated on vacuo. The residue was then purified with flash chromatography (15-50% EtOAc in hexanes as the eluent) to afford 11b as white solid (1.52 g, 74%).

### ((Benzyloxy)carbonyl)-L-valyl-L-phenylalanine (4b)

11b (1 mmol, 470 mg) was dissolved in 5 mL of THF. A solution of LiOH.H₂O (2 mmol, 84 mg) in 5 mL H₂O was added to the solution. The mixture was stirred at room temperature overnight. Then THF was removed on vacuo and the aqueous layer was acidified with 1 M HCl and extracted with dichloromethane (3×10 mL). The organic layer was dried over anhydrous Na₂SO₄ and evaporated to give 4b as white solid (312 mg, 76%). ¹H-NMR (400 MHz, CD₃OD): δ 7.29-7.42 (m, 5H), 7.16-7.29 (m, 5H), 5.11 (s, 2H), 4.73-4.68 (m, 1H), 3.94 (d, J=7.3 Hz, 1H), 3.21 (dd, J=13.9, 5.2 Hz, 1H), 3.01 (dd, J=13.9, 8.6 Hz, 1H), 2.06-1.95 (m, 1H), 0.91 (dd, J=8.5, 6.7 Hz, 6H).

### Methyl (5S,8S,11S)-8-benzyl-5-isopropyl-3,6,9-trioxo-11-(((S)-2-oxopyrrolidin-3-yl)methyl)-1-phenyl-2-oxa-4,7,10-triazadodecan-12-oate (6b)

To a solution of 4b (0.4 mmol, 160 mg) and 5 (0.4 mmol, 88 mg) in anhydrous DMF (2 mL) was added DIPEA (0.8 mmol, 103 mg) and was cooled to 0° C. HATU (0.44 mmol, 167 mg) was added to the solution under 0° C. and then stirred at room temperature overnight. The reaction mixture was then diluted with ethyl acetate (20 mL) and washed with saturated NaHCO₃ solution (2×10 mL), 1 M HCl solution (2×10 mL), and saturated brine solution (2×10 mL) sequentially. The organic layer was dried over anhydrous Na₂SO₄ and then concentrated on vacuo. The residue was then purified with flash chromatography (1-10% methanol in dichloromethane as the eluent) to afford 6b as white solid (151 mg, 67%). ¹H-NMR (400 MHz, CDCl₃): δ 7.71 (d, J=7.1 Hz, 1H), 7.42-7.30 (m, 5H), 7.25-7.13 (m, 5H), 6.78 (d, J=8.5 Hz, 1H), 5.83 (s, 1H), 5.26 (d, J=8.7 Hz, 1H), 5.10 (d, J=4.2 Hz, 2H), 4.83 (q, J=6.9 Hz, 1H), 4.51-4.41 (m, 1H), 3.96 (dd, J=8.6, 6.2 Hz, 1H), 3.70 (s, 3H), 3.37-3.25 (m, 2H), 3.11 (d, J=6.3 Hz, 2H), 2.44-2.31 (m, 1H), 2.26-2.13 (m, 1H), 2.13-1.99 (m, 2H), 1.93-1.74 (m, 2H), 0.91 (d, J=6.8 Hz, 3H), 0.82 (d, J=6.8 Hz, 3H).

### Benzyl ((S)-3-methyl-1-oxo-1-(((S)-1-oxo-1-(((S)-1-oxo-3-((S)-2-oxopyrrolidin-3-yl)propan-2-yl)amino)-3-phenylpropan-2-yl)amino)butan-2-yl)carbamate (MPI4)

To a solution of 6b (0.1 mmol, 57 mg) in anhydrous dichloromethane (5 mL) was added a solution of LiBH₄ in anhydrous THF (2 M, 0.1 mL, 0.2 mmol) at 0° C. The resulting solution was stirred at the same temperature for 3 h. Then a saturated solution of NH₄Cl (5 mL) was added dropwise to quench the reaction. The layers were separated, and the organic layer was washed with saturated brine solution (2×10 mL), dried over anhydrous Na₂SO₄, and evaporated to dryness. The residue was then dissolved in anhydrous dichloromethane (5 mL) and cooled to 0° C. Dess-Martin periodinane (0.2 mmol, 85 mg) was added to the solution. The reaction mixture was then stirred at room temperature overnight. Then the reaction was quenched with a saturated NaHCO₃ solution containing 10% Na₂S₂O₃. The layers were separated. The organic layer was then washed with saturated brine solution (2×10 mL), dried over anhydrous Na₂SO₄ and evaporated on vacuo. The residue was then purified with flash chromatography (1-10% methanol in dichloromethane as the eluent) to afford MPI4 as white solid (30 mg, 65%). ¹H-NMR (400 MHz, CDCl₃): δ 9.26 (s, 1H), 8.18 (s, 1H), 7.46-7.08 (m, 10H), 5.54 (s, 1H), 5.44 (d, J=9.2 Hz, 1H), 5.11 (s, 2H), 4.65-4.53 (m, 1H), 4.30-4.16 (m, 1H), 3.37-3.24 (m, 2H), 3.23-3.14 (m, 1H), 3.07 (dd, J=13.6, 6.5 Hz, 1H), 2.40-2.28 (m, 1H), 2.27-2.19 (m, 1H), 1.92-1.73 (m, 3H); ¹³C NMR (100 MHz, CDCl₃) δ 199.98, 180.16, 172.13, 155.96, 136.39, 129.62, 128.73, 128.65, 128.29, 128.15, 127.14, 67.11, 58.05, 56.14, 40.72, 38.96, 38.17, 31.08, 29.53, 28.88; ESI-MS calcd for C₂₄H₂₈N₃O₅ (M+H⁺): 438.2; found 438.3.

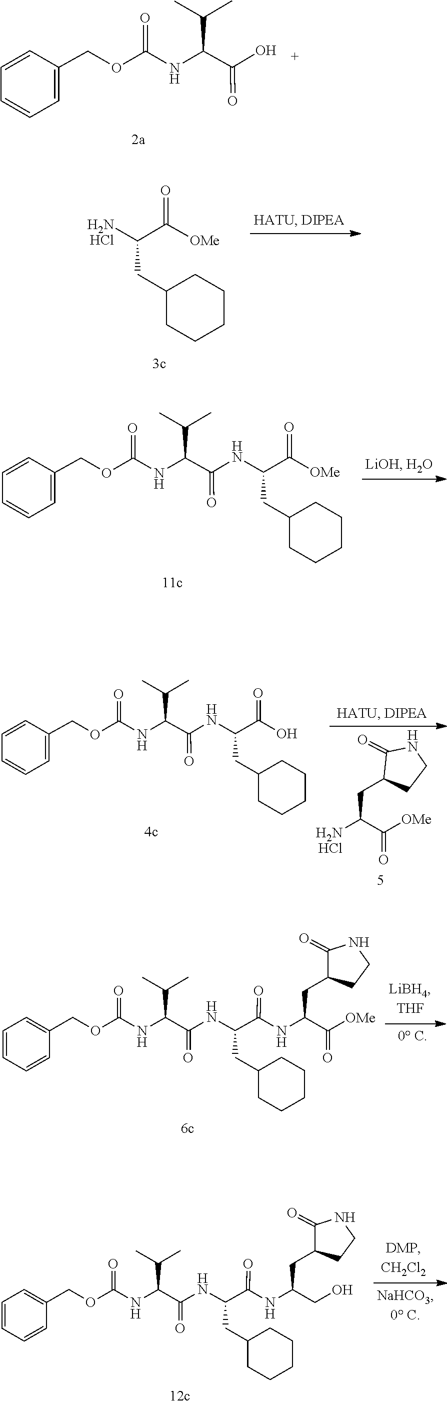

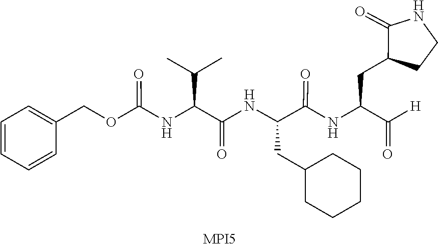 [SMILES: CC(C)[C@H](NC(=O)OCc1ccccc1)C(=O)N[C@@H](CC1CCCCC1)C(=O)N[C@H](C=O)C[C@@H]1CCNC1=O]

### Methyl (S)-2-((S)-2-(((benzyloxy)carbonyl)amino)-3-methylbutanamido)-3-cyclohexylpropanoate (11c)

To a solution of 2a (2 g, 7.95 mmol, 1.0 equiv) in anhydrous DMF (15 mL) at 0° C., and then 3c (1.8 g, 7.95 mmol, 1.0 equiv), HATU (4.5 g, 12.0 mmol, 1.5 equiv), DIPEA (7.0 mL, 40.0 mmol, 5.0 equiv) was added sequentially. The mixture was stirred at room temperature for 6 h. The mixture was diluted with EtOAc and washed with water, 1M HCl, sat. NaCl, dried over Na₂SO₄, and concentrated. The residue was purified by column chromatography (EtOAc:Hexane=1:2 v/v) to afford the pure product 11c as a white solid (2.7 g, 81%). ¹H NMR (400 MHz, CD₃OD-d₄) δ 7.4-7.3 (m, 5H), 5.1 (d, J=1.6 Hz, 2H), 4.5 (dd, J=9.6, 5.6 Hz, 1H), 4.0 (d, J=7.5 Hz, 1H), 3.7 (s, 3H), 2.2-2.0 (m, 1H), 1.8-1.6 (m, 7H), 1.4 (tdd, J=11.0, 6.9, 3.6 Hz, 1H), 1.3-1.1 (m, 3H), 1.0 (dd, J=11.7, 6.7 Hz, 7H), 0.9-0.8 (m, 1H). ¹³C NMR (100 MHz, CD₃OD-d₄) δ 174.5, 174.3, 158.5, 138.2, 129.4, 129.4, 129.0, 128.8, 128.8, 67.6, 61.9, 52.5, 51.3, 39.9, 35.2, 34.7, 33.1, 32.0, 27.5, 27.3, 27.1, 19.7, 18.7.

### (S)-2-((S)-2-(((benzyloxy)carbonyl)amino)-3-methylbutanamido)-3-cyclohexylpropanoic acid (4c)

To a solution of 11c (400 mg, 1.2 mmol, 1.0 equiv) in 1:1 THF/H₂O (8 mL) was added LiOH.H₂O (200 mg, 4.8 mmol, 4.0 equiv). The reaction was stirred at RT for 2 h. After completion, the reaction mixture was neutralized with 1M HCl solution and extracted with EtOAc. The organic layer was washed with sat. NaCl, dried over Na₂SO₄ and concentrated to afford the product 4c (310 mg, yield 80%) as a white solid. The residue was used in the next without further purification.

### Methyl (5S,8S,11S)-8-(cyclohexylmethyl)-5-isopropyl-3,6,9-trioxo-11-(((S)-2-oxopyrrolidin-3-yl)methyl)-1-phenyl-2-oxa-4,7,10-triazadodecan-12-oate (6c)

To a solution of 4c (300 mg, 0.74 mmol, 1.0 equiv) in anhydrous DMF (5 mL) at 0° C., and then 5 (165 mg, 0.74 mmol, 1.0 equiv), HATU (400 mg, 1.05 mmol, 1.5 equiv), DIPEA (610 μL, 3.7 mmol, 5.0 equiv) was added sequentially. The mixture was stirred at RT for 6 h. The mixture was diluted with EtOAc and washed with water, 1M HCl, sat. NaCl, dried over Na₂SO₄, and concentrated. The residue was purified by column chromatography (MeOH:DCM=1:20 v/v) to afford the pure product 6c as a white solid (250 mg, 60%).

### Benzyl ((S)-1-(((S)-3-cyclohexyl-1-(((S)-1-hydroxy-3-((S)-2-oxopyrrolidin-3-yl)propan-2-yl)amino)-1-oxopropan-2-yl)amino)-3-methyl-1-oxobutan-2-yl)carbamate (12c)

To a solution of 6c (250 mg, 0.44 mmol, 1.0 equiv) in anhydrous THF (10 mL) at 0° C. was added LiBH₄ (1.0 M in THF, 1.32 mL, 1.32 mmol, 3.0 equiv). The mixture was stirred at RT for 2 h. After the reaction was completed, excess reactants were consumed by slow addition of H₂O. The mixture was diluted with H₂O and extracted with EtOAc, washed with sat. NaCl, dried over Na₂SO₄ and concentrated. The residue was purified by column chromatography (MeOH:DCM=1:15 v/v) to afford the pure product 12c as a white solid (130 mg, 54%). ¹H NMR (400 MHz, CD₃OD-d₄) δ 7.3-7.1 (m, 5H), 5.1-4.9 (m, 2H), 4.3 (dd, J=9.0, 6.4 Hz, 1H), 3.9-3.8 (m, 2H), 3.5 (q, J=7.1 Hz, 1H), 3.5-3.3 (m, 2H), 3.2-3.1 (m, 2H), 2.4-2.3 (m, 1H), 2.2 (s, 1H), 2.0-1.8 (m, 2H), 1.7-1.4 (m, 9H), 1.1 (q, J=8.1, 7.1 Hz, 3H), 0.8 (dd, J=8.6, 6.7 Hz, 8H). ¹³C NMR (100 MHz, CD₃OD-d) δ 181.2, 173.5, 172.8, 157.4, 136.8, 128.1, 128.1, 127.6, 127.5, 127.5, 66.5, 64.2, 60.9, 51.4, 49.1, 40.1, 38.9, 38.1, 33.9, 33.5, 32.3, 32.0, 30.5, 27.6, 26.2, 26.0, 25.8, 18.4, 17.0.

### Benzyl ((S)-1-(((S)-3-cyclohexyl-1-oxo-1-(((S)-1-oxo-3-((S)-2-oxopyrrolidin-3-yl)propan-2-yl)amino)propan-2-yl)amino)-3-methyl-1-oxobutan-2-yl)carbamate (MPI5)

To a solution of 12c (130 mg, 0.24 mmol, 1.0 equiv) in anhydrous DCM (10 mL) was added Dess-Martin reagent (200 mg, 0.48 mmol, 2.0 equiv) slowly at 0° C. Then the reaction mixture was stirred at RT for 1 h. A solution of NaHCO₃ and Na₂S₂O₃ was added to quench the reaction. After 10 min, the mixture was washed with water, sat. NaCl, dried over Na₂SO₄ and concentrated. The residue was purified by column chromatography (MeOH:DCM=1:15 v/v). Dissolved the obtained aldehyde in abundant CCl₄ and hexane, then concentrate in vacuo. Re-dissolved the residue in least amount of CHCl₃ and add abundant hexane to precipitate out a white solid MPI5 (60 mg, 47%). ¹H NMR (400 MHz, CDCl₃-d) δ 9.4 (s, 1H), 8.1 (d, J=6.9 Hz, 1H), 7.3 (s, 5H), 7.1 (d, J=8.5 Hz, 1H), 6.7 (s, 1H), 5.6 (d, J=8.8 Hz, 1H), 5.0 (q, J=12.3 Hz, 2H), 4.6 (td, J=8.9, 5.7 Hz, 1H), 4.3 (p, J=5.0 Hz, 1H), 4.0 (t, J=7.8 Hz, 1H), 3.3-3.1 (m, 2H), 2.4-2.2 (m, 2H), 2.1-1.9 (m, 2H), 1.8 (ddd, J=13.6, 7.3, 4.1 Hz, 1H), 1.7-1.4 (m, 8H), 1.3-1.2 (m, 1H), 1.1-1.0 (m, 3H), 0.8 (dd, J=13.4, 6.9 Hz, 8H). ¹³C NMR (100 MHz, CDCl₃-d) δ 199.6, 180.1, 173.5, 171.5, 156.7, 136.3, 128.7, 128.7, 128.3, 128.1, 128.1, 67.2, 60.7, 57.4, 51.2, 40.7, 40.3, 38.0, 34.3, 33.6, 32.6, 31.1, 30.0, 28.4, 26.5, 26.3, 26.2, 19.3, 19.3. ESI-MS calcd for C₂₉H₄₃N₄O₆⁺ (M+H⁺): 543.3; found 543.3.

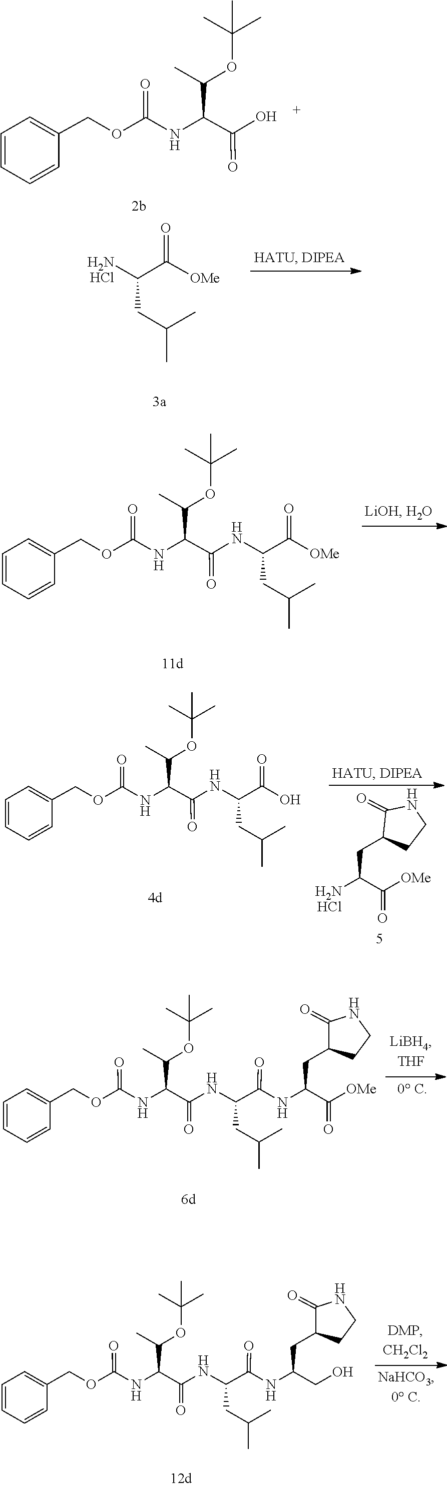

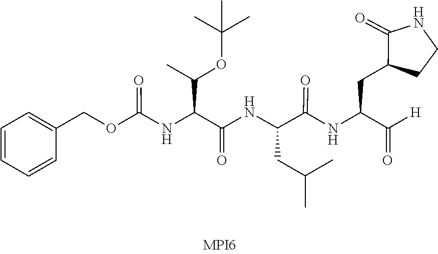 [SMILES: CC(C)C[C@H](NC(=O)[C@@H](NC(=O)OCc1ccccc1)C(C)OC(C)(C)C)C(=O)N[C@H](C=O)C[C@@H]1CCNC1=O]

### (S)-Methyl 2-((2S,3R)-2-(((benzyloxy)carbonyl)amino)-3-(tert-butoxy)butanamido)-4-methylpentanoate (11d)

The amino acid methyl ester hydrochloride 3a (1.0 g, 5.52 mmol) and the Cbz-protected amino acid 2b (1.52 g, 6.08 mmol) were dissolved in dry DMF (20 mL) and the reaction was cooled to 0° C. HATU (2.52 g, 6.62 mmol) and DIPEA (3.92 mL, 22.08 mmol) were added, and the reaction mixture was allowed warm up to room temperature and stirred for 12 h. The mixture was then poured into water (50 mL) and extracted with ethyl acetate (4×20 mL). The organic layer was washed with aqueous hydrochloric acid 10% v/v (2×20 mL), saturated aqueous NaHCO₃ (2×20 mL), brine (2×20 mL) and dried over Na₂SO₄. The organic phase was evaporated to dryness and the crude material purified by silica gel column chromatography (15-50% EtOAc in n-hexane as the eluent) to afford 11d as a gummy liquid (1.71 g, 71%). ¹H NMR (CDCl₃, 400 MHz): δ 7.58 (d, J=7.64 Hz, 1H), 7.28-7.21 (m, 5H), 5.88 (d, J=4.68 Hz, 1H), 5.04 (ABq, J=12.16 Hz, 2H), 4.47-4.41 (m, 1H), 4.15-4.08 (m, 2H), 3.65 (s, 3H), 1.63-1.46 (m, 3H), 1.23 (s, 9H), 1.03 (d, J=6.28, 3H), 0.86 (dd, J=3.84, 5.92 Hz, 6H); ¹³C NMR (CDCl₃, 100 MHz) δ 172.9, 169.4, 156.1, 136.3, 128.5 (2C), 128.0, 127.9, 75.5, 66.8, 60.4, 58.4, 52.2, 51.1, 41.3, 28.2 (3C), 25.0 22.8, 21.9, 16.4.

### (S)-2-((2S,3R)-2-(((Benzyloxy)carbonyl)amino)-3-(tert-butoxy)butanamido)-4-methylpentanoic acid (4d)

The peptide 11d (500 mg, 1.32 mmol) was dissolved in THF/H₂O (1:1, 6.0 mL), and LiOH (138 mg, 3.30 mmol) was added at 0° C. The mixture was stirred at room temperature overnight. Then THF was removed on vacuum and the aqueous layer was acidified with 1 M HCl and extracted with dichloromethane (3×10 mL). The organic layer was dried over anhydrous Na₂SO₄ and concentrated to yield 4d as white solid (350 mg, 70%). ¹H NMR (CDCl₃, 400 MHz): δ 7.62 (d, J=7.44 Hz, 1H), 7.29-7.22 (m, 5H), 5.94 (d, J=5.28 Hz, 1H), 5.04 (ABq, J=12.32 Hz, 2H), 4.45-4.40 (m, 1H), 4.17-4.08 (m, 2H), 1.68-1.50 (m, 3H), 1.21 (s, 9H), 1.02 (d, J=6.32 Hz, 3H), 0.87 (m, 6H); ¹³C NMR (CDCl₃, 100 MHz) δ 177.1, 169.9, 156.2, 136.2, 128.5 (2C), 128.2, 128.0 (2C), 75.6, 66.97, 66.92, 58.4, 51.1, 41.0, 28.2 (3C), 25.0, 22.8, 21.8, 16.5.

### (5S,8S,11S)-Methyl 5-((R)-1-(tert-butoxy)ethyl)-8-isobutyl-3,6,9-trioxo-11-(((S)-2-oxopyrrolidin-3-yl)methyl)-1-phenyl-2-oxa-4,7,10-triazadodecan-12-oate (6d)

The methyl (S)-2-amino-3-((S)-2-oxopyrrolidin-3-yl)propanoate hydrochloride 5 (140 mg, 0.606 mmol) and the peptide 4d (242 mg, 0.666 mmol) were dissolved in dry DMF (6 mL), and the reaction was cooled to 0° C. HATU (276 mg, 0.727 mmol) and DIPEA (0.43 mL, 2.42 mmol) were added, and the reaction mixture was allowed warm up to room temperature and stirred for 12 h. The mixture was then poured into water (20 mL) and extracted with ethyl acetate (4×20 mL). The organic layer was washed with aqueous hydrochloric acid 10% v/v (2×20 mL), saturated aqueous NaHCO₃ (2×20 mL), brine (2×20 mL) and dried over Na₂SO₄. The organic phase was evaporated to dryness and the crude material purified by silica gel column chromatography afford 6d as white solid (240 mg, 64%). ¹H NMR (CDCl₃, 400 MHz): δ 7.64 (d, J=7.08 Hz, 1H), 7.39 (d, J=7.92 Hz, 1H), 7.31-7.20 (m, 5H), 6.34 (Brs, 1H), 5.87 (d, J=5.0 Hz, 1H), 5.03 (ABq, J=12.36 Hz, 2H), 4.50-4.43 (m, 1H), 4.50-4.43 (m, 1H), 4.40-4.34 (m, 1H), 4.11-4.09 (m, 2H), 3.67 (s, 3H), 3.29-3.15 (m, 2H), 2.38-2.23 (m, 2H), 2.10-2.02 (m, 1H), 1.81-1.61 (m, 4H), 1.51-1.44 (m, 1H), 1.18 (s, 9H), 0.99 (d, J=5.76, 3H), 0.86 (dd, J=5.6, 14.32, 6H). ¹³C NMR (CDCl₃, 100 MHz): δ 179.7, 172.2, 169.5, 136.2, 128.6 (2C), 128.3, 128.1 (2C), 77.1, 75.4, 67.0, 66.7, 58.9, 52.4, 51.9, 51.1, 41.7, 40.5, 38.2, 33.0, 28.2 (3C), 28.2, 24.7, 22.8, 22.2, 17.2.

### Benzyl ((2S,3R)-3-(tert-butoxy)-1-(((S)-1-(((S)-1-hydroxy-3-((S)-2-oxopyrrolidin-3-yl)propan-2-yl)amino)-4-methyl-1-oxopentan-2-yl)amino)-1-oxobutan-2-yl)carbamate (12d)

To a stirring solution of compound 6d (120 mg, 0.225 mmol) in THF (5 mL) was added LiBH₄ (2.0 M in THF, 0.56 mL, 1.12 mmol) in several portions at 0° C. under a nitrogen atmosphere. The reaction mixture was stirred at 0° C. for 1 h, then allowed to warm up to room temperature, and stirred for an additional 2 h. The reaction was quenched by the drop wise addition of 1.0 M HCl(aq) (1.2 mL) with cooling in an ice bath. The solution was diluted with ethyl acetate and H₂O. The phases were separated, and the aqueous layer was extracted with ethyl acetate (3×15 mL). The organic phases were combined together, dried over Na₂SO₄, filtered, and concentrated on a rotorvap to give a yellow oily residue. Column chromatographic purification of the residue (6% MeOH in CH₂Cl₂ as the eluent) afforded a white solid 12d (85 mg, 68%). ¹H NMR (CDCl₃, 400 MHz) δ 7.66 (d, J=7.48 Hz, 1H), 7.41-7.30 (m, 5H), 6.13 (Brs, 1H), 6.02 (Brs, 1H), 5.13 (ABq, J=12.2 Hz, 2H), 4.42-4.36 (m, 1H), 4.18 (d, J=5.04, 2H), 4.07-3.97 (m, 1H), 3.65-3.58 (m, 2H), 3.35-3.28 (m, 2H), 2.46-2.37 (m, 2H), 2.10-2.02 (m, 1H), 1.97-1.78 (m, 2H), 1.71-1.53 (m, 3H), 1.28 (s, 9H), 1.09 (d, J=5.64 Hz, 3H), 0.94 (d, J=6.12, 10.36 Hz, 6H); ¹³C NMR (CDCl₃, 100 MHz): δ 180.9, 172.7, 169.8, 156.3, 136.1, 128.6 (2C), 77.2, 75.5, 66.9, 65.9, 59.1, 55.2, 50.5, 41.2, 40.5, 38.2, 32.4, 28.5, 28.2, 24.9, 22.8, 22.1, 17.4.

### Benzyl ((2S,3R)-3-(tert-butoxy)-1-(((S)-4-methyl-1-oxo-1-(((S)-1-oxo-3-((S)-2-oxopyrrolidin-3-yl)propan-2-yl)amino)pentan-2-yl)amino)-1-oxobutan-2-yl)carbamate (MPI6)

To a solution of 12d (70 mg, 0.138 mmol) in CH₂Cl₂ (6 mL) was added NaHCO₃ (46 mg, 4 equiv) and the Dess-Martin reagent (180 mg, 3 equiv). The resulting mixture was stirred at rt for 12 h. Then the reaction was quenched with a saturated NaHCO₃ solution containing 10% Na₂S₂O₃. The layers were separated. The organic layer was then washed with saturated brine solution, dried over anhydrous Na₂SO₄ and concentrated on vacuum. The residue was then purified with flash chromatography afford MPI6 as white solid (41 mg, 59%). ¹H NMR (400 MHz, CDCl₃): δ 9.44 (s, 1H), 8.04 (d, J=6.24 Hz, 1H), 7.41 (d, J=7.64 Hz, 1H), 7.31-7.27 (m, 5H), 6.14 (brs, 1H), 5.85 (d, J=4.68 Hz, 1H), 5.05 (ABq, J=12.2 Hz, 2H), 4.45-4.38 (m, 1H), 4.34-4.28 (m, 1H), 4.18-4.05 (m, 2H), 3.30-3.11 (m, 2H), 2.48-2.33 (m, 1H), 2.33-2.21 (m, 1H), 2.11-1.92 (m, 2H), 1.91-1.89 (m, 1H), 1.87-1.58 (m, 1H), 1.57-1.53 (m, 2H), 1.52-1.48 (m, 1H), 1.21 (s, 9H), 1.01 (d, J=5.92 Hz, 3H), 0.89 (dd, J=6.12, 11.56 Hz, 6H). ¹³C NMR (CDCl₃, 100 MHz): 199.6, 179.9, 172.8, 169.7, 156.2, 136.1, 128.6 (2C), 128.3, 128.1 (2C), 77.1, 75.4, 67.0, 66.7, 58.9, 57.6, 52.1, 41.6, 40.5, 37.9, 29.7, 28.5, 28.2 (3C), 24.9, 22.9, 22.1, 17.3.

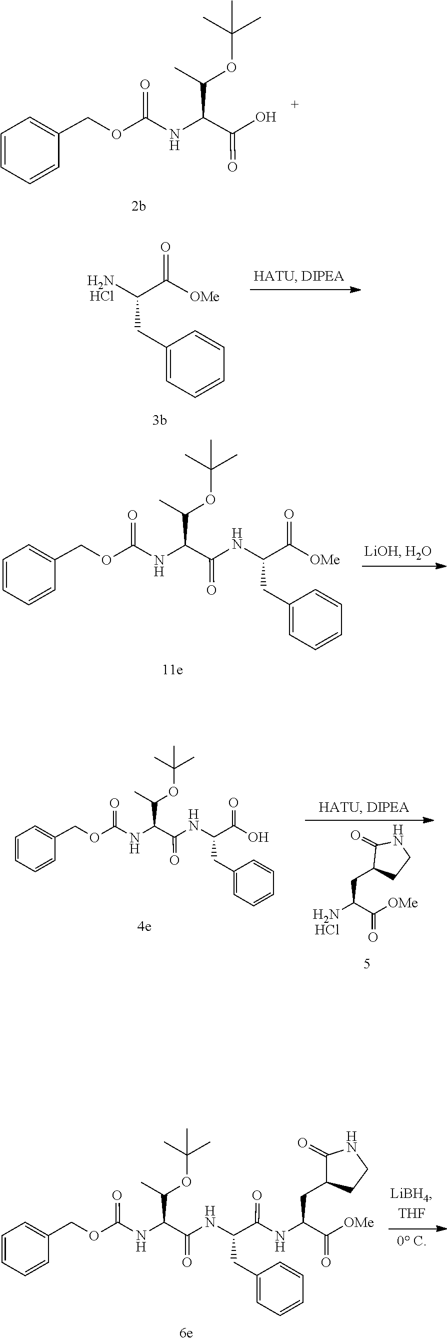

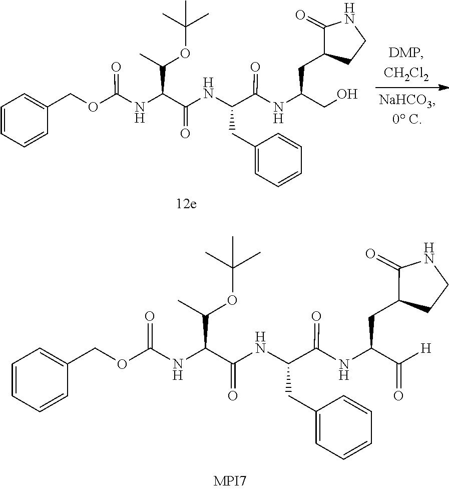 [SMILES: CC(OC(C)(C)C)[C@H](NC(=O)OCc1ccccc1)C(=O)N[C@@H](Cc1ccccc1)C(=O)N[C@H](CO)C[C@@H]1CCNC1=O ; CC(OC(C)(C)C)[C@H](NC(=O)OCc1ccccc1)C(=O)N[C@@H](Cc1ccccc1)C(=O)N[C@H](C=O)C[C@@H]1CCNC1=O]

### Methyl N-((benzyloxy)carbonyl)-O-(tert-butyl)-L-allothreonyl-L-phenylalaninate (11e)

To a solution of 2b (5 mmol, 1.55 g) and 3b (5 mmol, 1.07 g) in anhydrous DMF (20 mL) was added DIPEA (10 mmol, 1.29 g) and was cooled to 0° C. HATU (5.5 mmol, 2.09 g) was added to the solution under 0° C. and then stirred at room temperature overnight. The reaction mixture was then diluted with ethyl acetate (100 mL) and washed with saturated NaHCO₃ solution (2×50 mL), 1 M HCl solution (2×50 mL), and saturated brine solution (2×50 mL) sequentially. The organic layer was dried over anhydrous Na₂SO₄ and then concentrated on vacuo. The residue was then purified with flash chromatography (15-50% EtOAc in hexanes as the eluent) to afford 11e as colorless oil (1.64 g, 70%).

### N-((benzyloxy)carbonyl)-O-(tert-butyl)-L-allothreonyl-L-phenylalanine (4e)

11e (1 mmol, 470 mg) was dissolved in 5 mL of THF. A solution of LiOH.H₂O (2 mmol, 84 mg) in 5 mL H₂O was added to the solution. The mixture was stirred at room temperature overnight. Then THF was removed on vacuo and the aqueous layer was acidified with 1 M HCl and extracted with dichloromethane (3×10 mL). The organic layer was dried over anhydrous Na₂SO₄ and concentrated to yield 4e as white solid (330 mg, 72%). ¹H-NMR (400 MHz, CD₃OD): δ 7.29-7.43 (m, 5H), 7.11-7.29 (m, 5H), 5.11 (s, 2H), 4.71 (t, J=6.2 Hz, 1H), 4.14 (d, J=3.9 Hz, 1H), 3.98-4.07 (m, 1H), 3.17 (dd, J=13.9, 5.6 Hz, 1H), 3.05 (dd, J=13.8, 6.8 Hz, 1H), 0.98-1.16 (m, 12H).

### Methyl (5S,8S,11S)-8-benzyl-5-((R)-1-(tert-butoxy)ethyl)-3,6,9-trioxo-11-(((S)-2-oxopyrrolidin-3-yl)methyl)-1-phenyl-2-oxa-4,7,10-triazadodecan-12-oate (6e)

To a solution of 4e (0.4 mmol, 182 mg) and 5 (0.4 mmol, 88 mg) in anhydrous DMF (2 mL) was added DIPEA (0.8 mmol, 103 mg) and was cooled to 0° C. HATU (0.44 mmol, 167 mg) was added to the solution under 0° C. and then stirred at room temperature overnight. The reaction mixture was then diluted with ethyl acetate (20 mL) and washed with saturated NaHCO₃ solution (2×10 mL), 1 M HCl solution (2×10 mL), and saturated brine solution (2×10 mL) sequentially. The organic layer was dried over anhydrous Na₂SO₄ and then concentrated on vacuo. The residue was then purified with flash chromatography (1-10% methanol in dichloromethane as the eluent) to afford 6b as white solid (180 mg, 72%). ¹H-NMR (400 MHz, CDCl₃): δ 7.44-7.19 (m, 10H), 5.90 (d, J=5.4 Hz, 1H), 5.52 (s, 1H), 5.12 (q, J=12.3 Hz, 2H), 4.77 (q, J=6.8 Hz, 1H), 4.65-4.53 (m, 1H), 4.22-4.09 (m, 2H), 3.72 (s, 3H), 3.40-3.29 (m, 2H), 3.21 (dd, J=14.0, 6.4 Hz, 1H), 3.09 (dd, J=13.9, 6.1 Hz, 1H), 2.50-2.40 (m, 1H), 2.35-2.26 (m, 1H), 2.18-2.05 (m, 1H), 1.95-1.77 (m, 2H), 1.29 (s, 2H), 1.17 (s, 9H), 1.08 (d, J=6.2 Hz, 3H).

### Benzyl ((2S,3R)-3-(tert-butoxy)-1-oxo-1-(((S)-1-oxo-1-(((S)-1-oxo-3-((S)-2-oxopyrrolidin-3-yl)propan-2-yl)amino)-3-phenylpropan-2-yl)amino)butan-2-yl)carbamate (MPI7)

To a solution of 6e (0.1 mmol, 62 mg) in anhydrous dichloromethane (5 mL) was added a solution of LiBH₄ in anhydrous THF (2 M, 0.1 mL, 0.2 mmol) at 0° C. The resulting solution was stirred at the same temperature for 3 h. Then a saturated solution of NH₄Cl (5 mL) was added dropwise to quench the reaction. The layers were separated, and the organic layer was washed with saturated brine solution (2×10 mL), dried over anhydrous Na₂SO₄, and evaporated to dryness. The residue was then dissolved in anhydrous dichloromethane (5 mL) and cooled to 0° C. Dess-Martin periodinane (0.2 mmol, 85 mg) was added to the solution. The reaction mixture was then stirred at room temperature overnight. Then the reaction was quenched with a saturated NaHCO₃ solution containing 10% Na₂S₂O₃. The layers were separated. The organic layer was then washed with saturated brine solution (2×10 mL), dried over anhydrous Na₂SO₄ and evaporated on vacuo. The residue was then purified with flash chromatography (1-10% methanol in dichloromethane as the eluent) to afford MPI7 as white solid (27 mg, 45%). ¹H-NMR (400 MHz, CDCl₃): δ 9.28 (s, 1H), 7.80 (d, J=6.5 Hz, 1H), 7.44-7.09 (m, 10H), 5.87 (d, J=5.6 Hz, 1H), 5.71 (s, 1H), 5.08 (q, J=11.1 Hz, 2H), 4.76 (q, J=7.1 Hz, 1H), 4.32 (q, J=7.0 Hz, 1H), 4.19-4.04 (m, 2H), 3.38-3.26 (m, 2H), 3.19 (dd, J=13.8, 6.3 Hz, 1H), 3.07 (dd, J=13.8, 6.8 Hz, 1H), 2.41-2.22 (m, 2H), 1.88 (t, J=6.7 Hz, 2H), 1.84-1.73 (m, 1H), 1.30-1.22 (m, 1H), 1.16 (s, 9H), 1.05 (d, J=6.1 Hz, 3H); ¹³C NMR (100 MHz, CDCl₃) δ 199.60, 179.68, 171.55, 169.49, 156.32, 136.34, 136.28, 129.49, 128.87, 128.73, 128.40, 128.20, 127.35, 84.26, 75.61, 67.15, 66.80, 59.18, 57.74, 54.53, 40.53, 38.32, 37.82, 28.80, 28.23, 17.63. ESI-MS calcd for C₃₂H₄₃N₄O₇ (M+H⁺): 595.3; found: 595.4.

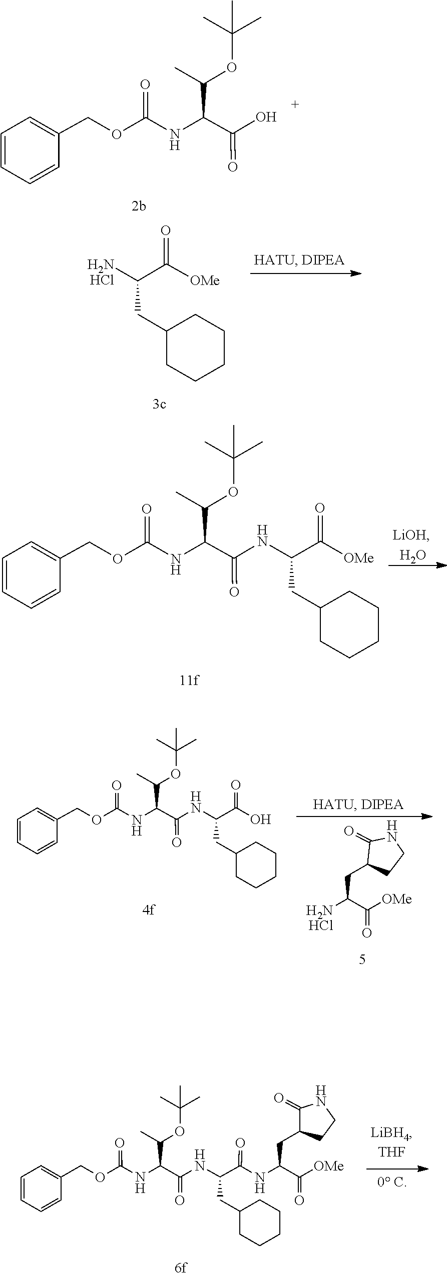

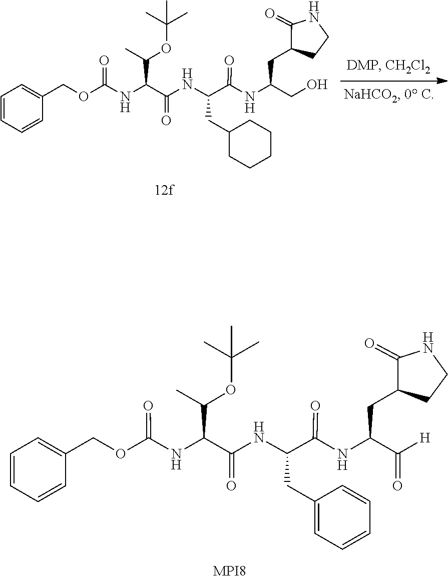 [SMILES: CC(OC(C)(C)C)[C@H](NC(=O)OCc1ccccc1)C(=O)N[C@@H](CC1CCCCC1)C(=O)N[C@H](CO)C[C@@H]1CCNC1=O ; CC(OC(C)(C)C)[C@H](NC(=O)OCc1ccccc1)C(=O)N[C@@H](Cc1ccccc1)C(=O)N[C@H](C=O)C[C@@H]1CCNC1=O]

### Methyl (S)-2-((2S,3R)-2-(((benzyloxy)carbonyl)amino)-3-(tert-butoxy)butanamido)-3-cyclohexylpropanoate (11f)

To a solution of 2b (2 g, 6.46 mmol, 1.0 equiv), in anhydrous DMF (15 mL) at 0° C., and then 3c (1.4 g, 6.46 mmol, 1.0 equiv), HATU (3.7 g, 9.70 mmol, 1.5 equiv) and DIPEA (5.7 mL, 32.3 mmol, 5.0 equiv) was added sequentially. The mixture was stirred at RT for 6 h. The mixture was diluted with EtOAc and washed with water, 1M HCl, sat. NaCl, dried over Na₂SO₄, and concentrated. The residue was purified by column chromatography (EtOAc:Hexane=1:4 v/v) to afford the pure product 11f as a colorless oil (2.5 g, 83%). ¹H NMR (400 MHz, CDCl₃-d) δ 7.6 (d, J=7.8 Hz, 1H), 7.3-7.1 (m, 5H), 5.9 (d, J=5.3 Hz, 1H), 5.1-4.9 (m, 2H), 4.4 (td, J=8.4, 5.1 Hz, 1H), 4.2-4.1 (m, 2H), 3.6 (s, 3H), 1.7-1.5 (m, 6H), 1.5 (ddd, J=14.2, 9.0, 5.7 Hz, 1H), 1.2 (s, 9H), 1.2-1.1 (m, 3H), 1.0 (d, J=6.3 Hz, 4H), 0.9-0.7 (m, 2H). ¹³C NMR (100 MHz, CDCl₃-d) δ 172.7, 169.2, 155.9, 136.2, 128.3, 128.3, 127.8, 127.7, 127.7, 75.2, 66.7, 66.5, 60.0, 51.8, 50.2, 39.5, 34.0, 33.3, 32.3, 28.0, 28.0, 28.0, 26.1, 26.0, 25.8, 20.7.

### (S)-2-((2S,3R)-2-(((benzyloxy)carbonyl)amino)-3-(tert-butoxy)butanamido)-3-cyclohexylpropanoic acid (4f)

To a solution of 11f (400 mg, 0.84 mmol, 1.0 equiv) in 1:1 THF/H₂O (8 mL) was added LiOH.H₂O (140 mg, 3.4 mmol, 4.0 equiv). The reaction was stirred at RT for 2 h. After completion, the reaction mixture was neutralized with 1M HCl solution and extracted with EtOAc. The organic layer was washed with sat. NaCl, dried over Na₂SO₄ and concentrated to afford the product 4f (330 mg, 85%) as a white solid. The residue was used in the next without further purification.

### Methyl (5S,8S,11S)-5-((R)-1-(tert-butoxy)ethyl)-8-(cyclohexylmethyl)-3,6,9-trioxo-11-(((S)-2-oxopyrrolidin-3-yl)methyl)-1-phenyl-2-oxa-4,7,10-triazadodecan-12-oate (6f)

To a solution of 4f (300 mg, 0.65 mmol, 1.0 equiv) in anhydrous DMF (5 mL) at 0° C., and then 5 (144 mg, 0.65 mmol, 1.0 equiv), HATU (370 mg, 0.98 mmol, 1.5 equiv), DIPEA (580 μL, 3.25 mmol, 5.0 equiv) was added sequentially. The mixture was stirred at RT for 6 h. The mixture was diluted with EtOAc and washed with water, 1M HCl, sat. NaCl, dried over Na₂SO₄, and concentrated. The residue was purified by column chromatography (MeOH:DCM=1:15 v/v) to afford the pure product 6f as a white solid (265 mg, 65%).

### Benzyl ((2S,3R)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (12f)

To a solution of 6f (250 mg, 0.40 mmol, 1.0 equiv) in anhydrous THF (10 mL) at 0° C. was added LiBH₄ (1.0 M in THF, 1.2 mL, 1.20 mmol, 3.0 equiv). The mixture was stirred at RT for 2 h. After the reaction was completed, excess reactants were consumed by slow addition of H₂O. The mixture was diluted with H₂O and extracted with EtOAc, washed with sat. NaCl, dried over Na₂SO₄ and concentrated. The residue was purified by column chromatography (MeOH:DCM=1:15 v/v) to afford the pure product 12f as a white solid (160 mg, 66%).

### Benzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate (MPI8)

To a solution of 12f (160 mg, 0.26 mmol, 1.0 equiv) in anhydrous DCM (10 mL) was added Dess-Martin reagent (225 mg, 0.52 mmol, 2.0 equiv) slowly at 0° C. Then the reaction mixture was stirred at RT for 1 h. A solution of NaHCO₃ and Na₂S₂O₃ was added to quench the reaction. After 10 min, the mixture was washed with water, sat. NaCl, dried over Na₂SO₄ and concentrated. The residue was purified by column chromatography (MeOH:DCM=1:15 v/v). Dissolved the obtained aldehyde in abundant CCl₄ and hexane, then concentrate in vacuo. Re-dissolved the residue in least amount of CHCl₃ and add abundant hexane to precipitate out a white solid MPI8 (82 mg, 53%). ¹H NMR (400 MHz, CDCl₃-d) δ 9.5 (s, 1H), 8.1 (d, J=6.8 Hz, 1H), 7.5 (q, J=8.4, 7.6 Hz, 1H), 7.4-7.3 (m, 5H), 6.8 (s, 1H), 5.9 (d, J=5.6 Hz, 1H), 5.1 (q, J=12.1 Hz, 2H), 4.5 (td, J=8.6, 5.6 Hz, 1H), 4.4 (q, J=9.9, 7.2 Hz, 1H), 4.2-4.1 (m, 2H), 3.2 (p, J=8.4, 7.1 Hz, 2H), 2.5-2.4 (m, 1H), 2.3-2.2 (m, 1H), 2.0 (ddt, J=16.2, 11.3, 5.6 Hz, 1H), 1.8 (ddd, J=13.3, 7.9, 4.2 Hz, 1H), 1.8-1.5 (m, 8H), 1.4-1.1 (m, 12H), 1.0 (d, J=6.2 Hz, 4H), 1.0-0.8 (m, 2H). ¹³C NMR (100 MHz, CDCl₃-d) δ 199.7, 180.0, 173.0, 169.8, 156.3, 136.2, 128.6, 128.6, 128.3, 128.1, 128.1, 75.4, 67.0, 66.8, 59.0, 57.3, 51.4, 40.6, 40.2, 37.9, 34.2, 34.2, 33.6, 32.7, 29.9, 28.3, 28.3, 28.3, 26.4, 26.2, 26.0, 17.4. ESI-MS calcd for C₃₂H₄₉N₄O₇⁺ (M+H⁺): 601.3; found 601.3.

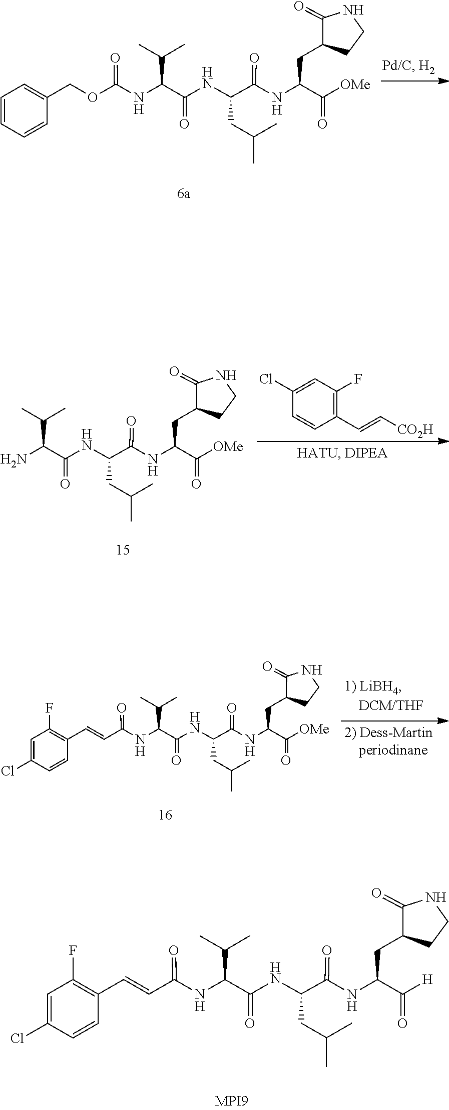 [SMILES: COC(=O)[C@H](C[C@@H]1CCNC1=O)NC(=O)[C@H](CC(C)C)NC(=O)[C@@H](NC(=O)OCc1ccccc1)C(C)C ; COC(=O)[C@H](C[C@@H]1CCNC1=O)NC(=O)[C@H](CC(C)C)NC(=O)[C@@H](N)C(C)C ; O=C(O)/C=C/c1ccc(Cl)cc1F ; COC(=O)[C@H](C[C@@H]1CCNC1=O)NC(=O)[C@H](CC(C)C)NC(=O)[C@@H](NC(=O)/C=C/c1ccc(Cl)cc1F)C(C)C ; CC(C)C[C@H](NC(=O)[C@@H](NC(=O)/C=C/c1ccc(Cl)cc1F)C(C)C)C(=O)N[C@H](C=O)C[C@@H]1CCNC1=O]

### Methyl (S)-2-((S)-2-((S)-2-((E)-3-(4-chloro-2-fluorophenyl)acrylamido)-3-methylbutanamido)-4-methylpentanamido)-3-((S)-2-oxopyrrolidin-3-yl)propanoate (16)

To a solution of 6a (106 mg, 0.2 mmol) in methanol (5 mL) was added 10% Pd/C (21 mg). The mixture was then stirred with hydrogen balloon at room temperature for 3 h. The reaction mixture was then filtered, and the filtrate was concentrated in vacuo to afford 15 as colorless oil, which was used without further purification.

To a solution of 15 in 2 mL anhydrous DMF was added 2-fluoro-4-chlorocinnamic acid (40 mg, 0.2 mmol) and DIPEA (0.4 mmol, 52 mg). The solution was cooled to 0° C., followed by the addition of HATU (0.24 mmol, 91 mg). The solution was then allowed to warm up to room temperature and stirred overnight. The reaction mixture was then diluted with ethyl acetate (20 mL) and washed with saturated NaHCO₃ solution (2×10 mL), 1 M HCl solution (2×10 mL), and saturated brine solution (2×10 mL) sequentially. The organic layer was dried over anhydrous Na₂SO₄ and then concentrated in vacuo. The residue was then purified with flash chromatography (1-10% methanol in dichloromethane as the eluent) to afford 16 as white solid (75 mg, 65%). ¹H NMR (400 MHz, DMSO-d₆) δ 8.46 (d, J=8.2 Hz, 1H), 8.25 (d, J=8.9 Hz, 1H), 8.11 (d, J=7.7 Hz, 1H), 7.66 (dd, J=17.7, 9.4 Hz, 2H), 7.54 (dd, J=10.7, 2.1 Hz, 1H), 7.44 (d, J=16.0 Hz, 1H), 7.38 (dd, J=8.4, 2.1 Hz, 1H), 7.00 (d, J=16.0 Hz, 1H), 4.42-4.24 (m, 3H), 3.62 (s, 3H), 3.16 (t, J=9.2 Hz, 1H), 3.06 (q, J=9.3, 8.9 Hz, 1H), 2.36-2.27 (m, 1H), 2.12-1.95 (m, 3H), 1.62 (dtd, J=20.8, 11.7, 10.8, 6.9 Hz, 3H), 1.46 (t, J=7.3 Hz, 2H), 0.95-0.81 (m, 12H).

### (S)-2-((S)-2-((E)-3-(4-chloro-2-fluorophenyl)acrylamido)-3-methylbutanamido)-4-methyl-N—((S)-1-oxo-3-((S)-2-oxopyrrolidin-3-yl)propan-2-yl)pentanamide (MPI9)

To a solution of 16 (0.1 mmol, 58 mg) in anhydrous tetrahydrofuran (5 mL) was dropwise added a solution of LiBH₄ in tetrahydrofuran (2 M, 0.1 mL, 0.2 mmol) at 0° C. The solution was stirred under the same temperature for 3 h. Then a saturated solution of NH₄Cl (5 mL) was added dropwise to quench the reaction. The layers were separated, and the organic layer was washed with saturated brine solution (2×10 mL), dried over anhydrous Na₂SO₄, and evaporated to dryness. The residue was then dissolved in anhydrous dichloromethane (5 mL) and cooled to 0° C. Dess-Martin periodinane (0.2 mmol, 85 mg) was added to the solution. The reaction mixture was then stirred at room temperature overnight. Then the reaction was quenched with a saturated NaHCO₃ solution containing 10% Na₂S₂O₃. The layers were separated. The organic layer was then washed with saturated brine solution (2×10 mL), dried over anhydrous Na₂SO₄ and evaporated in vacuo. The residue was then purified with flash chromatography (1-10% methanol in dichloromethane as the eluent) to afford MPI9 as white solid (32 mg, 58%). ¹H NMR (400 MHz, DMSO-d₆) δ 9.41 (s, 1H), 8.46 (d, J=7.9 Hz, 1H), 8.26 (d, J=8.7 Hz, 1H), 8.17 (d, J=7.6 Hz, 1H), 7.66 (t, J=8.3 Hz, 1H), 7.62 (s, 1H), 7.53 (dd, J=10.6, 2.1 Hz, 1H), 7.43 (d, J=15.9 Hz, 1H), 7.37 (dd, J=8.5, 2.0 Hz, 1H), 7.00 (d, J=15.9 Hz, 1H), 4.42-4.18 (m, 3H), 3.16 (t, J=9.1 Hz, 1H), 3.06 (td, J=9.4, 7.1 Hz, 1H), 2.36-2.21 (m, 1H), 2.13 (dt, J=14.0, 7.8 Hz, 1H), 2.01 (h, J=6.6 Hz, 1H), 1.88 (ddd, J=14.9, 11.4, 4.0 Hz, 1H), 1.70-1.56 (m, 3H), 1.55-1.39 (m, 2H), 0.97-0.73 (m, 12H); ¹³C NMR (101 MHz, DMSO) δ 201.24, 178.73, 173.08, 171.32, 165.00, 161.93, 159.41, 135.01, 134.90, 130.74, 130.63, 126.15, 125.84, 122.35, 122.23, 117.35, 117.09, 58.07, 56.54, 51.68, 41.08, 37.59, 31.34, 29.82, 27.75, 24.67, 23.30, 22.24, 19.59, 18.53. ESI-MS: calcd for C₂₇H₃₇ClFN₄O₅ (M+H⁺): 551.2; found 551.4.

### Benzyl ((S)-1-(((S)-3-(furan-2-yl)-1-oxo-1-(((S)-1-oxo-3-((S)-2-oxopyrrolidin-3-yl)propan-2-yl)amino)propan-2-yl)amino)-3-methyl-1-oxobutan-2-yl)carbamate (MPI12)

The synthesis of MPI12 was based on Representative synthetic procedure II. ¹H NMR (400 MHz, Chloroform-d) δ 9.34 (s, 1H), 8.22 (d, J=6.8 Hz, 1H), 7.27 (q, J=7.8, 5.8 Hz, 5H), 7.02 (d, J=8.1 Hz, 1H), 6.16 (d, J=2.5 Hz, 2H), 6.03 (d, J=3.1 Hz, 1H), 5.40 (t, J=11.0 Hz, 1H), 5.03 (d, J=6.8 Hz, 2H), 4.79 (q, J=6.7 Hz, 1H), 4.22 (d, J=10.4 Hz, 1H), 4.00-3.88 (m, 1H), 3.23 (s, 4H), 2.25 (s, 2H), 2.17-2.02 (m, 1H), 1.76 (d, J=35.2 Hz, 5H), 0.84 (dd, J=25.3, 6.8 Hz, 6H). ¹³C NMR (100 MHz, Chloroform-d) δ 199.96, 179.97, 171.32, 171.18, 150.71, 142.04, 136.11, 128.60, 128.29, 128.11, 110.41, 108.07, 67.26, 52.41, 50.86, 40.56, 30.70, 28.57, 19.26, 17.59.

### Benzyl ((S)-3-methyl-1-oxo-1-(((S)-1-oxo-1-(((S)-1-oxo-3-((S)-2-oxopyrrolidin-3-yl)propan-2-yl)amino)-3-(thiophen-2-yl)propan-2-yl)amino)butan-2-yl)carbamate (MPI13)

The synthesis of MPI13 was based on Representative synthetic procedure II. ¹H NMR (400 MHz, Chloroform-d) δ 9.30 (s, 1H), 8.24 (s, 1H), 7.38-7.22 (m, 4H), 7.04 (d, J=5.0 Hz, 1H), 6.91 (d, J=8.4 Hz, 1H), 6.87-6.65 (m, 2H), 5.97 (s, 1H), 5.33 (d, J=8.2 Hz, 1H), 5.03 (s, 2H), 4.80 (d, J=7.7 Hz, 1H), 4.20 (s, 1H), 3.97 (t, J=7.2 Hz, 1H), 3.49-3.03 (m, 4H), 2.24 (s, 2H), 2.14-1.93 (m, 2H), 1.76 (d, J=33.7 Hz, 3H), 0.83 (dd, J=25.9, 6.7 Hz, 6H).

### N—((S)-3-methyl-1-(((S)-4-methyl-1-oxo-1-(((S)-1-oxo-3-((S)-2-oxopyrrolidin-3-yl)propan-2-yl)amino)pentan-2-yl)amino)-1-oxobutan-2-yl)-1H-indole-2-carboxamide (MPI14)

The synthesis of MPI14 was based on Representative synthetic procedure I. ¹H NMR (400 MHz, DMSO-d₆) δ 11.59 (d, J=2.2 Hz, 1H), 9.42 (s, 1H), 8.48 (d, J=7.8 Hz, 1H), 8.23 (dd, J=19.8, 8.1 Hz, 2H), 7.71-7.57 (m, 2H), 7.50-7.39 (m, 1H), 7.29 (d, J=2.1 Hz, 1H), 7.18 (ddd, J=8.2, 6.9, 1.2 Hz, 1H), 7.03 (ddd, J=8.0, 6.9, 1.0 Hz, 1H), 4.44-4.32 (m, 2H), 4.24 (ddd, J=11.6, 7.8, 3.9 Hz, 1H), 3.21-3.11 (m, 1H), 3.07 (td, J=9.2, 7.0 Hz, 1H), 2.31 (qd, J=10.3, 3.9 Hz, 1H), 2.13 (dt, J=14.3, 7.9 Hz, 2H), 1.96-1.85 (m, 1H), 1.73-1.58 (m, 3H), 1.58-1.43 (m, 2H), 1.02-0.79 (m, 12H). ¹³C NMR (101 MHz, DMSO-d₆) δ 201.2, 178.8, 173.1, 171.5, 161.5, 137.0, 131.8, 127.5, 123.9, 122.0, 120.2, 112.7, 104.2, 58.8, 56.6, 51.6, 41.2, 37.6, 30.9, 29.8, 27.7, 24.7, 23.3, 22.3, 19.6, 19.2.

### Benzyl ((S)-1-(((S)-4,4-dimethyl-1-oxo-1-(((S)-1-oxo-3-((S)-2-oxopyrrolidin-3-yl)propan-2-yl)amino)pentan-2-yl)amino)-3-methyl-1-oxobutan-2-yl)carbamate (MPI15)

The synthesis of MPI15 was based on Representative synthetic procedure II. ¹H NMR (400 MHz, Chloroform-d): δ 9.42 (s, 1H), 7.32-7.23 (m, 5H), 6.64-6.41 (m, 1H), 5.91 (d, J=26.3 Hz, 1H), 5.43 (d, J=8.9 Hz, 1H), 5.31 (d, J=8.6 Hz, 1H), 5.03 (d, J=7.8 Hz, 2H), 4.51 (dt, J=8.6, 4.4 Hz, 1H), 4.26 (d, J=7.0 Hz, 1H), 4.18-4.01 (m, 1H), 3.92 (dd, J=8.4, 6.3 Hz, 1H), 3.29-3.26 (m, 2H), 2.44-2.25 (m, 2H), 2.16-2.05 (m, 1H), 1.91-1.87 (m, 2H), 1.80-1.73 (m, 1H), 1.58-1.54 (m, 2H), 1.02-0.73 (m, 15H). ¹³C NMR (101 MHz, CDCl₃): δ 199.5, 180.0, 173.8, 171.1, 156.6, 136.2, 128.5, 128.2, 128.0, 67.1, 60.6, 57.2, 50.9, 46.4, 40.6, 37.9, 31.0, 30.6, 29.9, 29.6, 28.2, 19.2, 18.1.

### Benzyl ((S)-3-methyl-1-(((S)-4-methyl-1-oxo-1-(((S)-1-oxo-3-((S)-2-oxopyrrolidin-3-yl)propan-2-yl)amino)pent-4-en-2-yl)amino)-1-oxobutan-2-yl)carbamate (MPI16)

The synthesis of MPI16 was based on Representative synthetic procedure II. ¹H NMR (400 MHz, Chloroform-d) δ 9.48 (s, 1H), 8.33-8.20 (m, 1H), 7.42-7.29 (m, 5H), 6.63 (d, J=7.8 Hz, 1H), 6.05 (s, 1H), 5.34 (d, J=8.2 Hz, 1H), 5.10 (s, 2H), 4.82 (t, J=1.7 Hz, 1H), 4.75 (s, 1H), 4.68 (td, J=8.5, 5.7 Hz, 1H), 4.33 (d, J=8.5 Hz, 1H), 4.02 (dd, J=8.2, 5.8 Hz, 1H), 3.33 (dt, J=8.9, 4.3 Hz, 2H), 2.62 (dd, J=14.1, 5.5 Hz, 1H), 2.53-2.32 (m, 3H), 2.21-2.09 (m, 1H), 2.05-1.88 (m, 2H), 1.87-1.82 (m, 1H), 1.75 (s, 3H), 0.93 (dd, J=23.8, 6.8 Hz, 6H). ¹³C NMR (101 MHz, CDCl₃): δ 199.7, 180.0, 172.4, 171.4, 156.6, 140.8, 136.2, 128.5, 128.2, 128.0, 114.4, 67.1, 60.6, 57.5, 51.4, 41.0, 40.6, 38.0, 30.9, 29.9, 28.3, 21.9, 19.2, 17.7.

### Benzyl ((S)-1-(((S)-4,4-dimethyl-1-oxo-1-(((S)-1-oxo-3-((S)-2-oxopyrrolidin-3-yl)propan-2-yl)amino)pentan-2-yl)amino)-3,3-dimethyl-1-oxobutan-2-yl)carbamate (MPI17)

The synthesis of MPI17 was based on Representative synthetic procedure II. ¹H NMR (400 MHz, Chloroform-d) δ 9.50 (s, 1H), 8.16-7.99 (m, 1H), 7.42-7.28 (m, 5H), 6.90-6.74 (m, 1H), 5.55 (dd, J=22.5, 9.6 Hz, 1H), 5.08 (d, J=2.7 Hz, 2H), 4.72-4.58 (m, 1H), 4.38 (dd, J=10.6, 5.9 Hz, 1H), 3.96 (dd, J=13.1, 9.4 Hz, 1H), 3.47-3.25 (m, 2H), 2.51-2.29 (m, 2H), 2.09-1.77 (m, 4H), 1.53-1.43 (m, 1H), 1.13-0.71 (m, 18H). ¹³C NMR (101 MHz, CDCl₃): δ 199.3, 18.0, 173.7, 170.3, 156.5, 136.3, 128.5, 128.2, 127.9, 67.1, 62.6, 57.0, 50.8, 46.6, 40.5, 37.8, 36.5, 34.7, 30.6, 29.9, 29.5, 28.0, 26.5.

### Benzyl ((S)-1-cyclopropyl-2-(((S)-4,4-dimethyl-1-oxo-1-(((S)-1-oxo-3-((S)-2-oxopyrrolidin-3-yl)propan-2-yl)amino)pentan-2-yl)amino)-2-oxoethyl)carbamate (MPI18)

The synthesis of MPI18 was based on Representative synthetic procedure II. ¹H NMR (400 MHz, Chloroform-d): δ 9.48 (s, 1H), 8.22 (s, 1H), 7.40-7.27 (m, 5H), 7.06-6.87 (m, 1H), 6.51-6.29 (m, 1H), 5.78 (s, 1H), 5.07 (s, 2H), 4.68-4.52 (m, 1H), 4.36-4.24 (m, 1H), 3.56 (ddd, J=9.2, 7.0, 2.4 Hz, 1H), 3.36-3.19 (m, 2H), 2.50-2.28 (m, 2H), 2.07-1.84 (m, 4H), 1.84-1.72 (m, 1H), 1.21-1.07 (m, 1H), 0.95 (s, 9H), 0.66-0.35 (m, 4H). ¹³C NMR (101 MHz, CDCl₃): δ 199.77, 180.13, 173.80, 171.01, 156.44, 136.16, 128.55, 128.21, 128.04, 67.10, 59.12, 57.47, 51.03, 45.90, 40.59, 38.03, 30.62, 30.01, 29.64, 28.29, 13.85, 3.29, 3.22.

### Benzyl ((2S,3R)-3-(tert-butoxy)-1-(((S)-3-cyclopropyl-1-oxo-1-(((S)-1-oxo-3-((S)-2-oxopyrrolidin-3-yl)propan-2-yl)amino)propan-2-yl)amino)-1-oxobutan-2-yl)carbamate (MPI19)

The synthesis of MPI19 was based on Representative synthetic procedure II. ¹H NMR (400 MHz, Chloroform-d) δ 9.4 (s, 1H), 8.1 (d, J=6.3 Hz, 1H), 7.5 (t, J=8.6 Hz, 1H), 7.3 (s, 5H), 6.4-6.1 (m, 1H), 5.9 (d, J=5.4 Hz, 1H), 5.0 (q, J=12.3 Hz, 2H), 4.4 (dt, J=34.9, 6.6 Hz, 1H), 4.3 (p, J=5.6, 4.6 Hz, 1H), 4.1 (t, J=5.9 Hz, 2H), 3.3 (d, J=8.6 Hz, 1H), 3.2-3.2 (m, 1H), 2.3 (d, J=14.0 Hz, 1H), 2.2 (s, 2H), 2.0-1.9 (m, 1H), 1.9 (t, J=6.8 Hz, 1H), 1.7 (dt, J=15.4, 8.5 Hz, 1H), 1.6 (dd, J=12.9, 6.3 Hz, 1H), 1.1 (d, J=72.7 Hz, 12H), 0.7 (d, J=9.1 Hz, 1H), 0.4 (q, J=8.5 Hz, 2H), 0.0 (d, J=5.1 Hz, 2H). ¹³C NMR (100 MHz, Chloroform-d) δ 199.7, 180.1, 172.4, 169.6, 156.3, 136.2, 128.7, 128.4, 128.2, 75.5, 67.1, 66.9, 59.2, 57.7, 55.0, 40.6, 38.0, 37.5, 29.9, 28.6, 28.3, 17.7, 7.4, 4.7, 4.5.

### Benzyl ((2S,3R)-3-(tert-butoxy)-1-(((S)-4,4-dimethyl-1-oxo-1-(((S)-1-oxo-3-((S)-2-oxopyrrolidin-3-yl)propan-2-yl)amino)pentan-2-yl)amino)-1-oxobutan-2-yl)carbamate (MPI20)

The synthesis of MPI20 was based on Representative synthetic procedure II. ¹H NMR (400 MHz, Chloroform-d) δ 9.4 (s, 1H), 8.1 (d, J=6.6 Hz, 1H), 7.5 (d, J=8.3 Hz, 1H), 7.3-7.2 (m, 5H), 6.8 (s, 1H), 5.9 (d, J=5.1 Hz, 1H), 5.0 (q, J=12.2 Hz, 2H), 4.5 (td, J=8.5, 4.1 Hz, 1H), 4.3 (dq, J=10.8, 5.4, 4.8 Hz, 1H), 4.1 (d, J=5.8 Hz, 2H), 3.3-3.1 (m, 2H), 2.3 (p, J=8.3 Hz, 1H), 2.3-2.2 (m, 1H), 1.9 (td, J=12.4, 10.2, 5.7 Hz, 1H), 1.8 (ddt, J=11.8, 8.3, 3.9 Hz, 2H), 1.7 (tt, J=12.3, 6.2 Hz, 1H), 1.4 (dd, J=14.4, 8.6 Hz, 1H), 1.1 (d, J=81.8 Hz, 12H), 0.9 (d, J=7.3 Hz, 9H). ¹³C NMR (100 MHz, Chloroform-d) δ 199.6, 180.0, 173.3, 169.3, 156.2, 136.2, 128.6, 128.2, 128.1, 75.6, 67.0, 66.8, 58.9, 57.3, 51.2, 46.1, 40.5, 37.8, 30.5, 29.9, 29.8, 29.7, 28.2, 17.0.

### Benzyl ((S)-1-(((S)-3-cyclopropyl-1-oxo-1-(((S)-1-oxo-3-((S)-2-oxopyrrolidin-3-yl)propan-2-yl)amino)propan-2-yl)amino)-3-methyl-1-oxobutan-2-yl)carbamate (MPI21)

The synthesis of MPI21 was based on Representative synthetic procedure II. ¹H NMR (400 MHz, Chloroform-d) δ 9.4 (s, 1H), 8.3 (d, J=6.5 Hz, 1H), 7.2 (s, 5H), 7.2 (s, 1H), 6.6 (d, J=23.9 Hz, 1H), 5.7-5.5 (m, 1H), 5.1-4.9 (m, 2H), 4.6 (dq, J=18.1, 9.9, 8.6 Hz, 1H), 4.4-4.2 (m, 1H), 4.0 (h, J=6.9, 5.7 Hz, 1H), 3.3 (s, 1H), 3.2 (d, J=10.8 Hz, 1H), 2.4 (t, J=8.4 Hz, 1H), 2.3 (s, 1H), 2.1 (s, 1H), 2.0 (dp, J=25.6, 9.2, 7.8 Hz, 2H), 1.9-1.6 (m, 2H), 1.5-1.4 (m, 1H), 0.8 (dd, J=12.2, 6.6 Hz, 6H), 0.7-0.5 (m, 1H), 0.3 (t, J=9.5 Hz, 2H). ¹³C NMR (100 MHz, Chloroform-d) δ 199.5, 180.0, 172.7, 171.3, 156.6, 136.2, 128.5, 128.2, 128.0, 67.1, 60.5, 53.7, 50.7, 40.6, 38.0, 37.6, 31.1, 29.8, 28.4, 19.2, 17.9, 7.3, 4.5, 4.4.

### Benzyl ((S)-1-(((S)-4,4-dimethyl-1-oxo-1-(((S)-1-oxo-3-((S)-2-oxopyrrolidin-3-yl)propan-2-yl)amino)pentan-2-yl)amino)-2,3-dimethyl-1-oxobutan-2-yl)carbamate (MPI22)

The synthesis of MP122 was based on Representative synthetic procedure II. ¹H NMR (400 MHz, Chloroform-d) δ 9.52 (s, 1H), 8.07 (d, J=7.7 Hz, 1H), 7.50-7.26 (m, 5H), 6.77 (d, J=7.4 Hz, 1H), 6.39-6.20 (m, 1H), 5.47 (s, 1H), 5.07 (d, J=3.2 Hz, 2H), 4.57-4.37 (m, 1H), 4.33-4.16 (m, 1H), 3.35-3.18 (m, 2H), 2.44-2.29 (m, 2H), 2.14-2.05 (m, 2H), 1.98-1.92 (m, 1H), 1.90-1.81 (m, 1H), 1.74 (t, J=9.8 Hz, 1H), 1.40 (s, 3H), 0.97-0.85 (m, 15H). ¹³C NMR (101 MHz, CDCl₃): δ 200.59, 173.83, 173.43, 173.17, 156.23, 136.06, 128.70, 128.63, 128.35, 128.16, 67.48, 67.28, 63.36, 57.12, 51.83, 45.78, 40.35, 37.64, 34.88, 30.65, 30.24, 29.67, 28.12, 17.44, 17.36, 17.09, 17.04.

### Benzyl (1-(((S)-4,4-dimethyl-1-oxo-1-(((S)-1-oxo-3-((S)-2-oxopyrrolidin-3-yl)propan-2-yl)amino)pentan-2-yl)amino)-2-methyl-1-oxopropan-2-yl)carbamate (MPI23)

The synthesis of MP123 was based on Representative synthetic procedure II. ¹H NMR (400 MHz, Chloroform-d) δ 9.52 (s, 1H), 8.15 (d, J=7.6 Hz, 1H), 7.33 (s, 5H), 6.83 (d, J=8.0 Hz, 1H), 6.12 (s, 1H), 5.71 (s, 1H), 5.05 (q, J=12.2 Hz, 2H), 4.58-4.42 (m, 1H), 4.23 (ddd, J=11.3, 7.6, 3.9 Hz, 1H), 3.33-3.16 (m, 2H), 2.49-2.29 (m, 2H), 2.13-1.97 (m, 2H), 1.96-1.82 (m, 2H), 1.82-1.68 (m, 1H), 1.47 (d, J=6.8 Hz, 6H), 0.94 (s, 9H). ¹³C NMR (101 MHz, CDCl₃) δ 200.5, 180.0, 173.8, 156.0, 136.0, 128.6, 128.3, 128.1, 67.2, 57.3, 57.1, 51.6, 45.5, 40.4, 37.7, 30.7, 30.2, 29.6, 28.3, 26.0.

### Benzyl (1-(((S)-3-cyclohexyl-1-oxo-1-(((S)-1-oxo-3-((S)-2-oxopyrrolidin-3-yl)propan-2-yl)amino)propan-2-yl)carbamoyl)cyclopropyl)carbamate (MPI26)

The synthesis of MP126 was based on Representative synthetic procedure II. ¹H NMR (400 MHz, Chloroform-d) δ 9.43 (d, J=24.1 Hz, 1H), 8.30 (s, 1H), 7.27 (d, J=4.2 Hz, 5H), 6.86 (d, J=56.9 Hz, 1H), 6.25 (s, 1H), 5.88 (d, J=34.9 Hz, 1H), 5.18-4.90 (m, 2H), 4.56 (q, J=4.1 Hz, 1H), 4.51-4.35 (m, 1H), 4.20 (s, 1H), 3.24 (d, J=10.0 Hz, 2H), 2.45-2.20 (m, 2H), 2.03-1.66 (m, 7H), 1.52-1.35 (m, 3H), 1.22 (d, J=9.4 Hz, 1H), 1.14-1.00 (m, 4H), 0.98-0.67 (m, 4H).

### 3-Chlorobenzyl ((2S,3S)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate (MPI27)

The synthesis of MP127 was based on Representative synthetic procedure II. ¹H NMR (400 MHz, Chloroform-d) δ 9.44 (s, 1H), 7.99 (d, J=6.4 Hz, 1H), 7.41 (d, J=7.8 Hz, 1H), 7.29 (s, 1H), 7.22 (d, J=4.6 Hz, 2H), 7.16 (t, J=4.5 Hz, 1H), 6.11 (s, 1H), 5.89 (d, J=5.1 Hz, 1H), 5.01 (q, J=12.7 Hz, 2H), 4.48-4.37 (m, 1H), 4.35-4.25 (m, 1H), 4.11 (d, J=5.5 Hz, 2H), 3.36-3.19 (m, 2H), 2.44-2.26 (m, 2H), 1.98-1.86 (m, 2H), 1.78-1.70 (m, 2H), 1.60-1.47 (m, 3H), 1.30-1.15 (m, 12H), 1.16-1.08 (m, 3H), 1.02 (d, J=6.0 Hz, 3H), 0.96-0.82 (m, 3H). ¹³C NMR (101 MHz, CDCl₃) δ 199.57, 179.96, 172.90, 169.62, 155.99, 138.26, 134.43, 129.88, 128.32, 127.95, 125.95, 75.51, 66.72, 66.02, 58.93, 57.58, 51.50, 40.54, 40.03, 37.92, 34.24, 33.65, 32.62, 29.81, 29.71, 28.62, 28.24, 26.35, 26.22, 26.02, 17.29.

### 3-Chlorobenzyl ((S)-1-(((S)-3-cyclohexyl-1-oxo-1-(((S)-1-oxo-3-((S)-2-oxopyrrolidin-3-yl)propan-2-yl)amino)propan-2-yl)amino)-3-methyl-1-oxobutan-2-yl)carbamate (MPI28)

The synthesis of MPI28 was based on Representative synthetic procedure II. ¹H NMR (400 MHz, Chloroform-d) δ 9.49 (s, 1H), 8.25 (d, J=6.6 Hz, 1H), 7.39-7.11 (m, 4H), 7.02 (d, J=8.3 Hz, 1H), 6.56 (s, 1H), 5.64 (d, J=8.5 Hz, 1H), 5.06 (q, J=12.6 Hz, 2H), 4.74-4.61 (m, 1H), 4.37 (s, 1H), 4.03 (t, J=7.6 Hz, 1H), 3.41-3.22 (m, 2H), 2.50-2.27 (m, 2H), 2.19-2.07 (m, 1H), 2.07-1.86 (m, 3H), 1.86-1.48 (m, 8H), 1.39-1.04 (m, 5H), 1.04-0.81 (m, 8H). ¹³C NMR (101 MHz, CDCl₃) δ 199.5, 180.0, 173.3, 171.2, 156.4, 138.2, 134.4, 129.9, 128.3, 128.0, 126.0, 66.2, 60.6, 57.5, 51.1, 40.6, 40.2, 38.0, 34.2, 33.5, 32.5, 31.0, 29.9, 29.7, 28.4, 26.3, 26.2, 26.0, 19.2, 17.8.

### Benzyl ((S)-1-(((S)-3-cyclopropyl-1-oxo-1-(((S)-1-oxo-3-((S)-2-oxopyrrolidin-3-yl)propan-2-yl)amino)propan-2-yl)amino)-3,3-dimethyl-1-oxobutan-2-yl)carbamate (MPI31)

The synthesis of MPI31 was based on Representative synthetic procedure II. ¹H NMR (400 MHz, CDCl₃) δ 9.51 (s, 1H), 8.20 (d, J=6.7 Hz, 1H), 7.32 (d, J=4.2 Hz, 5H), 7.17 (d, J=8.3 Hz, 1H), 6.67 (s, 1H), 5.68 (d, J=9.4 Hz, 1H), 5.08 (s, 2H), 4.67 (q, J=7.3 Hz, 1H), 4.42 (p, J=4.9 Hz, 1H), 4.02 (d, J=9.4 Hz, 1H), 3.31 (dq, J=17.4, 9.5 Hz, 2H), 2.44 (p, J=8.1 Hz, 1H), 2.34 (dt, J=14.9, 8.5 Hz, 1H), 2.07-1.86 (m, 2H), 1.86-1.74 (m, 1H), 1.65 (ddt, J=20.5, 13.6, 6.9 Hz, 2H), 0.98 (s, 9H), 0.70 (h, J=7.1, 6.6 Hz, 1H), 0.43 (d, J=8.0 Hz, 2H), 0.08 (d, J=5.1 Hz, 2H). ¹³C NMR (101 MHz, CDCl₃) δ 199.3, 180.0, 172.6, 170.4, 156.6, 136.3, 128.5, 128.2, 128.0, 77.3, 67.1, 62.9, 57.4, 53.7, 40.6, 37.9, 37.7, 34.7, 29.9, 28.4, 26.5, 7.3, 4.5, 4.3.

### Benzyl ((S)-1-cyclopropyl-2-(((S)-3-cyclopropyl-1-oxo-1-(((S)-1-oxo-3-((S)-2-oxopyrrolidin-3-yl)propan-2-yl)amino)propan-2-yl)amino)-2-oxoethyl)carbamate (MPI32)

The synthesis of MPI32 was based on Representative synthetic procedure II. ¹H NMR (400 MHz, DMSO-d₆): δ 9.35 (s, 1H), 8.38 (d, J=7.7 Hz, 1H), 7.91 (dd, J=22.3, 7.8 Hz, 1H), 7.57 (s, 1H), 7.52-7.42 (m, 1H), 7.35-7.16 (m, 5H), 4.94 (s, 2H), 4.34-4.20 (m, 1H), 4.20-4.07 (m, 1H), 3.57-3.41 (m, 1H), 3.15-2.91 (m, 2H), 2.30-2.14 (m, 1H), 2.14-1.99 (m, 1H), 1.81 (q, J=16.5, 14.7 Hz, 1H), 1.64-1.27 (m, 4H), 1.04-0.85 (m, 1H), 0.74-0.55 (m, 1H), 0.48-0.16 (m, 6H), 0.08-0.11 (m, 2H). ¹³C NMR (101 MHz, DMSO): δ 201.20, 178.70, 172.56, 171.22, 156.23, 137.50, 128.79, 128.24, 128.15, 65.84, 58.23, 56.67, 53.57, 37.63, 29.81, 27.74, 14.07, 8.05, 4.88, 4.69, 3.59, 2.87.

### 3-Chlorobenzyl ((2S,3S)-3-(tert-butoxy)-1-(((S)-4-methyl-1-oxo-1-(((S)-1-oxo-3-((S)-2-oxopyrrolidin-3-yl)propan-2-yl)amino)pentan-2-yl)amino)-1-oxobutan-2-yl)carbamate (MPI34)

The synthesis of MPI34 was based on Representative synthetic procedure II. ¹H NMR (400 MHz, Chloroform-d) δ 9.50 (s, 1H), 8.39-7.96 (m, 1H), 7.47 (d, J=7.6 Hz, 1H), 7.35 (s, 1H), 7.29 (d, J=4.7 Hz, 2H), 7.23 (s, 1H), 6.19-5.86 (m, 2H), 5.07 (q, J=12.6 Hz, 2H), 4.55-4.34 (m, 2H), 4.18 (d, J=5.3 Hz, 2H), 3.37-3.26 (m, 2H), 2.56-2.26 (m, 3H), 2.01-1.92 (m, 1H), 1.87-1.77 (m, 1H), 1.77-1.64 (m, 2H), 1.59 (t, J=9.0 Hz, 1H), 1.27 (s, 9H), 1.08 (d, J=5.9 Hz, 3H), 0.96 (dd, J=10.9, 5.7 Hz, 6H). ¹³C NMR (101 MHz, CDCl₃) δ 199.64, 172.82, 169.54, 169.33, 155.98, 138.29, 134.42, 129.88, 128.31, 127.95, 125.96, 75.53, 66.74, 66.00, 58.91, 57.66, 52.11, 41.48, 40.61, 28.60, 28.22, 24.93, 22.92, 22.08, 17.21.

### (S)-benzyl (1-(2-(3-amino-3-oxopropyl)-2-propionylhydrazinyl)-4-methyl-1-oxopentan-2-yl)carbamate (MPI36)

The synthesis of MPI36 was based on Representative synthetic procedure III. ¹H NMR (400 MHz, Chloroform-d) δ 9.86 (s, 1H), 7.47-7.21 (m, 5H), 6.94 (s, 1H), 6.09 (s, 1H), 5.89 (d, J=7.9 Hz, 1H), 5.07 (s, 2H), 4.28 (q, J=7.7 Hz, 1H), 3.76 (s, 2H), 2.60-2.34 (m, 2H), 2.30-2.12 (m, 2H), 1.75-1.54 (m, 3H), 1.04-0.85 (m, 9H). ¹³C NMR (101 MHz, CDCl₃): δ 176.42, 174.71, 172.59, 156.41, 136.06, 128.59, 128.33, 128.04, 67.21, 60.43, 52.45, 40.68, 33.73, 25.37, 24.73, 22.87, 21.76, 8.74.

### (S)-benzyl (1-(2-(3-amino-3-oxopropyl)-2-(2-chloroacetyl)hydrazinyl)-4-methyl-1-oxopentan-2-yl)carbamate (MPI37)

The synthesis of MPI37 was based on Representative synthetic procedure III. ¹H NMR (400 MHz, Chloroform-d) δ 9.43 (s, 1H), 7.49-7.29 (m, 5H), 6.12 (s, 1H), 5.76 (s, 1H), 5.33 (s, 1H), 5.12 (s, 2H), 4.29-4.15 (m, 1H), 4.11-3.75 (m, 4H), 2.65-2.43 (m, 2H), 1.80-1.51 (m, 3H), 1.09-0.82 (m, 6H).

### (S)-benzyl (1-(2-acryloyl-2-(3-amino-3-oxopropyl)hydrazinyl)-4-methyl-1-oxopentan-2-yl)carbamate (MPI38)

The synthesis of MPI38 was based on Representative synthetic procedure III. ¹H NMR (400 MHz, Methanol-d₄) δ 7.37-7.06 (m, 5H), 6.51 (s, 1H), 6.17 (dd, J=17.0, 2.0 Hz, 1H), 5.60 (d, J=18.0 Hz, 1H), 5.02 (d, J=4.0 Hz, 2H), 4.07 (dd, J=9.8, 5.3 Hz, 1H), 3.91-3.49 (m, 2H), 2.51-2.26 (m, 2H), 1.70-1.41 (m, 3H), 0.87 (dd, J=13.1, 6.5 Hz, 6H). ¹³C NMR (101 MHz, MeOD) δ 174.72, 173.40, 168.17, 157.23, 136.85, 128.60, 128.16, 127.71, 127.53, 126.36, 66.39, 53.52, 39.83, 32.77, 24.50, 21.97, 20.23.

### Benzyl ((S)-1-(2-(3-amino-3-oxopropyl)-2-((R)-2-chloropropanoyl)hydrazineyl)-4-methyl-1-oxopentan-2-yl)carbamate (MPI39)

The synthesis of MPI39 was based on Representative synthetic procedure III. ¹H NMR (400 MHz, Chloroform-d) δ 9.51 (d, J=70.4 Hz, 1H), 7.41-7.30 (m, 5H), 6.15 (d, J=44.0 Hz, 1H), 5.72 (s, 1H), 5.60-5.42 (m, 1H), 5.12 (s, 2H), 4.52 (s, 1H), 4.26 (d, J=10.2 Hz, 1H), 4.17-3.90 (m, 1H), 3.52 (d, J=33.7 Hz, 1H), 2.56 (s, 1H), 2.47 (s, 1H), 1.84-1.52 (m, 7H), 1.05-0.81 (m, 6H).

### Benzyl ((S)-1-(2-(3-amino-3-oxopropyl)-2-((S)-2-chloropropanoyl)hydrazineyl)-4-methyl-1-oxopentan-2-yl)carbamate (MPI40)

The synthesis of MPI40 was based on Representative synthetic procedure III. ¹H NMR (400 MHz, Chloroform-d) δ 9.70 (s, 1H), 7.42-7.28 (m, 5H), 6.45-6.24 (m, 1H), 5.83 (s, 1H), 5.65-5.42 (m, 1H), 5.11 (s, 2H), 4.56 (s, 1H), 4.34-4.21 (m, 1H), 4.01 (s, 1H), 3.55 (s, 1H), 2.56 (s, 1H), 2.46 (s, 1H), 1.95 (s, 1H), 1.81-1.60 (m, 3H), 1.54 (d, J=6.6 Hz, 3H), 1.11-0.84 (m, 6H).

### S)-benzyl (1-(2-(3-amino-3-oxopropyl)-2-(2-(4-nitrophenoxy)acetyl)hydrazinyl)-4-methyl-1-oxopentan-2-yl)carbamate (MPI44)

The synthesis of MPI44 was based on Representative synthetic procedure III. ¹H NMR (400 MHz, Chloroform-d) δ 9.31 (s, 1H), 8.16 (d, J=9.2 Hz, 2H), 7.42-7.27 (m, 5H), 6.98 (d, J=8.7 Hz, 2H), 5.72 (s, 1H), 5.46 (s, 1H), 5.23-5.08 (m, 3H), 4.71 (s, 2H), 4.30-4.18 (m, 1H), 3.67-3.25 (m, 2H), 2.59 (s, 2H), 1.78-1.61 (m, 3H), 1.08-0.92 (m, 6H).

### (S)-4-fluorophenyl 1-(3-amino-3-oxopropyl)-2-(2-(((benzyloxy)carbonyl)amino)-4-methylpentanoyl)hydrazinecarboxylate (MPI47)

The synthesis of MPI47 was based on Representative synthetic procedure III. ¹H NMR (400 MHz, DMSO-d₆) δ 9.39 (s, 1H), 7.95 (d, J=8.3 Hz, 1H), 7.53-7.29 (m, 6H), 7.10-6.88 (m, 3H), 6.86-6.68 (m, 2H), 5.06 (d, J=1.9 Hz, 2H), 4.57 (dt, J=13.7, 7.0 Hz, 1H), 3.81 (t, J=7.1 Hz, 2H), 2.47 (t, J=7.2 Hz, 2H), 1.71-1.54 (m, 3H), 0.88 (dd, J=11.2, 5.7 Hz, 6H). ¹³C NMR (101 MHz, DMSO) δ 171.58, 157.11, 156.25, 155.96, 154.80, 154.06, 154.04, 153.33, 137.20, 128.86, 128.40, 128.25, 116.57, 116.49, 116.19, 115.96, 66.24, 46.43, 42.08, 33.55, 24.37, 23.01, 21.85.

### Benzyl ((S)-1-(((S)-1-(2-acryloyl-2-(3-amino-3-oxopropyl)hydrazinyl)-4-methyl-1-oxopentan-2-yl)amino)-3-methyl-1-oxobutan-2-yl)carbamate (MPI51)

The synthesis of MPI51 was based on Representative synthetic procedure III. ¹H NMR (400 MHz, Methanol-d₄) δ 7.55-7.19 (m, 5H), 6.67 (s, 1H), 6.31 (dd, J=16.9, 2.1 Hz, 1H), 5.74 (d, J=10.3 Hz, 1H), 5.12 (s, 2H), 4.42 (dd, J=9.8, 5.3 Hz, 1H), 3.98 (d, J=7.0 Hz, 2H), 3.74 (s, 1H), 2.52 (s, 2H), 2.19-2.04 (m, 1H), 1.86-1.57 (m, 3H), 1.13-0.90 (m, 12H). ¹³C NMR (101 MHz, MeOD): δ 174.66, 173.30, 172.66, 168.19, 157.91, 128.10, 127.66, 127.50, 66.41, 60.89, 50.53, 39.48, 32.65, 30.51, 24.48, 21.89, 20.50, 18.35, 17.23.

### Representative Synthetic Procedure III

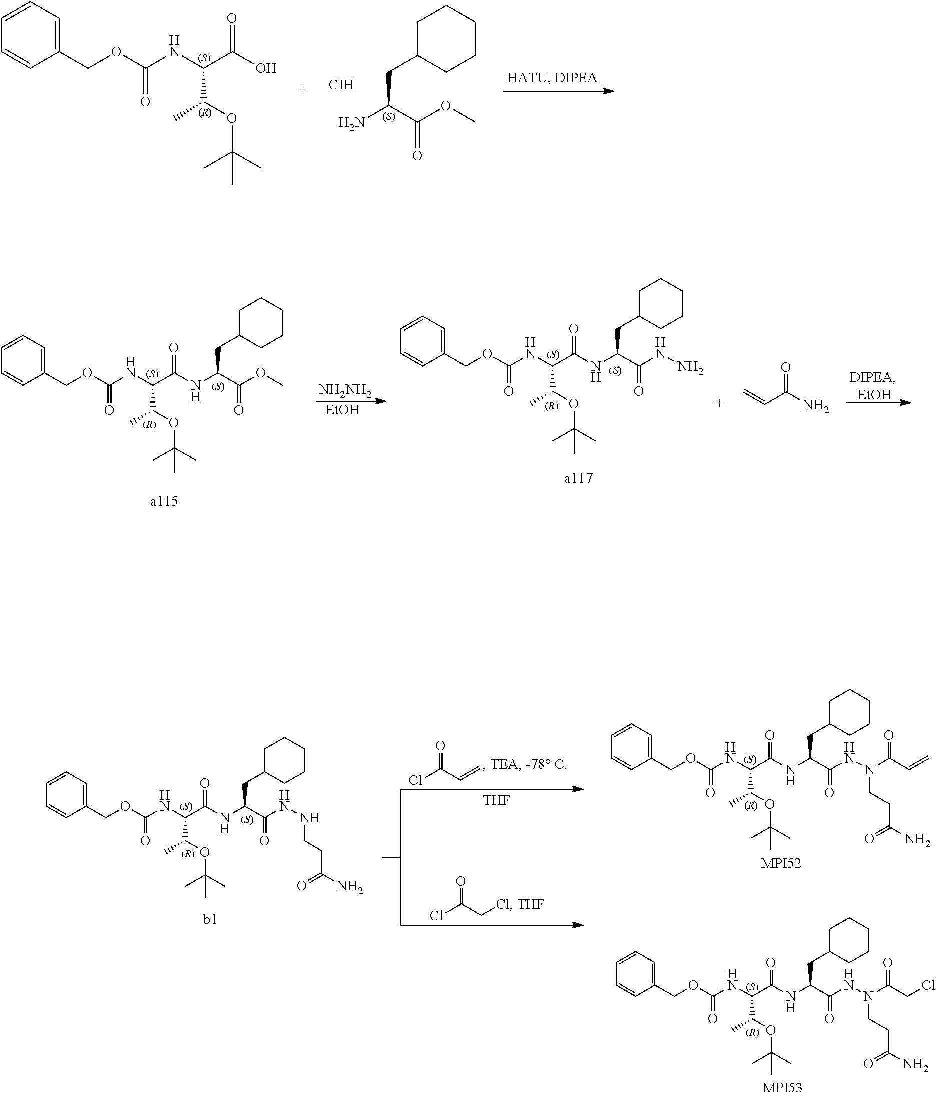

### Methyl (S)-2-((2S,3R)-2-(((benzyloxy)carbonyl)amino)-3-(tert-butoxy)butanamido)-3-cyclohexylpropanoate (a115)

To a solution of Z-Thr(tBu)—OH (500 mg, 1.6 mmol, 1.0 equiv) in anhydrous DMF (5 mL) at 0° C., and then H-L-Cha-OMe (360 mg, 1.6 mmol, 1.0 equiv), HATU (912 mg, 2.4 mmol, 1.5 equiv), DIPEA (1.1 mL, 6.4 mmol, 4.0 equiv) was added sequentially. The mixture was stirred at RT overnight. The mixture was diluted with EtOAc and washed with water, 1M HCl, sat. NaCl, dried over Na₂SO₄, and concentrated. The residue was purified by column chromatography (Hexane:EA=3:1 v/v) to afford the pure product a115 as a colorless oil (570 mg, yield 75%). ¹H NMR (400 MHz, Chloroform-d) δ 7.7 (d, J=7.8 Hz, 1H), 7.4-7.3 (m, 5H), 6.1 (d, J=5.3 Hz, 1H), 5.3-5.1 (m, 2H), 4.6 (td, J=8.4, 5.1 Hz, 1H), 4.3-4.2 (m, 2H), 3.7 (s, 3H), 1.8-1.7 (m, 6H), 1.6 (ddd, J=14.2, 9.0, 5.7 Hz, 1H), 1.3 (s, 9H), 1.2 (dd, J=24.2, 7.4 Hz, 7H), 1.0-0.9 (m, 2H). ¹³C NMR (100 MHz, Chloroform-d) δ 172.7, 169.2, 155.9, 136.2, 128.3, 128.3, 127.8, 127.7, 127.7, 75.2, 66.7, 66.5, 60.0, 51.8, 50.2, 39.5, 34.0, 33.3, 32.3, 28.0, 28.0, 28.0, 26.1, 26.0, 25.8, 20.7.

### Benzyl ((2S,3R)-3-(tert-butoxy)-1-(((S)-3-cyclohexyl-1-hydrazineyl-1-oxopropan-2-yl)amino)-1-oxobutan-2-yl)carbamate (a117)

a115 (500 mg, 1.0 mmol, 1.0 equiv) and hydrazine (350 mg, 11.0 mmol, 11.0 equiv) were dissolved in ethanol. The reaction mixture was stirred at RT overnight. After the reaction was completed, the solvent was removed on vacuo. The residue was used in the next step without further purification. ¹H NMR (400 MHz, Methanol-d₄) δ 7.4 (ddt, J=19.6, 13.8, 6.8 Hz, 5H), 5.1 (d, J=3.4 Hz, 2H), 4.5 (dd, J=9.4, 5.5 Hz, 1H), 4.2-4.1 (m, 2H), 1.9-1.6 (m, 7H), 1.4 (tq, J=5.1, 2.8 Hz, 1H), 1.2 (d, J=36.1 Hz, 15H), 1.0-0.9 (m, 2H). ¹³C NMR (100 MHz, Methanol-d₄) δ 173.6, 172.2, 158.5, 138.0, 129.5, 129.1, 129.0, 76.0, 68.8, 67.9, 61.3, 51.2, 40.7, 35.2, 34.8, 33.5, 28.6, 27.5, 27.3, 27.1, 19.6.

### Benzyl ((2S,3R)-1-(((S)-1-(2-(3-amino-3-oxopropyl)hydrazineyl)-3-cyclohexyl-1-oxopropan-2-yl)amino)-3-(tert-butoxy)-1-oxobutan-2-yl)carbamate (b1)

To a solution of a117 (300 mg, 0.63 mmol, 1.0 equiv), acrylamide (54 mg, 0.76 mmol, 1.2 equiv) and DIPEA (550 μL, 3.15 mmol, 5.0 equiv) in ethanol. The reaction mixture was refluxed for 48 h. After the reaction was completed, the solution was removed on vacuo. The resulted residue was purified by column chromatography (EA:MeOH=8:1 v/v) to afford the pure product b1 as a white solid (150 mg, yield 44%). ¹H NMR (400 MHz, Methanol-d₄) δ 7.4-7.3 (m, 5H), 5.2-5.1 (m, 2H), 4.4 (dd, J=9.2, 5.8 Hz, 1H), 4.2-4.1 (m, 2H), 3.0 (t, J=6.8 Hz, 2H), 2.4 (t, J=6.8 Hz, 2H), 1.8-1.6 (m, 7H), 1.4-1.4 (m, 1H), 1.2 (d, J=25.5 Hz, 15H), 1.0-0.9 (m, 2H). ¹³C NMR (100 MHz, Methanol-d₄) δ 175.8, 171.8, 171.2, 157.2, 136.7, 128.1, 127.7, 127.6, 74.5, 67.4, 66.6, 60.2, 50.0, 47.2, 39.2, 33.9, 33.4, 33.2, 32.2, 27.3, 26.2, 26.0, 25.7, 18.4.

### Benzyl ((2S,3R)-1-(((S)-1-(2-acryloyl-2-(3-amino-3-oxopropyl)hydrazineyl)-3-cyclohexyl-1-oxopropan-2-yl)amino)-3-(tert-butoxy)-1-oxobutan-2-yl)carbamate (MPI52)

To a solution of b1 (100 mg, 0.18 mmol, 1.0 equiv) in anhydrous THF at −78° C. Added TEA (50 μL, 0.36 mmol, 2.0 equiv) and acryloyl chloride (20 mg, 0.22 mmol, 1.2 equiv) at this temperature. The reaction was quenched by slow addition of H₂O and removed THF on vacuo. The mixture was diluted with H₂O and extracted with EtOAc, washed with sat. NaCl, dried over Na₂SO₄ and concentrated. The residue was purified by column chromatography (EA:MeOH=8:1 v/v) to afford the pure product 65 (40 mg, yield 37%) as a white solid. ¹H NMR (400 MHz, Methanol-d₄) δ 7.4-7.3 (m, 5H), 6.3 (dd, J=16.9, 2.0 Hz, 1H), 5.8 (d, J=10.6 Hz, 1H), 5.2-5.1 (m, 2H), 4.4 (t, J=7.5 Hz, 1H), 4.2-4.1 (m, 2H), 3.7 (dq, J=13.2, 6.9 Hz, 1H), 2.5 (s, 2H), 1.9-1.6 (m, 7H), 1.5 (s, 1H), 1.2 (d, J=21.1 Hz, 15H), 1.1-0.9 (m, 4H). ¹³C NMR (100 MHz, Methanol-d₄) δ 174.6, 172.5, 171.7, 168.1, 157.3, 136.6, 128.8, 128.1, 127.7, 127.6, 126.3, 74.5, 67.4, 66.6, 60.2, 50.0, 44.8, 38.6, 33.9, 33.3, 32.7, 32.1, 27.4, 26.1, 26.0, 25.8, 18.4.

### Benzyl ((2S,3R)-1-(((S)-1-(2-(3-amino-3-oxopropyl)-2-(2-chloroacetyl)hydrazineyl)-3-cyclohexyl-1-oxopropan-2-yl)amino)-3-(tert-butoxy)-1-oxobutan-2-yl)carbamate (MPI53)

To a solution of b1 (100 mg, 0.18 mmol, 1.0 equiv) in anhydrous THF at 0° C. and then added chloroacetyl chloride (25 mg, 0.22 mmol, 1.2 equiv). The resulted mixture was stirred at RT for 2 h. After the reaction was completed, removed the solvent on vacuo and purified by column chromatography (EA:MeOH=8:1 v/v) to afford the pure product MPI53 (55 mg, yield 50%) as a white solid. ¹H NMR (400 MHz, Methanol-d₄) δ 7.4 (tdd, J=12.4, 8.4, 6.6 Hz, 5H), 5.2 (d, J=6.0 Hz, 2H), 4.4 (s, 1H), 4.2-4.1 (m, 3H), 4.0 (s, 1H), 2.5 (s, 2H), 1.9-1.7 (m, 7H), 1.5-1.4 (m, 1H), 1.2 (d, J=18.4 Hz, 17H), 1.0 (dddd, J=26.3, 23.5, 11.3, 2.9 Hz, 2H). ¹³C NMR (100 MHz, Methanol-d₄) δ 175.7, 174.0, 172.9, 170.2, 158.6, 137.9, 129.5, 129.1, 128.9, 75.8, 68.7, 68.0, 61.5, 54.8, 51.5, 46.7, 39.8, 35.2, 34.7, 33.9, 33.4, 28.7, 27.4, 27.3, 27.1, 20.9.

### Representative Synthetic Procedure IV

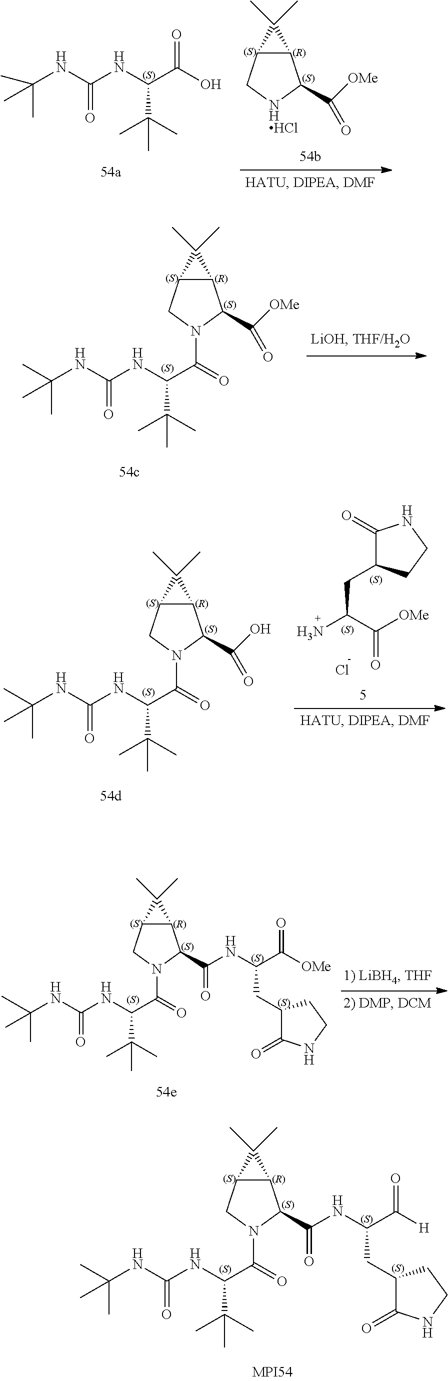

### Methyl (1R,2S,5S)-3-((S)-2-(3-(tert-butyl)ureido)-3,3-dimethylbutanoyl)-6,6-dimethyl-3-azabicyclo[3.1.0]hexane-2-carboxylate (54c)

To a solution of 54a (4 mmol, 0.92 g) and 54b (4 mmol, 0.82 mg) in anhydrous DMF (20 mL) was added DIPEA (10 mmol, 1.29 g) and was cooled to 0° C. HATU (4.4 mmol, 1.67 g) was added to the solution under 0° C. and then stirred at room temperature overnight. The reaction mixture was then diluted with ethyl acetate (100 mL) and washed with saturated NaHCO₃ solution (2×50 mL), 1 M HCl solution (2×50 mL), and saturated brine solution (2×50 mL) sequentially. The organic layer was dried over anhydrous Na₂SO₄ and then concentrated in vacuo. The residue was then purified with flash chromatography (15-50% EtOAc in hexanes as the eluent) to afford 54c as colorless oil (1.14 g, 75%).

### (1R,2S,5S)-3-((S)-2-(3-(tert-butyl)ureido)-3,3-dimethylbutanoyl)-6,6-dimethyl-3-azabicyclo[3.1.0]hexane-2-carboxylic acid (54d)

54c (3 mmol, 1.14 g) was dissolved in 10 mL of THF. A solution of LiOH.H₂O (6 mmol, 250 mg) in 5 mL H₂O was added to the solution. The mixture was stirred at room temperature for 3 h. Then THF was removed in vacuo and the aqueous layer was acidified with 1 M HCl and extracted with dichloromethane (3×20 mL). The organic layer was dried over anhydrous Na₂SO₄ and concentrated to yield 54d as white solid (1.01 g, 92%). ¹H NMR (400 MHz, DMSO-d₆) δ 12.57 (s, 1H), 5.95 (s, 1H), 5.88 (d, J=10.0 Hz, 1H), 4.15 (d, J=10.0 Hz, 1H), 4.11 (s, 1H), 3.99 (d, J=10.4 Hz, 1H), 3.74 (dd, J=10.3, 5.3 Hz, 1H), 1.48 (dd, J=7.6, 5.1 Hz, 1H), 1.38 (d, J=7.5 Hz, 1H), 1.17 (s, 9H), 1.00 (s, 3H), 0.91 (s, 9H), 0.82 (s, 3H).

### Methyl (S)-2-((1R,2S,5S)-3-((S)-2-(3-(tert-butyl)ureido)-3,3-dimethylbutanoyl)-6,6-dimethyl-3-azabicyclo[3.1.0]hexane-2-carboxamido)-3-((S)-2-oxopyrrolidin-3-yl)propanoate (54e)

To a solution of 54d (1 mmol, 367 mg) and 5 (1 mmol, 222 mg) in anhydrous DMF (5 mL) was added DIPEA (2 mmol, 258 mg) and was cooled to 0° C. HATU (1.2 mmol, 456 mg) was added to the solution under 0° C. and then stirred at room temperature overnight. The reaction mixture was then diluted with ethyl acetate (50 mL) and washed with saturated NaHCO₃ solution (2×20 mL), 1 M HCl solution (2×20 mL), and saturated brine solution (2×20 mL) sequentially. The organic layer was dried over anhydrous Na₂SO₄ and then concentrated in vacuo. The residue was then purified with flash chromatography (1-10% methanol in dichloromethane as the eluent) to afford 54e as white solid (321 mg, 60%). ¹H NMR (400 MHz, Chloroform-d) δ 7.42 (d, J=8.0 Hz, 1H), 6.01 (s, 1H), 5.17-4.89 (m, 1H), 4.64 (ddd, J=11.6, 7.9, 4.0 Hz, 1H), 4.37 (s, 1H), 4.34 (s, 1H), 4.08 (d, J=10.3 Hz, 1H), 3.88 (dt, J=10.4, 2.7 Hz, 1H), 3.73 (s, 3H), 3.37-3.22 (m, 2H), 2.56-2.35 (m, 2H), 2.25-2.13 (m, 1H), 1.93-1.76 (m, 3H), 1.50 (d, J=2.3 Hz, 2H), 1.26 (s, 9H), 1.02 (s, 3H), 0.97 (s, 9H), 0.89 (s, 3H).

### (1R,2S,5S)-3-((S)-2-(3-(tert-butyl)ureido)-3,3-dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide (MPI54)

To a solution of 54e (0.25 mmol, 133 mg) in anhydrous dichloromethane (5 mL) was added a solution of LiBH₄ in anhydrous THF (2 M, 0.25 mL, 0.5 mmol) at 0° C. The resulting solution was stirred at the same temperature for 3 h. Then a saturated solution of NH₄Cl (5 mL) was added dropwise to quench the reaction. The layers were separated, and the organic layer was washed with saturated brine solution (2×10 mL), dried over anhydrous Na₂SO₄ and evaporated to dryness. The residue was then dissolved in anhydrous dichloromethane (5 mL) and cooled to 0° C. Dess-Martin periodinane (0.5 mmol, 212 mg) was added to the solution. The reaction mixture was then stirred at room temperature overnight. Then the reaction was quenched with a saturated NaHCO₃ solution containing 10% Na₂S₂O₃. The layers were separated. The organic layer was then washed with saturated brine solution (2×10 mL), dried over anhydrous Na₂SO₄ and evaporated in vacuo. The residue was then purified with flash chromatography (1-10% methanol in dichloromethane as the eluent) to afford MPI54 as white solid (64 mg, 51%). ¹H NMR (400 MHz, Chloroform-d) δ 9.54 (s, 1H), 6.34 (s, 1H), 5.20 (d, J=10.0 Hz, 1H), 4.64 (s, 1H), 4.54-4.44 (m, 1H), 4.36 (t, J=5.0 Hz, 2H), 4.10 (d, J=10.4 Hz, 1H), 3.91 (dd, J=10.4, 5.2 Hz, 1H), 3.31 (dq, J=17.5, 9.3, 8.0 Hz, 2H), 2.53 (q, J=8.1 Hz, 1H), 2.40 (ddd, J=17.9, 9.3, 4.8 Hz, 1H), 1.98 (ddt, J=21.6, 13.8, 5.3 Hz, 2H), 1.89-1.76 (m, 1H), 1.58-1.43 (m, 2H), 1.25 (s, 9H), 1.03 (s, 3H), 0.95 (s, 9H), 0.90 (s, 3H). ¹³C NMR (101 MHz, CDCl₃) δ 199.8, 180.1, 172.8, 172.2, 157.3, 60.8, 57.8, 57.3, 50.2, 48.4, 40.5, 37.7, 34.7, 30.6, 30.2, 29.4, 28.5, 28.0, 26.6, 26.3, 19.3, 12.7.

### (S)-2-((S)-2-(3-(tert-butyl)ureido)-3,3-dimethylbutanamido)-4,4-dimethyl-N—((S)-1-oxo-3-((S)-2-oxopyrrolidin-3-yl)propan-2-yl)pentanamide (MPI55)

The synthesis of MPI55 was based on Representative synthetic procedure II. ¹H NMR (400 MHz, Chloroform-d) δ 9.45 (s, 1H), 4.63-4.24 (m, 2H), 3.92 (d, J=25.5 Hz, 1H), 3.36-3.25 (m, 2H), 2.55-2.33 (m, 2H), 1.97-1.72 (m, 3H), 1.54-1.41 (m, 2H), 1.23 (s, 9H), 0.91 (s, 9H), 0.87 (s, 9H). ¹³C NMR (101 MHz, DMSO): δ 201.04, 178.62, 173.44, 172.54, 171.37, 157.54, 157.45, 60.02, 56.54, 50.49, 49.39, 45.72, 37.48, 34.78, 30.68, 29.98, 29.85, 29.73, 27.69, 27.04.

### (S)-5-((S)-2-(3-(tert-butyl)ureido)-3,3-dimethylbutanoyl)-N—((S)-1-oxo-3-((S)-2-oxopyrrolidin-3-yl)propan-2-yl)-5-azaspiro[2.4]heptane-6-carboxamide (MPI56)

The synthesis of MPI56 was based on Representative synthetic procedure IV. ¹H NMR (400 MHz, Chloroform-d) δ 9.48 (s, 1H), 7.86 (d, J=7.7 Hz, 1H), 6.53 (s, 1H), 5.46 (d, J=9.6 Hz, 1H), 4.89 (s, 1H), 4.57-4.42 (m, 1H), 4.34 (d, J=9.7 Hz, 1H), 3.77-3.52 (m, 2H), 3.38-3.10 (m, 2H), 2.70-2.46 (m, 8H), 2.13-1.71 (m, 1H), 1.21 (s, 12H), 0.91 (d, J=5.2 Hz, 15H), 0.62-0.38 (m, 6H). ¹³C NMR (100 MHz, Chloroform-d) δ 199.99, 180.49, 172.67, 172.56, 157.18, 60.89, 57.21, 57.01, 56.12, 50.17, 40.50, 37.69, 37.41, 35.52, 30.45, 29.49, 28.19, 26.45, 21.57.

### Representative Synthetic Procedure V

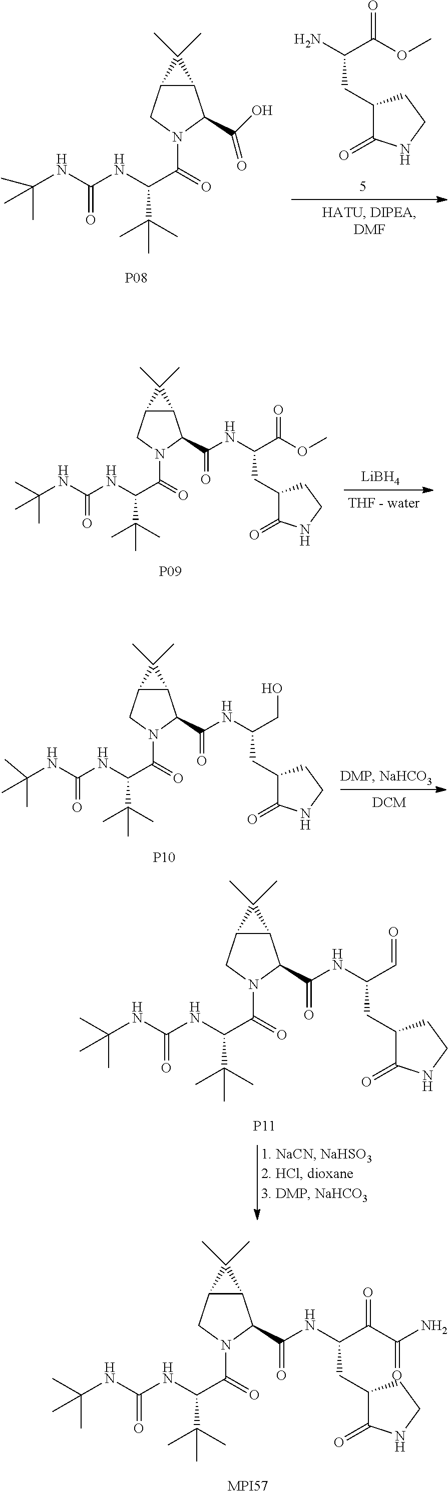

### Methyl (S)-2-((1R,2S,5S)-3-((S)-2-(3-(tert-butyl)ureido)-3,3-dimethylbutanoyl)-6,6-dimethyl-3-azabicyclo[3.1.0]hexane-2-carboxamido)-3-((S)-2-oxopyrrolidin-3-yl)propanoate (P09)

To a solution of P08 (4.4 mmol, 1.8 g) and 5 (4.9 mmol, 1.09 g) in anhydrous DMF (15 mL) was added DIPEA (17.8 mmol, 2.5 g, 3.2 mL) and was cooled to 0° C. HATU (5.8 mmol, 2.2 g) was added to the solution under 0° C. and then stirred at room temperature overnight. The reaction mixture was then diluted with ethyl acetate (50 mL) and washed with saturated NaHCO₃ solution (2×20 mL), 1 M HCl solution (2×20 mL), and saturated brine solution (2×20 mL) sequentially. The organic layer was dried over anhydrous Na₂SO₄ and then concentrated on vacuo. The residue was purified by column chromatography (MeOH:DCM=1:10 v/v) to afford the pure product P09 (2 g, 78%). ¹H NMR (400 MHz, Chloroform-d) δ 7.61 (d, J=8.0 Hz, 1H), 6.81 (s, 1H), 4.67-4.49 (m, 1H), 4.29 (d, J=5.8 Hz, 2H), 4.00 (d, J=10.5 Hz, 1H), 3.84 (dd, J=10.4, 5.2 Hz, 1H), 3.67 (s, 3H), 3.33-3.16 (m, 2H), 2.56-2.42 (m, 1H), 2.40-2.26 (m, 1H), 2.19-2.02 (m, 1H), 1.87-1.66 (m, 2H), 1.49-1.33 (m, 2H), 1.19 (s, 9H), 1.01-0.71 (m, 15H). ¹³C NMR (100 MHz, Chloroform-d) δ 180.39, 172.82, 172.76, 171.53, 157.63, 139.90, 135.30, 129.26, 120.44, 60.79, 58.00, 52.54, 50.75, 50.27, 48.47, 40.79, 38.23, 34.86, 33.36, 30.43, 29.30, 28.01, 27.85, 26.52, 26.27, 19.22, 12.63.

### (1R,2S,5S)-3-((S)-2-(3-(tert-butyl)ureido)-3,3-dimethylbutanoyl)-N—((S)-1-hydroxy-3-((S)-2-oxopyrrolidin-3-yl)propan-2-yl)-6,6-dimethyl-3-azabicyclo[3.1.0]hexane-2-carboxamide (P10)

To a solution of P09 (2 g, 3.5 mmol, 1.0 equiv) in anhydrous THF (10 mL) at 0° C. was added LiBH₄ (2.0 M in THF, 8.8 mL, 17.47 mmol, 5.0 equiv). The mixture was stirred at RT for 2 h. After the reaction was completed, excess reactants were consumed by slow addition of H₂O. The mixture was diluted with H₂O and extracted with EtOAc, washed with sat. NaCl, dried over Na₂SO₄ and concentrated. The residue was purified by column chromatography (MeOH:DCM=1:12 v/v) to afford the pure product P10 as a white solid (900 mg, yield 48%). ¹H NMR (400 MHz, Chloroform-d) δ 7.32 (d, J=8.4 Hz, 1H), 6.64 (s, 1H), 5.49-5.35 (m, 1H), 4.32-4.25 (m, 1H), 4.06-3.93 (m, 2H), 3.85 (dd, J=10.3, 5.3 Hz, 1H), 3.61-3.44 (m, 2H), 3.28-3.11 (m, 3H), 2.49-2.40 (m, 1H), 2.40-2.29 (m, 1H), 2.05-1.94 (m, 1H), 1.77-1.65 (m, 1H), 1.50-1.39 (m, 2H), 1.36 (d, J=7.6 Hz, 1H), 1.18 (s, 9H), 0.95 (s, 3H), 0.89 (s, 9H), 0.82 (s, 3H). ¹³C NMR (100 MHz, Chloroform-d) δ 181.61, 172.92, 171.95, 157.53, 65.21, 61.15, 57.86, 50.03, 49.31, 48.52, 40.79, 38.26, 34.70, 32.46, 30.84, 29.35, 28.14, 28.00, 26.60, 26.27, 19.24, 12.59.

### (1R,2S,5S)-3-((S)-2-(3-(tert-butyl)ureido)-3,3-dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide (P11)

To a solution of P10 (900 mg, 1.65 mmol, 1.0 equiv) in anhydrous DCM (10 mL) was added Dess-Martin reagent (2.1 g, 5 mmol, 3.0 equiv) slowly at 0° C. Then the reaction mixture was stirred at RT for 2 h. A solution of NaHCO₃ and Na₂S₂O₃ was added to quench the reaction. After 10 min, the mixture was washed with water, sat. NaCl, dried over Na₂SO₄ and concentrated. The residue was purified by column chromatography (MeOH:DCM=1:10 v/v) to yield P11 as a white solid (500 mg, yield 56%). ¹H NMR (400 MHz, Chloroform-d) δ 9.48 (s, 1H), 7.88-7.65 (m, 1H), 6.01 (d, J=10.2 Hz, 1H), 5.05 (t, J=10.9 Hz, 1H), 4.55 (d, J=14.6 Hz, 1H), 4.40 (tdd, J=13.1, 6.1, 3.4 Hz, 1H), 4.34-4.25 (m, 1H), 4.23-4.19 (m, 1H), 4.04 (q, J=11.9, 10.6 Hz, 1H), 3.89-3.72 (m, 1H), 3.34-3.16 (m, 3H), 2.56-2.27 (m, 2H), 2.00-1.84 (m, 1H), 1.82-1.71 (m, 3H), 1.53-1.34 (m, 3H), 1.19 (d, J=2.0 Hz, 9H), 1.03-0.76 (m, 15H).

### (1R,2S,5S)—N—((S)-4-amino-3,4-dioxo-1-((S)-2-oxopyrrolidin-3-yl) butan-2-yl)-3-((S)-2-(3-(tert-butyl)ureido)-3,3-dimethylbutanoyl)-6,6-dimethyl-3-azabicyclo[3.1.0]hexane-2-carboxamide (MPI57)

To a solution of P11 (500 mg, 0.9 mmol) in dichloromethane (25 mL) was added NaHSO₃ (480 mg, 4.6 mmol) slowly. The reaction was allowed to stir at RT for 30 min. Then NaCN (230 mg, 4.6 mmol), dissolved in 5 mL water was added to the reaction mixture slowly. The reaction mixture was stirred at RT for overnight. The mixture was washed with water, sat. NaCl dried over Na₂SO₄ and concentrated. ESI-MS was used to confirm the formation of cyanohydrin intermediate, which was carried forward to the next step without further purification. To a solution of the cyanohydrin intermediate (350 mg, 0.65 mmol) in 1,4-dioxane (10 mL) was added dropwise a HCl solution in 1,4-dioxane (4 M, 10 mL). The resulting solution was stirred at room temperature for 3 h. Then residue was then concentrated on vacuo to afford the hydroxyamide intermediate. ESI-MS was used to confirm the formation of cyanohydrin intermediate, which was carried forward to the next step without further purification. In the final step, to a solution of the hydroxyamide intermediate (260 mg, 0.47 mmol, 1.0 equiv) in anhydrous DCM (10 mL) was added Dess-Martin reagent (616 g, 1.42 mmol, 5.0 equiv) slowly at 0° C. Then the reaction mixture was stirred at RT for 2 h. The formation of the desired product was confirmed by ESI-MS study. A solution of NaHCO₃ and Na₂S₂O₃ was added to quench the reaction. After 10 min, the mixture was washed with water, sat. NaCl, dried over Na₂SO₄ and concentrated. The residue was purified by column chromatography (MeOH:DCM=1:10 v/v) to yield MPI57 as a white solid (156 mg, yield 60%). ¹H NMR (400 MHz, DMSO-d₆) δ 7.49-7.31 (m, 2H), 5.90-5.72 (m, 2H), 4.19-3.96 (m, 3H), 3.92-3.79 (m, 1H), 3.76-3.62 (m, 1H), 3.14-2.82 (m, 3H), 2.29 (q, J=10.2 Hz, 1H), 2.12 (dd, J=12.4, 7.4 Hz, 1H), 1.88-1.73 (m, 1H), 1.63-1.46 (m, 1H), 1.44-1.31 (m, 2H), 1.31-1.24 (m, 1H), 1.22-1.15 (m, 1H), 1.14-1.03 (m, 9H), 0.96-0.89 (m, 3H), 0.86-0.73 (m, 11H). ¹³C NMR (100 MHz, DMSO-d₆) δ 197.87, 178.66, 172.32, 171.66, 171.31, 171.23, 163.53, 157.88, 60.01, 57.28, 49.41, 34.57, 34.52, 29.58, 27.77, 26.88, 19.11, 13.05.

### Representative Synthetic Procedure VI

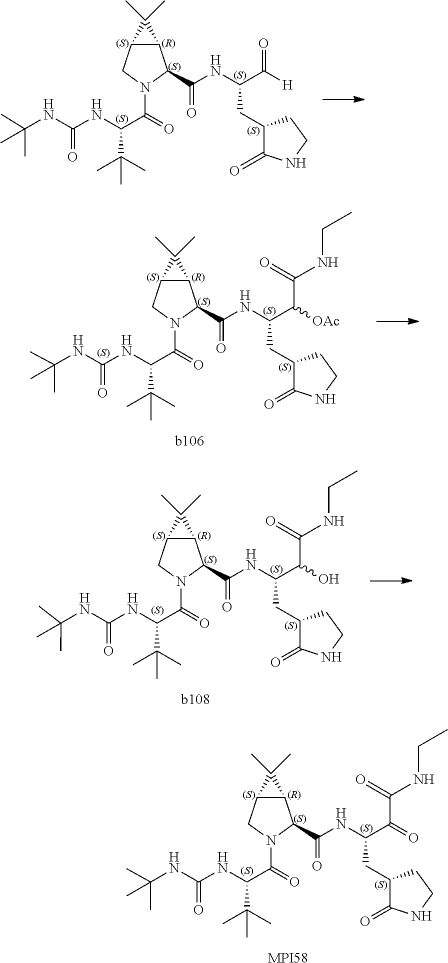

(3S)-3-((1R,2S,5S)-3-((S)-2-(3-(tert-butyl)ureido)-3,3-dimethylbutanoyl)-6,6-dimethyl-3-azabicyclo[3.1.0]hexane-2-carboxamido)-1-(ethylamino)-1-oxo-4-((S)-2-oxopyrrolidin-3-yl)butan-2-yl acetate (b106)

To a solution of aldehyde (120 mg, 0.24 mmol, 1.0 equiv) in anhydrous DCM (10 mL) at 0° C. Then added ethyl isocyanide (21 μL, 0.28 mmol, 1.2 equiv) and acetic acid (30 μL, 0.48 mmol, 2.0 equiv). Then the reaction mixture was stirred at RT overnight. After the reaction was completed, remove the solvent in vacuum and purified by column chromatography (MeOH:DCM=1:15 v/v) to yield b106 as a white solid (140 mg, yield 96%). ¹H NMR (400 MHz, Chloroform-d) δ 7.0 (d, J=9.6 Hz, 1H), 6.9 (s, 1H), 6.8 (s, 1H), 6.6 (t, J=5.7 Hz, 1H), 5.4-5.3 (m, 1H), 5.1 (dd, J=46.6, 4.6 Hz, 1H), 4.9 (d, J=10.2 Hz, 1H), 4.5-4.3 (m, 1H), 4.3 (d, J=9.9 Hz, 1H), 4.1 (d, J=7.5 Hz, 1H), 4.0 (d, J=10.3 Hz, 1H), 3.9-3.7 (m, 1H), 3.2 (qd, J=11.8, 9.9, 5.4 Hz, 4H), 2.5-2.3 (m, 2H), 2.1 (d, J=21.9 Hz, 3H), 1.7 (dq, J=11.9, 9.0 Hz, 1H), 1.5-1.3 (m, 2H), 1.3 (dd, J=7.7, 2.1 Hz, 1H), 1.2 (d, J=1.5 Hz, 9H), 1.1-1.0 (m, 3H), 0.9 (d, J=1.5 Hz, 3H), 0.9 (d, J=4.4 Hz, 9H), 0.8 (s, 3H). ¹³C NMR (100 MHz, Chloroform-d) δ 180.5, 172.6, 171.6, 169.7, 167.6, 157.4, 74.8, 61.0, 57.6, 50.0, 48.3, 40.4, 37.8, 34.7, 34.3, 32.8, 31.9, 30.6, 29.4, 28.2, 27.9, 26.5, 20.9, 19.1, 14.7, 12.6.

### (1R,2S,5S)-3-((S)-2-(3-(tert-butyl)ureido)-3,3-dimethylbutanoyl)-N-((2S)-4-(ethylamino)-3-hydroxy-4-oxo-1-((S)-2-oxopyrrolidin-3-yl)butan-2-yl)-6,6-dimethyl-3-azabicyclo[3.1.0]hexane-2-carboxamide(b108)

To a solution of b106 (140 mg, 0.23 mmol, 1.0 equiv) in 3:1 MeOH/H₂O (8 mL) was added LiOH.H₂O (20 mg, 0.46 mmol, 2.0 equiv) at 0° C. The reaction was stirred at RT for 1 h. After completion, the reaction mixture was neutralized with 0.5 M HCl solution and remove the MeOH in vacuum, then extracted with DCM. The organic layer was washed with sat. NaCl, dried over Na₂SO₄ and concentrated. The residue was purified by column chromatography (MeOH:DCM=1:10 v/v) to yield b108 as a white solid (90 mg, yield 70%). ¹H NMR (400 MHz, Chloroform-d) δ 7.1 (s, 2H), 6.9 (s, 1H), 5.6 (s, 1H), 5.4 (s, 1H), 5.0 (s, 1H), 4.3 (t, J=11.4 Hz, 2H), 4.1 (d, J=21.6 Hz, 2H), 4.0 (d, J=10.4 Hz, 1H), 3.8 (dd, J=10.4, 5.4 Hz, 1H), 3.2 (dt, J=18.8, 8.6 Hz, 4H), 2.5-2.4 (m, 1H), 2.3 (s, 1H), 2.1 (d, J=13.8 Hz, 1H), 1.7 (q, J=10.8, 10.3 Hz, 1H), 1.5 (t, J=11.6 Hz, 1H), 1.4-1.4 (m, 1H), 1.3 (d, J=7.6 Hz, 1H), 1.2 (s, 9H), 1.1 (t, J=7.2 Hz, 3H), 0.9 (s, 3H), 0.9 (s, 9H), 0.8 (s, 3H). ¹³C NMR (100 MHz, Chloroform-d) δ 181.3, 172.7, 172.1, 171.5, 157.5, 77.3, 73.1, 67.1, 61.0, 57.6, 50.0, 49.8, 48.4, 40.6, 38.1, 34.8, 34.1, 29.4, 28.1, 27.9, 26.6, 19.2, 14.9, 12.6.

### (1R,2S,5S)-3-((S)-2-(3-(tert-butyl)ureido)-3,3-dimethylbutanoyl)-N—((S)-4-(ethylamino)-3,4-dioxo-1-((S)-2-oxopyrrolidin-3-yl)butan-2-yl)-6,6-dimethyl-3-azabicyclo[3.1.0]hexane-2-carboxamide (MPI58)

To a solution of b108 (90 mg, 0.16 mmol, 1.0 equiv) in anhydrous DCM (10 mL) was added Dess-Martin reagent (130 mg, 0.32 mmol, 2.0 equiv) slowly at 0° C. Then the reaction mixture was stirred at RT for 2 h. A solution of NaHCO₃ and Na₂S₂O₃ was added to quench the reaction. After 10 min, the mixture was washed with water, sat. NaCl, dried over Na₂SO₄ and concentrated. The residue was purified by column chromatography (MeOH:DCM=1:10 v/v) to yield MPI58 as a white solid (50 mg, yield 54%). ¹H NMR (400 MHz, Methanol-d₄) δ 4.4-4.2 (m, 3H), 4.1-3.9 (m, 2H), 3.3-3.2 (m, 4H), 2.7-2.5 (m, 1H), 2.5-2.3 (m, 1H), 2.1 (qd, J=13.8, 3.3 Hz, 1H), 2.0-1.6 (m, 2H), 1.6 (ddd, J=18.5, 7.7, 5.2 Hz, 1H), 1.5-1.3 (m, 1H), 1.3 (d, J=2.0 Hz, 9H), 1.2 (td, J=7.2, 4.2 Hz, 3H), 1.1 (d, J=10.3 Hz, 3H), 1.0-1.0 (m, 9H), 1.0 (d, J=7.8 Hz, 3H). ¹³C NMR (100 MHz, Methanol-d₄) δ 195.4, 181.5, 172.3, 171.9, 170.5, 158.3, 60.6, 57.4, 52.1, 49.3, 48.5, 40.0, 37.6, 34.4, 34.1, 31.0, 29.3, 28.3, 27.8, 27.8, 25.7, 19.0, 13.6, 11.8.

### (1R,2S,5S)-3-((S)-2-(3-(tert-butyl)ureido)-3,3-dimethylbutanoyl)-N—((R)-4-(cyclopropylamino)-3,4-dioxo-1-((R)-2-oxopyrrolidin-3-yl)butan-2-yl)-6,6-dimethyl-3-azabicyclo[3.1.0]hexane-2-carboxamide (MPI59)

The synthesis of MPI59 was based on Representative synthetic procedure VI. ¹H NMR (400 MHz, Chloroform-d) δ 7.78 (s, 1H), 6.97 (d, J=3.8 Hz, 1H), 5.70 (s, 1H), 5.43-5.30 (m, 1H), 4.87 (s, 1H), 4.36 (s, 2H), 4.08 (d, J=10.3 Hz, 1H), 3.86 (ddd, J=17.3, 10.2, 5.0 Hz, 1H), 3.34 (td, J=18.6, 16.9, 8.0 Hz, 2H), 2.76 (tq, J=7.6, 3.8 Hz, 1H), 2.65-2.53 (m, 1H), 2.55-2.43 (m, 1H), 2.16-1.88 (m, 3H), 1.49 (d, J=4.5 Hz, 2H), 1.26 (s, 9H), 1.05 (d, J=3.2 Hz, 1H), 1.03 (s, 3H), 0.98 (d, J=4.8 Hz, 9H), 0.88 (s, 3H), 0.86-0.81 (m, 2H), 0.65-0.54 (m, 2H).

### (1R,2S,5S)-3-((S)-2-(3-(tert-butyl)ureido)-3,3-dimethylbutanoyl)-N—((S)-4-((cyclopropylmethyl)amino)-3,4-dioxo-1-((S)-2-oxopyrrolidin-3-yl)butan-2-yl)-6,6-dimethyl-3-azabicyclo[3.1.0]hexane-2-carboxamide (MPI60)

The synthesis of MPI60 was based on Representative synthetic procedure VII. ¹H NMR (400 MHz, Chloroform-d) δ 7.60 (s, 1H), 6.88 (t, J=5.8 Hz, 1H), 6.23 (s, 1H), 5.23-5.12 (m, 1H), 5.04 (d, J=10.1 Hz, 1H), 4.59-4.46 (m, 1H), 4.19-4.03 (m, 1H), 3.95-3.77 (m, 1H), 3.20-2.97 (m, 2H), 2.97-2.89 (m, 2H), 2.45-2.01 (m, 3H), 1.82-1.71 (m, 2H), 1.33-1.20 (m, 3H), 1.03 (s, 9H), 0.80 (s, 3H), 0.74 (s, 9H), 0.66 (s, 3H), 0.40-0.22 (m, 2H), 0.12-0.08 (m, 2H). ¹³C NMR (101 MHz, CDCl₃) δ 195.55, 179.99, 172.74, 171.50, 159.14, 157.35, 77.37, 60.57, 57.79, 55.30, 53.30, 50.19, 50.15, 44.27, 40.49, 38.51, 34.72, 30.36, 29.35, 27.86, 27.67, 26.54, 26.36, 26.30, 19.20, 12.64, 10.33, 3.62, 3.57.

### (1R,2S,5S)-3-((S)-2-(3-(tert-butyl)ureido)-3,3-dimethylbutanoyl)-N—((S)-4-(butylamino)-3,4-dioxo-1-((S)-2-oxopyrrolidin-3-yl)butan-2-yl)-6,6-dimethyl-3-azabicyclo[3.1.0]hexane-2-carboxamide (MPI61)

The synthesis of MPI61 was based on Representative synthetic procedure VI. ¹H NMR (400 MHz, Chloroform-d) δ 7.77 (d, J=7.0 Hz, 1H), 6.99 (t, J=6.1 Hz, 1H), 6.27 (s, 1H), 5.37 (ddd, J=10.5, 6.9, 3.4 Hz, 1H), 5.13 (s, 1H), 4.35 (s, 1H), 4.31 (s, 1H), 4.06 (d, J=10.4 Hz, 1H), 3.87 (dt, J=10.4, 2.7 Hz, 1H), 3.46 (s, 1H), 3.38-3.17 (m, 4H), 2.65-2.54 (m, 1H), 2.45 (tdd, J=10.8, 7.5, 2.1 Hz, 1H), 2.09-1.90 (m, 3H), 1.58-1.43 (m, 4H), 1.39-1.29 (m, 2H), 1.25 (s, 9H), 1.01 (s, 3H), 0.98-0.82 (m, 15H). ¹³C NMR (101 MHz, CDCl₃) δ 195.6, 179.9, 172.7, 171.5, 159.3, 157.3, 60.6, 57.8, 53.3, 50.7, 50.2, 48.3, 40.4, 39.1, 38.4, 34.7, 32.7, 31.2, 30.3, 29.4, 28.2, 27.8, 26.5, 26.3, 20.0, 19.2, 13.7, 12.6.

### 1R,2S,5S)-3-((S)-2-(3-(tert-butyl)ureido)-3,3-dimethylbutanoyl)-N—((S)-4-(hexylamino)-3,4-dioxo-1-((S)-2-oxopyrrolidin-3-yl)butan-2-yl)-6,6-dimethyl-3-azabicyclo[3.1.0]hexane-2-carboxamide (MPI62)

The synthesis of MPI62 was based on Representative synthetic procedure VI. ¹H NMR (400 MHz, Chloroform-d) δ 7.75 (d, J=6.9 Hz, 1H), 6.95 (t, J=6.2 Hz, 1H), 6.21 (s, 1H), 5.44-5.31 (m, 1H), 4.39-4.30 (m, 2H), 4.06 (d, J=9.9 Hz, 1H), 3.86 (dd, J 10.4, 5.0 Hz, 1H), 3.28 (dq, J=13.5, 7.2, 6.7 Hz, 5H), 2.69-2.54 (m, 1H), 2.54-2.45 (m, 1H), 2.12-1.86 (m, 3H), 1.60-1.41 (m, 4H), 1.36-1.15 (m, 17H), 1.02 (s, 3H), 0.96 (s, 9H), 0.90-0.87 (m, 6H). ¹³C NMR (101 MHz, CDCl₃) δ 195.54, 179.77, 172.68, 171.41, 159.28, 157.23, 60.50, 57.82, 53.33, 50.78, 50.25, 48.30, 40.40, 39.45, 38.39, 34.73, 32.67, 31.37, 30.23, 29.36, 29.14, 28.28, 27.82, 26.53, 26.32, 22.50, 19.17, 14.00, 12.64.

### Representative Synthetic Procedure VII

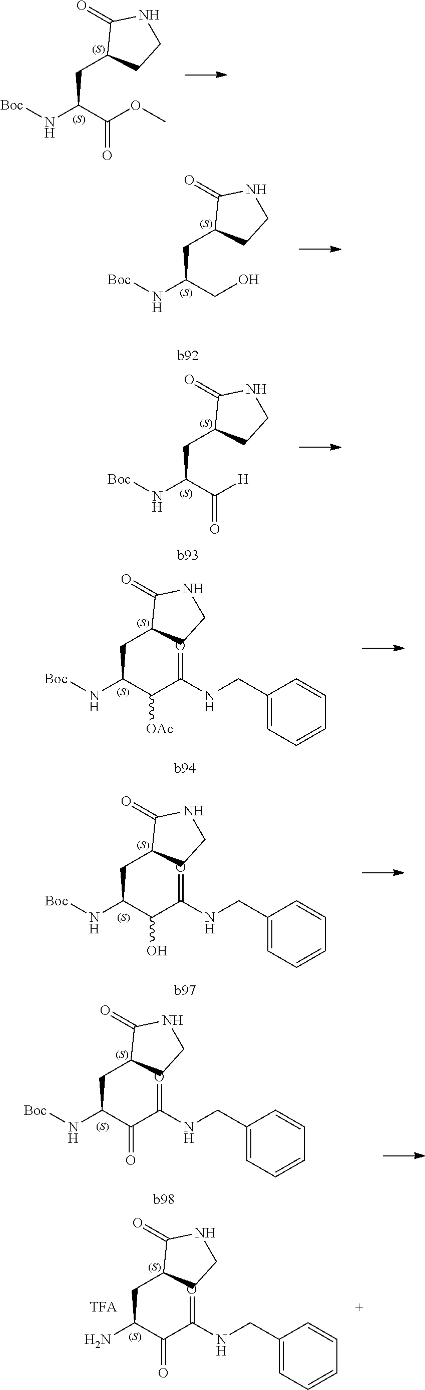

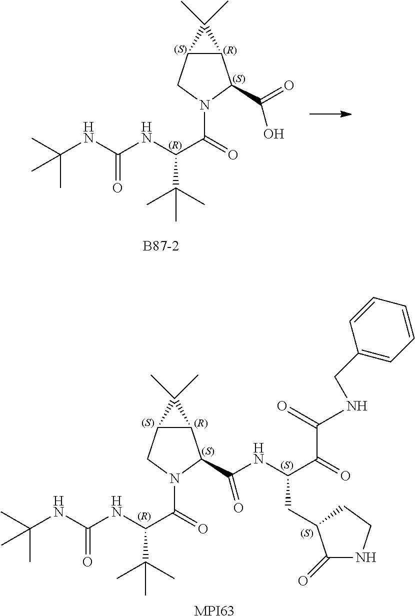

### Tert-butyl ((S)-1-hydroxy-3-((S)-2-oxopyrrolidin-3-yl)propan-2-yl)carbamate (b92)

To a solution of methyl (S)-2-((tert-butoxycarbonyl)amino)-3-((S)-2-oxopyrrolidin-3-yl)propanoate (400 mg, 1.4 mmol, 1.0 equiv) in anhydrous THF (10 mL) at 0° C. was added LiBH₄ (2.0 M in THF, 2.0 mL, 4.2 mmol, 3.0 equiv). The mixture was stirred at RT for 2 h. After the reaction was completed, excess reactants were consumed by slow addition of H₂O. The mixture was diluted with H₂O and extracted with EtOAc, washed with sat. NaCl, dried over Na₂SO₄ and concentrated. The residue was purified by column chromatography (MeOH:EA=1:10 v/v) to afford the pure product b92 as a white solid (320 mg, yield 88%). ¹H NMR (400 MHz, Methanol-d₄) δ 3.7-3.6 (m, 1H), 3.6-3.4 (m, 2H), 3.4 (s, 2H), 2.5-2.3 (m, 2H), 1.9 (q, J=12.3, 10.4 Hz, 2H), 1.5 (s, 10H). ¹³C NMR (100 MHz, Methanol-d₄) δ 182.8, 158.3, 80.0, 65.8, 51.8, 41.5, 39.7, 34.0, 28.8, 28.7.

### Tert-butyl ((S)-1-oxo-3-((S)-2-oxopyrrolidin-3-yl)propan-2-yl)carbamate (b93)

To a solution of b92 (320 mg, 1.2 mmol, 1.0 equiv) in anhydrous DCM (10 mL) was added Dess-Martin reagent (1.0 g, 2.4 mmol, 2.0 equiv) slowly at 0° C. Then the reaction mixture was stirred at RT for 2 h. A solution of NaHCO₃ and Na₂S₂O₃ was added to quench the reaction. After 10 min, the mixture was washed with water, sat. NaCl, dried over Na₂SO₄ and concentrated. The residue was purified by column chromatography (MeOH:DCM=1:15 v/v) to yield b93 as a white solid (230 mg, yield 72%). ¹H NMR (400 MHz, Chloroform-d) δ 9.6 (s, 1H), 6.4 (s, 1H), 6.1 (d, J=6.7 Hz, 1H), 4.2 (p, J=6.2, 5.7 Hz, 1H), 3.4-3.3 (m, 2H), 2.5 (dd, J=17.6, 11.1 Hz, 2H), 2.0-1.9 (m, 2H), 1.4 (d, J=6.7 Hz, 10H). ¹³C NMR (100 MHz, Chloroform-d) δ 200.4, 180.2, 156.3, 80.2, 68.1, 58.8, 40.7, 38.0, 30.5, 28.4.

### (3S)-1-(benzylamino)-3-((tert-butoxycarbonyl)amino)-1-oxo-4-((S)-2-oxopyrrolidin-3-yl)butan-2-yl acetate (b94)

To a solution of b93 (230 mg, 0.9 mmol, 1.0 equiv) in anhydrous DCM (10 mL) at 0° C. Then added benzyl isocyanide (129 mg, 1.1 mmol, 1.2 equiv) and acetic acid (103 μL, 1.8 mmol, 2.0 equiv). Then the reaction mixture was stirred at RT overnight. After the reaction was completed, remove the solvent in vacuum and purified by column chromatography (MeOH:DCM=1:15 v/v) to yield b94 as a white solid (200 mg, yield 51%). ¹H NMR (400 MHz, Chloroform-d) δ 7.3-7.1 (m, 5H), 6.8 (t, J=5.8 Hz, 1H), 6.6 (s, 1H), 5.3 (d, J=9.8 Hz, 1H), 5.2 (d, J=3.6 Hz, 1H), 4.4-4.3 (m, 2H), 4.2 (ddt, J=13.8, 7.2, 3.5 Hz, 1H), 3.3-3.1 (m, 2H), 2.4-2.2 (m, 2H), 2.1 (s, 3H), 1.9 (ddd, J=17.3, 9.5, 3.5 Hz, 1H), 1.8-1.6 (m, 1H), 1.3 (s, 10H). ¹³C NMR (100 MHz, Chloroform-d) δ 180.2, 169.6, 167.9, 155.7, 137.9, 128.7, 127.7, 127.5, 79.7, 74.9, 53.5, 50.0, 43.3, 40.4, 38.0, 33.4, 28.3, 20.8.

### Tert-butyl ((2S)-4-(benzylamino)-3-hydroxy-4-oxo-1-((S)-2-oxopyrrolidin-3-yl)butan-2-yl)carbamate (b97)

To a solution of b94 (200 mg, 0.5 mmol, 1.0 equiv) in 3:1 MeOH/H₂O (8 mL) was added LiOH.H₂O (42 mg, 1.0 mmol, 2.0 equiv) at 0° C. The reaction was stirred at RT for 1 h. After completion, the reaction mixture was neutralized with 0.5 M HCl solution and remove the MeOH in vacuum, then extracted with DCM. The organic layer was washed with sat. NaCl, dried over Na₂SO₄ and concentrated. The residue was purified by column chromatography (MeOH:DCM=1:15 v/v) to yield b97 as a white solid (160 mg, yield 89%). ¹H NMR (400 MHz, Chloroform-d) δ 7.4 (t, J=6.1 Hz, 1H), 7.2 (dq, J=15.5, 8.2, 7.4 Hz, 5H), 6.6 (s, 1H), 5.6 (d, J=5.2 Hz, 1H), 5.5 (d, J=9.3 Hz, 1H), 4.4 (dd, J=14.9, 6.3 Hz, 1H), 4.3 (dd, J=14.9, 5.5 Hz, 1H), 4.2-4.0 (m, 2H), 3.2 (dq, J=17.2, 9.1 Hz, 2H), 2.4-2.2 (m, 2H), 2.1-1.9 (m, 1H), 1.7 (dq, J=17.3, 9.1 Hz, 1H), 1.6 (p, J=5.8 Hz, 1H), 1.3 (s, 9H). ¹³C NMR (100 MHz, Chloroform-d) δ 181.0, 172.4, 156.1, 138.1, 128.6, 127.7, 127.4, 79.6, 73.3, 53.5, 51.1, 43.1, 40.6, 38.0, 32.9, 28.3.

### Tert-butyl ((S)-4-(benzylamino)-3,4-dioxo-1-((S)-2-oxopyrrolidin-3-yl)butan-2-yl)carbamate (b98)

To a solution of b97 (160 mg, 0.4 mmol, 1.0 equiv) in anhydrous DCM (10 mL) was added Dess-Martin reagent (340 mg, 0.8 mmol, 2.0 equiv) slowly at 0° C. Then the reaction mixture was stirred at RT for 2 h. A solution of NaHCO₃ and Na₂S₂O₃ was added to quench the reaction. After 10 min, the mixture was washed with water, sat. NaCl, dried over Na₂SO₄ and concentrated. The residue was purified by column chromatography (MeOH:DCM=1:15 v/v) to yield b98 as a white solid (150 mg, yield 94%). ¹H NMR (400 MHz, Chloroform-d) δ 7.4 (t, J=6.4 Hz, 1H), 7.3-7.2 (m, 5H), 6.7 (s, 1H), 5.9 (d, J=7.7 Hz, 1H), 5.0 (ddd, J=11.4, 7.7, 3.4 Hz, 1H), 4.4 (d, J=6.2 Hz, 2H), 3.3-3.2 (m, 2H), 2.5 (qd, J=8.8, 5.4 Hz, 1H), 2.4 (d, J=8.5 Hz, 1H), 2.3-2.2 (m, 1H), 1.9-1.8 (m, 2H), 1.3 (d, J=3.8 Hz, 9H). ¹³C NMR (100 MHz, Chloroform-d) δ 196.3, 180.1, 159.4, 155.8, 136.9, 128.8, 127.9, 127.9, 79.9, 53.5, 50.6, 43.4, 40.5, 38.5, 33.1, 28.3.

### (1R,2S,5S)—N—((S)-4-(benzylamino)-3,4-dioxo-1-((S)-2-oxopyrrolidin-3-yl)butan-2-yl)-3-((R)-2-(3-(tert-butyl)ureido)-3,3-dimethylbutanoyl)-6,6-dimethyl-3-azabicyclo[3.1.0]hexane-2-carboxamide (MPI63)

To a solution of b98 (70 mg, 0.18 mmol, 1.0 equiv) in anhydrous DCM (5 mL) at 0° C., and then TFA (140 μL, 1.8 mmol, 10 equiv) was added. The mixture was stirred for 2 h. After the reaction was completed, remove the solvent in vacuum. The residue was dissolved in anhydrous DMF at 0° C., and then (1R,2S,5S)-3-((R)-2-(3-(tert-butyl)ureido)-3,3-dimethylbutanoyl)-6,6-dimethyl-3-azabicyclo[3.1.0]hexane-2-carboxylic acid (81 mg, 0.22 mmol, 1.2 equiv), HATU (103 mg, 0.27 mmol, 1.5 equiv), DIPEA (160 μL, 0.9 mmol, 5.0 equiv) was added sequentially. The mixture was stirred at RT overnight. The mixture was diluted with EtOAc and washed with water, 1M HCl, sat. NaCl, dried over Na₂SO₄, and concentrated. The residue was purified by column chromatography (MeOH:DCM=1:15 v/v) to afford the pure product MPI63 as a white solid (75 mg, yield 65%). ¹H NMR (400 MHz, Methanol-d₄) δ 7.4-7.3 (m, 4H), 7.3-7.2 (m, 1H), 6.0 (d, J=8.7 Hz, 1H), 5.8 (d, J=10.0 Hz, 1H), 4.4 (s, 1H), 4.3 (dt, J=10.4, 2.6 Hz, 1H), 4.2 (s, 1H), 4.0 (dd, J=10.2, 4.2 Hz, 1H), 3.9 (dt, J=10.1, 4.9 Hz, 1H), 3.3 (s, 1H), 3.2 (dt, J=13.7, 5.3 Hz, 1H), 2.6 (p, J=11.3 Hz, 1H), 2.4-2.3 (m, 1H), 2.3-2.0 (m, 2H), 1.7-1.6 (m, 1H), 1.5 (dtd, J=15.1, 7.7, 3.6 Hz, 1H), 1.3 (s, 10H), 1.1-0.9 (m, 15H). ¹³C NMR (100 MHz, Chloroform-d) δ 195.5, 180.0, 172.8, 171.5, 159.3, 157.4, 136.8, 128.9, 128.7, 127.9, 60.6, 57.8, 53.3, 50.1, 48.3, 44.1, 43.4, 40.5, 38.4, 34.7, 32.7, 29.4, 28.2, 26.6, 26.3, 19.2, 12.7.

### Benzyl ((2S,3R)-3-(tert-butoxy)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-1-oxobutan-2-yl)carbamate (MPI67)

The synthesis of MPI67 was based on Representative synthetic procedure IV. ¹H NMR (400 MHz, Chloroform-d) δ 9.47 (s, 0.7H), 9.35 (s, 0.3H), 7.37-7.19 (m, 5H), 5.10-4.95 (m, 2H), 4.47-4.14 (m, 3H), 4.10-3.96 (m, 1H), 3.92-3.83 (m, 1H), 3.77-3.57 (m, 2H), 3.22 (pd, J=9.0, 5.4 Hz, 2H), 2.49-2.35 (m, 1H), 2.35-2.23 (m, 1H), 1.97-1.82 (m, 1H), 1.80-1.66 (m, 2H), 1.58-1.38 (m, 2H), 1.14 (s, 9H), 1.04 (d, J=6.2 Hz, 3H), 0.96 (dd, J=10.8, 5.3 Hz, 4H), 0.84 (s, 2H). ¹³C NMR (101 MHz, CDCl₃) δ 200.62, 199.51, 180.39, 179.80, 173.04, 172.09, 169.20, 168.94, 155.83, 155.37, 136.52, 136.24, 128.54, 128.49, 128.20, 128.14, 128.07, 75.09, 75.07, 68.54, 66.93, 62.00, 61.00, 58.73, 57.39, 57.26, 57.14, 48.41, 40.76, 40.39, 37.61, 31.07, 30.37, 29.67, 28.98, 28.41, 28.21, 27.94, 27.31, 26.40, 26.18, 25.50, 20.04, 19.36, 18.64, 18.08, 13.70, 12.77.

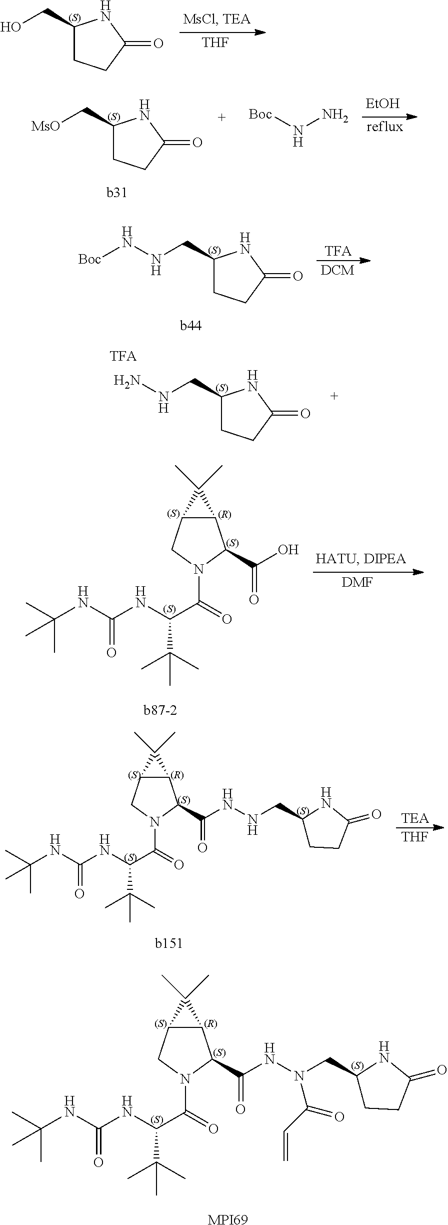

### (S)-(5-(((methylsulfonyl)oxy)methyl)pyrrolidin-2-ylidene)oxonium (b31)

(S)-5-(Hydroxymethyl)-2-pyrrolidinone (500 mg, 4.35 mmol, 1.0 equiv) and TEA (1.2 mL, 8.7 mmol, 2.0 equiv) were dissolved in anhydrous THF at 0° C. Then MsCl (600 μL, 5.22 mmol, 1.2 equiv) was added slowly. The resulting reaction mixture was stirred at RT overnight. After the reaction was completed, the solvent was removed in vacuo. The residue was purified by column chromatography (MeOH:EA=1:5 v/v) to afford the pure product b31 as a white solid (585 mg, yield 70%). ¹H NMR (400 MHz, Chloroform-d) δ 7.5 (s, 1H), 4.2-4.0 (m, 2H), 3.9 (d, J=4.7 Hz, 1H), 3.0 (s, 3H), 2.4-2.1 (m, 3H), 1.8 (qd, J=12.0, 10.9, 4.1 Hz, 1H). ¹³C NMR (100 MHz, Chloroform-d) δ 178.8, 71.5, 52.9, 37.4, 29.6, 22.6.

### Tert-butyl (S)-2-((5-oxopyrrolidin-2-yl)methyl)hydrazine-1-carboxylate (b44)

To a solution of b31 (500 mg, 2.58 mmol, 1.0 equiv) in EtOH was added tert-butyl carbazate (6.8 g, 51.6 mmol, 20 equiv). The resulting solution mixture was heated to reflux 48 h. The solvent was removed in vacuo. The residue was purified by column chromatography (MeOH:EA=1:5 v/v) to afford the pure product b44 as a white solid (265 mg, yield 45%). ¹H NMR (400 MHz, Chloroform-d) δ 3.7 (d, J=7.9 Hz, 1H), 3.1-2.9 (m, 1H), 2.7-2.5 (m, 1H), 2.3 (t, J=8.2 Hz, 2H), 2.1 (dq, J=15.2, 7.6 Hz, 1H), 1.6 (dd, J=13.7, 6.3 Hz, 1H), 1.4 (s, 9H). ¹³C NMR (100 MHz, Chloroform-d) δ 178.5, 157.3, 80.7, 57.4, 52.7, 30.0, 28.3, 24.4.

### 1-(tert-butyl)-3-((S)-1-((1R,2S,5S)-6,6-dimethyl-2-(2-(((S)-5-oxopyrrolidin-2-yl)methyl)hydrazine-1-carbonyl)-3-azabicyclo[3.1.0]hexan-3-yl)-3,3-dimethyl-1-oxobutan-2-yl)urea (b151)

To a solution of b44 (200 mg, 0.87 mmol, 1.0 equiv) in anhydrous DCM at 0° C., then added TFA (660 μL, 8.7 mmol, 10.0 equiv). After 10 minutes, the reaction mixture was warmed to RT for 2 h. After the reaction was completed, removed the solvent and TFA in vacuo. The residue was re-dissolved in anhydrous DMF was added TEA dropwise to adjust the pH to 7.0 at 0° C. Then b87-2 (320 mg, 0.87 mmol, 1.0 equiv), HATU (496 mg, 1.31 mmol, 1.5 equiv) and TEA (176 μL, 1.74 mmol, 2.0 equiv) were sequentially added. The reaction mixture was stirred at RT overnight. The mixture was diluted with EtOAc and washed with water, 1M HCl, sat. NaCl, dried over Na₂SO₄, and concentrated. The residue was purified by column chromatography (DCM:MeOH=7:1 v/v) and the product was used in the next step despite the presence of some impurity.

### 1-((S)-1-((1R,2S,5S)-2-(2-acryloyl-2-(((S)-5-oxopyrrolidin-2-yl)methyl)hydrazine-1-carbonyl)-6,6-dimethyl-3-azabicyclo[3.1.0]hexan-3-yl)-3,3-dimethyl-1-oxobutan-2-yl)-3-(tert-butyl)urea (MPI69)

To a solution of b151 (100 mg, 0.21 mmol, 1.0 equiv) in anhydrous THF at ° C., then TEA (60 μL, 0.42 mmol, 2.0 equiv) and acryloyl chloride (23 mg, 0.25 mmol, 1.2 equiv) were added. The resulting solution mixture was stirred at RT overnight. After the reaction was completed, it was quenched by addition of water. Then the THF was removed in vacuo, and extracted with DCM, washed with brine, dried over Na₂SO₄ and concentrated. The residue was purified by column chromatography (MeOH:DCM=1:10 v/v) to afford the pure product MPI69 as a white solid (230 mg, yield 75%). ¹H NMR (400 MHz, Chloroform-d) δ 6.6 (dd, J=16.7, 10.4 Hz, 1H), 6.3 (d, J=16.9 Hz, 1H), 5.6 (d, J=10.9 Hz, 1H), 4.3-4.1 (m, 3H), 4.1-3.9 (m, 1H), 3.9-3.8 (m, 2H), 3.4-3.1 (m, 1H), 2.3 (td, J=18.9, 18.2, 11.0 Hz, 3H), 1.8-1.7 (m, 1H), 1.6-1.5 (m, 1H), 1.2 (d, J=6.9 Hz, 1H), 1.2 (s, 9H), 1.0 (s, 3H), 0.9 (d, J=3.8 Hz, 12H). ¹³C NMR (100 MHz, Chloroform-d) δ 179.0, 173.8, 171.1, 168.6, 157.8, 130.1, 126.3, 59.3, 58.0, 53.5, 50.0, 49.9, 48.5, 34.5, 30.5, 30.3, 29.5, 29.5, 28.6, 26.6, 26.4, 19.6, 12.7.

### benzyl ((S)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-3,3-dimethyl-1-oxobutan-2-yl)carbamate (MPI73)

The synthesis of MPI73 was based on Representative synthetic procedure IV. ¹H NMR (400 MHz, Chloroform-d) δ 9.52 (s, 1H), 7.99 (d, J=7.4 Hz, 1H), 7.46-7.17 (m, 5H), 6.30 (s, 1H), 5.66 (s, 1H), 5.15-5.00 (m, 2H), 4.44 (ddd, J=9.7, 7.1, 4.7 Hz, 1H), 4.35 (s, 1H), 4.26 (d, J=9.9 Hz, 1H), 4.03-3.84 (m, 2H), 3.35-3.21 (m, 2H), 2.62-2.51 (m, 1H), 2.40-2.28 (m, 1H), 2.07-1.98 (m, 1H), 1.96-1.86 (m, 1H), 1.84-1.71 (m, 1H), 1.59-1.47 (m, 2H), 1.03 (s, 3H), 0.96 (s, 9H), 0.86 (s, 3H). ¹³C NMR (101 MHz, CDCl₃) δ 199.66, 180.22, 172.18, 170.71, 156.42, 136.37, 128.52, 128.47, 128.14, 128.00, 66.91, 60.91, 59.29, 57.27, 53.45, 48.33, 40.47, 37.52, 35.32, 30.90, 27.90, 26.36, 26.24, 19.30, 12.67.

### (2S,3R)-3-(tert-butoxy)-2-(3-(tert-butyl)ureido)-N—((S)-3-cyclohexyl-1-oxo-1-(((S)-1-oxo-3-((S)-2-oxopyrrolidin-3-yl)propan-2-yl)amino)propan-2-yl)butanamide (MPI74)

The synthesis of MPI74 was based on Representative synthetic procedure II. ¹H NMR (400 MHz, Chloroform-d) δ 9.5 (s, 1H), 8.1 (d, J=6.4 Hz, 1H), 7.6 (d, J=8.0 Hz, 1H), 6.9 (s, 1H), 5.7 (d, J=6.0 Hz, 1H), 4.5 (td, J=8.5, 5.4 Hz, 1H), 4.3 (ddd, J=10.8, 7.0, 4.4 Hz, 1H), 4.2 (dd, J=6.0, 3.7 Hz, 1H), 4.1-4.0 (m, 1H), 3.4-3.1 (m, 2H), 2.5-2.3 (m, 2H), 2.1-1.9 (m, 1H), 1.8 (ddd, J=13.2, 8.1, 4.2 Hz, 1H), 1.8-1.5 (m, 8H), 1.5 (ddd, J=14.1, 8.9, 5.6 Hz, 1H), 1.3-1.1 (m, 21H), 1.1 (d, J=12.0 Hz, 1H), 1.0 (d, J=6.3 Hz, 3H), 1.0-0.8 (m, 2H). ¹³C NMR (100 MHz, Chloroform-d) δ 199.9, 180.1, 173.0, 171.6, 157.3, 75.1, 67.6, 58.0, 57.2, 51.3, 50.1, 40.5, 39.8, 37.9, 34.1, 33.6, 32.6, 30.0, 29.5, 28.3, 28.2, 26.3, 26.2, 26.0, 17.6.

### (S)-2-(3-(tert-butyl)ureido)-N—((S)-3-cyclopropyl-1-oxo-1-(((S)-1-oxo-3-((S)-2-oxopyrrolidin-3-yl)propan-2-yl)amino)propan-2-yl)-3-methylbutanamide (MPI75)

The synthesis of MPI75 was based on Representative synthetic procedure II. ¹H NMR (400 MHz, Chloroform-d) δ 9.5 (s, 1H), 7.8-7.7 (m, 1H), 6.6 (d, J=23.0 Hz, 1H), 5.5 (s, 1H), 5.2-5.1 (m, 1H), 4.7-4.4 (m, 2H), 4.2-4.0 (m, 1H), 3.3 (dd, J=10.5, 5.4 Hz, 3H), 2.4 (s, 2H), 2.0 (td, J=12.0, 11.1, 6.2 Hz, 2H), 1.7 (dd, J=46.1, 5.3 Hz, 4H), 1.3 (s, 9H), 1.0-0.8 (m, 6H), 0.7 (s, 1H), 0.4 (t, J=7.6 Hz, 2H), 0.2-0.0 (m, 2H).

### (1R,2S,5S)-3-((S)-3,3-dimethyl-2-(3-methylbutanamido)butanoyl)-6,6-dimethyl-N—((S)-1-oxo-3-((S)-2-oxopyrrolidin-3-yl)propan-2-yl)-3-azabicyclo[3.1.0]hexane-2-carboxamide (MPI76)

The synthesis of MPI76 was based on Representative synthetic procedure IV. ¹H NMR (400 MHz, Chloroform-d) δ 9.47 (s, 1H), 7.83 (d, J=6.8 Hz, 1H), 6.07 (td, J=17.2, 16.6, 5.9 Hz, 2H), 4.55 (dd, J=9.7, 1.4 Hz, 1H), 4.40 (ddd, J=8.9, 6.8, 5.2 Hz, 1H), 4.29 (s, 1H), 3.90 (d, J=2.8 Hz, 2H), 3.35-3.21 (m, 2H), 2.57-2.42 (m, 1H), 2.39-2.30 (m, 1H), 2.01-1.96 (m, 3H), 1.94-1.89 (m, 2H), 1.77 (ddt, J=17.2, 8.3, 4.9 Hz, 1H), 1.51-1.42 (m, 2H), 0.98 (s, 3H), 0.93 (s, 9H), 0.87-0.82 (m, 6H), 0.81 (s, 3H). ¹³C NMR (101 MHz, CDCl₃) δ 199.47, 179.95, 172.23, 172.07, 170.84, 60.82, 57.46, 56.85, 48.37, 45.86, 40.45, 37.64, 35.28, 30.73, 30.02, 28.60, 27.80, 26.47, 26.23, 22.40, 22.34, 19.27, 12.59.

### (1R,2S,5S)—N—((S)-1-cyano-2-((S)-2-oxopyrrolidin-3-yl)ethyl)-3-((S)-2-(3,3-dimethylbutanamido)-3,3-dimethylbutanoyl)-6,6-dimethyl-3-azabicyclo[3.1.0]hexane-2-carboxamide (MPI84)

The synthesis of MPI84 was based on representative synthetic procedure IV. ¹H NMR (400 MHz, Chloroform-d) δ 7.75 (d, J=8.5 Hz, 1H), 5.06 (q, J=8.1 Hz, 1H), 4.67-4.50 (m, 1H), 4.39-4.10 (m, 3H), 3.77-3.60 (m, 2H), 3.34 (ddt, J=15.5, 8.6, 4.5 Hz, 2H), 2.56-2.37 (m, 2H), 2.35-2.23 (m, 1H), 2.14-2.04 (m, 3H), 2.00-1.83 (m, 2H), 1.48 (t, J=3.6 Hz, 2H), 1.05-0.98 (m, 24H).

### Synthesis of (1R,2S,5S)—N—((S)-4-amino-3,4-dioxo-1-((S)-2-oxopyrrolidin-3-yl)butan-2-yl)-3-((S)-2-(3,3-dimethylbutanamido)-3,3-dimethylbutanoyl)-6,6-dimethyl-3-azabicyclo[3.1.0]hexane-2-carboxamide (MPI85)

The synthesis of MPI85 was based on representative synthetic procedure V. ¹H NMR (400 MHz, Chloroform-d) δ 8.23-7.92 (m, 1H), 6.67 (s, 1H), 6.26 (s, 1H), 5.82 (s, 1H), 5.41 (s, 1H), 5.19-4.97 (m, 1H), 4.67-3.96 (m, 4H), 3.71-3.47 (m, 1H), 3.39-3.02 (m, 2H), 2.61-2.47 (m, 1H), 2.47-2.30 (m, 1H), 2.13-1.79 (m, 4H), 1.52-1.31 (m, 2H), 1.31-1.11 (m, 1H), 1.07-0.85 (m, 11H), 0.85-0.68 (m, 1H).

### Synthesis of benzyl ((2S,3R)-1-(((S)-1-(((S)-4-amino-3,4-dioxo-1-((S)-2-oxopyrrolidin-3-yl)butan-2-yl)amino)-3-cyclohexyl-1-oxopropan-2-yl)amino)-3-(tert-butoxy)-1-oxobutan-2-yl)carbamate (MPI86)

The synthesis of MPI86 was based on representative synthetic procedure V. ¹H NMR (400 MHz, Chloroform-d) δ 7.37 (q, J=5.7, 4.6 Hz, 1H), 7.34-7.22 (m, 5H), 6.10 (d, J=27.2 Hz, 1H), 6.02-5.94 (m, 1H), 5.94-5.82 (m, 1H), 5.28-5.15 (m, 1H), 5.04 (q, J=12.3 Hz, 2H), 4.45-4.32 (m, 1H), 4.32-4.17 (m, 1H), 4.17-4.04 (m, 2H), 3.74-3.62 (m, 1H), 3.29-3.16 (m, 2H), 3.14 (s, 1H), 2.51-2.39 (m, 1H), 2.39-2.13 (m, 2H), 2.05-1.88 (m, 3H), 1.83-1.72 (m, 2H), 1.72-1.52 (m, 7H), 1.52-1.39 (m, 1H), 1.19 (s, 9H), 1.02 (dd, J=10.6, 6.0 Hz, 4H), 0.97-0.73 (m, 2H)

### (1R,2S,5S)-3-((S)-2-(3,3-dimethylbutanamido)-3,3-dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide (MPI92)

The synthesis of MP192 was based on representative synthetic procedure IV. ¹H NMR (400 MHz, Chloroform-d) δ 9.40 (s, 1H), 7.67 (d, J=7.6 Hz, 1H), 6.37-6.11 (m, 2H), 4.46-4.39 (m, 1H), 4.33 (d, J=7.6 Hz, 2H), 4.26 (ddd, J=10.4, 4.0, 1.4 Hz, 1H), 3.66 (d, J=10.4 Hz, 1H), 3.37-3.29 (m, 2H), 2.50-2.36 (m, 2H), 2.08-2.01 (m, 3H), 1.96-1.81 (m, 2H), 1.54-1.47 (m, 2H), 1.05 (s, 3H), 1.02 (s, 9H), 1.00 (s, 9H), 0.98 (s, 3H).

### Synthesis of benzyl ((S)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-1-oxo-3-phenylpropan-2-yl)carbamate (MPI94)

The synthesis of MPI94 was based on representative synthetic procedure IV. ¹H NMR (400 MHz, Chloroform-d) δ 9.59-9.36 (m, 1H), 7.27 (d, J=6.1 Hz, 6H), 7.15-7.05 (m, 5H), 5.65 (s, 2H), 4.17-3.90 (m, 3H), 3.27 (d, J=7.6 Hz, 3H), 2.95-2.75 (m, 2H), 2.39 (d, J=7.2 Hz, 2H), 1.86-1.77 (m, 3H), 1.44 (d, J=7.0 Hz, 2H), 1.18 (dd, J=7.1, 3.0 Hz, 3H), 0.83 (d, J=5.3 Hz, 3H).

### 3-Fluorobenzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate (MPI102)

The synthesis of MPI102 was based on representative synthetic procedure II. ¹H NMR (400 MHz, Chloroform-d) δ 9.50 (s, 1H), 8.04 (d, J=6.4 Hz, 1H), 7.46 (d, J=7.7 Hz, 1H), 7.32 (td, J=7.9, 5.8 Hz, 1H), 7.16-6.96 (m, 3H), 6.10 (d, J=22.8 Hz, 1H), 5.95 (d, J=5.2 Hz, 1H), 5.10 (q, J=12.7 Hz, 2H), 4.49 (q, J=7.3 Hz, 1H), 4.40-4.33 (m, 1H), 4.24-4.11 (m, 2H), 3.42-3.25 (m, 2H), 2.41 (dddd, J=27.2, 13.2, 9.1, 6.7 Hz, 2H), 2.07-1.88 (m, 2H), 1.87-1.52 (m, 8H), 1.36 (dh, J=8.7, 3.0 Hz, 1H), 1.27 (s, 9H), 1.23-1.11 (m, 4H), 1.08 (d, J=6.1 Hz, 3H), 0.96 (dtd, J=24.1, 11.8, 3.1 Hz, 2H).

### 4-Fluorobenzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate (MPI103)

The synthesis of MPI103 was based on representative synthetic procedure II. ¹H NMR (400 MHz, Chloroform-d) δ 9.50 (s, 1H), 8.01 (d, J=6.4 Hz, 1H), 7.45 (d, J=7.7 Hz, 1H), 7.33 (dd, J=8.4, 5.4 Hz, 2H), 7.03 (t, J=8.6 Hz, 2H), 6.19 (s, 1H), 5.91 (s, 1H), 5.06 (q, J=12.2 Hz, 2H), 4.49 (q, J=7.5 Hz, 1H), 4.38 (dt, J=10.4, 5.7 Hz, 1H), 4.17 (d, J=5.6 Hz, 2H), 3.31 (dt, J=9.4, 4.9 Hz, 2H), 2.50-2.28 (m, 2H), 1.97 (dtd, J=28.8, 14.5, 7.5 Hz, 2H), 1.86-1.55 (m, 9H), 1.35 (q, J=7.7, 6.4 Hz, 1H), 1.26 (s, 11H), 1.22-1.11 (m, 4H), 1.07 (d, J=6.0 Hz, 4H), 1.00-0.88 (m, 2H).

SARS-CoV-2 Inhibition Analysis of GC376, MPI1-8, and 11a.

To evaluate the molecules' ability to inhibit SARS-CoV-2, a live virus-based microneutralization assay was performed in Vero E6 cells. Vero E6 is a kidney epithelial cell line isolated from African Green Monkey. It has been used widely as a model system for human CoV studies. 10 molecules including GC376, MPI1-8, and 11a were tested in a concentration range from 80 nM to 10 μM and recorded cytopathogenic effect (CPE) observed in SARS-CoV-2-infected Vero E6 cells that were cultured in the presence of different concentrations of inhibitors. 11a was included as a positive control. For each condition, two repeats were conducted. MPI3 was not able to completely prevent CPE at all tested concentrations. Several other inhibitors abolished CPE: GC376, MPI2, MPI6, and MPI10 at 10 μM, MPI5 at 5 μM, MPI7 at 2.5-5 μM, and MPI8 at 2.5 μM. Three compounds MPI5, MPI7, and MPI8 performed better than GC376. Since only complete abolition of CPE was recorded, the real EC₅₀ values for these compounds are expected to be much lower than lowest observed concentrations for CPE abolishment. Encouraged by these results in Vero E6 cells, the three most potent compounds MPI5, MPI7, and MPI8 and also MPI10 were tested in A549/ACE2 cells. The A549/ACE2 cell line was derived from human alveolar epithelial cells. It mimics the SARS-CoV-2 infection of the human respiratory tract system better than Vero E6. The same concentration range for all four compounds was tested. MPI7 was not able to completely abolish CPE at all tested conditions. However, both MPI5 and MPI8 performed much better than in Vero E6 cells with complete abolition of CPE at 160-310 nM and much better than MPI10. MPI10 displayed potency similar to that shown in Vero E6 cells. Given that real EC₅₀ values are expected to be lower than the lowest observed concentration for CPE abolishment, MPI5 and MPI8 are, are potent anti-SARS-CoV-2 small molecules in infected cells.

The Establishment of a Cellular M^{Pro} Inhibition Assay.

A typical antiviral assay for SARS-CoV-2 is its triggering of strong cytopathogenic effect (CPE) in host cells leading to death that can be quantified by counting formed viral plaques. An M^{Pro} inhibitor with high cellular potency will suppress this strong CPE leading to host cell survival. A suitable cellular M^{Pro} inhibition assay will need to mimic this CPE suppression process to a large extent. To this end, two constructs were built. The first construct pLVX-M^{Pro}-eGFP-1 encodes M^{Pro}-eGFP with a N-terminal methionine that relies on host methionine aminopeptidases for its cleavage. The second construct pLVX-M^{Pro}-eGFP-2 encodes M^{Pro}-eGFP containing a short N-terminal peptide that has an M^{Pro} cleavage site at the end for its autocatalytic release. Transfection of 293T cells with two constructs showed that pLVX-M^{pro}-eGFP-2 led to more potent toxicity to cells and this toxicity was effectively suppressed when we provided 10 μM MPI8 in the growth media. Therefore, pLVX-M^{Pro}-eGFP-2 was selected for the following studies. To demonstrate that cellular fluorescence is positively correlated to the concentration of provided MPI8, 293T cells were transfected with pLVX-M^{Pro}-eGFP-2, grew transfected cells in the presence of four MPI8 concentrations (0, 20, 40, and 160 nM) for 72 h, and then sorted cells using fluorescent flow cytometry. Both the number and intensity of fluorescent cells (FL1-A signal>1×10⁶) were positively dependent on the provided MPI8 concentration, indicating the feasibility of using the system to characterize cellular potency of an M^{Pro} inhibitor. To demonstrate this feasibility, 293T cells were transiently transfected with pLVX-M^{Pro}-eGFP-2 and grew transfected cells in the presence of a cascade of MPI8 concentrations that started from 10 μM and descended 5 folds consecutively. After 72 h, we sorted cells according to their eGFP fluorescent intensity. Cells with FL1-A signal above 1×10⁶ were analyzed. A METLAB script was built to calculate average eGFP fluorescent intensity of all analyzed cells and plotted average eGFP fluorescent intensity against the MPI8 concentration. The data showed obvious MPI8-induced saturation of M^{Pro}-eGFP expression and fit nicely to a three-parameter dose dependent inhibition mechanism in Prism 9 for IC₅₀ determination. The determined cellular M^{Pro} inhibition IC₅₀ value of MPI8 is 31 nM. As described above, an antiviral assay in Vero E6 cells showed an EC₅₀ value of 30 nM for MPI8 in inhibiting SARS-CoV-2. This high similarity between cellular M^{Pro} inhibition IC₅₀ and antiviral EC₅₀ values of MPI8 validates that cellular M^{Pro} inhibition potency of an inhibitor represents closely its antiviral potency through M^{Pro} inhibition.

Cellular M^{Pro} Inhibition Assay for MPI1-7, MPI9, GC376, and MPI10.

MPI8 was one of 9 β-(S-2-oxopyrrolidin-3-yl)-alaninal (Opal)-based, reversible covalent M^{Pro} inhibitors MPI1-9 described herein. GC376 is a prodrug that dissociates quickly in water to release its Opal component. MPI10 is another Opal-based, reversible covalent M^{Pro} inhibitor that was developed in 2020. All 11 compounds showed high potency in inhibiting M^{Pro} in an enzymatic assay. Besides MPI8, cellular potency of all other 10 Opal inhibitors were evaluated in their cellular inhibition of M^{Pro} as well by following the exact same procedure that we did for MPI8. All tested Opal inhibitors promoted cell survival and the expression of M^{Pro}-eGFP significantly at 10 μM. However, data collected at different concentrations showed that only three inhibitors, MPI5, 6, and 7 induced saturation of M^{Pro}-eGFP expression at or below 10 μM. Determined IC₅₀ values for MPI5, 6, and 7 are 0.66, 0.12, and 0.19 μM, respectively. Based on collected data, MPI2-4, MPI9, GC376, and MPI10 have IC₅₀ values higher than 2 μM and MPI1 that displayed the lowest inhibition of M^{Pro} at 10 μM among all Opal inhibitors has an IC₅₀ value higher than 10 μM.

The Determination of Antiviral EC₅₀ Values for MPI5-8.

The previous antiviral assay for Opal inhibitors were based on on-off observation of CPE in Vero E6 and ACE2⁺ A549 cells. To quantify antiviral EC₅₀ values of MPI5-8, plaque reduction neutralization tests of SARS-CoV-2 in Vero E6 cells were performed in the presence of MPI5-8. Vero E6 cells were infected with SARS-CoV-2, grew infected cells in the presence of different concentrations of each inhibitor for 3 days, and then quantified SARS-CoV-2 plaque reduction. Based on SARS-CoV-2 plaque reduction in the presence of MPI5-8, antiviral EC₅₀ values were measured for MPI5-8 as 73, 209, 170, and 30 nM, respectively.

Based on medicinal chemistry of SC2M^{Pro} inhibitors, the crystal structure analysis, and cellular M^{Pro} inhibition assay further developed a series of new potent SC2M^{Pro} inhibitors were prepared. The representative of SC2M^{Pro} inhibitors and their enzymatic and cellular IC₅₀ values in inhibiting SC2M^{Pro} are in Table 1.

Recombinant SC2M^{Pro} Protein Expression and Purification

The construct pET28a-His-SUMO-SC2M^{Pro} construct was made based on a pET28a plasmid modified with N-terminal His-SUMO tag. The gene encoding SC2M^{pro} was amplified from a previous plasmid pBAD-sfGFP-M^{pro} using the forward primer 5′-CGCGGATCCGGGTTTCGCAAG-3′ and the reverse primer 5′-CCGCTCGAGTTACTGAAAAGTTACGCC-3′. The amplified PCR product was digested with BamHI and XhoI and ligated into the vector pET28a-His-SUMO plasmid digested with the same restriction enzymes. The gene sequence of His-SUMO-SC2M^{pro} was verified by sequencing at Eton Bioscience Inc.

The pET28a-His-SUMO-SC2M^{Pro} construct was transformed into E. coli strain BL21(DE3). Cells were cultured at 37° C. in 6 L 2×YT medium with kanamycin (50 μg/mL) for 3 h and induced with isopropylβ-D-1-thiogalactoside (IPTG) at final concentration of 1 mM when the OD₆₀₀ reached 0.8. After 3 h, cells were harvested by centrifugation at 12,000 rpm, 4° C. for 30 min. Cell pellets were resuspended in 150 mL buffer A (20 mM Tris, 100 mM NaCl, 10 mM imidazole, pH 8.0) and then lysed by sonication on ice. The lysate was clarified by centrifugation at 16,000 rpm, 4° C. for 30 min. The supernatant was loaded onto a nickel-chelating column with High Affinity Ni-Charged Resin (GenScript) and washed with 10 column volumes of buffer A to remove unspecific binding proteins, followed by elution using buffer B (20 mM Tris, 100 mM NaCl, 250 mM imidazole, pH 8.0). The protein eluates were subjected to buffer exchange with buffer C (20 mM Tris, 10 mM NaCl, 1 mM dithiothreitol (DTT), pH 8.0) by using HiPrep 26/10 desalting column (GE Healthcare). The His-SUMO-SC2M^{pro} proteins were digested with SUMO protease overnight at 4° C. The digested protein was applied to nickel-chelating column again to remove the His-tagged SUMO protease, the His-SUMO tag, and protein with uncleaved His-SUMO tag. The tag-free SC2M^{pro} protein was loaded onto an anion-exchange column with Q Sepharose, Fast Flow (GE Healthcare) equilibrated with buffer C for further purification. The column was eluted by buffer D (20 mM Tris, 1 M NaCl, 1 mM DTT, pH 8.0) with a linear gradient ranging from 0 to 500 mM NaCl (10 column volumes buffer). Fractions eluted from the anion-exchange column were condensed and loaded to size exclusion column with HiPrep 16/60 Sephacryl 5-100 HR (GE Healthcare) pre-equilibrated with buffer E (20 mM Tris, 100 mM NaCl, 1 mM DTT, 1 mM EDTA, pH 7.8). The eluted SC2M^{pro} protein in buffer E was concentrated to 20 mg/mL and stored in −80° C. for further use.

The Determination of Kₘ for Sub3

The assays were carried out with 20 nM enzyme and various concentration of Sub3, a fluorogenic substrate we purchased from BaChem (DABCYL-Lys-Thr-Ser-Ala-Val-Leu-Gln-Ser-Gly-Phe-Arg-Lys-Met-Glu-EDANS) at 37° C. Aliquot was taken out at indicated time points and diluted 10 times to stop the reaction. Fluorescent intensity was recorded immediately. Data treatment were done with Graph Pad Prism 8.0 software. First 14 min were analyzed by linear regression for initial reaction rate analyses. The initial reaction rates were used to determine the Kₘ value by fitting with Michaelis-Menten non-linear regression.

IC₅₀ Analysis

The assays were carried out with 20 nM enzyme (except for MPI3, for which 10 nM enzyme was used) and 10 μM substrate at 37° C. with continuous shaking. All the analyses were carried out in triplicate. The substrate (DABCYL-Lys-Thr-Ser-Ala-Val-Leu-Gln-Ser-Gly-Phe-Arg-Lys-Met-Glu-EDANS) was purchased from Bachem and stored as 1 mM solution in 100% DMSO. Enzyme activity was monitored by fluorescence with excitation at 336 nm and emission at 455 nm wavelength. The dilution buffer (used for enzyme and substrate dilution) is 10 mM NaₓH_{y}PO₄, 10 mM NaCl, 0.5 mM EDTA, pH 7.6. Final composition of the assay buffer is 10 mM NaₓH_{y}PO₄, 10 mM NaCl, 0.5 mM EDTA, 2 μM DTT (coming from enzyme stock solution), pH 7.6 with 1.25% DMSO. All the inhibitors were stored as 10 mM in 100% DMSO solutions in −20° C. freezer.

For IC₅₀ analysis, the inhibitor was diluted to 400-fold times higher than the highest working concentration to make the secondary stock solution (i.e. if the highest working concentration of inhibitor is 2 μM, then the inhibitor was diluted from its 10 mM stock solution to 800 μM in DMSO). 10 μL from this secondary stock solution was added to the 990 μL of dilution buffer. Serial dilutions were carried out in dilution buffer containing 1% DMSO to ensure all the inhibitor serial dilutions are at 1% DMSO. 25 μL of each inhibitor solution were added to 96-well plate with multichannel pipette. Next, 25 μL of 80 nM enzyme solution (diluted from 10 μM enzyme storage solution in 10 mM NaₓH_{y}PO₄, 10 mM NaCl, 0.5 mM EDTA, pH 7.6, 1 mM DTT with dilution buffer) were added by multichannel pipette and mixed by pipetting up and down three times. Then, the enzyme-inhibitor solution was incubated at 37° C. for 30 minutes. During incubation period, 20 μM of the substrate solution is prepared by diluting from 1 mM stock solution with dilution buffer. When the incubation period is over, 50 μL of the 20 μM substrate solution added to each well by multichannel pipette and the assay started. Data recording were stopped after 30 minutes. Data treatment were done with Graph Pad Prism 8.0 software. First 0-300 seconds were analyzed by linear regression for initial slope analyses. Then, the initial slopes were normalized and IC₅₀ values were determined by inhibitor vs response—Variable slope (four parameters).

Crystallization of SC2M^{pro}

A freshly prepared SC2M^{pro} protein solution at a concentration of 10 mg/mL was cleared by centrifugation at 14,000 rpm, 10 min. Next, a basic screen with the commercially available screening kits (Hampton Research Index™, Crystal Screen™ 1 and 2, PEGRx™ 1 and 2, PEG/Ion™ 1 and 2) were performed employing the sitting-drop vapor-diffusion method at 18° C. 1.0 μL of SC2M^{pro} protein solution and 1.0 μL of reservoir buffer were mixed to equilibrate against 100 μL reservoir solution. Crystals appeared overnight under over 50 conditions. The most promising crystal was found under condition No. 44 of PEG/Ion™ (0.2 M Ammonium phosphate dibasic, 20% w/v PEG3350, pH8.0). Subsequent optimization was performed by adjusting the temperature and concentration of protein and precipitant. The best plate-like crystals were obtained at 25° C. from 0.2 M Ammonium phosphate dibasic, 17% w/v PEG3350, pH8.0, with a SC2M^{pro} protein concentration of 14 mg/ml. Overnight growing crystals were washed with cryo-protectant containing mother liquor plus gradually increasing glycerol (5%, 10%, 15%, 20%, 25% and 30%). Cryo-protected crystals were fished for data collection.

Crystallization of SC2M^{pro} in Complex with Inhibitors

Soaking was performed to produce SC2M^{pro}-inhibitor complex crystals. Overnight growing SC2M^{pro} crystals were washed with reservoir solution three times in situ. Subsequently, the crystals were washed three times with reservoir solution plus 0.5 mM inhibitor and 2% DMSO (Inhibitors were dissolved to 25 mM in 100% DMSO). The mixture was incubated at 25° C. for 48 h. The cryo-protectant solution contained mother liquor plus 30% glycerol, 0.5 mM inhibitor and 2% DMSO. Cryo-protected crystals were fished for data collection.

Data Collection and Structure Determination

The data of SC2M^{pro} with MPI6 and MPI8 were collected on a Rigaku R-AXIS IV++ image plate detector. All the other data were collected at the Advanced Light Source (ALS) beamline 5.0.2 using a Pilatus3 6M detector. The diffraction data were indexed, integrated and scaled with iMosfim. All crystals are in space group C121. All the structures were determined by molecular replacement using the structure model of the free enzyme of the SARS-CoV-2 (2019-nCoV) main protease [Protein Data Bank (PDB) ID code 6Y2E] as the search model using Phaser in the Phenix package. JLigand and Sketcher from the CCP4 suite were employed for the generation of PDB and geometric restraints for the inhibitors. The inhibitors were built into the F_{O}-F_{C} density by using Coot. Refinement of all the structures was performed with Real-space Refinement in Phenix. All structural figures were generated with PyMOL (https://www.pymol.org).

SARS-CoV-2 Inhibition by a Cell-Based Assay

A slightly modified cytopathic effect (CPE)-based microneutralization assay was used to evaluate the drug efficacy against SARS-CoV-2 infection. Briefly, confluent African green monkey kidney cells (Vero E6) or human alveolar epithelial A549 cells stably expressing human ACE2 viral receptor, designated A549/hACE2, grown in 96-wells microtiter plates were pre-treated with serially 2-folds diluted individual drugs for two hours before infection with 100 or 500 infectious SARS-CoV-2 (US_WA-1 isolate) particles in 100 μL EMEM supplemented with 2% FBS, respectively. Cells pre-treated with parallelly diluted DMSO with or without virus were included as positive and negative controls, respectively. After cultivation at 37° C. for 3 (Vero E6) or 4 days (A549/hACE2), individual wells were observed under the microcopy for the status of virus-induced formation of CPE. The efficacy of individual drugs was calculated and expressed as the lowest concentration capable of completely preventing virus-induced CPE in 100% (EC100) or 50% (EC50) of the wells. All compounds were dissolved in 100% DMSO as 10 mM stock solutions before subjecting to dilutions with culture media.

TABLE 1
Determined enzymatic and cellular IC₅₀ values in inhibiting
SARS-CoV-2 M^{Pro} for different inhibitors

| Compound | Enzymatic IC₅₀ | Cellular IC₅₀ | Anti-SARS-CoV-2 |
|---|---|---|---|
| ID | (μM) | (μM) | EC₅₀ (μM) |
| MPI1 | 0.100 ± 0.023 | >10 | |
| MPI2 | 0.103 ± 0.014 | >2 | |
| MPI3 | 0.0085 ± 0.0015 | >2 | |
| MPI4 | 0.015 ± 0.005 | >2 | |
| MPI5 | 0.033 ± 0.002 | 0.66 ± 0.15 | 0.073 ± 0.007 |
| MPI6 | 0.060 ± 0.004 | 0.12 ± 0.03 | 0.209 ± 0.022 |
| MPI7 | 0.047 ± 0.003 | 0.19 ± 0.03 | 0.170 ± 0.022 |
| MPI8 | 0.105 ± 0.022 | 0.031 ± 0.002 | 0.030 ± 0.003 |
| MPI9 | 0.056 ± 0.014 | >2 | |
| MPI12 | 0.025 | >5 | |
| MPI13 | (insoluble) | | |
| MPI14 | 0.053 | >10 | |
| MPI15 | 0.009 | 1.43 | |
| MPI16 | 0.0109 ± 0.0044 | 4 | |
| MPI17 | 0.0364 ± 0.0029 | 0.91 | |
| MPI18 | 0.0163 ± 0.0011 | 2.53 | |
| MPI19 | 0.0586 ± 0.0022 | 0.097 | |
| MPI20 | 0.126 ± 0.006 | 0.056 | |
| MPI21 | 0.0048 ± 0.00023 | 3.43 | |
| MPI22 | 0.0765 ± 0.0124 | 2.88 | |
| MPI23 | 0.0495 ± 0.0035 | >10 | |
| MPI26 | 0.346 | >10 | |
| MPI27 | 0.649 ± 0.025 | 0.109 | |
| MPI28 | 0.160 ± 0.017 | 0.701 | |
| MPI31 | 0.0124 ± 0.0006 | 1.48 | |
| MPI32 | 0.0083 ± 0.0016 | 6.05 | |
| MPI33 | >1 | | |
| MPI34 | 0.525 ± 0.002 | 0.22 | |
| MPI35 | 4.277 ± 0.278 | | |
| MPI36 | >10 | | |
| MPI37 | 0.064 | 0.755 | |
| MPI38 | 0.640 | >10 | |
| MPI39 | 5.038 ± 0.013 | | |
| MPI40 | 1.616 ± 0.153 | | |
| MPI41 | 39 | | |
| MPI42 | >10 | | |
| MPI43 | >10 | | |
| MPI44 | >10 | | |
| MPI45 | >10 | | |
| MPI46 | >10 | | |
| MPI47 | 1.04 | | |
| MPI50 | 0.109 | 0.949 | |
| MPI51 | 0.036 | >5 | |
| MPI52 | 0.061 ± 0.0012 | 0.376 | |
| MPI53 | 0.189 | 0.069 | |
| MPI54 | 0.0093 ± 0.0008 | >5 | |
| MPI55 | 0.0563 ± 0.0038 | 2.80 | |
| MPI56 | 0.0234 ± 0.0002 | >5 | |
| MPI57 | 0.0402 ± 0.0042 | 10.46 | |
| MPI58 | 0.075 ± 0.009 | >5 | |
| MPI59 | 0.365 ± 0.052 | 14.04 | |
| MPI60 | 0.617 ± 0.167 | >5 | |
| MPI61 | 0.366 ± 0.028 | >5 | |
| MPI62 | 0.716 ± 0.075 | >5 | |
| MPI63 | 0.1027 ± 0.0015 | >5 | |
| MPI64 | 0.0261 ± 0.0014 | >5 | |
| MPI65 | 0.294 ± 0.015 | 0.066 | |
| MPI66 | 0.0188 ± 0.0008 | 3.45 | |
| MPI67 | 0.120 ± 0.014 | 0.147 | |
| MPI68 | 0.097 | 0.82 | |
| MPI69 | >2 | | |
| MPI70 | 0.136 ± 0.017 | | |
| MPI72 | 0.0447 ± 0.0045 | 0.373 | |
| MPI73 | 0.059 ± 0.0072 | 0.308 | |
| MPI74 | 0.097 ± 0.0106 | 1.6-2.8 | |
| MPI75 | 0.0261 ± 0.0014 | >5 | |
| MPI76 | 0.0221 ± 0.0039 | 2.63 | |
| MPI84 | 0.718 ± 0.089 | 1.13 | |
| MPI85 | 0.147 ± 0.019 | >10 | |
| MPI86 | 0.167 ± 0.014 | 7.74 | |
| MPI88 | | | |
| MPI89 | | | |
| MPI92 | 0.181 ± 0.020 | 0.84 | |
| MPI94 | 0.267 ± 0.044 | | |
| MPI95 | | | |
| MPI100 | | | |
| MPI101 | | | |
| MPI102 | 0.307 | | |
| MPI103 | 0.196 ± 0.031 | | |

1. A compound of formula 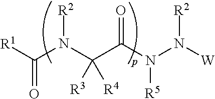
or a pharmaceutically acceptable salt thereof, wherein
R¹ is C₁-C₆ alkyl, C₂-C₆ alkenyl, C₁-C₆ alkoxy, 5-10 membered heteroaryl, —NH₂, —NHR^{a}, —NR^{a}R^{b}, or —C₁-C₆ alkyl-NR^{a}R^{b}, wherein each hydrogen atom in C₁-C₆ alkyl, C₂-C₆ alkenyl, C₁-C₆ alkoxy, and 5-10 membered heteroaryl is optionally substituted by an R^{a};
each of R² and R³ is independently H or C₁-C₆ alkyl; or R² and R³ together with the atoms to which they are attached combine to form 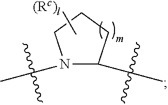
each R⁴ is H, C₁-C₆ alkyl, C₂-C₆ alkenyl, or C₃-C₈ cycloalkyl, wherein each hydrogen atom in C₁-C₆ alkyl, C₂-C₆ alkenyl, and C₃-C₈ cycloalkyl is optionally substituted by R^{b};
R⁵ is H;
R⁶ is C₁-C₆ alkyl, wherein each hydrogen atom in C₁-C₆ alkyl is optionally substituted by C(O)NR^{a}R^{b}, 3- to 8-membered heterocycloalkyl, or 5-10 membered heteroaryl;
W is C₁-C₆ alkyl-SO₄, C₁-C₆ alkyl-CN, —C(O)H, —C(O)OR^{d}, —C(O)C₁-C₆ alkyl, —C(O)C₂-C₆ alkenyl, —C(O)C₆-C₁₀ aryl, —C(O)C(O)NR^{a}R^{b}, C(O)COC(O)C₁-C₁₀ alkyl, —C(O)COC(O)C₆-C₁₀ aryl, —C(O)COC(O)-5-10 membered heteroaryl, or —CN, wherein each hydrogen atom in C₁-C₆ alkyl, C₂-C₆ alkenyl, and C₆-C₁₀ aryl is optionally substituted by halo, —OSO₄, —CN, —OR^{d}, —NO₂, C₆-C₁₀ aryl, or —NR^{a}R^{b};
each R^{a} and R^{b}, when present, is independently H, C₁-C₆ alkyl, C₃-C₈ cycloalkyl, C₁-C₆ alkoxy, or C₆-C₁₀ aryl, wherein each hydrogen atom in C₁-C₆ alkyl, C₃-C₈ cycloalkyl, C₁-C₆ alkoxy, and C₆-C₁₀ aryl is optionally substituted by halo, OR^{d}, 5-10 membered heteroaryl, C₂-C₆ alkenyl, C₆-C₁₀ aryl, or C₃-C₈ cycloalkyl;
each R^{c}, when present, is C₁-C₆ alkoxy or C₃-C₈ cycloalkyl, or two R^{c} combine together with the atom or atoms to which they are attached to form a C₃-C₈ cycloalkyl, wherein each hydrogen atom in C₃-C₈ cycloalkyl is optionally substituted by C₁-C₆ alkyl;
R^{d}, when present, is H, C₁-C₆ alkyl, —C(O)C₁-C₆ alkyl, or C₆-C₁₀ aryl, wherein each hydrogen atom in C₁-C₆ alkyl and C₆-C₁₀ aryl is optionally substituted by halo or —NO₂;
l is 0, 1, 2, or 3;
m is 1, 2, or 3; and
p is 1 or 2;
or a pharmaceutically acceptable salt thereof.

2. The compound or pharmaceutically acceptable salt of claim 1, wherein at least one R⁴ is C₁-C₆ alkyl, wherein each hydrogen atom in C₁-C₆ alkyl is optionally substituted by C₁-C₆ alkoxy or C₃-C₈ cycloalkyl.

3. The compound or pharmaceutically acceptable salt of claim 2, wherein at least one R⁴ is C₁-C₆ alkyl, wherein at least one hydrogen atom in C₁-C₆ alkyl is substituted by C₁-C₆ alkoxy.

4. The compound or pharmaceutically acceptable salt of claim 3, wherein the compound is of formula

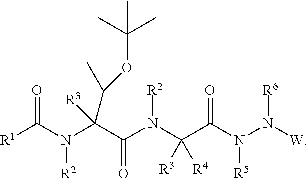

5. The compound or pharmaceutically acceptable salt of claim 2, wherein at least one R⁴ is C₁-C₆ alkyl, wherein at least one hydrogen atom in C₁-C₆ alkyl is substituted by C₃-C₈ cycloalkyl.

6. The compound or pharmaceutically acceptable salt of claim 5, wherein the compound is of formula

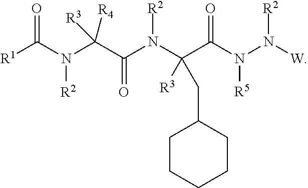

7. The compound or pharmaceutically acceptable salt of claim 1, wherein the compound is of formula

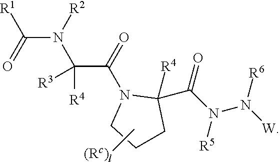

8. A compound of formula 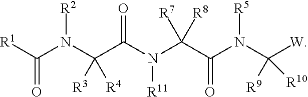
or a pharmaceutically acceptable salt thereof, wherein
R¹ is C₁-C₆ alkyl, C₂-C₆ alkenyl, C₁-C₆ alkoxy, 5-10 membered heteroaryl, —NH₂, —NHR^{a}, —NR^{a}R^{b}, or —C₁-C₆ alkyl-NR^{a}R^{b}, wherein each hydrogen atom in C₁-C₆ alkyl, C₂-C₆ alkenyl, C₁-C₆ alkoxy, and 5-10 membered heteroaryl is optionally substituted by an R^{a};
each of R² and R³ is independently H or C₁-C₆ alkyl; or R² and R³ together with the atoms to which they are attached combine to form 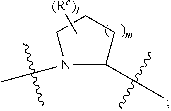
R⁴ is C₁-C₆ alkyl, C₆-C₁₀ aryl, or C₃-C₈ cycloalkyl, wherein each hydrogen atom in C₁-C₆ alkyl, C₆-C₁₀ aryl, and C₃-C₈ cycloalkyl is optionally substituted by R^{b} or C₁-C₆ alkoxy; or R³ and R⁴ together with the carbon atom to which they are attached form C₃-C₈ cycloalkyl, wherein each hydrogen atom in C₃-C₈ cycloalkyl is optionally substituted by R^{b};
R⁵ is H or C₁-C₆ alkyl, wherein each hydrogen atom in C₁-C₆ alkyl is optionally substituted by —C(O)NR^{a}R^{b};
each of R⁷ and R⁸ is independently H, C₁-C₆ alkyl, C₂-C₆ alkenyl, or C₃-C₈ cycloalkyl, wherein each hydrogen atom in C₁-C₆ alkyl, C₂-C₆ alkenyl, and C₃-C₈ cycloalkyl is optionally substituted by R^{b};
each of R⁹ and R¹⁰ is independently H or C₁-C₆ alkyl, wherein each hydrogen atom in C₁-C₆ alkyl is optionally substituted by C(O)NR^{a}R^{b}, 3- to 8-membered heterocycloalkyl, or 5-10 membered heteroaryl;
R¹¹ is H or R¹¹ and one of R⁷ or R⁸ together with the atoms to which they are attached combine to form 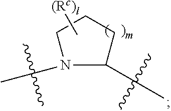
W is C₁-C₆ alkyl-SO₄, C₁-C₆ alkyl-CN, —C(O)H, —C(O)C₁-C₆ alkyl, —C(O)C(O)NR^{a}R^{b}, C(O)COC(O)C₁-C₁₀ alkyl, —C(O)COC(O)C₆-C₁₀ aryl, —C(O)COC(O)-5-10 membered heteroaryl, —CN, wherein each hydrogen atom in C₁-C₆ alkyl, and C₆-C₁₀ aryl is optionally substituted by halo, —OSO₄, —OR^{d}, —CN, —NO₂, C₆-C₁₀ aryl, or —NR^{a}R^{b};
each R^{a} and R^{b}, when present, is independently H, C₁-C₆ alkyl, C₃-C₈ cycloalkyl, C₁-C₆ alkoxy, C₆-C₁₀ aryl, or 5-10 membered heteroaryl, wherein each hydrogen atom in C₁-C₆ alkyl, C₃-C₈ cycloalkyl, C₁-C₆ alkoxy, C₆-C₁₀ aryl, and 5-10 membered heteroaryl is optionally substituted by halo, —OR^{d}, 5-10 membered heteroaryl, C₆-C₁₀ aryl, C₂-C₆ alkenyl, or C₃-C₈ cycloalkyl;
each R^{c}, when present, is C₁-C₆ alkoxy or C₃-C₈ cycloalkyl, or two R^{c} combine together with the atom or atoms to which they are attached to form a C₃-C₈ cycloalkyl, wherein each hydrogen atom in C₃-C₈ cycloalkyl is optionally substituted by C₁-C₆ alkyl;
R^{d}, when present, is H, C₁-C₆ alkyl, —C(O)C₁-C₆ alkyl, or C₆-C₁₀ aryl, wherein each hydrogen atom in C₁-C₆ alkyl and C₆-C₁₀ aryl is optionally substituted by halo or —NO₂;
l is 0, 1, 2, or 3; and
m is 1, 2, or 3;
provided that if R⁴ is 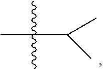 [SMILES: *C(C)C]
R¹ is —OCH₂-phenyl, one of R⁹ and R¹⁰ is is 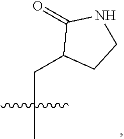 [SMILES: *CC1CCNC1=O]
and one of R⁷ and R⁸ is 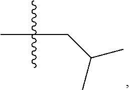 [SMILES: *CC(C)C]
then W is not —C(O)H,
provided that if R⁴ is 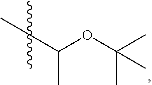 [SMILES: *C(C)OC(C)(C)C]
R¹ is —OCH₂-phenyl, one of R⁹ and R¹⁰ is 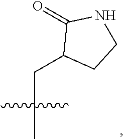 [SMILES: *CC1CCNC1=O]
and one of R⁷ and R⁸ is 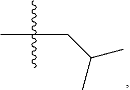 [SMILES: *CC(C)C]
then W is not —C(O)H, and
provided that if R⁴ is 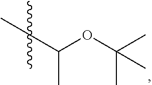 [SMILES: *C(C)OC(C)(C)C]
R¹ is —OCH₂-phenyl, one of R⁹ and R¹⁰ is 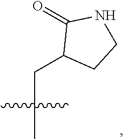 [SMILES: *CC1CCNC1=O]
and one of R⁷ and R⁸ is —CH₂-cyclohexyl, then W is not —C(O)H.

9. The compound or pharmaceutically acceptable salt of claim 8, wherein R⁴ is C₁-C₆ alkyl, wherein at least one hydrogen atom in C₁-C₆ alkyl is substituted by C₁-C₆ alkoxy.

10. The compound or pharmaceutically acceptable salt of claim 9, wherein the compound is of formula

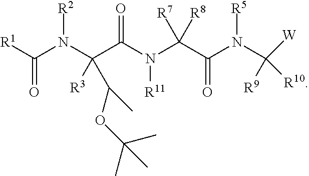

11. The compound or pharmaceutically acceptable salt of claim 8, wherein R⁷ is C₁-C₆ alkyl, wherein at least one hydrogen atom in C₁-C₆ alkyl is substituted by C₃-C₈ cycloalkyl, C₆-C₁₀ aryl, or 5-10 membered heteroaryl.

12. The compound or pharmaceutically acceptable salt of claim 11, wherein the compound is of formula

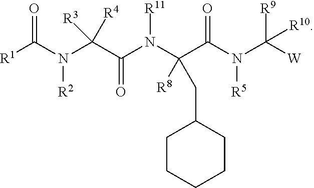

13. The compound or pharmaceutically acceptable of claim 8, wherein the compound is of formula

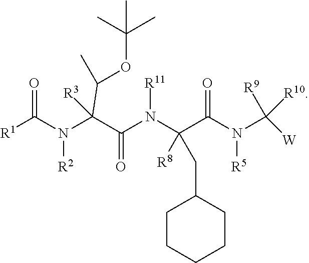

14. The compound or pharmaceutically acceptable salt of claim 8, wherein the compound is of formula

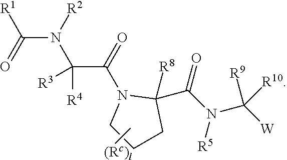

15. The compound or pharmaceutically acceptable salt of claim 10, wherein the compound is of formula

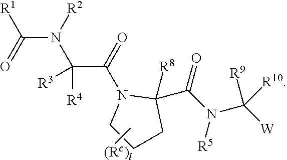

16. A compound selected from the group consisting of:
benzyl ((S)-3-methyl-1-oxo-1-(((S)-1-oxo-1-(((S)-1-oxo-3-((S)-2-oxopyrrolidin-3-yl)propan-2-yl)amino)-3-phenylpropan-2-yl)amino)butan-2-yl)carbamate,
benzyl ((S)-1-(((S)-3-cyclohexyl-1-oxo-1-(((S)-1-oxo-3-((S)-2-oxopyrrolidin-3-yl)propan-2-yl)amino)propan-2-yl)amino)-3-methyl-1-oxobutan-2-yl)carbamate,
benzyl ((2S,3R)-3-(tert-butoxy)-1-oxo-1-(((S)-1-oxo-1-(((S)-1-oxo-3-((S)-2-oxopyrrolidin-3-yl)propan-2-yl)amino)-3-phenylpropan-2-yl)amino)butan-2-yl)carbamate,
(S)-2-((S)-2-((E)-3-(4-chloro-2-fluorophenyl)acrylamido)-3-methylbutanamido)-4-methyl-N—((S)-1-oxo-3-((S)-2-oxopyrrolidin-3-yl)propan-2-yl)pentanamide,
benzyl ((S)-1-(((S)-3-(furan-2-yl)-1-oxo-1-(((S)-1-oxo-3-((S)-2-oxopyrrolidin-3-yl)propan-2-yl)amino)propan-2-yl)amino)-3-methyl-1-oxobutan-2-yl)carbamate,
benzyl ((S)-1-(((S)-3-(furan-2-yl)-1-oxo-1-(((S)-1-oxo-3-((S)-2-oxopyrrolidin-3-yl)propan-2-yl)amino)propan-2-yl)amino)-3-methyl-1-oxobutan-2-yl)carbamate,
N—((S)-3-methyl-1-(((S)-4-methyl-1-oxo-1-(((S)-1-oxo-3-((S)-2-oxopyrrolidin-3-yl)propan-2-yl)amino)pentan-2-yl)amino)-1-oxobutan-2-yl)-1H-indole-2-carboxamide,
benzyl ((S)-1-(((S)-4,4-dimethyl-1-oxo-1-(((S)-1-oxo-3-((S)-2-oxopyrrolidin-3-yl)propan-2-yl)amino)pentan-2-yl)amino)-3-methyl-1-oxobutan-2-yl)carbamate,
benzyl ((S)-3-methyl-1-(((S)-4-methyl-1-oxo-1-(((S)-1-oxo-3-((S)-2-oxopyrrolidin-3-yl)propan-2-yl)amino)pent-4-en-2-yl)amino)-1-oxobutan-2-yl)carbamate,
benzyl ((S)-1-(((S)-4,4-dimethyl-1-oxo-1-(((S)-1-oxo-3-((S)-2-oxopyrrolidin-3-yl)propan-2-yl)amino)pentan-2-yl)amino)-3,3-dimethyl-1-oxobutan-2-yl)carbamate,
benzyl ((S)-1-cyclopropyl-2-(((S)-4,4-dimethyl-1-oxo-1-(((S)-1-oxo-3-((S)-2-oxopyrrolidin-3-yl)propan-2-yl)amino)pentan-2-yl)amino)-2-oxoethyl)carbamate,
benzyl ((2S,3R)-3-(tert-butoxy)-1-(((S)-3-cyclopropyl-1-oxo-1-(((S)-1-oxo-3-((S)-2-oxopyrrolidin-3-yl)propan-2-yl)amino)propan-2-yl)amino)-1-oxobutan-2-yl)carbamate,
benzyl ((2S,3R)-3-(tert-butoxy)-1-(((S)-4,4-dimethyl-1-oxo-1-(((S)-1-oxo-3-((S)-2-oxopyrrolidin-3-yl)propan-2-yl)amino)pentan-2-yl)amino)-1-oxobutan-2-yl)carbamate,
benzyl ((S)-1-(((S)-3-cyclopropyl-1-oxo-1-(((S)-1-oxo-3-((S)-2-oxopyrrolidin-3-yl)propan-2-yl)amino)propan-2-yl)amino)-3-methyl-1-oxobutan-2-yl)carbamate,
benzyl ((S)-1-(((S)-4,4-dimethyl-1-oxo-1-(((S)-1-oxo-3-((S)-2-oxopyrrolidin-3-yl)propan-2-yl)amino)pentan-2-yl)amino)-2,3-dimethyl-1-oxobutan-2-yl)carbamate,
benzyl (1-(((S)-4,4-dimethyl-1-oxo-1-(((S)-1-oxo-3-((S)-2-oxopyrrolidin-3-yl)propan-2-yl)amino)pentan-2-yl)amino)-2-methyl-1-oxopropan-2-yl)carbamate,
benzyl (1-(((S)-3-cyclohexyl-1-oxo-1-(((S)-1-oxo-3-((S)-2-oxopyrrolidin-3-yl)propan-2-yl)amino)propan-2-yl)carbamoyl)cyclopropyl)carbamate,
3-chlorobenzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate,
3-chlorobenzyl ((S)-1-(((S)-3-cyclohexyl-1-oxo-1-(((S)-1-oxo-3-((S)-2-oxopyrrolidin-3-yl)propan-2-yl)amino)propan-2-yl)amino)-3-methyl-1-oxobutan-2-yl)carbamatebenzyl ((S)-3-cyclohexyl-1-oxo-1-(((S)-1-oxo-3-((S)-2-oxopyrrolidin-3-yl)propan-2-yl)amino)propan-2-yl)carbamate,
benzyl ((S)-1-(((S)-3-cyclopropyl-1-oxo-1-(((S)-1-oxo-3-((S)-2-oxopyrrolidin-3-yl)propan-2-yl)amino)propan-2-yl)amino)-3,3-dimethyl-1-oxobutan-2-yl)carbamate,
benzyl ((S)-1-cyclopropyl-2-(((S)-3-cyclopropyl-1-oxo-1-(((S)-1-oxo-3-((S)-2-oxopyrrolidin-3-yl)propan-2-yl)amino)propan-2-yl)amino)-2-oxoethyl)carbamate,
benzyl ((S)-1-(((S)-1-cyclopropyl-3-(((S)-1-oxo-3-((S)-2-oxopyrrolidin-3-yl)propan-2-yl)amino)propan-2-yl)amino)-3,3-dimethyl-1-oxobutan-2-yl)carbamate,
3-chlorobenzyl ((2S,3R)-3-(tert-butoxy)-1-(((S)-4-methyl-1-oxo-1-(((S)-1-oxo-3-((S)-2-oxopyrrolidin-3-yl)propan-2-yl)amino)pentan-2-yl)amino)-1-oxobutan-2-yl)carbamate,
benzyl ((S)-1-(((S)-1-(2-acryloyl-2-((2,6-dioxo-1,2,3,6-tetrahydropyrimidin-4-yl)methyl)hydrazineyl)-4-methyl-1-oxopentan-2-yl)amino)-3-methyl-1-oxobutan-2-yl)carbamate,
benzyl (S)-(1-(2-(3-amino-3-oxopropyl)-2-propionylhydrazineyl)-4-methyl-1-oxopentan-2-yl)carbamate,
benzyl (S)-(1-(2-(3-amino-3-oxopropyl)-2-(2-chloroacetyl)hydrazineyl)-4-methyl-1-oxopentan-2-yl)carbamate,
benzyl (S)-(1-(2-acryloyl-2-(3-amino-3-oxopropyl)hydrazineyl)-4-methyl-1-oxopentan-2-yl)carbamate,
benzyl ((S)-1-(2-(3-amino-3-oxopropyl)-2-((R)-2-chloropropanoyl)hydrazineyl)-4-methyl-1-oxopentan-2-yl)carbamate,
benzyl ((S)-1-(2-(3-amino-3-oxopropyl)-2-((S)-2-chloropropanoyl)hydrazineyl)-4-methyl-1-oxopentan-2-yl)carbamate,
benzyl (S)-(1-(2-(3-amino-3-oxopropyl)-2-(2-fluoro-5-nitrobenzoyl)hydrazineyl)-4-methyl-1-oxopentan-2-yl)carbamate,
benzyl (S)-(1-(2-(3-amino-3-oxopropyl)-2-(2-benzylacryloyl)hydrazineyl)-4-methyl-1-oxopentan-2-yl)carbamate,
benzyl (S,E)-(1-(2-(3-amino-3-oxopropyl)-2-(4-(dimethylamino)but-2-enoyl)hydrazineyl)-4-methyl-1-oxopentan-2-yl)carbamate,
benzyl (S)-(1-(2-(3-amino-3-oxopropyl)-2-(2-(4-nitrophenoxy)acetyl)hydrazineyl)-4-methyl-1-oxopentan-2-yl)carbamate,
benzyl (S)-(1-(2-(3-amino-3-oxopropyl)-2-(2-(4-fluorophenoxy)acetyl)hydrazineyl)-4-methyl-1-oxopentan-2-yl)carbamate,
2,2,2-trifluoroethyl 1-(3-amino-3-oxopropyl)-2-(((benzyloxy)carbonyl)-L-leucyl)hydrazine-1-carboxylate,
4-fluorophenyl 1-(3-amino-3-oxopropyl)-2-(((benzyloxy)carbonyl)-L-leucyl)hydrazine-1-carboxylate,
benzyl ((2S,3R)-1-(((S)-1-(2-acryloyl-2-(3-amino-3-oxopropyl)hydrazineyl)-4-methyl-1-oxopentan-2-yl)amino)-3-(tert-butoxy)-1-oxobutan-2-yl)carbamate,
benzyl ((S)-1-(((S)-1-(2-acryloyl-2-(3-amino-3-oxopropyl)hydrazineyl)-4-methyl-1-oxopentan-2-yl)amino)-3-methyl-1-oxobutan-2-yl)carbamate,
benzyl ((2S,3R)-1-(((S)-1-(2-acryloyl-2-(3-amino-3-oxopropyl)hydrazineyl)-3-cyclohexyl-1-oxopropan-2-yl)amino)-3-(tert-butoxy)-1-oxobutan-2-yl)carbamate,
benzyl ((2S,3R)-1-(((S)-1-(2-(3-amino-3-oxopropyl)-2-(2-chloroacetyl)hydrazineyl)-3-cyclohexyl-1-oxopropan-2-yl)amino)-3-(tert-butoxy)-1-oxobutan-2-yl)carbamate,
(1R,2S,5S)-3-((S)-2-(3-(tert-butyl)ureido)-3,3-dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide
(S)-2-((S)-2-(3-(tert-butyl)ureido)-3,3-dimethylbutanamido)-4,4-dimethyl-N—((S)-1-,oxo-3-((S)-2-oxopyrrolidin-3-yl)propan-2-yl)pentanamide,
(S)-5-((S)-2-(3-(tert-butyl)ureido)-3,3-dimethylbutanoyl)-N—((S)-1-oxo-3-((S)-2-oxopyrrolidin-3-yl)propan-2-yl)-5-azaspiro[2.4]heptane-6-carboxamide,
(1R,2S,5S)—N—((S)-4-amino-3,4-dioxo-1-((S)-2-oxopyrrolidin-3-yl)butan-2-yl)-3-((S)-2-(3-(tert-butyl)ureido)-3,3-dimethylbutanoyl)-6,6-dimethyl-3-azabicyclo[3.1.0]hexane-2-carboxamide,
(1R,2S,5S)-3-((S)-2-(3-(tert-butyl)ureido)-3,3-dimethylbutanoyl)-N—((S)-4-(ethylamino)-3,4-dioxo-1-((S)-2-oxopyrrolidin-3-yl)butan-2-yl)-6,6-dimethyl-3-azabicyclo[3.1.0]hexane-2-carboxamide,
(1R,2S,5S)-3-((S)-2-(3-(tert-butyl)ureido)-3,3-dimethylbutanoyl)-N—((S)-4-(cyclopropylamino)-3,4-dioxo-1-((S)-2-oxopyrrolidin-3-yl)butan-2-yl)-6,6-dimethyl-3-azabicyclo[3.1.0]hexane-2-carboxamide,
(1R,2S,5S)-3-((S)-2-(3-(tert-butyl)ureido)-3,3-dimethylbutanoyl)-N—((S)-4-((cyclopropylmethyl)amino)-3,4-dioxo-1-((S)-2-oxopyrrolidin-3-yl)butan-2-yl)-6,6-dimethyl-3-azabicyclo[3.1.0]hexane-2-carboxamide,
(1R,2S,5S)-3-((S)-2-(3-(tert-butyl)ureido)-3,3-dimethylbutanoyl)-N—((S)-4-(butylamino)-3,4-dioxo-1-((S)-2-oxopyrrolidin-3-yl)butan-2-yl)-6,6-dimethyl-3-azabicyclo[3.1.0]hexane-2-carboxamide,
(1R,2S,5S)-3-((S)-2-(3-(tert-butyl)ureido)-3,3-dimethylbutanoyl)-N—((S)-4-(hexylamino)-3,4-dioxo-1-((S)-2-oxopyrrolidin-3-yl)butan-2-yl)-6,6-dimethyl-3-azabicyclo[3.1.0]hexane-2-carboxamide,
(1R,2S,5S)—N—((S)-4-(benzylamino)-3,4-dioxo-1-((S)-2-oxopyrrolidin-3-yl)butan-2-yl)-3-((S)-2-(3-(tert-butyl)ureido)-3,3-dimethylbutanoyl)-6,6-dimethyl-3-azabicyclo[3.1.0]hexane-2-carboxamide,
(S)-2-(3-(tert-butyl)ureido)-N—((S)-3-cyclopropyl-1-oxo-1-(((S)-1-oxo-3-((S)-2-oxopyrrolidin-3-yl)propan-2-yl)amino)propan-2-yl)-3-methylbutanamide,
sodium (5S,8S,11S)-5-((R)-1-(tert-butoxy)ethyl)-8-(cyclohexylmethyl)-12-hydroxy-3,6,9-trioxo-11-(((S)-2-oxopyrrolidin-3-yl)methyl)-1-phenyl-2-oxa-4,7,10-triazadodecane-12-sulfonate,
benzyl ((2S)-1-(((2S)-1-(((2S)-1-cyano-1-hydroxy-3-((S)-2-oxopyrrolidin-3-yl)propan-2-yl)amino)-4-methyl-1-oxopentan-2-yl)amino)-3-methyl-1-oxobutan-2-yl)carbamate,
benzyl ((2S,3R)-3-(tert-butoxy)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-1-oxobutan-2-yl)carbamate,
(1R,2S,5S)-3-(O-(tert-butyl)-N-(tert-butylcarbamoyl)-L-threonyl)-6,6-dimethyl-N—((S)-1-oxo-3-((S)-2-oxopyrrolidin-3-yl)propan-2-yl)-3-azabicyclo[3.1.0]hexane-2-carboxamide,
1-((2S,3R)-1-((1R,2S,5S)-2-(2-acryloyl-2-(((S)-5-oxopyrrolidin-2-yl)methyl)hydrazine-1-carbonyl)-6,6-dimethyl-3-azabicyclo[3.1.0]hexan-3-yl)-3-(tert-butoxy)-1-oxobutan-2-yl)-3-(tert-butyl)urea,
3-(1-acryloyl-2-((1R,2S,5S)-3-(O-(tert-butyl)-N-(tert-butylcarbamoyl)-L-threonyl)-6,6-dimethyl-3-azabicyclo[3.1.0]hexane-2-carbonyl)hydrazineyl)propanamide,
tert-butyl ((S)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-3,3-dimethyl-1-oxobutan-2-yl)carbamate,
benzyl ((S)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-3,3-dimethyl-1-oxobutan-2-yl)carbamate,
(2S,3R)-3-(tert-butoxy)-2-(3-(tert-butyl)ureido)-N—((S)-3-cyclohexyl-1-oxo-1-(((S)-1-oxo-3-((S)-2-oxopyrrolidin-3-yl)propan-2-yl)amino)propan-2-yl)butanamide,
(S)-2-(3-(tert-butyl)ureido)-N—((S)-3-cyclopropyl-1-oxo-1-(((S)-1-oxo-3-((S)-2-oxopyrrolidin-3-yl)propan-2-yl)amino)propan-2-yl)-3-methylbutanamide,
(1R,2S,5S)-3-((S)-3,3-dimethyl-2-(3-methylbutanamido)butanoyl)-6,6-dimethyl-N—((S)-1-oxo-3-((S)-2-oxopyrrolidin-3-yl)propan-2-yl)-3-azabicyclo[3.1.0]hexane-2-carboxamide,
(1R,2S,5S)—N—((S)-1-cyano-2-((S)-2-oxopyrrolidin-3-yl)ethyl)-3-((S)-2-(3,3-dimethylbutanamido)-3,3-dimethylbutanoyl)-6,6-dimethyl-3-azabicyclo[3.1.0]hexane-2-carboxamide,
(1R,2S,5S)—N—((S)-4-amino-3,4-dioxo-1-((S)-2-oxopyrrolidin-3-yl)butan-2-yl)-3-((S)-2-(3,3-dimethylbutanamido)-3,3-dimethylbutanoyl)-6,6-dimethyl-3-azabicyclo[3.1.0]hexane-2-carboxamide,
benzyl ((2S,3R)-1-(((S)-1-(((S)-4-amino-3,4-dioxo-1-((S)-2-oxopyrrolidin-3-yl)butan-2-yl)amino)-3-cyclohexyl-1-oxopropan-2-yl)amino)-3-(tert-butoxy)-1-oxobutan-2-yl)carbamate,
(1R,2S,5S)-3-(2,2-bis(4-chlorophenoxy)acetyl)-6,6-dimethyl-N—((S)-1-oxo-3-((S)-2-oxopyrrolidin-3-yl)propan-2-yl)-3-azabicyclo[3.1.0]hexane-2-carboxamide,
benzyl ((S)-2-(((S)-3-cyclohexyl-1-oxo-1-(((S)-1-oxo-3-((S)-2-oxopyrrolidin-3-yl)propan-2-yl)amino)propan-2-yl)amino)-2-oxo-1-phenylethyl)carbamate,
benzyl ((S)-1-cyclohexyl-2-(((S)-3-cyclohexyl-1-oxo-1-(((S)-1-oxo-3-((S)-2-oxopyrrolidin-3-yl)propan-2-yl)amino)propan-2-yl)amino)-2-oxoethyl)carbamate,
3-hydroxybenzyl ((S)-3-cyclohexyl-1-oxo-1-(((S)-1-oxo-3-((S)-2-oxopyrrolidin-3-yl)propan-2-yl)amino)propan-2-yl)carbamate,
3-(((((S)-3-cyclohexyl-1-oxo-1-(((S)-1-oxo-3-((S)-2-oxopyrrolidin-3-yl)propan-2-yl)amino)propan-2-yl)carbamoyl)oxy)methyl)phenyl acetate,
(1R,2S,5S)-3-((S)-2-(3,3-dimethylbutanamido)-3,3-dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide,
benzyl ((S)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-1-oxo-3-phenylpropan-2-yl)carbamate,
benzyl ((S)-3-(4-chlorophenyl)-1-((1R,2S,5S)-6,6-dimethyl-2-(((S)-1-oxo-3-((S)-2-oxopyrrolidin-3-yl)propan-2-yl)carbamoyl)-3-azabicyclo[3.1.0]hexan-3-yl)-1-oxopropan-2-yl)carbamate,
benzyl ((2S,3R)-1-(((2S)-1-(2-acryloyl-2-((2-oxopyrrolidin-3-yl)methyl)hydrazineyl)-3-cyclohexyl-1-oxopropan-2-yl)amino)-3-(tert-butoxy)-1-oxobutan-2-yl)carbamate,
benzyl ((2S,3R)-3-(tert-butoxy)-1-(((2S)-1-(2-(2-chloroacetyl)-2-((2-oxopyrrolidin-3-yl)methyl)hydrazineyl)-3-cyclohexyl-1-oxopropan-2-yl)amino)-1-oxobutan-2-yl)carbamate,
3-fluorobenzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate, and
4-fluorobenzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate.

17. A pharmaceutical composition comprising at least one compound of claim 1, or a pharmaceutically acceptable salt thereof, and optionally one or more pharmaceutically acceptable excipients.

18. A pharmaceutical composition comprising at least one compound of claim 8, or a pharmaceutically acceptable salt thereof, and optionally one or more pharmaceutically acceptable excipients.

19. A method of treating disease, such as a viral infection, comprising administering to a subject in need of such treatment an effective amount of a compound of claim 1, or a pharmaceutically acceptable salt thereof.

20. A method of treating disease, such as a viral infection, comprising administering to a subject in need of such treatment an effective amount of a compound of claim 8, or a pharmaceutically acceptable salt thereof.


SEQUENCE LISTING (2 sequences)

SEQ ID NO: 1 - DNA, 21 bp, Artificial Sequence
  PCR primer
cgcggatccgggtttcgcaag

SEQ ID NO: 2 - DNA, 27 bp, Artificial Sequence
  PCR primer
ccgctcgagttactgaaaagttacgcc